(19) United States
(12) Patent Application Publication
(10) Pub. No.: US 2025/0243194 A1
(43) Pub. Date: Jul. 31, 2025

(54) ANTAGONIST OF ADENOSINE RECEPTORS

(71) Applicant: AdoRx Therapeutics Limited, Edinburgh Lothian (GB)

(72) Inventors: Clive MCCARTHY, Edinburgh Lothian (GB); Ben MOULTON, Mereside Cheshire (GB)

(21) Appl. No.: 18/834,185

(22) PCT Filed: Jan. 27, 2023
(86) PCT No.: PCT/GB2023/050195

§ 371 Date: Jul. 30, 2024

(30) Foreign Application Priority Data
Jan. 28, 2022 (GB) 2201151.4

(51) Int. Cl. C07D 417/14; A61K 31/427; A61K 31/437; A61K 31/454; A61K 31/4709; A61K 31/501; A61K 31/517; A61K 31/5377; A61K 31/5383; A61K 31/5386; A61K 31/551; C07D 417/04; C07D 471/04; C07D 471/10; C07D 487/04; C07D 487/10; C07D 491/107; C07D 498/04; C07D 498/10; C07D 519/00

(52) U.S. Cl. CPC C07D 417/14; A61K 31/427; A61K 31/437; A61K 31/454; A61K 31/4709; A61K 31/501; A61K 31/517; A61K 31/5377; A61K 31/5383; A61K 31/5386; A61K 31/551; C07D 417/04; C07D 471/04; C07D 471/10; C07D 487/04; C07D 487/10; C07D 491/107; C07D 498/04; C07D 498/10; C07D 519/00

(57) ABSTRACT

The present invention relates to compounds of formula I shown below:

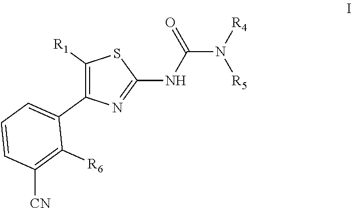

wherein R₁, R₄, R₅ and R₆ are each as defined in the application. The present invention also relates to processes for the preparation of these compounds, to pharmaceutical compositions comprising them, and to their use in the treatment of diseases or conditions in which adenosine A2a and/or A2b receptor activity is implicated, such as, for example, cancer.

### CROSS REFERENCE TO RELATED APPLICATIONS

This application is a national stage application of Patent Application No. PCT/GB2023/050195, filed Jan. 27, 2023 which application claims priority from GB Patent Application No. 2201151.4, filed Jan. 28, 2022, each of which is hereby incorporated by reference.

### INTRODUCTION

The present invention relates to certain compounds that function as antagonists of the adenosine A2a receptor. Additionally, some of the compounds are also antagonists of both A2a and the A2b receptor. The present invention also relates to processes for the preparation of these compounds, to pharmaceutical compositions comprising them, and to their use in the treatment of diseases or conditions in which adenosine A2a and/or A2b receptor activity is implicated, such as, for example, cancer.

### BACKGROUND OF THE INVENTION

A number of immunosuppressive pathways are active in the tumour microenvironment which enable tumour cells to evade elimination by cytotoxic T cells and can diminish the clinical response of patients to immunotherapy with anti-checkpoint antibodies. The anti-PD-1 antibodies pembrolizumab and nivolumab and anti-PD-L1 antibodies durvalumab, avelumab and atezolizumab are approved for the treatment of number of solid tumours including non-small cell lung cancer, head and neck squamous cancer and urothelial cancer. However, only 20-30% of patients respond to checkpoint blockade and the side effects of such treatments are significant (Sukari et al, 2016). Consequently, other approaches to enhance the cytotoxic potential of the tumour microenvironment are actively being investigated. This includes agents that could be used as monotherapies or, more likely, used in combination with checkpoint inhibitors and cytotoxic agents to enhance their efficacy.

One approach that has attracted attention is to interfere with the production and/or action of adenosine in the tumour microenvironment (Vijayan et al, 2017). Adenosine has immunosuppressive properties and is present in the tumour microenvironment at high concentrations. Recent studies estimate the concentration of adenosine to be about 10 μM in human tumours compared to <1 μM in normal tissue (Houthuys et al 2017). Adenosine is formed at both intracellular and extracellular sites by two distinct pathways that involve two different substrates. Intracellular adenosine is derived from AMP and S-adenosyl homocysteine whilst the high extracellular adenosine concentrations observed during metabolic stress are associated with the release and degradation of precursor adenine nucleotides (ATP, ADP and AMP) by the concerted action of CD39 and CD73 (Vijayan et al, 2017).

CD39 and CD73 are upregulated in the tumour microenvironment in response to hypoxia. CD73 represents a putative patient stratification method for adenosine antagonists as its expression on tumour cells is also associated with poor overall prognosis in many different cancer types suggesting that adenosine production contributes to the undesirable immunosuppressive phenotype of the tumour microenvironment (Gao et al 2014; Loi et al, 2013). CD73 expression by tumour-infiltrating immune cells is also important in promoting tumour immune suppression as CD73 negative Treg cells fail to suppress effector T cell functions (Deaglio et al, 2007; Reinhardt et al, (2017). Furthermore, patients resistant to anti-PD1 treatment have elevated levels of CD73 (Reinhardt et al, 2017).

Adenosine regulates cell function via occupancy of specific GPCRs on the cell surface of the P1 purinoceptor subtypes. The P1 receptor family is further subdivided into A1, A2a, A2b and A3.

A2 receptors are subdivided into A2a and A2b, based on high and low affinity for adenosine, respectively. A2a is expressed by lymphocytes and activation of A2a leads to suppression of cytokine production and other effector functions. Tumour growth is inhibited by genetic ablation of A2a in syngeneic mouse models and this effect has been demonstrated to be due to enhanced lymphocyte activation and cytotoxic function (Ohta et al, 2006; Waickman et al 2012; Beavis et al, 2013; Mittal et al, 2014; Cekic et al, 2014). A2a−/− mice show an increased response to inhibition of checkpoint pathways such as PD-1, with an improvement in both tumour free survival and overall survival. Adenosine-mediated A2a activation also limits the efficacy of anti-CTLA4 treatment (Iannone et al, 2014).

The effects of genetic deficiency of A2a in mouse models is mimicked by pharmacological blockade of A2a. A2a antagonists have been shown to enhance the cytotoxic CD8+ T cells and to enhance the ability of NK cells prevent metastasis of CD73-expressing tumours (Beavis et al, 2013). Importantly, A2a antagonists enhance the efficacy of anti-PD1 antibodies (Beavis et al, 2015).

These findings have prompted the development of selective A2a antagonists for use in cancer immunotherapy and clinical trials are ongoing with CPI-444, the first selective A2a antagonist to be evaluated in cancer, being used as both as a monotherapy and in combination with the anti-PDL1 antibody atezolizumab. The preliminary data indicated that the compound was well tolerated and showed early indications of reducing tumour size and enhancing CD8+T infiltration into tumour tissue.

The adenosine A2b receptor also plays a key role in cancer progression through modulation of the tumour microenvironment. A2b is expressed by cells of myeloid origin and the high concentrations of adenosine found within the tumour microenvironment can modify the behaviour of tumour associated macrophages, myeloid-derived suppressor cells and dendritic cells. Activation of A2b leads to polarisation of immunosuppressive M2 macrophages (Csoka et al, 2012), formation of myeloid-derived suppressive cells (Rhyzov et al, 2011) and promotes the immunosuppressive and pro-angiogenic phenotype of dendritic cells (Novitskiy et al, 2008; Wilson et al, 2009). Blockade of A2b has been shown to reduce tumour size in a number of mouse cancer models (Cekic et al, 2012; Sorrentino et al, 2015; Iannone et al, 2013), an effect associated with diminished infiltration of MDSCs and production of VEGF (Sorrentino et al, 2015; Iannone et al, 2013).

Consequently, dual inhibition of both A2a and A2b is an attractive approach to reduce immunosuppression within the tumour microenvironment to inhibit growth.

### SUMMARY OF THE INVENTION

According to a first aspect of the present invention, there is provided a compound, or a pharmaceutically acceptable salt, hydrate or solvate thereof, as defined herein.

According to a further aspect of the present invention, there is provided a pharmaceutical composition comprising a compound as defined herein, or a pharmaceutically acceptable salt, hydrate or solvate thereof, in admixture with a pharmaceutically acceptable diluent or carrier.

According to a further aspect of the present invention, there is provided a method of antagonising adenosine A2a receptors, or A2a and A2b receptors, in vitro or in vivo, said method comprising contacting a cell with an effective amount of a compound or a pharmaceutically acceptable salt, hydrate or solvate thereof as defined herein.

According to a further aspect of the present invention, there is provided a method of selectively antagonising adenosine A2a receptors, or A2a and A2b receptors in vitro or in vivo, said method comprising contacting a cell with an effective amount of a compound, or a pharmaceutically acceptable salt, hydrate or solvate thereof, as defined herein.

According to a further aspect of the present invention, there is provided a method of inhibiting cell proliferation, in vitro or in vivo, said method comprising contacting a cell with an effective amount of a compound or a pharmaceutically acceptable salt, hydrate or solvate thereof as defined herein, or a pharmaceutical composition as defined herein. Suitably, the compound or pharmaceutical composition is administered in combination with one or more additional antiproliferative agents (e.g. checkpoint inhibitors and/or cytotoxic agents).

According to a further aspect of the present invention, there is provided a method of treating a disease or disorder associated with adenosine A2a and/or A2b receptor activity in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a method of treating a proliferative disorder in a patient in need of such treatment, said method comprising administering to said patient a therapeutically effective amount of a compound or a pharmaceutically acceptable salt, hydrate or solvate thereof as defined herein, or a pharmaceutical composition as defined herein. Suitably, the compound or pharmaceutical composition is administered in combination with one or more additional antiproliferative agents (e.g. checkpoint inhibitors and/or cytotoxic agents).

According to a further aspect of the present invention, there is provided a method of treating cancer in a patient in need of such treatment, said method comprising administering to said patient a therapeutically effective amount of a compound or a pharmaceutically acceptable salt, hydrate or solvate thereof as defined herein, or a pharmaceutical composition as defined herein. Suitably, the compound or pharmaceutical composition is administered in combination with one or more additional anticancer agents (e.g. checkpoint inhibitors and/or cytotoxic agents).

According to a further aspect of the present invention, there is provided a compound, or a pharmaceutically acceptable salt, hydrate or solvate thereof, or a pharmaceutical composition as defined herein for use in therapy.

According to a further aspect of the present invention, there is provided a compound or a pharmaceutically acceptable salt, hydrate or solvate thereof as defined herein, or a pharmaceutical composition as defined herein, for use in the treatment of a proliferative condition. Suitably, the compound or pharmaceutical composition is administered in combination with one or more additional antiproliferative agents (e.g. checkpoint inhibitors and/or cytotoxic agents).

According to a further aspect of the present invention, there is provided a compound, or a pharmaceutically acceptable salt, hydrate or solvate thereof, or a pharmaceutical composition as defined herein for use in the treatment of cancer. In a particular embodiment, the cancer is human cancer. Suitably, the compound or pharmaceutical composition is administered in combination with one or more additional anticancer agents (e.g. checkpoint inhibitors and/or cytotoxic agents).

According to a further aspect of the present invention, there is provided a compound, or a pharmaceutically acceptable salt, hydrate or solvate thereof, as defined herein for use as an adenosine A2a and/or A2b antagonist. In an embodiment, the compounds of the invention are selective adenosine A2a and A2b antagonists.

According to a further aspect of the present invention, there is provided a compound, or a pharmaceutically acceptable salt, hydrate or solvate thereof, as defined herein for use in the treatment of a disease or disorder in which adenosine A2a and/or A2b is implicated.

According to a further aspect of the present invention, there is provided the use of a compound, or a pharmaceutically acceptable salt, hydrate or solvate thereof, as defined herein in the manufacture of a medicament for the treatment of a proliferative condition. Suitably, the compound or pharmaceutical composition is administered in combination with one or more additional antiproliferative agents (e.g. checkpoint inhibitors and/or cytotoxic agents).

According to a further aspect of the present invention, there is provide the use of a compound, or a pharmaceutically acceptable salt, hydrate or solvate thereof, as defined herein in the manufacture of a medicament for the treatment of cancer. Suitably, the cancer is a human cancer. Suitably, the compound or pharmaceutical composition is administered in combination with one or more additional anticancer agents (e.g. checkpoint inhibitors and/or cytotoxic agents).

According to a further aspect of the present invention, there is provided a use of a compound, or a pharmaceutically acceptable salt, hydrate or solvate thereof, as defined herein in the manufacture of a medicament for use as an adenosine A2a and/or A2b antagonist.

According to a further aspect of the present invention, there is provided a use of a compound, or a pharmaceutically acceptable salt, hydrate or solvate thereof, as defined herein in the manufacture of a medicament for the treatment of a disease or disorder in which adenosine A2a is implicated.

According to a further aspect of the present invention, there is provided a process for preparing a compound, or a pharmaceutically acceptable salt, hydrate or solvate thereof, as defined herein.

According to a further aspect of the present invention, there is provided a compound, or a pharmaceutically acceptable salt, hydrate or solvate thereof, obtainable by, or obtained by, or directly obtained by a process of preparing a compound as defined herein.

According to a further aspect of the present invention, there are provided novel intermediates as defined herein which are suitable for use in any one of the synthetic methods set out herein.

Features, including optional, suitable, and preferred features in relation to one aspect of the invention may also be features, including optional, suitable and preferred features in relation to any other aspect of the invention.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

In this specification the term “alkyl” includes both straight and branched chain alkyl groups. References to individual alkyl groups such as “propyl” are specific for the straight chain version only and references to individual branched chain alkyl groups such as “isopropyl” are specific for the branched chain version only. For Example, “(1-6C)alkyl” includes (1-4C)alkyl, (1-3C)alkyl, propyl, isopropyl and t-butyl. A similar convention applies to other radicals, for example “phenyl(1-6C)alkyl” includes phenyl(1-4C)alkyl, benzyl, 1-phenylethyl and 2-phenylethyl.

The term “(m-nC)” or “(m-nC) group” used alone or as a prefix, refers to any group having m to n carbon atoms.

An “alkylene,” “alkenylene,” or “alkynylene” group is an alkyl, alkenyl, or alkynyl group that is positioned between and serves to connect two other chemical groups. Thus, “(1-6C)alkylene” means a linear saturated divalent hydrocarbon radical of one to six carbon atoms or a branched saturated divalent hydrocarbon radical of three to six carbon atoms, for example, methylene, ethylene, propylene, 2-methylpropylene, pentylene, and the like.

“(2-6C)alkenylene” means a linear divalent hydrocarbon radical of two to six carbon atoms or a branched divalent hydrocarbon radical of three to six carbon atoms, containing at least one double bond, for example, as in ethenylene, 2,4-pentadienylene, and the like.

“(2-6C)alkynylene” means a linear divalent hydrocarbon radical of two to six carbon atoms or a branched divalent hydrocarbon radical of three to six carbon atoms, containing at least one triple bond, for example, as in ethynylene, propynylene, and butynylene and the like.

“(3-8C)cycloalkyl” means a hydrocarbon ring containing from 3 to 8 carbon atoms, for example, cyclopropyl, cyclobutyl, cyclopentyl, cyclohexyl, cycloheptyl or bicyclo[2.2.1]heptyl.

“(3-8C)cycloalkenyl” means a hydrocarbon ring containing at least one double bond, for example, cyclobutenyl, cyclopentenyl, cyclohexenyl or cycloheptenyl, such as 3-cyclohexen-1-yl, or cyclooctenyl.

“(3-8C)cycloalkyl-(1-6C)alkylene” means a (3-8C)cycloalkyl group covalently attached to a (1-6C)alkylene group, both of which are defined herein.

The term “halo” or “halogeno” refers to fluoro, chloro, bromo and iodo.

The term “heterocyclyl”, “heterocyclic” or “heterocycle” means a non-aromatic saturated or partially saturated monocyclic, fused, bridged, or spiro bicyclic heterocyclic ring system(s). Monocyclic heterocyclic rings contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles contain from 7 to 17 member atoms, suitably 7 to 12 member atoms, in the ring. Bicyclic heterocyclic(s) rings may be fused, spiro, or bridged ring systems. Examples of heterocyclic groups include cyclic ethers such as oxiranyl, oxetanyl, tetrahydrofuranyl, dioxanyl, and substituted cyclic ethers. Heterocycles containing nitrogen include, for example, azetidinyl, pyrrolidinyl, piperidinyl, piperazinyl, tetrahydrotriazinyl, tetrahydropyrazolyl, and the like. Typical sulfur containing heterocycles include tetrahydrothienyl, dihydro-1,3-dithiol, tetrahydro-2H-thiopyran, and hexahydrothiepine. Other heterocycles include dihydrooxathiolyl, tetrahydrooxazolyl, tetrahydro-oxadiazolyl, tetrahydrodioxazolyl, tetrahydrooxathiazolyl, hexahydrotriazinyl, tetrahydrooxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. A suitable value for a heterocyclyl group which bears 1 or 2 oxo (═O) or thioxo (═S) substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. However, reference herein to piperidino or morpholino refers to a piperidin-1-yl or morpholin-4-yl ring that is linked via the ring nitrogen.

By “bridged ring systems” is meant ring systems in which two rings share more than two atoms, see for example Advanced Organic Chemistry, by Jerry March, 4^{th} Edition, Wiley Interscience, pages 131-133, 1992. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane and quinuclidine.

By “spiro bicyclic ring systems” we mean that the two ring systems share one common spiro carbon atom, i.e. the heterocyclic ring is linked to a further carbocyclic or heterocyclic ring through a single common spiro carbon atom. Examples of spiro ring systems include 6-azaspiro[3.4]octane, 2-oxa-6-azaspiro[3.4]octane, 2-azaspiro[3.3]heptanes, 2-oxa-6-azaspiro[3.3]heptanes, 7-oxa-2-azaspiro[3.5]nonane, 6-oxa-2-azaspiro[3.4]octane, 2-oxa-7-azaspiro[3.5]nonane and 2-oxa-6-azaspiro[3.5]nonane.

“Heterocyclyl(1-6C)alkyl” means a heterocyclyl group covalently attached to a (1-6C)alkylene group, both of which are defined herein.

The term “heteroaryl” or “heteroaromatic” means an aromatic mono-, bi-, or polycyclic ring incorporating one or more (for example 14, particularly 1, 2 or 3) heteroatoms selected from nitrogen, oxygen or sulfur. The term heteroaryl includes both monovalent species and divalent species. Examples of heteroaryl groups are monocyclic and bicyclic groups containing from five to twelve ring members, and more usually from five to ten ring members.

The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically, the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general, the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3b]-furanyl-, 2H-furo[3,2b]-pyranyl-, 5H-pyrido[2,3-d]-ooxazinyl-, 1H-pyrazolo[4,3-d]-oxazolyl, 4H-imidazo[4,5d]thiazolyl, pyrazino[2,3d]pyridazinyl, -imidazo[2,1b]thiazolyl, -imidazo[1,2b][1,2,4]-triazinyl. “Heteroaryl” also covers partially aromatic bi- or polycyclic ring systems wherein at least one ring is an aromatic ring and one or more of the other ring(s) is a nonaromatic, saturated or partially saturated ring, provided at least one ring contains one or more heteroatoms selected from nitrogen, oxygen or -sulfur-. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl, 3,4-dihydro-2H-pyrido[3,2-b][1,4]oxazinyl and 6,8-dihydro-5H-[1,2,4]triazolo[4,3-a]pyrazinyl.

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

A bicyclic heteroaryl group may be, for example, a group selected from:
a benzene ring fused to a 5- or 6-membered ring containing 1, 2 or 3 ring heteroatoms;
a pyridine ring fused to a 5- or 6-membered ring containing 1, 2 or 3 ring heteroatoms;
a pyrimidine ring fused to a 5- or 6-membered ring containing 1 or 2 ring heteroatoms;
a pyrrole ring fused to a 5- or 6-membered ring containing 1, 2 or 3 ring heteroatoms;
a pyrazole ring fused to a 5- or 6-membered ring containing 1 or 2 ring heteroatoms;
a pyrazine ring fused to a 5- or 6-membered ring containing 1 or 2 ring heteroatoms;
an imidazole ring fused to a 5- or 6-membered ring containing 1 or 2 ring heteroatoms;
an oxazole ring fused to a 5- or 6-membered ring containing 1 or 2 ring heteroatoms;
an isoxazole ring fused to a 5- or 6-membered ring containing 1 or 2 ring heteroatoms;
a thiazole ring fused to a 5- or 6-membered ring containing 1 or 2 ring heteroatoms;
an isothiazole ring fused to a 5- or 6-membered ring containing 1 or 2 ring heteroatoms;
a thiophene ring fused to a 5- or 6-membered ring containing 1, 2 or 3 ring heteroatoms;
a furan ring fused to a 5- or 6-membered ring containing 1, 2 or 3 ring heteroatoms;
a cyclohexyl ring fused to a 5- or 6-membered heteroaromatic ring containing 1, 2 or 3 ring heteroatoms; and
a cyclopentyl ring fused to a 5- or 6-membered heteroaromatic ring containing 1, 2 or 3 ring heteroatoms.

Particular Examples of bicyclic heteroaryl groups containing a six membered ring fused to a five membered ring include but are not limited to benzfuranyl, benzthiophenyl, benzimidazolyl, benzoxazolyl, benzisoxazolyl, benzthiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl and pyrazolopyridinyl groups.

Particular Examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

“Heteroaryl(1-6C)alkyl” means a heteroaryl group covalently attached to a (1-6C)alkylene group, both of which are defined herein. Examples of heteroaralkyl groups include pyridin-3-ylmethyl, 3-(benzofuran-2-yl)propyl, and the like.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like. In particular embodiment, an aryl is phenyl.

The term “aryl(1-6C)alkyl” means an aryl group covalently attached to a (1-6C)alkylene group, both of which are defined herein. Examples of aryl-(1-6C)alkyl groups include benzyl, phenylethyl, and the like.

This specification also makes use of several composite terms to describe groups comprising more than one functionality. Such terms will be understood by a person skilled in the art. For Example heterocyclyl(m-nC)alkyl comprises (m-nC)alkyl substituted by heterocyclyl.

The term “optionally substituted” refers to either groups, structures, or molecules that are substituted and those that are not substituted. The term “wherein a/any CH, CH₂, CH₃ group or heteroatom (i.e. NH) within a R¹ group is optionally substituted” suitably means that (any) one of the hydrogen radicals of the R¹ group is substituted by a relevant stipulated group.

Where optional substituents are chosen from “one or more” groups it is to be understood that this definition includes all substituents being chosen from one of the specified groups or the substituents being chosen from two or more of the specified groups.

The phrase “compound of the invention” means those compounds which are disclosed herein, both generically and specifically.

### Compounds of the Invention

In a first aspect, the present invention relates to compounds, or pharmaceutically acceptable salts, hydrates or solvates thereof, having the structural formula I shown below:

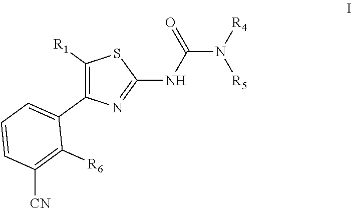

wherein:
R₁ is selected from:

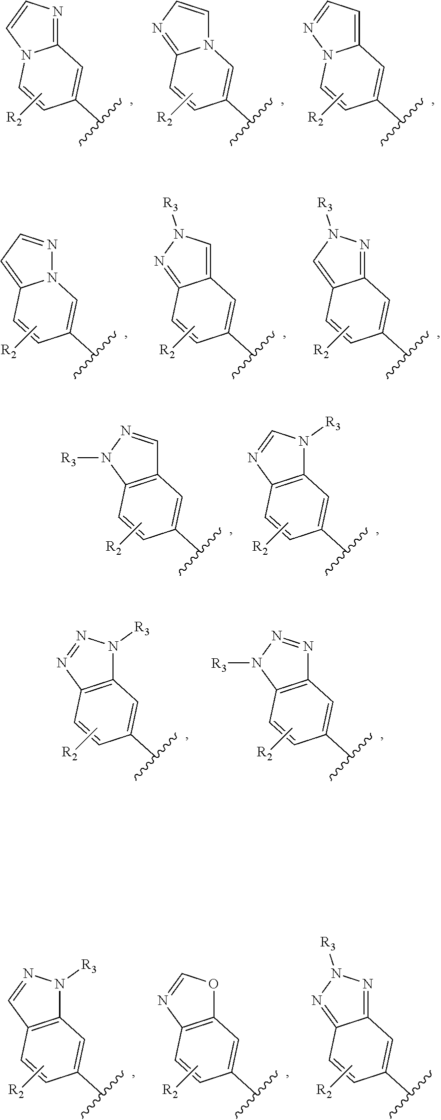

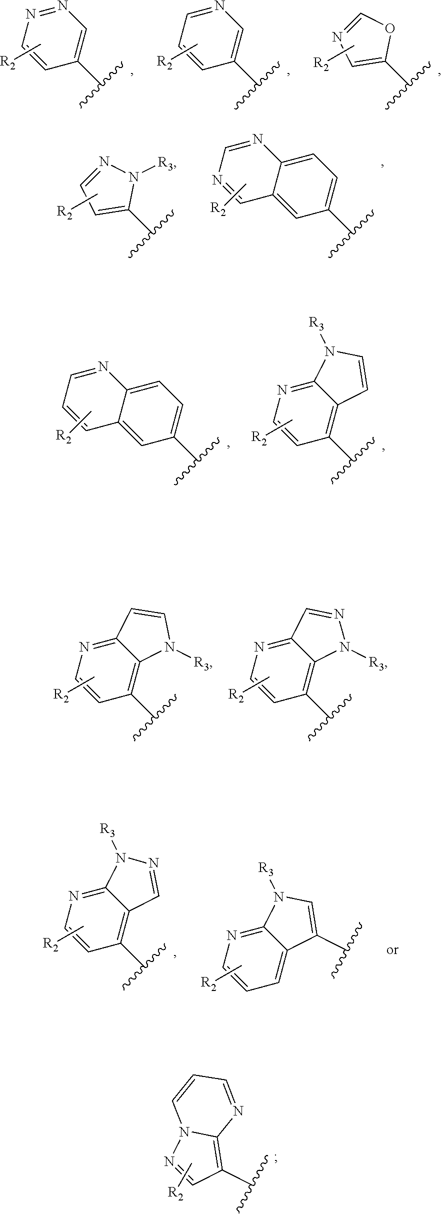

wherein:
any available C atoms in R₁ are optionally substituted by one or more R₂ substituent groups, and
any available N atoms in R₁ are optionally in the form of a N-oxide or substituted by an R₃ group;
wherein
each R₂ is independently selected from (1-6C)alkyl, (3-6C)cycloalkyl, (3-6C)cycloalkyl(1-2C)alkyl, aryl, aryl(1-2C)alkyl, heterocyclyl, heterocyclyl(1-2C)alkyl, heteroaryl (e.g. a 5-membered heteroaryl), heteroaryl(1-2C)alkyl, halo, (1-3C)haloalkyl, (1-3C)haloalkoxy, (1-3C)hydroxyalkyl, cyano, —(CR_{1C}R_{1D})_{q1}—OR_{1A}, —(CR_{1C}R_{1D})_{q1}—C(O)R_{1A}, —(CR_{1C}R_{1D})_{q1}—C(O)OR_{1A}, —(CR_{1C}R_{1D})_{q1}—OC(O)R_{1A}, —(CR_{1C}R_{1D})_{q1}—C(O)N(R_{1B})R_{1A}, —(CR_{1C}R_{1D})_{q1}—N(R_{1B})C(O)R_{1A}, —(CR_{1C}R_{1D})_{q1}—S(O)ₚR_{1A} (where p is 0, 1 or 2), —(CR_{1C}R_{1D})_{q1}—SO₂N(R_{1B})R_{1A}, or —(CR_{1C}R_{1D})_{q1}—N(R_{1B})SO₂R_{1A},
wherein q1 is 0, 1, or 2; and
wherein R_{1A} and R_{1B} are each independently selected from hydrogen, (1-2C)alkyl, (3-4C)cycloalkyl or (3-4C)cycloalkyl(1-2C)alkyl;
wherein R_{1C} and R_{1D} are each independently selected from hydrogen or (1-2C)alkyl; and
R₃ is selected from hydrogen, (1-3C)alkyl, (3-6C)cycloalkyl, (3-6C)cycloalkyl(1-2C)alkyl, aryl, aryl(1-2C)alkyl, a C-linked heterocyclyl, heterocyclyl(1-2C)alkyl, heteroaryl, heteroaryl(1-2C)alkyl, (1-3C)haloalkyl, (1-3C)haloalkoxy, (1-3C)hydroxyalkyl, —(CR₃CR_{3D})₁₋₂—OR_{3A}, —(CR_{3C}R_{3D})_{q3}—C(O)R_{3A}, —(CR_{3C}R_{3D})_{q3}—C(O)OR_{3A}, —(CR_{3C}R_{3D})₁₋₂—OC(O)R_{3A}, —(CR_{3C}R_{3D})_{q3}—C(O)N(R_{3B})R_{q3}, —(CR_{1C}R_{1D})₁₋₂—N(R_{3B})C(O)R_{3A}, —(CR_{3C}R_{3D})—S(O)ₚ₃R_{3A} (where p3 is 0, 1 or 2), —(CR₃CR_{3D})₁₋₂—SO₂N(R_{3B})R_{3A}, or —(CR_{3C}R_{3D})₁₋₂—N(R_{3B})SO₂R_{3A},
wherein R_{3A} and R_{3B} are each independently selected from hydrogen, (1-2C)alkyl, (3-4C)cycloalkyl or (3-4C)cycloalkyl(1-2C)alkyl;
wherein R_{3C} and R_{3D} are each independently selected from hydrogen or (1-2C)alkyl; and
wherein q3 is 0, 1, or 2; and
R₄ and R₅ are linked such that, together with the nitrogen atom to which they are attached, they form a heterocyclic ring which is optionally substituted on any available carbon atom by one or more R_{10C} substituent groups,
   wherein R_{10C} is selected from oxo, thioxo, halo, or cyano substituents, or a group of the formula:

—[CR₇ₐR_{7b}]ₙ-L-Z
wherein
n is an integer from 0 to 6;
R₇ₐ and R_{7b} are each independently selected from hydrogen, fluoro or (1-2C)alkyl;
L is absent or selected from —O—, —S—, —SO—, —SO₂—, —N(Rₐ)—, —C(O)—, —C(O)O—, —OC(O)—, —C(O)N(Rₐ)—, —N(Rₐ)C(O)—, —N(Rₐ)C(O)N(R_{b})—, —C(S)N(Rₐ)—, —N(Rₐ)C(S)—, —N(Rₐ)C(S)N(R_{b})—, —S(O)₂N(Rₐ)—, or —N(Rₐ)SO₂—, wherein Rₐ and R_{b} are each independently selected from hydrogen or (1-2C)alkyl; and
Z is selected from hydrogen, (1-6C)alkyl, (3-6C)cycloalkyl, (3-6C)cycloalkyl(1-2C)alkyl, aryl, aryl(1-2C)alkyl, heterocyclyl, heterocyclyl(1-2C)alkyl, heteroaryl or heteroaryl(1-2C)alkyl; and wherein Z is optionally substituted by one or more substituents selected from (1-6C)alkyl, halo, (1-6C)haloalkyl, (1-6C)haloalkoxy, cyano, nitro, —NR_{c}R_{d}, —OR_{c}, —C(O)R_{c}, —C(O)OR_{c}, —OC(O)R_{c}, —C(O)N(R_{c})R_{d}, —N(R_{c})C(O)R_{d}, —S(O)_{y}R_{c} (where y is 0, 1 or 2), —SO₂N(R_{c})R_{d}, —N(R_{c})SO₂R_{d}, —(CH₂)_{z}NR_{c}R_{d} (where z is 1, 2 or 3) or oxo,
wherein any alkyl moiety in a substituent group on Z is optionally further substituted by cyano, halo, hydroxy, amino, oxo, (1-2C)alkyl or (1-2C)alkoxy and R_{c} and R_{d} are each independently selected from hydrogen, (1-6C)alkyl, (1-6C)haloalkyl or (3-6C)cycloalkyl;
and wherein any available N atoms in the ring formed by R₄ and R₅ are optionally in the form of a N-oxide or are optionally substituted by one or more R_{10N},
wherein R_{10N} is —S(O)₂NH₂ or selected from:
(i) —Z₁;
(ii) -L₁ₐ-Z₁; or
(iii) —[CR₈ₐR_{8b}]₁₋₆-L_{1b}-Z₁;
wherein
R₈ₐ and R_{8b} are each independently selected from hydrogen, fluoro or (1-2C)alkyl;
L₁ₐ is selected from —C(O)—, —S(O)₂—, —C(O)O—, —C(O)N(Rₐ₁)—, —S(O)₂N(Rₐ₁)— or —N(Rₐ₁)—, wherein Rₐ₁ is selected from hydrogen or (1-2C)alkyl;
L_{1b} is absent or selected from —O—, —S—, —SO—, —SO₂—, —N(Rₐ₂)—, —C(O)—, —C(O)O—, —OC(O)—, —C(O)N(Rₐ₂)—, —N(Rₐ₂)C(O)—, —N(Rₐ₂)C(O)N(R_{b2})—, —C(S)N(Rₐ₂)—, —N(Rₐ₂)C(S)—, —N(Rₐ₂)C(S)N(R_{b2})—, —S(O)₂N(Rₐ₂)—, or —N(Rₐ₂)SO₂, wherein Rₐ₂ and R_{b2} are each independently selected from hydrogen or (1-2C)alkyl; and
Z₁ is selected from (1-6C)alkyl, (3-6C)cycloalkyl, aryl, heterocyclyl, heteroaryl, (3-6C)cycloalkyl(1-2C)alkyl, aryl(1-2C)alkyl, heterocyclyl(1-2C)alkyl or heteroaryl(1-2C)alkyl, with the proviso that when Z₁ is a heterocyclyl group directly linked to a N atom it is a carbon-linked heterocyclyl; and wherein Z₁ is optionally substituted by one or more substituents selected from (1-6C)alkyl, halo, (1-6C)haloalkoxy, cyano, nitro, —NRₑR_{f}, —ORₑ, —C(O)Rₑ, —C(O)ORₑ, —OC(O)Rₑ, —C(O)N(Rₑ)R_{f}, —N(Rₑ)C(O)R_{f}, —S(O)_{y}Rₑ (where y is 0, 1 or 2), —SO₂N(Rₑ)R_{f}, —N(Rₑ)SO₂R_{f}, —(CH₂)_{z}NRₑR_{f} (where z is 1, 2 or 3) or oxo, and wherein Rₑ and R_{f} are each independently selected from hydrogen, (1-6C)alkyl, (1-6C)haloalkyl or (3-6C)cycloalkyl;
and wherein any S atoms present in the heterocyclic ring may optionally be present as S(═O), S(═O)₂ or S(═O)(═NRₑ), wherein Rₑ is selected from hydrogen, (1-3C)alkyl or (2-3C)alkanoyl; and
R₆ is selected from hydrogen, halo, methyl, methoxy and trifluoromethyl.

In another aspect, the present invention relates to compounds, or pharmaceutically acceptable salts, hydrates or solvates thereof, having the structural formula I shown below:

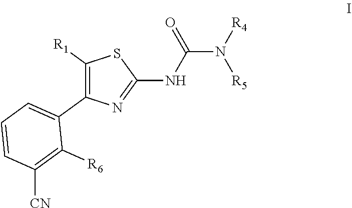

wherein:
R₁ is selected from:

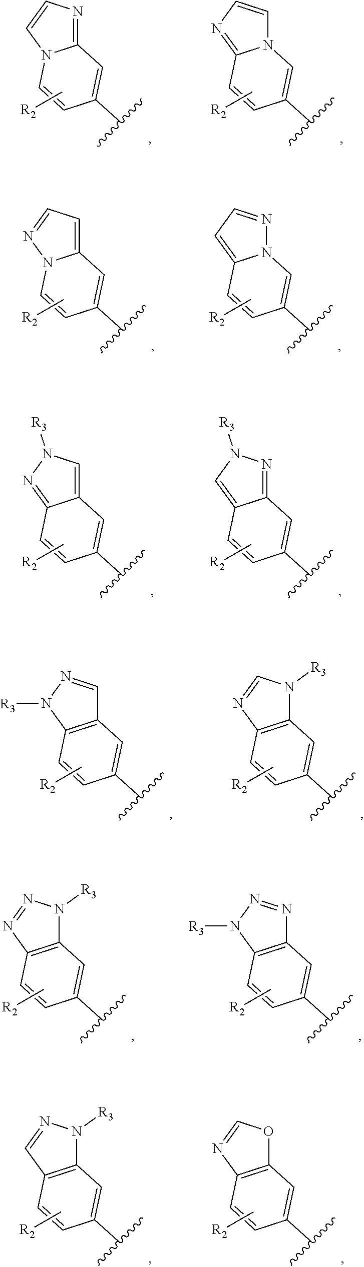

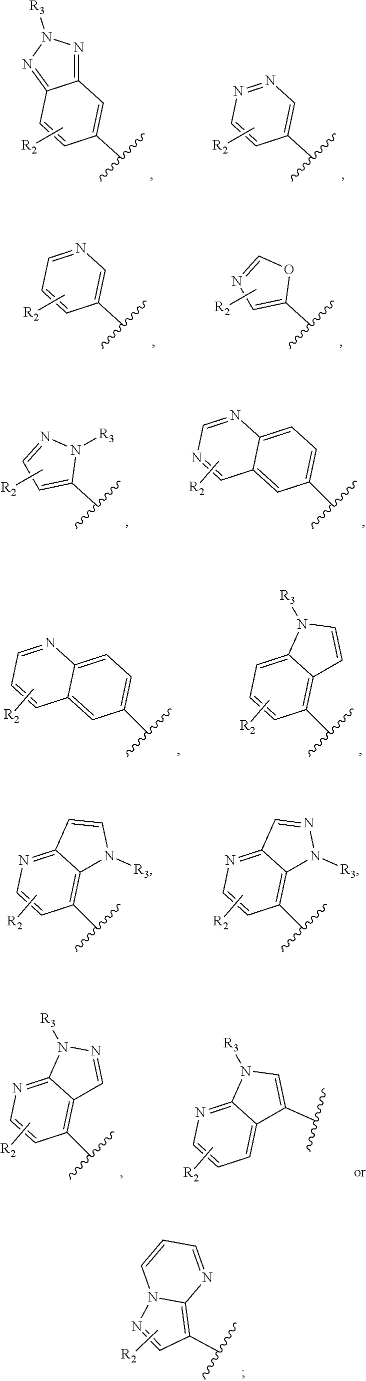

wherein:
any available C atoms in R₁ are optionally substituted by one or more R₂ substituent groups, and
any available N atoms in R₁ are optionally in the form of a N-oxide or substituted by an R₃ group;
wherein
each R₂ is independently selected from (1-6C)alkyl, (3-6C)cycloalkyl, (3-6C)cycloalkyl(1-2C)alkyl, aryl, aryl(1-2C)alkyl, heterocyclyl, heterocyclyl(1-2C)alkyl, heteroaryl (e.g. a 5-membered heteroaryl), heteroaryl(1-2C)alkyl, halo, (1-3C)haloalkyl, (1-3C)haloalkoxy, (1-3C)hydroxyalkyl, cyano, —(CR_{1C}R_{1D})_{q1}—OR_{1A}, —(CR_{1C}R_{1D})_{q1}—C(O)R_{1A}, —(CR_{1C}R_{1D})_{q1}—C(O)OR_{1A}, —(CR_{1C}R_{1D})_{q1}—OC(O)R_{1A}, —(CR_{1C}R_{1D})_{q1}—C(O)N(R_{1B})R_{1A}, —(CR_{1C}R_{1D})_{q1}—N(R_{1B})C(O)R_{1A}, —(CR_{1C}R_{1D})_{q1}—S(O)ₚR_{1A} (where p is 0, 1 or 2), —(CR_{1C}R_{1D})_{q1}—SO₂N(R_{1B})R_{1A}, or —(CR_{1C}R_{1D})_{q1}—N(R_{1B})SO₂R_{1A},
wherein q1 is 0, 1, or 2; and
wherein R_{1A} and R_{1B} are each independently selected from hydrogen, (1-2C)alkyl, (3-4C)cycloalkyl or (3-4C)cycloalkyl(1-2C)alkyl;
wherein R_{1C} and R_{1D} are each independently selected from hydrogen or (1-2C)alkyl; and
R₃ is selected from hydrogen, (1-3C)alkyl, (3-6C)cycloalkyl, (3-6C)cycloalkyl(1-2C)alkyl, aryl, aryl(1-2C)alkyl, a C-linked heterocyclyl, heterocyclyl(1-2C)alkyl, heteroaryl, heteroaryl(1-2C)alkyl, (1-3C)haloalkyl, (1-3C)haloalkoxy, (1-3C)hydroxyalkyl, —(CR₃CR_{3D})₁₋₂—OR_{3A}, —(CR_{3C}R_{3D})_{q3}—C(O)R_{3A}, —(CR_{3C}R_{3D})_{q3}—C(O)OR_{3A}, —(CR_{3C}R_{3D})₁₋₂—OC(O)R_{3A}, —(CR_{3C}R_{3D})_{q3}—C(O)N(R_{3B})R_{q3}, —(CR_{1C}R_{1D})₁₋₂—N(R_{3B})C(O)R_{3A}, —(CR_{3C}R_{3D})—S(O)ₚ₃R_{3A} (where p3 is 0, 1 or 2), —(CR₃CR_{3D})₁₋₂—SO₂N(R_{3B})R_{3A}, or —(CR_{3C}R_{3D})₁₋₂—N(R_{3B})SO₂R_{3A},
wherein q3 is 0, 1, or 2; and
R₄ and R₅ are linked such that, together with the nitrogen atom to which they are attached, they form a heterocyclic ring which is optionally substituted on any available carbon atom by one or more R_{10C} substituent groups,
   wherein R_{10C} is selected from oxo, thioxo, halo, or cyano substituents, or a group of the formula:

—[CR₇ₐR_{7b}]ₙ-L-Z
wherein
n is an integer from 0 to 6;
R₇ₐ and R_{7b} are each independently selected from hydrogen, fluoro or (1-2C)alkyl; L is absent or selected from —O—, —S—, —SO—, —SO₂—, —N(Rₐ)—, —C(O)—, —C(O)O—, —OC(O)—, —C(O)N(Rₐ)—, —N(Rₐ)C(O)—, —N(Rₐ)C(O)N(R_{b})—, —C(S)N(Rₐ)—, —N(Rₐ)C(S)—, —N(Rₐ)C(S)N(R_{b})—, —S(O)₂N(Rₐ)—, or —N(Rₐ)SO₂—, wherein Rₐ and R_{b} are each independently selected from hydrogen or (1-2C)alkyl; and Z is selected from hydrogen, (1-6C)alkyl, (3-6C)cycloalkyl, (3-6C)cycloalkyl(1-2C)alkyl, aryl, aryl(1-2C)alkyl, heterocyclyl, heterocyclyl(1-2C)alkyl, heteroaryl or heteroaryl(1-2C)alkyl; and wherein Z is optionally substituted by one or more substituents selected from (1-6C)alkyl, halo, (1-6C)haloalkyl, (1-6C)haloalkoxy, cyano, nitro, —NR_{c}R_{d}, —OR₀, —C(O)Rₑ, —C(O)ORₑ, —OC(O)Rₑ, —C(O)N(R_{c})R_{d}, —N(R_{c})C(O)R_{d}, —S(O)_{y}Rₑ(where y is 0, 1 or 2), —SO₂N(R_{c})R_{d}, —N(R_{c})SO₂R_{d}, —(CH₂)_{z}NR_{c}R_{d} (where z is 1, 2 or 3) or oxo,
wherein any alkyl moiety in a substituent group on Z is optionally further substituted by cyano, halo, hydroxy, amino, oxo, (1-2C)alkyl or (1-2C)alkoxy and R_{c} and R_{d} are each independently selected from hydrogen, (1-6C)alkyl, (1-6C)haloalkyl or (3-6C)cycloalkyl;
and wherein any available N atoms in the ring formed by R₄ and R₅ are optionally in the form of a N-oxide or are optionally substituted by one or more R_{10N},
   wherein R_{10N} is —S(O)₂NH₂ or selected from:
   (i) —Z₁;
   (ii) -L₁ₐ-Z₁; or
   (iii) —[CR₈ₐR_{8b}]₁₋₆-L_{1b}-Z₁;
wherein
R₂ₐ and R_{2b} are each independently selected from hydrogen, fluoro or (1-2C)alkyl;
L₁ₐ is selected from —C(O)—, —S(O)₂—, —C(O)O—, —C(O)N(Rₐ₁)—, —S(O)₂N(Rₐ₁)— or —N(Rₐ₁)—, wherein Rₐ₁ is selected from hydrogen or (1-2C)alkyl;
L_{1b} is absent or selected from —O—, —S—, —SO—, —SO₂—, —N(Rₐ₂)—, —C(O)—, —C(O)O—, —OC(O)—, —C(O)N(Rₐ₂)—, —N(Rₐ₂)C(O)—, —N(Rₐ₂)C(O)N(R_{b2})—, —C(S)N(Rₐ₂)—, —N(Rₐ₂)C(S)—, —N(Rₐ₂)C(S)N(R_{b2})—, —S(O)₂N(Rₐ₂)—, or —N(Rₐ₂)SO₂, wherein Rₐ₂ and R_{b2} are each independently selected from hydrogen or (1-2C)alkyl; and
Z₁ is selected from (1-6C)alkyl, (3-6C)cycloalkyl, aryl, heterocyclyl, heteroaryl, (3-6C)cycloalkyl(1-2C)alkyl, aryl(1-2C)alkyl, heterocyclyl(1-2C)alkyl or heteroaryl(1-2C)alkyl, with the proviso that when Z₁ is a heterocyclyl group directly linked to a N atom it is a carbon-linked heterocyclyl; and wherein Z₁ is optionally substituted by one or more substituents selected from (1-6C)alkyl, halo, (1-6C)haloalkoxy, cyano, nitro, —NRₑR_{f}, —ORₑ, —C(O)Rₑ, —C(O)ORₑ, —OC(O)Rₑ, —C(O)N(Rₑ)R_{f}, —N(Rₑ)C(O)R_{f}, —S(O)_{y}Rₑ (where y is 0, 1 or 2), —SO₂N(Rₑ)R_{f}, —N(Rₑ)SO₂R_{f}, —(CH₂)_{z}NRₑR_{f} (where z is 1, 2 or 3) or oxo, and wherein Rₑ and R_{f} are each independently selected from hydrogen, (1-6C)alkyl, (1-6C)haloalkyl or (3-6C)cycloalkyl;
and wherein any S atoms present in the heterocyclic ring may optionally be present as S(═O), S(═O)₂ or S(═O)(═NRₑ), wherein Rₑ is selected from hydrogen, (1-3C)alkyl or (2-3C)alkanoyl; and
R₆ is selected from hydrogen, halo, methyl, methoxy and trifluoromethyl.

Particular compounds of the invention include, for example, compounds of the formula I, or pharmaceutically acceptable salts, hydrates and/or solvates thereof, wherein, unless otherwise stated, each of R₁, R₂, R₃, R₄, R₅, R₆ and any associated substituent group have any of the meanings defined hereinbefore or in any of paragraphs (1) to (36) hereinafter:—
(1) R₁ is selected from:

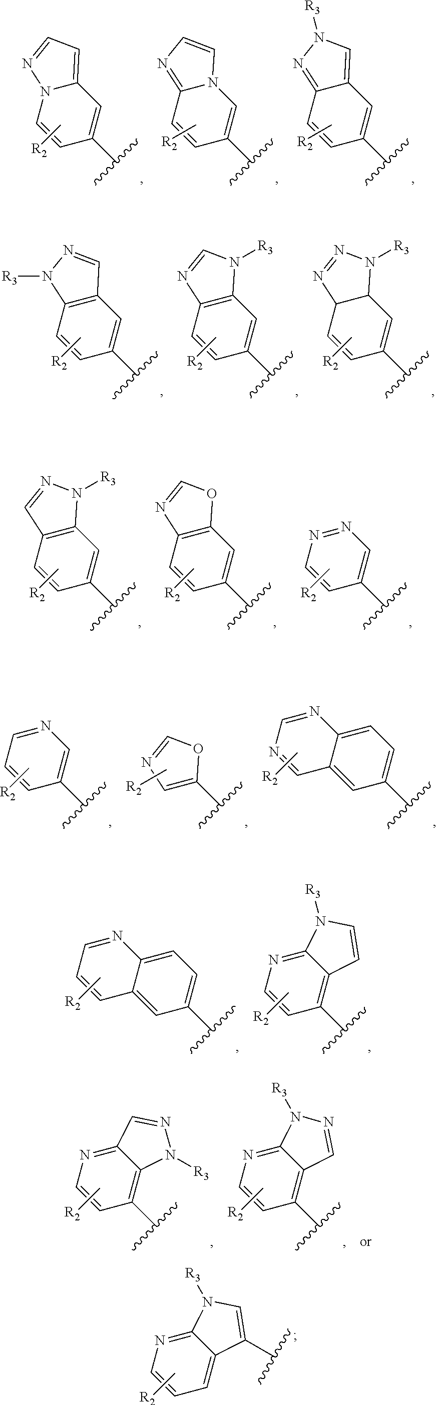

wherein each R₁ is optionally substituted on any available carbon atom by one or more R₂, and R₂ and R₃ are as defined herein.
(2) R₁ is selected from:

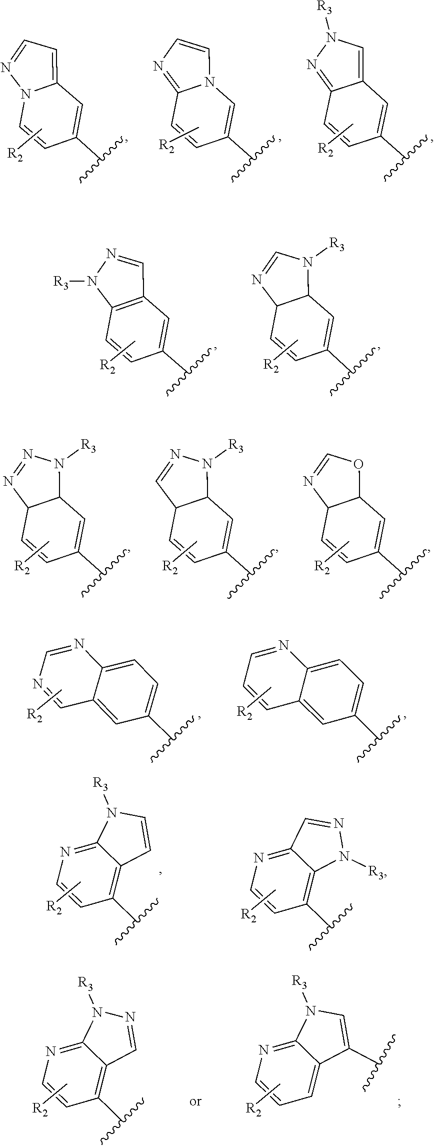

wherein R₁ is optionally substituted on any available carbon atom by one or more R₂ substituent groups; and R₂ and R₃ are as defined herein.
(3) R₁ is selected from:

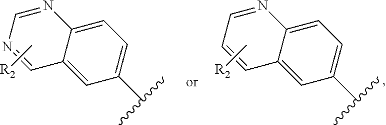

wherein R₁ is optionally substituted on any available carbon atom by one or more R₂ substituent groups, and R₂ is as defined herein.
(4) R₁ is:

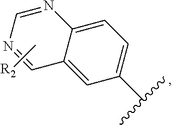

wherein R₁ is optionally substituted on any available carbon atom by one or more R₂ substituent groups, and R₂ is as defined herein.
(5) R₁ is:

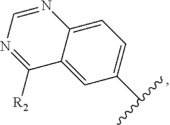

wherein R₂ is as defined herein.
(6) R₁ is selected from:

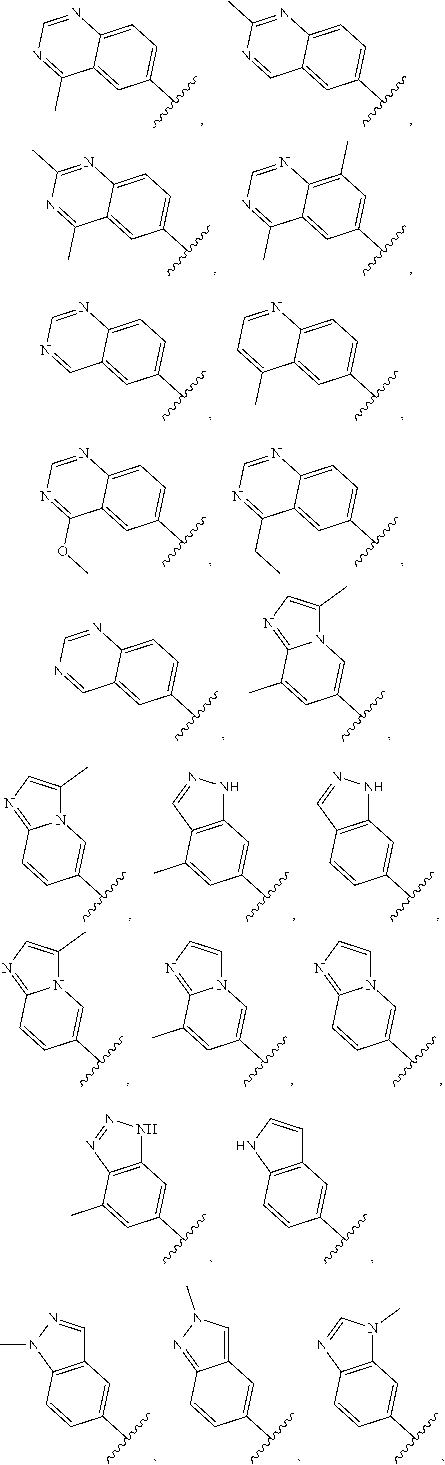

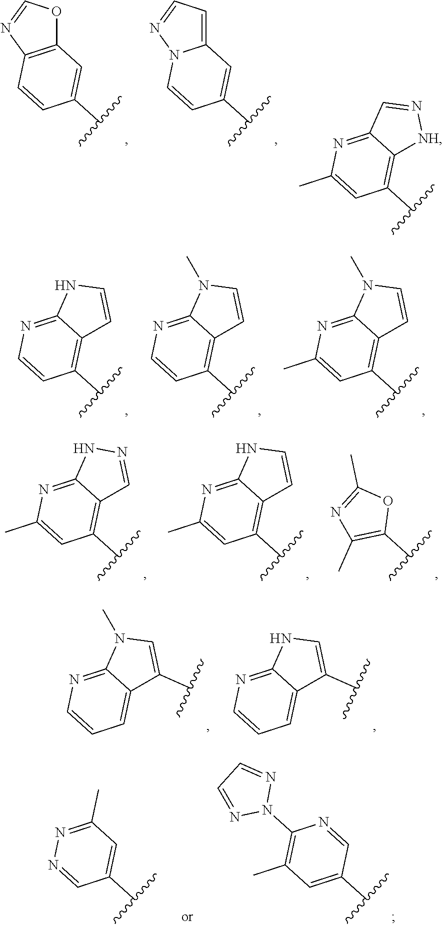

(7) R₁ is:

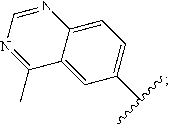 [SMILES: *c1ccc2ncnc(C)c2c1]

(8) each R₂, if present is independently selected from (1-3C)alkyl, (3-6C)cycloalkyl, a 4- or 5-membered heterocyclyl, a 5-membered heteroaryl, halo, (1-3C)haloalkyl, (1-3C)haloalkoxy, (1-3C)hydroxyalkyl, cyano, —(CR_{1C}R_{1D})_{q1}—OR_{1A}, —(CR_{1C}R_{1D})_{q1}—C(O)R_{1A};
   wherein q1 is 0 or 1; and
      wherein R_{1A} and R_{1B} are each independently selected from hydrogen, (1-2C)alkyl;
      wherein R_{1C} and R_{1D} are each independently selected from hydrogen or (1-2C)alkyl;
(9) each R₂, if present, is independently selected from (1-2C)alkyl, a 5-membered heteroaryl, halo, (1-2C)haloalkyl, (1-2C)haloalkoxy, (1-2C)hydroxyalkyl, cyano, —(CR_{1C}R_{1D})_{q1}—OR_{1A};
   wherein q1 is 0 or 1; and
      wherein R_{1A} and R_{1B} are each independently selected from hydrogen, (1-2C)alkyl;
(10) each R₂, if present, is independently selected from methyl, ethyl or methoxy;
(11) each R₂, if present, is methyl;
(11a) R₃ is selected from hydrogen, (1-3C)alkyl, (3-6C)cycloalkyl, (3-6C)cycloalkyl(1-2C)alkyl, aryl, aryl(1-2C)alkyl, a C-linked heterocyclyl, heterocyclyl(1-2C)alkyl, heteroaryl, heteroaryl(1-2C)alkyl, (1-3C)haloalkyl, (1-3C)haloalkoxy, (1-3C)hydroxyalkyl, —(CR₃CR_{3D})₁₋₂—OR_{3A}, —(CR_{3C}R_{3D})_{q3}—C(O)R_{3A}, —(CR_{3C}R_{3D})_{q3}—C(O)OR_{3A}, —(CR_{3C}R_{3D})₁₋₂—OC(O)R_{3A}, —(CR_{3C}R_{3D})_{q3}—C(O)N(R_{3B})R_{q3}, —(CR_{1C}R_{1D})₁₋₂—N(R_{3B})C(O)R_{3A}, —(CR_{3C}R_{3D})—S(O)ₚ₃R_{3A} (where p3 is 0, 1 or 2), —(CR₃CR_{3D})₁₋₂—SO₂N(R_{3B})R_{3A}, or —(CR_{3C}R_{3D})₁₋₂—N(R_{3B})SO₂R_{3A};
   wherein R_{3A} and R_{3B} are each independently selected from hydrogen or (1-2C)alkyl;
   wherein R_{3C} and R_{3D} are each independently selected from hydrogen or (1-2C)alkyl; and wherein q3 is 0, 1, or 2;
(11b) R₃ is selected from hydrogen, (1-3C)alkyl, (3-6C)cycloalkyl, (3-6C)cycloalkyl(1-2C)alkyl, aryl, aryl(1-2C)alkyl, a C-linked heterocyclyl, heterocyclyl(1-2C)alkyl, heteroaryl, heteroaryl(1-2C)alkyl, (1-3C)haloalkyl, (1-3C)haloalkoxy or (1-3C)hydroxyalkyl;
(12) R₃ is selected from hydrogen or (1-3C)alkyl;
(13) R₃ is selected from hydrogen or methyl;
(14) R₄ and R₅ are linked such that, together with the nitrogen atom to which they are attached, they form a heterocyclic ring which is optionally substituted on any available carbon atom by one or more R_{10C} substituent groups, wherein R_{10C} is selected from oxo, thioxo, halo, or cyano substituents, or a group of the formula:

—[CH₂]ₙ-L-Z
wherein
   n is 0 to 4;
   L is absent or selected from —O—, —S—, —SO—, —SO₂—, —N(Rₐ)—, —C(O)—, —C(O)O—, —OC(O)—, —C(O)N(Rₐ)—, —N(Rₐ)C(O)—, —N(Rₐ)C(O)N(R_{b})—, —S(O)₂N(Rₐ)—, or —N(Rₐ)SO₂—, wherein Rₐ and R_{b} are each independently selected from hydrogen or (1-2C)alkyl; and
   Z is selected from hydrogen, (1-6C)alkyl, (3-6C)cycloalkyl, (3-6C)cycloalkyl(1-2C)alkyl, aryl, aryl(1-2C)alkyl, heterocyclyl, heterocyclyl(1-2C)alkyl, heteroaryl or heteroaryl(1-2C)alkyl; and wherein Z is optionally substituted by one or more substituents selected from (1-4C)alkyl, halo, (1-4C)haloalkyl, (1-4C)haloalkoxy, cyano, nitro, —NR_{c}R_{d}, —OR_{c}, —C(O)R_{c}, —C(O)OR_{c}, —OC(O)R_{c}, —C(O)N(R_{c})R_{d}, —N(R_{c})C(O)R_{d}, —S(O)_{y}R_{c}(where y is 0, 1 or 2), —SO₂N(R_{c})R_{d}, —N(R_{c})SO₂R_{d}, —(CH₂)_{z}NR_{c}R_{d} (where z is 1, 2 or 3) or oxo,
wherein any alkyl moiety in a substituent group on Z is optionally further substituted by cyano, halo, hydroxy, amino, oxo, (1-2C)alkyl or (1-2C)alkoxy and R_{c} and R_{d} are each independently selected from hydrogen, (1-4C)alkyl, (1-4C)haloalkyl or (3-6C)cycloalkyl;
and wherein any available N atoms are optionally in the form of a N-oxide or are optionally substituted by one or more R_{10N}, wherein R_{10N} is —S(O)₂NH₂ or selected from:
(i) —Z₁;
(ii) -L₁ₐ-Z₁; or
(iii) —[CR₈ₐR_{8b}]₁₋₄-L_{1b}-Z₁;
   wherein
   R₈ₐ and R_{8b} are both hydrogen;
   L₁ₐ is selected from —C(O)—, —S(O)₂—, —C(O)O—, —C(O)N(Rₐ₁)— or —S(O)₂N(Rₐ₁)—, wherein Rₐ₁ is hydrogen or methyl;
   L_{1b} is absent or selected from —O—, —S—, —SO—, —SO₂—, —N(Rₐ₂)—, —C(O)—, —C(O)O—, —OC(O)—, —C(O)N(Rₐ₂)—, —N(Rₐ₂)C(O)—, —N(Rₐ₂)C(O)N(R_{b2})—, —C(S)N(Rₐ₂)—, —N(Rₐ₂)C(S)—, —N(Rₐ₂)C(S)N(R_{b2})—, —S(O)₂N(Rₐ₂)—, or —N(Rₐ₂)SO₂, wherein Rₐ₂ and R_{b2} are each independently selected from hydrogen or methyl; and
   Z₁ is selected from (1-6C)alkyl, (3-6C)cycloalkyl, aryl, heterocyclyl, heteroaryl, (3-6C)cycloalkyl(1-2C)alkyl, aryl(1-2C)alkyl, heterocyclyl(1-2C)alkyl or heteroaryl(1-2C)alkyl, with the proviso that when Z₁ is a heterocyclyl group directly linked to a N atom it is a carbon-linked heterocyclyl; and wherein Z₁ is optionally substituted by one or more substituents selected from (1-4C)alkyl, halo, (1-4C)haloalkoxy, cyano, nitro, —NRₑR_{f}, —ORₑ, —C(O)Rₑ, —C(O)ORₑ, —OC(O)Rₑ, —C(O)N(Rₑ)R_{f}, —N(Rₑ)C(O)R_{f}, —S(O)_{y}Rₑ (where y is 0, 1 or 2), —SO₂N(Rₑ)R_{f}, —N(Rₑ)SO₂R_{f}, —(CH₂)_{z}NRₑR_{f} (where z is 1, 2 or 3) or oxo, and wherein Rₑ and R_{f} are each independently selected from hydrogen, (1-4C)alkyl, (1-4C)haloalkyl or (3-6C)cycloalkyl;
and wherein any S atoms present in the heterocyclic ring may optionally be present as S(═O), S(═O)₂ or S(═O)(═NRₑ) wherein Rₑ is selected from hydrogen, (1-3C)alkyl or (2-3C)alkanoyl;
(15) R₄ and R₅ are linked such that, together with the nitrogen atom to which they are attached, they form a heterocyclic ring which is optionally substituted on any available carbon atom by one or more R_{10C} substituent groups, wherein R_{10C} is selected from oxo, halo, or cyano substituents, or a group of the formula:

—[CH₂]ₙ-L-Z
wherein
   n is 0 to 2 (i.e. n is 0, 1 or 2);
   L is absent or selected from —O—, —S—, —SO—, —SO₂—, —N(Rₐ₂)—, —C(O)—, —C(O)O—, —OC(O)—, —C(O)N(Rₐ₂)—, —N(Rₐ₂)C(O)—, —N(Rₐ₂)C(O)N(R_{b2})—, —S(O)₂N(Rₐ₂)—, or —N(Rₐ₂)SO₂—, wherein Rₐ₂ and R_{b2} are each independently selected from hydrogen or methyl; and
   Z is selected from hydrogen, (1-6C)alkyl, (3-6C)cycloalkyl, (3-6C)cycloalkyl(1-2C)alkyl, aryl, heterocyclyl, heterocyclyl(1-2C)alkyl or heteroaryl; and wherein Z is optionally substituted by one or more substituents selected from (1-2C)-alkyl, halo, (1-2C)haloalkyl, (1-2C)haloalkoxy, cyano, —NR_{c}R_{d}, —OR_{c}, —C(O)R_{c}, —C(O)OR_{c}, —OC(O)R_{c}, —C(O)N(R_{c})R_{d}, —N(R_{c})C(O)R_{d}, —S(O)_{y}R_{c}(where y is 0, 1 or 2), —SO₂N(R_{c})R_{d}, —N(R_{c})SO₂R_{d}, —(CH₂)_{z}NR_{c}R_{d} (where z is 1 or 2) or oxo; wherein any alkyl moiety in a substituent group on Z₁ is optionally further substituted by cyano, halo, hydroxy, amino, oxo, (1-2C)alkyl or (1-2C)alkoxy and R_{c} and R_{d} are each independently selected from hydrogen, (1-2C)alkyl, (1-2C)haloalkyl or (3-6C)cycloalkyl;
and wherein any available N atoms are optionally in the form of a N-oxide or are optionally substituted by one or more R_{10N}, wherein R_{10N} is —S(O)₂NH₂ or selected from:
(i) —Z₁;
(ii) -L₁ₐ-Z₁; or
(iii) —[CR₈ₐR_{8b}]₁₋₂-L_{1b}-Z₁;
   wherein
   R₈ₐ and R_{8b} are both hydrogen;
   L₁ₐ is selected from —C(O)—, —S(O)₂— or —S(O)₂N(Rₐ₁)—, wherein Rₐ₁ is hydrogen or methyl;
   L_{1b} is absent or selected from —O—, —S—, —SO—, —SO₂—, —N(Rₐ₂)—, —C(O)—, —C(O)O—, —OC(O)—, —C(O)N(Rₐ₂)—, —N(Rₐ₂)C(O)—, —N(Rₐ₂)C(O)N(R_{b2})—, —S(O)₂N(Rₐ₂)—, or —N(Rₐ₂)SO₂, wherein Rₐ₂ and R_{b2} are each independently selected from hydrogen or methyl; and
   Z₁ is selected from (1-6C)alkyl, (3-6C)cycloalkyl, aryl, heterocyclyl, heteroaryl, (3-6C)cycloalkyl(1-2C)alkyl or aryl(1-2C)alkyl, with the proviso that when Z₁ is a heterocyclyl group directly linked to a N atom it is a carbon-linked heterocyclyl; and wherein Z₁ is optionally substituted by one or more substituents selected from (1-2C)alkyl, halo, (1-2C)haloalkoxy, cyano, nitro, —NRₑR_{f}, —ORₑ, —C(O)Rₑ, —C(O)ORₑ, —OC(O)Rₑ, —C(O)N(Rₑ)R_{f}, —N(Rₑ)C(O)R_{f}, —S(O)_{y}Rₑ (where y is 0, 1 or 2), —SO₂N(Rₑ)R_{f}, —N(Rₑ)SO₂R_{f}, —(CH₂)_{z}NRₑR_{f} (where z is 1, 2 or 3) or oxo, and wherein Rₑ and R_{f} are each independently selected from hydrogen or (1-2C)alkyl;
and wherein any S atoms present in the heterocyclic ring may optionally be present as S(═O), S(═O)₂ or S(═O)(═NRₑ) wherein Rₑ is selected from hydrogen, (1-3C)alkyl or (2-3C)alkanoyl;
(16) R₄ and R₅ are linked such that, together with the nitrogen atom to which they are attached, they form a heterocyclic ring which is optionally substituted on any available carbon atom by one or more R_{10C} substituent groups, wherein R_{10C} is selected from oxo, halo, or cyano substituents, or a group of the formula:

—[CH₂]ₙ-L-Z
wherein
   n is 0 to 2 (i.e. n is 0, 1 or 2);
   L is absent or selected from —O—, —S—, —SO—, —SO₂—, —N(Rₐ)—, —C(O)—, —C(O)O—, —OC(O)—, —C(O)N(Rₐ)—, —N(Rₐ)C(O)—, —N(Rₐ)C(O)N(R_{b})—, —S(O)₂N(Rₐ)—, or —N(Rₐ)SO₂—, wherein Rₐ and R_{b} are hydrogen or methyl; and Z is selected from hydrogen, (1-6C)alkyl, (3-6C)cycloalkyl, (3-6C)cycloalkyl(1-2C)alkyl, aryl, heterocyclyl, heterocyclyl(1-2C)alkyl or heteroaryl; and wherein Z is optionally substituted by one or more substituents selected from (1-2C)-alkyl, halo, (1-2C)haloalkyl, (1-2C)haloalkoxy, cyano, —NR_{c}R_{d}, —ORₑ, —C(O)Rₑ, —C(O)ORₑ, —OC(O)Rₑ, —C(O)N(R_{c})R_{d}, —N(R_{c})C(O)R_{d}, —S(O)_{y}Rₑ(where y is 0, 1 or 2), —SO₂N(R_{c})R_{d}, —N(R_{c})SO₂R_{d}, —(CH₂)_{z}NR_{c}R_{d} (where z is 1 or 2) or oxo;
   wherein R_{c} and R_{d} are each independently selected from hydrogen, (1-2C)alkyl or (1-2C)haloalkyl;
and wherein any available N atoms are optionally in the form of a N-oxide or are optionally substituted by one or more R_{10N}, wherein R_{10N} is —S(O)₂NH₂ or selected from:
(i) —Z₁;
(ii) -L₁ₐ Z₁; or
(iii) —[CR₈ₐR_{8b}]₁₋₂-L_{1b}-Z₁;
   wherein
   R₈ₐ and R_{8b} are both hydrogen;
   L₁ₐ is selected from —C(O)—, —S(O)₂— or —S(O)₂N(Rₐ₁)—, wherein Rₐ₁ is hydrogen;
   L_{1b} is absent or selected from —O—, —S—, —SO—, —SO₂—, —N(Rₐ₂)—, —C(O)N(Rₐ₂)—, —N(Rₐ₂)C(O)—, —S(O)₂N(Rₐ₂)— or —N(Rₐ₂)SO₂, wherein Rₐ₂ is hydrogen; and
   Z₁ is selected from (1-6C)alkyl, (3-6C)cycloalkyl, aryl, heterocyclyl, heteroaryl, with the proviso that when Z₁ is a heterocyclyl group directly linked to a N atom it is a carbon-linked heterocyclyl; and wherein Z₁ is optionally substituted by one or more substituents selected from (1-2C)alkyl, halo, (1-2C)haloalkoxy, cyano, —NRₑR_{f}, —ORₑ, —C(O)Rₑ, —C(O)ORₑ, —OC(O)Rₑ, —C(O)N(Rₑ)R_{f}, —N(Rₑ)C(O)R_{f}, —S(O)_{y}Rₑ (where y is 0, 1 or 2), —SO₂N(Rₑ)R_{f}, —N(Rₑ)SO₂R_{f}, —(CH₂)_{z}NR_{c}R_{d} (where z is 1 or 2); and wherein Rₑ and R_{f} are each independently selected from hydrogen or methyl;
and wherein any S atoms present in the heterocyclic ring may optionally be present as S(═O), S(═O)₂ or S(═O)(═NRₑ) wherein Rₑ is selected from hydrogen, (1-2C)alkyl or (2C)alkanoyl;
(17) R₄ and R₅ are linked such that, together with the nitrogen atom to which they are attached, they form a heterocyclic ring which is optionally substituted on any available carbon atom by one or more R_{10C} substituent groups, wherein R_{10C} is selected from oxo, halo, or cyano, or a group of the formula:

—[CH₂]ₙ-L-Z
wherein
   n is 0 to 2 (i.e. n is 0, 1 or 2);
   L is absent or selected from —O—, —S—, —SO—, —SO₂—, —N(Rₐ)—, —C(O)—, —C(O)O—, —OC(O)—, —C(O)N(Rₐ)—, —N(Rₐ)C(O)—, —S(O)₂N(Rₐ)—, or —N(Rₐ)SO₂—, wherein Rₐ and R_{b} are each independently selected from hydrogen or methyl; and
   Z is selected from hydrogen, (1-4C)alkyl, (3-6C)cycloalkyl, (3-6C)cycloalkyl(1-2C)alkyl, phenyl, a 4-7 membered heterocyclyl, a 4-7 membered heterocyclyl(1-2C)alkyl or a 5 or 6-membered heteroaryl; and wherein Z is optionally substituted by one or more substituents selected from (1-2C)alkyl, halo, (1-2C)haloalkyl, (1-2C)haloalkoxy, cyano, —NR_{c}R_{d}, —ORₑ, —C(O)Rₑ, —C(O)ORₑ, —OC(O)Rₑ, —C(O)N(R_{c})R_{d}, —N(R_{c})C(O)R_{d}, —S(O)_{y}Rₑ(where y is 0, 1 or 2), —SO₂N(R_{c})R_{d}, —N(R_{c})SO₂R_{d}, —(CH₂)_{z}NR_{c}R_{d} (where z is 1 or 2) or oxo; wherein R_{c} and R_{d} are each independently selected from hydrogen or (1-2C)alkyl;
and wherein any available N atoms are optionally in the form of a N-oxide or are optionally substituted by one or more R_{10N}, wherein R_{10N} is —S(O)₂NH₂ or selected from:
(i) —Z₁;
(ii) -L₁ₐ-Z₁; or
(iii) —[CR₈ₐR_{8b}]₁₋₂-L_{1b}-Z₁;
   wherein
   R₈ₐ and R_{8b} are both hydrogen;
   L₁ₐ is selected from —C(O)—, —S(O)₂— or —S(O)₂N(Rₐ₁)—, wherein Rₐ₁ is hydrogen;
   L_{1b} is absent or selected from —O—, —S—, —SO—, —SO₂—, —N(Rₐ₂)—, —C(O)N(Rₐ₂)—, —N(Rₐ₂)C(O)—, —S(O)₂N(Rₐ₂)— or —N(Rₐ₂)SO₂, wherein Rₐ₂ is hydrogen; and
   Z₁ is selected from (1-4C)alkyl, phenyl, a 4-7 membered heterocyclyl or a 5 or 6-membered heteroaryl, with the proviso that when Z₁ is a heterocyclyl group directly linked to a N atom it is a carbon-linked heterocyclyl; and wherein Z₁ is optionally substituted by one or more substituents selected from (1-2C)alkyl, halo, (1-2C)haloalkoxy, cyano, —NRₑR_{f}, —ORₑ, —C(O)Rₑ, —C(O)ORₑ, —OC(O)Rₑ, —C(O)N(Rₑ)R_{f}, —N(Rₑ)C(O)R_{f}, —S(O)_{y}Rₑ (where y is 0, 1 or 2), —SO₂N(Rₑ)R_{f}, —N(Rₑ)SO₂R_{f}, —(CH₂)_{z}NR_{c}R_{d} (where z is 1 or 2); and wherein Rₑ and R_{f} are each independently selected from hydrogen or methyl;
and wherein any S atoms present in the heterocyclic ring may optionally be present as S(═O), S(═O)₂ or S(═O)(═NRₑ) wherein Rₑ is selected from hydrogen, (1-2C)alkyl or (2C)alkanoyl;
(18) R₄ and R₅ are linked such that, together with the nitrogen atom to which they are attached, they form a heterocyclic ring which is optionally substituted on any available carbon atom by one or more R_{10C} substituent groups, wherein R_{10C} is selected from oxo, halo, or cyano substituents, or a group of the formula:

—[CH₂]ₙ-L-Z
wherein
   n is 0 to 2 (i.e. n is 0, 1 or 2);
   L is absent or selected from —O—, —S—, —SO—, —SO₂—, —N(Rₐ)—, —C(O)—, —C(O)O—, —OC(O)—, —C(O)N(Rₐ)—, —N(Rₐ)C(O)—, —S(O)₂N(Rₐ)—, or —N(Rₐ)SO₂—, wherein wherein Rₐ and R_{b} are each independently selected from hydrogen or methyl; and
   Z is selected from hydrogen, (1-4C)alkyl, (3-6C)cycloalkyl, (3-6C)cycloalkyl(1-2C)alkyl, phenyl, a 4-7 membered heterocyclyl, a 4-7 membered heterocyclyl(1-2C)alkyl or a 5 or 6-membered heteroaryl; and wherein Z is optionally substituted by one or more substituents selected from (1-2C)alkyl, halo, (1-2C)haloalkyl, (1-2C)haloalkoxy, cyano, —NR_{c}R_{d}, —OR_{c}, —C(O)R_{c}, —C(O)N(R_{c}) R_{d}, —N(R_{c})C(O)R_{d} or —S(O)_{y}R_{c} (where y is 0, 1 or 2) or oxo;
   wherein R_{c} and R_{d} are each independently selected from hydrogen or (1-2C)alkyl;
and wherein any available N atoms are optionally in the form of a N-oxide or are optionally substituted by one or more R_{10N}, wherein R_{10N} is —S(O)₂NH₂ or selected from:
(i) —Z₁;
(ii) -L₁ₐ Z₁; or
(iii) —[CR₈ₐR_{8b}]₁₋₂-L_{1b}-Z₁;
   wherein
   R₈ₐ and R_{8b} are both hydrogen;
   L₁ₐ is selected from —C(O)—, —S(O)₂— or —S(O)₂N(Rₐ₁)—, wherein Rₐ₁ is hydrogen;
   L_{1b} is absent or selected from —O—, —S—, —SO—, —SO₂—, —N(Rₐ₂)—, —C(O)N(Rₐ₂)—, —N(Rₐ₂)C(O)—, —S(O)₂N(Rₐ₂)— or —N(Rₐ₂)SO₂, wherein Rₐ₂ is hydrogen; and
   Z₁ is selected from (1-4C)alkyl, phenyl, a 4-7 membered heterocyclyl or a 5 or 6-membered heteroaryl, with the proviso that when Z₁ is a heterocyclyl group directly linked to a N atom it is a carbon-linked heterocyclyl; and wherein Z₁ is optionally substituted by one or more substituents selected from (1-2C)alkyl, halo, (1-2C)haloalkoxy, cyano, —NRₑR_{f}, —ORₑ, —C(O)Rₑ, —C(O)N(Rₑ)R_{f}, —N(Rₑ)C(O)R_{f}, —S(O)_{y}Rₑ (where y is 0, 1 or 2); and wherein Rₑ and R_{f} are each independently selected from hydrogen or methyl;
and wherein any S atoms present in the heterocyclic ring may optionally be present as S(═O), S(═O)₂ or S(═O)(═NRₑ) wherein Rₑ is selected from hydrogen, methyl or (2C)alkanoyl;
(19) R₄ and R₅ are linked such that, together with the nitrogen atom to which they are attached, they form a heterocyclic ring which is optionally substituted on any available carbon atom by one or more R_{10C} substituent groups, wherein R_{10C} is selected from oxo, halo, or cyano substituents, or a group of the formula:

—[CH₂]ₙ-L-Z
wherein
   n is 0 to 2 (i.e. n is 0, 1 or 2);
   L is absent or selected from —O—, —S—, —SO—, —SO₂—, —N(Rₐ)—, —C(O)—, —C(O)O—, —OC(O)—, —C(O)N(Rₐ)—, —N(Rₐ)C(O)—, —S(O)₂N(Rₐ)—, or —N(Rₐ)SO₂—, wherein wherein Rₐ and R_{b} are each independently selected from hydrogen or methyl; and
   Z is selected from hydrogen, (1-4C)alkyl, (3-6C)cycloalkyl, (3-6C)cycloalkyl(1-2C)alkyl, a 4-7 membered heterocyclyl, a 4-7 membered heterocyclyl(1-2C)alkyl or a 5 or 6-membered heteroaryl; and wherein Z is optionally substituted by one or more substituents selected from (1-2C)alkyl, halo, (1-2C)haloalkyl, (1-2C)haloalkoxy, cyano, —NR_{c}R_{d}, —OR_{c}, —C(O)R_{c}, —C(O)N(R_{c})R_{d}, —N(R_{c})C(O)R_{d} or —S(O)_{y}R_{c} (where y is 0, 1 or 2) or oxo;
   wherein R_{c} and R_{d} are each independently selected from hydrogen or (1-2C)alkyl;
and wherein any available N atoms are optionally in the form of a N-oxide or are optionally substituted by one or more R_{10N}, wherein R_{10N} is —S(O)₂NH₂ or selected from:
(i) —Z₁;
(ii) -L₁ₐ-Z₁; or
(iii) —[CR₈ₐR_{8b}]₁₋₂-L_{1b}-Z₁;
   wherein
   R₈ₐ and R_{8b} are both hydrogen;
   L₁ₐ is selected from —C(O)—, —S(O)₂— or —S(O)₂N(Rₐ₁)—, wherein Rₐ₁ is hydrogen;
   L_{1b} is absent or selected from —O—, —S—, —SO—, —SO₂—, —N(Rₐ₂)—, —C(O)N(Rₐ₂)—, —N(Rₐ₂)C(O)—, —S(O)₂N(Rₐ₂)— or —N(Rₐ₂)SO₂, wherein Rₐ₂ is hydrogen; and
   Z₁ is selected from (1-4C)alkyl or a 4-7 membered heterocyclyl, with the proviso that when Z₁ is a heterocyclyl group directly linked to a N atom it is a carbon-linked heterocyclyl;
   wherein Z₁ is optionally substituted by one or more substituents selected from (1-2C)alkyl, halo, (1-2C)haloalkoxy, cyano, —NRₑR_{f}, —ORₑ, —C(O)Rₑ, —C(O)N(Rₑ) R_{f}, —N(Rₑ)C(O)R_{f}, —S(O)_{y}Rₑ (where y is 0, 1 or 2); and wherein Rₑ and R_{f} are each independently selected from hydrogen or methyl;
and wherein any S atoms present in the heterocyclic ring may optionally be present as S(═O), S(═O)₂ or S(═O)(═NRₑ) wherein Rₑ is selected from hydrogen, methyl or (2C)alkanoyl;
(20) R₄ and R₅ are linked such that, together with the nitrogen atom to which they are attached, they form a heterocyclic ring which is optionally substituted on any available carbon atom by one or more R_{10C} substituent groups, wherein R_{10C} is selected from oxo, halo, or cyano substituents, or a group of the formula:

-L-Z
wherein
   L is absent or selected from —O—, —S—, —SO—, —SO₂—, —N(Rₐ)—, —C(O)—, —C(O)O—, —OC(O)—, —C(O)N(Rₐ)—, —N(Rₐ)C(O)—, —S(O)₂N(Rₐ)—, or —N(Rₐ)SO₂—, wherein wherein Rₐ and R_{b} are each independently selected from hydrogen or (1-2C)alkyl; and
Z is selected from hydrogen, (1-4C)alkyl, (3-6C)cycloalkyl, (3-6C)cycloalkyl(1-2C)alkyl, a 4-7 membered heterocyclyl or heterocyclyl(1-2C)alkyl; and Z is optionally substituted by one or more substituents selected from cyano, —NR_{c}R_{d} or —OR₀, wherein R_{c} and R_{d} are each independently selected from hydrogen or methyl, wherein any alkyl moiety in a substituent group on Z is optionally further substituted by cyano, halo, hydroxy, amino, oxo, (1-2C)alkyl or (1-2C)alkoxy and R_{c} and R_{d} are each independently selected from hydrogen, (1-6C)alkyl;
   and wherein any available N atoms are optionally in the form of a N-oxide or are optionally substituted by one or more R_{10N}, wherein R_{10N} is —S(O)₂NH₂ or (1-4C)alkyl, and wherein any S atoms present in the heterocyclic ring may optionally be present as S(═O), S(═O)₂ or S(═O)(═NRₑ) wherein Rₑ is selected from hydrogen, methyl or (2C)alkanoyl;
(21) R₄ and R₅ are linked such that, together with the nitrogen atom to which they are attached, they form a heterocyclic ring which is optionally substituted on any available carbon atom by one or more R_{10C} substituent groups, wherein R_{10C} is selected from oxo, halo, cyano, or a group of the formula:

-L-Z
wherein
L is absent, —O— or —N(Rₐ)—, wherein Rₐ is hydrogen or methyl; and Z is selected from (1-4C)alkyl, 4-7 membered heterocyclyl, (3-6C)cycloalkyl(1-2C)alkyl,
wherein any (1-4C)alkyl, 4-7 membered heterocyclyl or (3-6C)cycloalkyl(1-2C)alkyl, is optionally substituted by one or more substituents selected from cyano, —NR_{c}R_{d} or —OR_{c}, wherein R_{c} and R_{d} are each independently selected from hydrogen or methyl;
and wherein any available N atoms are optionally in the form of a N-oxide or are optionally substituted by one or more R_{10N}, wherein R_{10N} is (1-4C)alkyl,
and wherein any S atoms present in the heterocyclic ring may optionally be present as S(═O);
(22) R₄ and R₅ are linked such that, together with the nitrogen atom to which they are attached, they form a heterocyclic ring which is optionally substituted on any available carbon atom by one or more R_{10C} substituent groups, wherein each R_{10C} is independently selected from (1-4C)alkyl or cyano, wherein the (1-4C)alkyl is optionally substituted by cyano, —NR_{c}R_{d} or —OR₀, wherein R_{c} and R_{d} are each independently selected from hydrogen or methyl;
and wherein any available N atoms are optionally substituted by one or more R_{10N}, wherein R_{10N} is methyl.
(23) R₄ and R₅ are linked such that, together with the nitrogen atom to which they are attached, they form a 4 to 12 membered heterocyclic ring and wherein the heterocyclic ring is optionally substituted with one or more R_{10C} or R_{10N} substituent groups, wherein R_{10C} and R_{10N} are as defined in any one of paragraphs (14) to (22) above;
(24) R₄ and R₅ are linked such that, together with the nitrogen atom to which they are attached, they form a 4 to 8 membered monocyclic or bridged heterocyclic ring or a 7 to 12-membered bicyclic or spiro bicyclic heterocyclic ring, and wherein the heterocyclic ring is optionally substituted with one or more R_{10C} or R_{10N} substituent groups, wherein R_{10C} and R_{10N} are as defined in any one of paragraphs (14) to (22) above;
(25) R₄ and R₅ are linked such that, together with the nitrogen atom to which they are attached, they form a 4 to 7 membered monocyclic or bridged heterocyclic ring or a 7 to 11-membered bicyclic or spiro bicyclic heterocyclic ring, and wherein the heterocyclic ring is optionally substituted with one or more R_{10C} or R_{10N} substituent groups, wherein R_{10C} and R_{10N} are as defined in any one of paragraphs (14) to (22) above;
(26) R₄ and R₅ are linked such that, together with the nitrogen atom to which they are attached, they form a 5 or 6 membered monocyclic heterocyclic ring or a 7 to 10-membered bicyclic or spiro bicyclic heterocyclic ring, and wherein the heterocyclic ring is optionally substituted with one or more R_{10C} or R_{10N} substituent groups, wherein R_{10C} and R_{10N} are as defined in any one of paragraphs (14) to (22) above;
(27) R₄ and R₅ are linked such that, together with the nitrogen atom to which they are attached, they form a heterocyclic ring selected from any one of the following options:

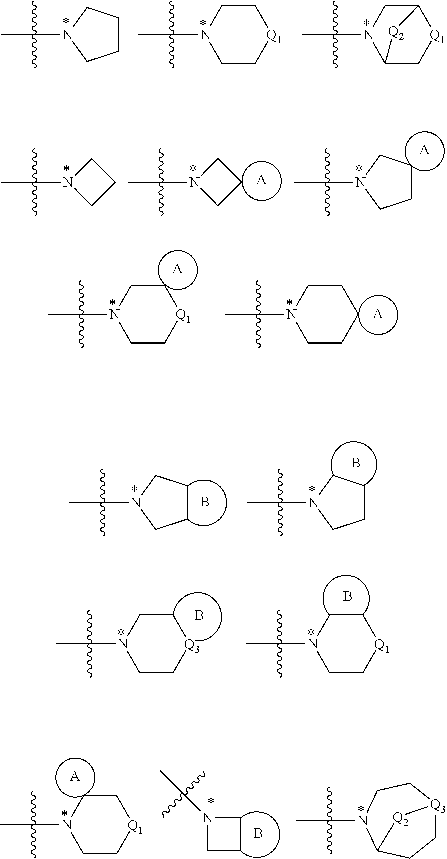

wherein * denotes the N atom to which R₄ and R₅ are attached;
Q₁ is O, NH, S, S(O), S(O)₂, S(O)(═NRₑ), N—R_{10N}, CH₂, CHR_{10C} or C(R_{10c})₂;
Q₂ is —CH₂—, —CHR_{10C}—, —C(R_{10c})₂—, —CHR_{10C}—CH₂—, —CH₂—CHR_{10C}—, —CHR_{10C}—CHR_{10C}—, —C(R_{10c})₂—CH₂—, or —CH₂—C(R_{10c})₂—;
Q₃ is CH, CR_{10C} or N;
Ring A is a spiro-fused 4, 5 or 6 membered carbocyclic or heterocyclic ring;
Ring B is a fused 4, 5 or 6 membered carbocyclic or heterocyclic ring;
wherein Rₑ is selected from hydrogen or methyl;
and wherein each of the heterocyclic ring systems is optionally substituted with one or more R_{10C} or R_{10N} substituent groups as defined in any one of paragraphs (14) to (22) above;
(28) R₄ and R₅ are linked such that, together with the nitrogen atom to which they are attached, they form a heterocyclic ring selected from any one of the following options:

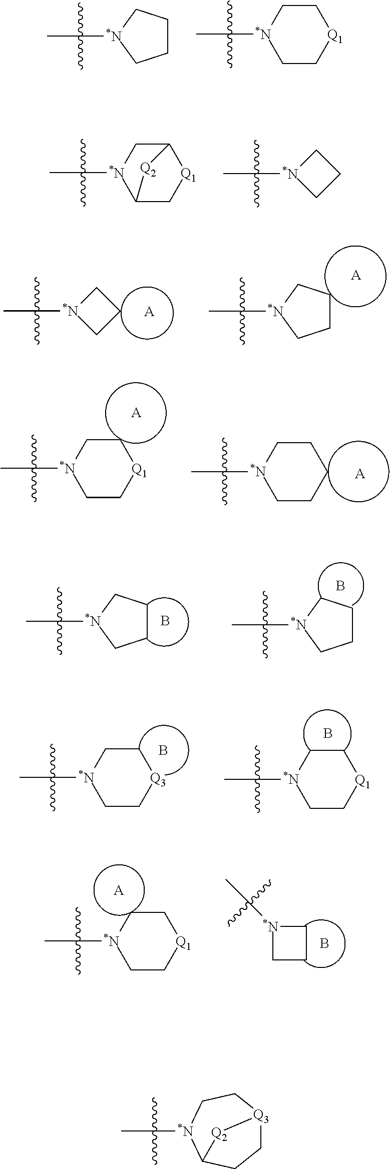

wherein * denotes the N atom to which R₄ and R₅ are attached;
Q₁ is O, NH, S, S(O), S(O)₂, S(O)(═NRₑ), N—R_{10N}, CH₂, CHR_{10C} or C(R_{10c})₂;
Q₂ is —CH₂—, —CHR_{10C}—, or —C(R_{10c})₂—;
Q₃ is CH, CR_{10C} or N;
Ring A is a spiro-fused 4, 5 or 6 membered carbocyclic or heterocyclic ring;
Ring B is a fused 4, 5 or 6 membered carbocyclic or heterocyclic ring;
wherein Rₑ is selected from hydrogen, (1-3C)alkyl or (2-3C)alkanoyl;
and wherein each of the heterocyclic ring systems is optionally substituted with one or more R_{10C} or R_{10N} substituent groups as defined in any one of paragraphs (14) to (22) above;
(29) R₄ and R₅ are linked such that, together with the nitrogen atom to which they are attached, they form a heterocyclic ring selected from any one of the following options:

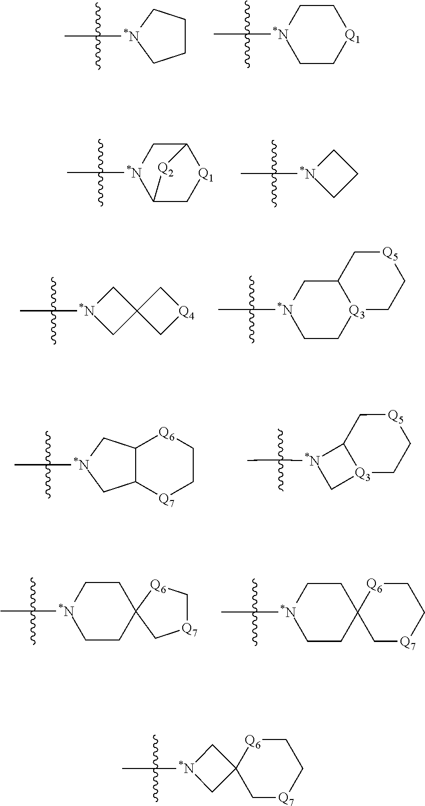

wherein * denotes the N atom to which R₄ and R₅ are attached;
Q₁ is O, NH, S, S(O), S(O)₂, S(O)(═NRₑ), N—R_{10N}, CH₂, CHR_{10C} or C(R_{10c})₂;
Q₂ is —CH₂—;
Q₃ is CH, CR_{10C} or N;
Q₄ is O, NH, S, S(O), S(O)₂, S(O)(═NRₑ), N—R_{10N}, CH₂, CHR_{10C} or C(R_{10c})₂;
Q₅ is O, NH, S, S(O), S(O)₂, S(O)(═NRₑ), N—R_{10N}, CH₂, CHR_{10C} or C(R_{10c})₂;
Q₆ is O, NH, S, S(O), S(O)₂, S(O)(═NRₑ), N—R_{10N}, CH₂, CHR_{10C} or C(R_{10c})₂;
Q₇ is O, NH, S, S(O), S(O)₂, S(O)(═NRₑ), N—R_{10N}, CH₂, CHR_{10C} or C(R_{10c})₂;
wherein Rₑ is selected from hydrogen, (1-2C)alkyl or (2C)alkanoyl;
and wherein each of the heterocyclic ring systems is optionally substituted with one or more R_{10C} or R_{10N} substituent groups as defined in any one of paragraphs (14) to (22) above;
(30) R₄ and R₅ are linked such that, together with the nitrogen atom to which they are attached, they form a heterocyclic ring selected from any one of the following options:

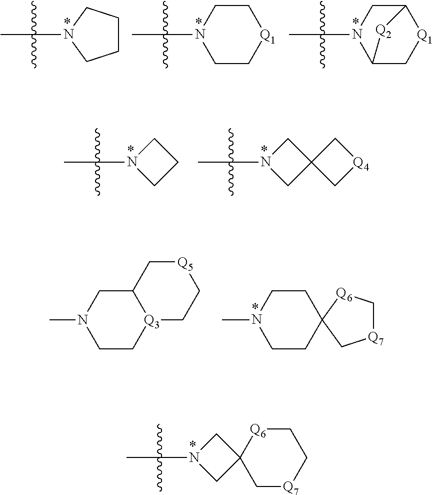

wherein * denotes the N atom to which R₄ and R₅ are attached;
Q₁ is O, NH, S, S(O), S(O)₂, S(O)(═NRₑ), N—R_{10N}, CH₂, CHR_{10C} or C(R_{10c})₂;
Q₂ is —CH₂—;
Q₃ is CH, CR_{10C} or N;
Q₄ is O, NH, S, S(O), S(O)₂, S(O)(═NRₑ), N—R_{10N}, CH₂, CHR_{10C} or C(R_{10c})₂;
Q₅ is O, NH, S, S(O), S(O)₂, S(O)(═NRₑ), N—R_{10N}, CH₂, CHR_{10C} or C(R_{10c})₂;
Q₆ is O, NH, S, S(O), S(O)₂, S(O)(═NRₑ), N—R_{10N}, CH₂, CHR_{10C} or C(R_{10c})₂;
Q₇ is O, NH, S, S(O), S(O)₂, S(O)(═NRₑ), N—R_{10N}, CH₂, CHR_{10C} or C(R_{10c})₂;
wherein Rₑ is selected from hydrogen, (1-2C)alkyl or (2C)alkanoyl;
and wherein each of the heterocyclic ring systems is optionally substituted with one or more R_{10C} or R_{10N} substituent groups as defined in any one of paragraphs (14) to (22) above;
(33) R₄ and R₅ are linked such that, together with the nitrogen atom to which they are attached, they form a heterocyclic ring selected from any one of the following options:

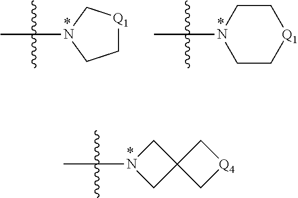

wherein * denotes the N atom to which R₄ and R₅ are attached;
Q₁ is O, NH, S, S(O), S(O)₂, S(O)(═NRₑ), N—R_{10N}, CH₂, CHR_{10C} or C(R_{10c})₂;
Q₄ is O, NH, S, S(O), S(O)₂, S(O)(═NRₑ), N—R_{10N}, CH₂, CHR_{10C} or C(R_{10c})₂;
wherein Rₑ is selected from hydrogen, (1-2C)alkyl or (2C)alkanoyl;
and wherein each of the heterocyclic ring systems is optionally substituted with one or more R_{10C} or R_{10N} substituent groups as defined in any one of paragraphs (14) to (22) above;
(34) R₄ and R₅ are linked such that, together with the nitrogen atom to which they are attached, they form a heterocyclic ring selected from any one of the following options:

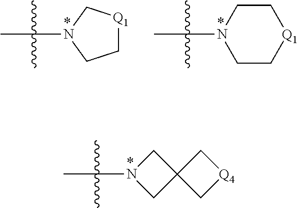

wherein * denotes the N atom to which R₄ and R₅ are attached;
Q₁ is O, NH, N—R_{10N}, CH₂, CHR_{10C} or C(R_{10c})₂;
Q₄ is O, NH, N—R_{10N}, CH₂, CHR_{10C} or C(R_{10c})₂;
wherein Rₑ is selected from hydrogen, methyl or (2C)alkanoyl;
and R_{10C} or R_{10N} are as defined in any one of paragraphs (14) to (22) above;
(35) R₄ and R₅ are linked such that, together with the nitrogen atom to which they are attached, they form a heterocyclic ring selected from any one of the following options:

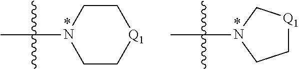

wherein * denotes the N atom to which R₄ and R₅ are attached;
Q₁ is O, NH, S(O), S(O)₂, S(O)(═NRₑ), N—R_{10N}, CH₂, CHR_{10C} or C(R_{10c})₂;
wherein Rₑ is selected from hydrogen, methyl or (2C)alkanoyl;
and R_{10C} or R_{10N} are as defined in any one of paragraphs (14) to (22) above;
(36) R₄ and R₅ are linked such that, together with the nitrogen atom to which they are attached, they form a heterocyclic ring of the structure:

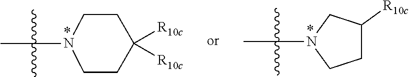

wherein * denotes the N atom to which R₄ and R₅ are attached;
and each R_{10c} is independently selected from the options defined in any one of paragraphs (14) to (22) above;
(37) R₆ is selected from hydrogen, halo or methyl;
(38) R₆ is selected from hydrogen or methyl;
(39) R₆ is hydrogen.

Suitably, a heteroaryl or heterocyclyl group as defined herein is a monocyclic heteroaryl or mono, bicyclic, spiro bicyclic or bridged heterocyclyl group comprising one, two or three heteroatoms selected from N, O or S.

Suitably, a heteroaryl is a 5- or 6-membered heteroaryl ring comprising one, two or three heteroatoms selected from N, O or S.

Suitably, a heterocyclyl group is a 4-, 5-, 6-, 7-, 8-, 9-, 10-, 11-, or 12-membered heterocyclyl ring comprising one, two or three heteroatoms selected from N, O or S. Most suitably, a heterocyclyl group is a 5-, 6- or 7-membered ring comprising one, two or three heteroatoms selected from N, O or S [e.g. morpholinyl (e.g. 4-morpholinyl), pyridinyl, piperazinyl, homopiperazinyl or pyrrolidinonyl] or a 7-, 8-, 9- or 10-membered spiro bicyclic ring system comprising one, two or three heteroatoms selected from N, O or S.

Suitably, an aryl group is phenyl.

Suitably, R₁ is as defined in any one of paragraphs (1) to (7) above. More suitably, R₁ is as defined in paragraphs (3) to (7) above. Most suitably, R₁ is as defined in paragraph (5) or (7) above.

Suitably, R₂ is as defined in any one of paragraphs (8) to (11) above. More suitably, R₂ is as defined in any one of paragraphs (9) to (11). Most suitably, R₂ is as defined in paragraph (10) or (11) above.

Suitably, R₃ is as defined in paragraph (11a), (11 b), (12) or (13) above. More suitably, R₃ is as defined in paragraph (12) or (13) above. Most suitably, R₃ is as defined in paragraph (13) above.

Suitably, R₄ and R₅ are linked such that, together with the nitrogen atom to which they are attached, they form a heterocyclic ring which is optionally substituted as defined in any one of paragraphs (14) to (22) above. More suitably the heterocyclic ring formed by R₄ and R₅ is optionally substituted as defined in any one of paragraphs (19) to (22) above. Most suitably the heterocyclic ring formed by R₄ and R₅ is optionally substituted as defined in paragraph (21) or (22) above.

Suitably, R₄ and R₅ are as defined in any one of paragraphs (23) to (36) above. More suitably, R₄ and R₅ are as defined in any one of paragraphs (27) to (36) above. Most suitably, R₄ and R₅ are as defined in paragraph (35) or (36) above.

Suitably, R_{10C} and R_{10N} are as defined in any one of paragraphs (14) to (22) above. More suitably, R_{10C} and R_{10N} are as defined in any one of paragraphs (19) to (22) above. Even more suitably, R_{10C} and R_{10N} are as defined in any one of paragraphs (20), (21) or (22) above.

Suitably, R₆ is as defined in any one of paragraphs (37) to (39) above. More suitably, R₆ is as defined in paragraph (39) above, i.e. R₆ is hydrogen.

In a particular group of compounds of Formula I above, R₁ is as defined in any one of paragraphs (1), (2), (3), (4), (5), (6) or (7), and R₂, R₃, R₄, R₅ and R₆ each have any one of the definitions set out herein.

In a particular group of compounds of Formula I above, R₁ is as defined in paragraph (4), i.e. the compounds have the structural formula IA (a sub-formula of Formula I) shown below:

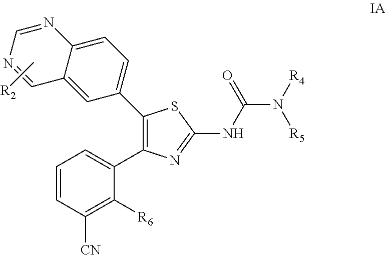

wherein R₂, R₄, R₅ and R₆ each have any one of the definitions set out herein.

In an embodiment of the compounds of formula IA:
R₂ is as defined in any one of paragraphs (8) to (11) above;
R₄ and R₅ are as defined in any one of paragraphs (23) to (36) above;
the heterocyclic ring formed by R₄ and R₅ is optionally substituted as defined in any one of paragraphs (14) to (22) above; and
R₆ is as defined in any one of paragraphs (37) to (39) above.

In an embodiment of the compounds of formula IA:
R₂ is as defined in any one of paragraphs (9) to (11) above;
R₄ and R₅ are as defined in any one of paragraphs (27) to (36) above;
the heterocyclic ring formed by R₄ and R₅ is optionally substituted as defined in any one of paragraphs (19) to (22) above; and
R₆ is as defined in paragraph (38) or (39) above.

In an embodiment of the compounds of formula IA:
R₂ is as defined in paragraph (10) or (11) above;
R₄ and R₅ are as defined in paragraph (35) or (36) above;
the heterocyclic ring formed by R₄ and R₅ is optionally substituted as defined in paragraph (21) or (22) above; and
R₆ is as defined in paragraph (38) or (39) above.

In an embodiment of the compounds of formula IA:
R₂ is as defined in paragraph (11) above;
R₄ and R₅ are as defined in paragraph (35) above;
the heterocyclic ring formed by R₄ and R₅ is optionally substituted as defined in any one of paragraphs (19) to (22) above; and
R₆ is as defined in paragraph (38) or (39) above.

In an embodiment of the compounds of formula IA:
R₂ is as defined in paragraph (11) above;
R₄ and R₅ are as defined in any one of paragraphs (27) to (36) above; the heterocyclic ring formed by R₄ and R₅ is optionally substituted as defined in paragraph (21) or (22) above; and
R₆ is as defined in paragraph (38) or (39) above.

In a particular group of compounds of Formula I above, R₁ is as defined in paragraph (5), i.e. the compounds have the structural formula IB (a sub-formula of Formula I) shown below:

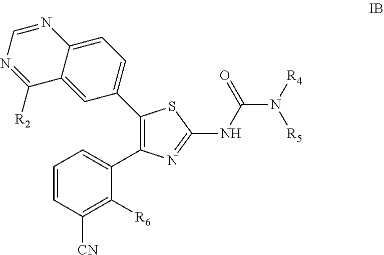

wherein R₂, R₄, R₅ and R₆ each have any one of the definitions set out herein.

In an embodiment of the compounds of formula IB:
R₂ is as defined in any one of paragraphs (8) to (11) above;
R₄ and R₅ are as defined in any one of paragraphs (23) to (36) above; the heterocyclic ring formed by R₄ and R₅ is optionally substituted as defined in any one of paragraphs (14) to (22) above; and
R₆ is as defined in any one of paragraphs (37) to (39) above.

In an embodiment of the compounds of formula IB:
R₂ is as defined in any one of paragraphs (9) to (11) above;
R₄ and R₅ are as defined in any one of paragraphs (27) to (36) above; the heterocyclic ring formed by R₄ and R₅ is optionally substituted as defined in any one of paragraphs (19) to (22) above; and
R₆ is as defined in paragraph (38) or (39) above.

In an embodiment of the compounds of formula IB:
R₂ is as defined in paragraph (10) or (11) above;
R₄ and R₅ are as defined in paragraph (35) or (36) above;
the heterocyclic ring formed by R₄ and R₅ is optionally substituted as defined in paragraph (21) or (22) above; and
R₆ is as defined in paragraph (38) or (39) above.

In an embodiment of the compounds of formula IB:
R₂ is as defined in paragraph (11) above;
R₄ and R₅ are as defined in paragraph (35) above;
the heterocyclic ring formed by R₄ and R₅ is optionally substituted as defined in any one of paragraphs (19) to (22) above; and
R₆ is as defined in paragraph (38) or (39) above.

In an embodiment of the compounds of formula IB:
R₂ is as defined in paragraph (11) above;
R₄ and R₅ are as defined in any one of paragraphs (27) to (36) above; the heterocyclic ring formed by R₄ and R₅ is optionally substituted as defined in paragraph (21) or (22) above; and
R₆ is as defined in paragraph (38) or (39) above.

In a particular group of compounds of Formula I above, R₁ is as defined in paragraph (7), i.e. the compounds have the structural formula IC (a sub-formula of Formula I) shown below:

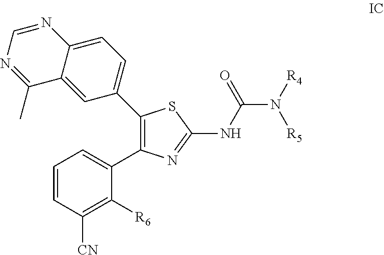

wherein R₁, R₄, R₅ and R₆ each have any one of the definitions set out herein.

In an embodiment of the compounds of formula IC:
R₄ and R₅ are as defined in any one of paragraphs (23) to (36) above; the heterocyclic ring formed by R₄ and R₅ is optionally substituted as defined in any one of paragraphs (14) to (22) above; and
R₆ is as defined in any one of paragraphs (37) to (39) above.

In an embodiment of the compounds of formula IC:
R₄ and R₅ are as defined in any one of paragraphs (27) to (36) above; the heterocyclic ring formed by R₄ and R₅ is optionally substituted as defined in any one of paragraphs (19) to (22) above; and
R₆ is as defined in paragraph (38) or (39) above.

In an embodiment of the compounds of formula IC:
R₄ and R₅ are as defined in paragraph (35) or (36) above;
the heterocyclic ring formed by R₄ and R₅ is optionally substituted as defined in paragraph (21) or (22) above; and
R₆ is as defined in paragraph (38) or (39) above.

In an embodiment of the compounds of formula IC:
R₄ and R₅ are as defined in paragraph (35) above;
the heterocyclic ring formed by R₄ and R₅ is optionally substituted as defined in any one of paragraphs (19) to (22) above; and
R₆ is as defined in paragraph (38) or (39) above.

In an embodiment of the compounds of formula IC:
R₄ and R₅ are as defined in any one of paragraphs (27) to (36) above; the heterocyclic ring formed by R₄ and R₅ is optionally substituted as defined in paragraph (21) or (22) above; and
R₆ is as defined in paragraph (38) or (39) above.

In a particular group of compounds of Formula I above, R₆ is hydrogen, i.e. the compounds have the structural formula ID (a sub-formula of Formula I′) shown below:

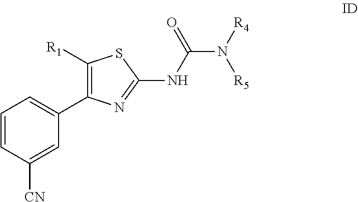

wherein R₁, R₄ and R₅ each have any one of the definitions set out herein.

In an embodiment of the compounds of formula ID:
Suitably, R₁ is as defined in any one of paragraphs (1) to (7) above;
R₄ and R₅ are as defined in any one of paragraphs (23) to (36) above;
the heterocyclic ring formed by R₄ and R₅ is optionally substituted as defined in any one of paragraphs (14) to (22) above; and
R₆ is as defined in any one of paragraphs (37) to (39) above.

In an embodiment of the compounds of formula ID:
R₁ is as defined in paragraphs (3) to (7) above;
R₄ and R₅ are as defined in any one of paragraphs (27) to (36) above;
the heterocyclic ring formed by R₄ and R₅ is optionally substituted as defined in any one of paragraphs (19) to (22) above; and
R₆ is as defined in paragraph (38) or (39) above.

In an embodiment of the compounds of formula ID:
R₁ is as defined in paragraphs (3) to (7) above R₄ and R₅ are as defined in paragraph (35) or (36) above;
the heterocyclic ring formed by R₄ and R₅ is optionally substituted as defined in paragraph (21) or (22) above; and
R₆ is as defined in paragraph (38) or (39) above.

In an embodiment of the compounds of formula ID:
R₁ is as defined in paragraph (5), (6) or (7) above;
R₄ and R₅ are as defined in paragraph (35) above;
the heterocyclic ring formed by R₄ and R₅ is optionally substituted as defined in any one of paragraphs (19) to (22) above; and
R₆ is as defined in paragraph (38) or (39) above.

In an embodiment of the compounds of formula ID:
R₁ is as defined in paragraphs (3) to (7) above;
R₄ and R₅ are as defined in any one of paragraphs (27) to (36) above;
the heterocyclic ring formed by R₄ and R₅ is optionally substituted as defined in paragraph (21) or (22) above; and
R₆ is as defined in paragraph (38) or (39) above.

In a particular group of compounds of Formula I above, R₁ is as defined in paragraph (4), and R₄ and R₅ are as defined in paragraph (36), i.e. the compounds have the structural formula IE or IF (a sub-formula of Formula I) shown below:

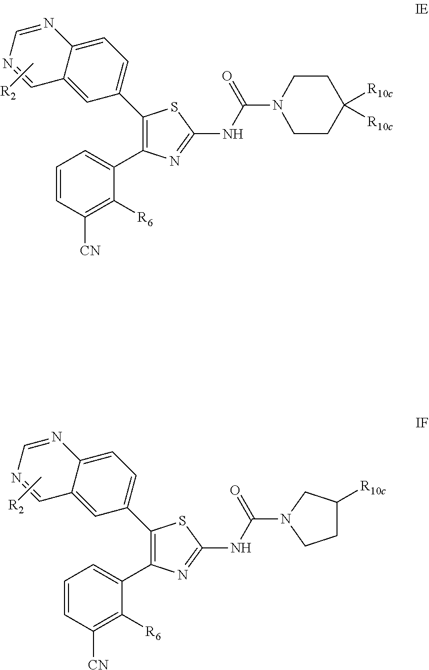

wherein R₂, R₆ and R_{10c} each have any one of the definitions set out herein.

In an embodiment of the compounds of formula IE or IF:
R₂ is as defined in any one of paragraphs (8) to (11) above;
R₆ is as defined in any one of paragraphs (37) to (39) above; and
R_{10c} is as defined in any one of paragraphs (14) to (22) above.

In an embodiment of the compounds of formula IE or IF:
R₂ is as defined in any one of paragraphs (9) to (11) above;
R₆ is as defined in paragraph (38) or (39) above; and
R_{10c} is as defined in any one of paragraphs (19) to (22) above.

In an embodiment of the compounds of formula IE or IF:
R₂ is as defined in paragraph (10) or (11) above;
R₆ is as defined in paragraph (38) or (39) above; and
R_{10c} is as defined in any one of paragraphs (20), (21) or (22) above.

Particular compounds of the present invention include any of the compounds described in the example section of the present application, or a pharmaceutically acceptable salt or solvate thereof, and, in particular, any of the following:
(3S)-3-Cyano-N-[4-(3-cyanophenyl)-5-(4-methylquinazolin-6-yl)thiazol-2-yl]-3-methyl-pyrrolidine-1-carboxamide;
N-[4-(3-Cyanophenyl)-5-(4-methylquinazolin-6-yl)thiazol-2-yl]-4-oxa-1,9-diazaspiro[5.5]undecane-9-carboxamide;
N-[4-(3-Cyanophenyl)-5-(4-methylquinazolin-6-yl)thiazol-2-yl]-2-(cyclopropylmethyl)piperazine-1-carboxamide;
N-[4-(3-Cyanophenyl)-5-(4-methyl-1H-indazol-6-yl)thiazol-2-yl]-2-oxa-6-azaspiro[3.3]heptane-6-carboxamide;
N-[4-(3-Cyanophenyl)-5-(7-methyl-3H-benzotriazol-5-yl)thiazol-2-yl]-2-oxa-6-azaspiro[3.3]heptane-6-carboxamide;
N-[4-(3-Cyanophenyl)-5-(1H-pyrrolo[2,3-b]pyridin-4-yl)thiazol-2-yl]-2-oxa-6-azaspiro[3.3]heptane-6-carboxamide;
N-[4-(3-Cyanophenyl)-5-(6-methyl-1H-pyrazolo[3,4-b]pyridin-4-yl)thiazol-2-yl]-2-oxa-6-azaspiro[3.3]heptane-6-carboxamide;
N-[4-(3-Cyanophenyl)-5-(4-methylquinazolin-6-yl)thiazol-2-yl]-4-piperazin-1-yl-piperidine-1-carboxamide;
N-[4-(3-Cyanophenyl)-5-(4-methylquinazolin-6-yl)thiazol-2-yl]-1-oxa-4,9-diazaspiro[5.5]undecane-9-carboxamide;
N-[4-(3-Cyanophenyl)-5-(4-methylquinazolin-6-yl)thiazol-2-yl]-6-oxa-2,9-diazaspiro[4.5]decane-2-carboxamide;
N-[4-(3-Cyanophenyl)-5-(4-methylquinazolin-6-yl)thiazol-2-yl]-5-oxa-2,8-diazaspiro[3.5]nonane-2-carboxamide;
N-[4-(3-Cyanophenyl)-5-(4-methylquinazolin-6-yl)thiazol-2-yl]-8-oxa-2,5-diazaspiro[3.5]nonane-2-carboxamide;
N-[4-(3-Cyanophenyl)-5-(4-methylquinazolin-6-yl)thiazol-2-yl]-9-oxa-2,6-diazaspiro[4.5]decane-2-carboxamide;
(3S)—N-[4-(3-Cyanophenyl)-5-(4-methylquinazolin-6-yl)thiazol-2-yl]-3-(1-hydroxy-1-methyl-ethyl)pyrrolidine-1-carboxamide;
4-Cyano-N-[4-(3-cyanophenyl)-5-(4-methylquinazolin-6-yl)thiazol-2-yl]-4-methyl-piperidine-1-carboxamide;
(3R)—N-[4-(3-Cyanophenyl)-5-(4-methylquinazolin-6-yl)thiazol-2-yl]-3-(1-hydroxy-1-methyl-ethyl)pyrrolidine-1-carboxamide;
(3R)-3-Cyano-N-[4-(3-cyanophenyl)-5-(4-methylquinazolin-6-yl)thiazo-2-yl]-3-methyl-pyrrolidine-1-carboxamide;
4-(1-Cyano-1-methyl-ethyl)-N-[4-(3-cyanophenyl)-5-(4-methylquinazolin-6-yl)thiazol-2-yl]piperazine-1-carboxamide;
N-[4-(3-Cyanophenyl)-5-(4-methylquinazolin-6-yl)thiazol-2-yl]-3-methyl-3,6-diazabicyclo[3.2.0]heptane-6-carboxamide;
N-[4-(3-Cyanophenyl)-5-(4-methylquinazolin-6-yl)thiazol-2-yl]-6-methyl-2,6-diazaspiro[3.3]heptane-2-carboxamide;
4-(1-Cyanoethyl)-N-[4-(3-cyanophenyl)-5-(4-methylquinazolin-6-yl)thiazol-2-yl]piperazine-1-carboxamide;
N-[4-(3-Cyanophenyl)-5-(4-methylquinazolin-6-yl)thiazol-2-yl]-4-(dimethylamino)piperidine-1-carboxamide;
N-[4-(3-Cyanophenyl)-5-(4-methylquinazolin-6-yl)thiazol-2-yl]-2-(1-hydroxy-1-methyl-ethyl)morpholine-4-carboxamide;
N-[4-(3-Cyanophenyl)-5-(4-methylquinazolin-6-yl)thiazol-2-yl]-2-[(dimethylamino)methyl]morpholine-4-carboxamide;
N-[4-(3-Cyanophenyl)-5-(4-methylquinazolin-6-yl)thiazol-2-yl]-3-(2-pyrrolidin-1-ylethoxy)pyrrolidine-1-carboxamide;
N-[4-(3-Cyanophenyl)-5-(4-methylquinazolin-6-yl)thiazol-2-yl]-4-methyl-1-oxa-4,9-diazaspiro[5.5]undecane-9-carboxamide;
N-[4-(3-Cyanophenyl)-5-(4-methylquinazolin-6-yl)thiazol-2-yl]-1-methyl-3,4,4a,5,7,7a-hexahydro-2H-pyrrolo[3,4-b]pyridine-6-carboxamide;
N-[4-(3-Cyanophenyl)-5-(4-methylquinazolin-6-yl)thiazol-2-yl]-4-(4-methylpiperazin-1-yl)piperidine-1-carboxamide;
N-[4-(3-Cyanophenyl)-5-(4-methylquinazolin-6-yl)thiazol-2-yl]-4-(1-methyl-4-piperidyl)piperazine-1-carboxamide;
N-[4-(3-Cyanophenyl)-5-(4-methylquinazolin-6-yl)thiazol-2-yl]-4-morpholino-piperidine-1-carboxamide;
4-(Cyanomethyl)-N-[4-(3-cyanophenyl)-5-(4-methylquinazolin-6-yl)thiazol-2-yl]piperazine-1-carboxamide;
(3S)—N-[4-(3-Cyanophenyl)-5-(4-methylquinazolin-6-yl)thiazol-2-yl]-3-(1-hydroxy-1-methyl-ethyl)piperazine-1-carboxamide;
N-[4-(3-Cyanophenyl)-5-(4-methylquinazolin-6-yl)thiazol-2-yl]-3-oxo-2,8-diazaspiro[4.5]decane-8-carboxamide;
N-[4-(3-Cyanophenyl)-5-(4-methylquinazolin-6-yl)thiazol-2-yl]-1,4-diazepane-1-carboxamide;
N-[4-(3-Cyanophenyl)-5-(4-methylquinazolin-6-yl)thiazol-2-yl]-1-oxa-6-azaspiro[3.3]heptane-6-carboxamide;
(4aR,7aS)—N-[4-(3-Cyanophenyl)-5-(4-methylquinazolin-6-yl)thiazol-2-yl]-4-methyl-2,3,4a,5,7,7a-hexahydropyrrolo[3,4-b][1,4]oxazine-6-carboxamide;
N-[4-(3-Cyanophenyl)-5-(4-methylquinazolin-6-yl)thiazol-2-yl]piperazine-1-carboxamide;
N-[4-(3-Cyanophenyl)-5-(4-methylquinazolin-6-yl)thiazol-2-yl]-4-methyl-piperazine-1-carboxamide;
N-[4-(3-Cyanophenyl)-5-(4-methylquinazolin-6-yl)thiazol-2-yl]-2-oxo-1,8-diazaspiro[4.5]decane-8-carboxamide;
N-[4-(3-Cyanophenyl)-5-(4-methylquinazolin-6-yl)thiazol-2-yl]-6-hydroxy-6-methyl-2-azaspiro[3.3]heptane-2-carboxamide;
3-Cyano-N-[4-(3-cyanophenyl)-5-(4-methylquinazolin-6-yl)thiazol-2-yl]-3-methyl-pyrrolidine-1-carboxamide;
N-[4-(3-Cyanophenyl)-5-(4-methylquinazolin-6-yl)thiazol-2-yl]-3-methyl-2-oxo-1-oxa-3,8-diazaspiro[4.5]decane-8-carboxamide;
N-[4-(3-Cyanophenyl)-5-(4-methylquinazolin-6-yl)thiazol-2-yl]-4-(2-hydroxyethyl)piperazine-1-carboxamide;
N-[4-(3-Cyanophenyl)-5-(4-methylquinazolin-6-yl)thiazol-2-yl]-4-(oxetan-3-yl)piperazine-1-carboxamide;
N-[4-(3-Cyanophenyl)-5-(4-methylquinazolin-6-yl)thiazol-2-yl]-2-oxa-6-azaspiro[3.3]heptane-6-carboxamide;
(2S)—N-[4-(3-Cyanophenyl)-5-(4-methylquinazolin-6-yl)thiazol-2-yl]-2-(1-hydroxy-1-methyl-ethyl)morpholine-4-carboxamide;
(2R)—N-[4-(3-cyanophenyl)-5-(4-methylquinazolin-6-yl)thiazol-2-yl]-2-(1-hydroxy-1-methyl-ethyl)morpholine-4-carboxamide;
(2S)—N-[4-(3-Cyanophenyl)-5-(4-methylquinazolin-6-yl)thiazol-2-yl]-2-(1-hydroxy-1-methyl-ethyl)morpholine-4-carboxamide;
(2R)—N-[4-(3-cyanophenyl)-5-(4-methylquinazolin-6-yl)thiazol-2-yl]-2-(1-hydroxy-1-methyl-ethyl)morpholine-4-carboxamide;
N-[4-(3-Cyanophenyl)-5-(2-methylquinazolin-7-yl)thiazol-2-yl]-2-oxa-6-azaspiro[3.3]heptane-6-carboxamide;
N-[4-(3-Cyanophenyl)-5-(2,4-dimethylquinazolin-6-yl)thiazol-2-yl]-2-oxa-6-azaspiro[3.3]heptane-6-carboxamide;
N-[4-(3-Cyanophenyl)-5-quinazolin-6-yl-thiazol-2-yl]-2-oxa-6-azaspiro[3.3]heptane-6-carboxamide;
N-[4-(3-Cyanophenyl)-5-(4-methyl-6-quinolyl)thiazol-2-yl]-2-oxa-6-azaspiro[3.3]heptane-6-carboxamide;
N-[4-(3-Cyanophenyl)-5-(4-methoxyquinazolin-6-yl)thiazol-2-yl]-2-oxa-6-azaspiro[3.3]heptane-6-carboxamide;
N-[4-(3-Cyanophenyl)-5-(4-ethylquinazolin-6-yl)thiazol-2-yl]-2-oxa-6-azaspiro[3.3]heptane-6-carboxamide;
4-Cyano-N-[4-(3-cyanophenyl)-5-(2,4-dimethyloxazol-5-yl)thiazol-2-yl]-4-methyl-piperidine-1-carboxamide;
N-[4-(3-Cyanophenyl)-5-(2,4-dimethyloxazol-5-yl)thiazol-2-yl]-2-oxa-6-azaspiro[3.3]heptane-6-carboxamide;
N-[4-(3-Cyanophenyl)-5-(1-methylpyrrolo[2,3-b]pyridin-3-yl)thiazol-2-yl]-2-oxa-6-azaspiro[3.3]heptane-6-carboxamide;
N-[4-(3-Cyanophenyl)-5-(1H-pyrrolo[2,3-b]pyridin-3-yl)thiazol-2-yl]-2-oxa-6-azaspiro[3.3]heptane-6-carboxamide;
N-[4-(3-Cyanophenyl)-5-(3,8-dimethylimidazo[1,2-a]pyridin-6-yl)thiazol-2-yl]-2-oxa-6-azaspiro[3.3]heptane-6-carboxamide;
4-Cyano-N-[4-(3-cyanophenyl)-5-(5-methyl-1H-pyrazolo[4,3-b]pyridin-7-yl)thiazol-2-yl]-4-methyl-piperidine-1-carboxamide;
N-[4-(3-Cyanophenyl)-5-(4,8-dimethylquinazolin-6-yl)thiazol-2-yl]-2-oxa-6-azaspiro[3.3]heptane-6-carboxamide;
N-[4-(3-Cyanophenyl)-5-(1-methylpyrrolo[2,3-b]pyridin-4-yl)thiazol-2-yl]-2-oxa-6-azaspiro[3.3]heptane-6-carboxamide;
N-[4-(3-Cyanophenyl)-5-(3-methylimidazo[1,2-a]pyridin-6-yl)thiazol-2-yl]-2-oxa-6-azaspiro[3.3]heptane-6-carboxamide;
N-[4-(3-Cyanophenyl)-5-(5-methyl-1H-pyrazolo[4,3-b]pyridin-7-yl)thiazol-2-yl]-2-oxa-6-azaspiro[3.3]heptane-6-carboxamide;
N-[4-(3-Cyanophenyl)-5-(1,6-dimethylpyrrolo[2,3-b]pyridin-4-yl)thiazol-2-yl]-2-oxa-6-azaspiro[3.3]heptane-6-carboxamide;
N-[4-(3-Cyanophenyl)-5-(3-methylbenzimidazol-5-yl)thiazol-2-yl]-2-oxa-6-azaspiro[3.3]heptane-6-carboxamide;
N-[4-(3-Cyanophenyl)-5-imidazo[1,2-a]pyridin-6-yl-thiazol-2-yl]-2-oxa-6-azaspiro[3.3]heptane-6-carboxamide;
N-[4-(3-Cyanophenyl)-5-(7-methyl-1H-indazol-5-yl)thiazol-2-yl]-2-oxa-6-azaspiro[3.3]heptane-6-carboxamide;
N-[4-(3-Cyanophenyl)-5-(2,7-dimethylindazol-5-yl)thiazol-2-yl]-2-oxa-6-azaspiro[3.3]heptane-6-carboxamide;
N-[4-(3-Cyanophenyl)-5-(8-methylimidazo[1,2-a]pyridin-6-yl)thiazol-2-yl]-2-oxa-6-azaspiro[3.3]heptane-6-carboxamide;
N-[4-(3-Cyanophenyl)-5-(1,7-dimethylindazol-5-yl)thiazol-2-yl]-2-oxa-6-azaspiro[3.3]heptane-6-carboxamide;
N-[4-(3-Cyanophenyl)-5-(6-methylpyridazin-4-yl)thiazol-2-yl]-2-oxa-6-azaspiro[3.3]heptane-6-carboxamide;
N-[4-(3-Cyanophenyl)-5-[5-methyl-6-(triazol-2-yl)-3-pyridyl]thiazol-2-yl]-2-oxa-6-azaspiro[3.3]heptane-6-carboxamide;
N-[4-(3-Cyanophenyl)-5-pyrazolo[1,5-a]pyridin-5-yl-thiazol-2-yl]morpholine-4-carboxamide;
N-[4-(3-Cyanophenyl)-5-(6-methyl-1H-pyrrolo[2,3-b]pyridin-4-yl)thiazol-2-yl]-2-oxa-6-azaspiro[3.3]heptane-6-carboxamide; or
N-[5-(1,3-benzoxazol-6-yl)-4-(3-cyanophenyl)thiazol-2-yl]-2-oxa-6-azaspiro[3.3]heptane-6-carboxamide.

The various functional groups and substituents making up the compounds of the formula (I) are typically chosen such that the molecular weight of the compound of the formula (I) does not exceed 1000. More usually, the molecular weight of the compound will be less than 900, for example less than 800, or less than 750, or less than 700, or less than 650. More preferably, the molecular weight is less than 600 and, for example, is 550 or less.

A suitable pharmaceutically acceptable salt of a compound of the invention is, for example, an acidaddition salt of a compound of the invention which is sufficiently basic, for example, an acidaddition salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, trimethylamine, piperidine, morpholine or tris(2hydroxyethyl)amine.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are nonsuperimposable mirror images of each other are termed “enantiomers”. When a compound has an asymmetric center-, for example, it is bonded to four different groups, a pair of enantiomers is possible. An enantiomer can be characterized by the absolute configuration of its asymmetric center and is described by the R and S sequencing rules of Cahn and Prelog, or by the manner in which the molecule rotates the plane of polarized light and designated as dextrorotatory or -levorotatory—(i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R) or (S)stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are wellknown in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E and Z isomers). It is to be understood that the present invention encompasses all optical, diastereoisomers and geometric isomers and mixtures thereof that possess antiproliferative activity.

The present invention also encompasses compounds of the invention as defined herein which comprise one or more isotopic substitutions. For Example, H may be in any isotopic form, including 1H, 2H(D), and 3H (T); C may be in any isotopic form, including 12C, 13C, and 14C; and O may be in any isotopic form, including 160 and 180; and the like.

It is also to be understood that certain compounds of the formula (I) may exist in solvated as well as unsolvated forms such as, for example, hydrated forms. It is to be understood that the invention encompasses all such solvated forms that possess antiproliferative activity.

It is also to be understood that certain compounds of the formula I may exhibit polymorphism, and that the invention encompasses all such forms that possess antiproliferative activity.

Compounds of the formula I may exist in a number of different tautomeric forms and references to compounds of the formula I include all such forms. For the avoidance of doubt, where a compound can exist in one of several tautomeric forms, and only one is specifically described or shown, all others are nevertheless embraced by formula I. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

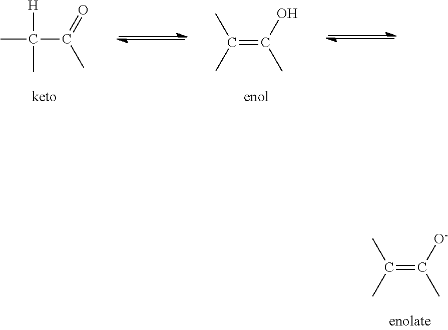

Compounds of the formula I containing an amine function may also form N-oxides.

A reference herein to a compound of the formula I that contains an amine function also includes the N-oxide. Where a compound contains several amine functions, one or more than one nitrogen atom may be oxidised to form an N-oxide. Particular Examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (mCPBA), for example, in an inert solvent such as dichloromethane.

The compounds of formula (I) may be administered in the form of a prodrug which is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached.

Examples of pro-drugs include in vivo cleavable ester derivatives that may be formed at a carboxy group or a hydroxy group in a compound of the formula (I) and in-vivo cleavable amide derivatives that may be formed at a carboxy group or an amino group in a compound of the formula (I).

Accordingly, the present invention includes those compounds of the formula (I) as defined hereinbefore when made available by organic synthesis and when made available within the human or animal body by way of cleavage of a pro-drug thereof. Accordingly, the present invention includes those compounds of the formula I that are produced by organic synthetic means and also such compounds that are produced in the human or animal body by way of metabolism of a precursor compound, that is a compound of the formula (I) may be a synthetically-produced compound or a metabolically-produced compound.

A suitable pharmaceutically acceptable pro-drug of a compound of the formula (I) is one that is based on reasonable medical judgement as being suitable for administration to the human or animal body without undesirable pharmacological activities and without undue toxicity.

Various forms of pro-drug have been described, for example in the following documents:—
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and
H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988); f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically acceptable pro-drug of a compound of the formula I that possesses a carboxy group is, for example, an in vivo cleavable ester thereof. An in vivo cleavable ester of a compound of the formula I containing a carboxy group is, for example, a pharmaceutically acceptable ester which is cleaved in the human or animal body to produce the parent acid. Suitable pharmaceutically acceptable esters for carboxy include C1-6alkyl esters such as methyl, ethyl and tert-butyl, C1-6alkoxymethyl esters such as methoxymethyl esters, C1-6alkanoyloxymethyl esters such as pivaloyloxymethyl esters, 3-phthalidyl esters, C3-8cycloalkylcarbonyloxy-C1-6alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and C1-6alkoxycarbonyloxy-C1-6alkyl esters such as methoxycarbonyloxymethyl and 1-methoxycarbonyloxyethyl esters.

A suitable pharmaceutically acceptable pro-drug of a compound of the Formula (I) that possesses a hydroxy group is, for example, an in vivo cleavable ester or ether thereof.

An in vivo cleavable ester or ether of a compound of the formula I containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C1-10alkanoyl groups such as acetyl, benzoyl, phenylacetyl and substituted benzoyl and phenylacetyl groups, C1-10alkoxycarbonyl groups such as ethoxycarbonyl, N,N-(C1-6)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C1-4alkyl)piperazin-1-ylmethyl. Suitable pharmaceutically acceptable ether forming groups for a hydroxy group include Q-acyloxyalkyl groups such as acetoxymethyl and pivaloyloxymethyl groups.

A suitable pharmaceutically acceptable pro-drug of a compound of the formula (I) that possesses a carboxy group is, for example, an in vivo cleavable amide thereof, for example an amide formed with an amine such as ammonia, a C1-4alkylamine such as methylamine, a (C1-4alkyl)₂amine such as dimethylamine, N-ethyl-N-methylamine or diethylamine, a C1-4alkoxy-C2-4alkylamine such as 2-methoxyethylamine, a phenyl-C1-4alkylamine such as benzylamine and amino acids such as glycine or an ester thereof.

A suitable pharmaceutically acceptable pro-drug of a compound of the formula I that possesses an amino group is, for example, an in vivo cleavable amide derivative thereof.

Suitable pharmaceutically acceptable amides from an amino group include, for example an amide formed with C1-10alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C1-4alkyl)piperazin-1-ylmethyl.

The in vivo effects of a compound of the formula (I) may be exerted in part by one or more metabolites that are formed within the human or animal body after administration of a compound of the formula (I). As stated hereinbefore, the in vivo effects of a compound of the formula (I) may also be exerted by way of metabolism of a precursor compound (a pro-drug).

Though the present invention may relate to any compound or particular group of compounds defined herein by way of optional, preferred or suitable features or otherwise in terms of particular embodiments, the present invention may also relate to any compound or particular group of compounds that specifically excludes said optional, preferred or suitable features or particular embodiments.

Suitably, the present invention excludes any individual compounds not possessing the biological activity defined herein.

### Synthesis

The compounds of the present invention can be prepared by any suitable technique known in the art. Particular methods for forming compounds of formula I defined herein are shown in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the invention in the processes defined herein, or during the synthesis of certain starting materials, it may be desirable to protect certain substituent groups to prevent their undesired reaction.

The skilled chemist will appreciate when such protection is required, and how such protecting groups may be put in place, and later removed.

For Examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of Example, a suitable protecting group for an amino or alkylamino group is, for example, an acyl group, for example an alkanoyl group such as acetyl, an alkoxycarbonyl group, for example a methoxycarbonyl, ethoxycarbonyl or tbutoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tertbutoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladiumoncarbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively, an arylmethyl group such as a benzyl group may be removed, for example, by hydrogenation over a catalyst such as palladiumoncarbon.

A suitable protecting group for a carboxy group is, for example, an esterifying group, for example a methyl or an ethyl group which may be removed, for example, by hydrolysis with a base such as sodium hydroxide, or for example a tbutyl group which may be removed, for example, by treatment with an acid, for example an organic acid such as trifluoroacetic acid, or for example a benzyl group which may be removed, for example, by hydrogenation over a catalyst such as palladiumoncarbon.

Resins may also be used as a protecting group.

The methodology employed to synthesise a compound of formula (I) will vary depending on the nature of R₁, R₂, R₃, R₄, R₅ and R₆ and any substituent groups associated therewith. Suitable processes for their preparation are described further in the accompanying Examples.

Once a compound of formula (I) has been synthesised by any one of the processes defined herein, the processes may then further comprise one or more of the additional steps of:
(i) removing any protecting groups present;
(ii) converting the compound formula (I) into another compound of formula (I);
(iii) forming a pharmaceutically acceptable salt, hydrate or solvate of the compound of formula I; and/or
(iv) forming a prodrug of the compound of formula I.

An Example of (ii) above is when a compound of formula (I) is synthesised and then one or more of the groups of R₁, R₂, R₃, R₄, R₅ and R₆ may be further reacted to change the nature of the group and provide an alternative compound of formula (I).

The resultant compounds of formula (I) can be isolated and purified using techniques well known in the art.

### Biological Activity

The biological assays described in the example section (Biological Examples 1 to 3) may be used to measure the pharmacological effects of the compounds of the present invention.

Although the pharmacological properties of the compounds of formula I vary with structural change, as expected, the compounds of the invention were found to be active in the assays described in Biological Examples 1, 2 and 3.

In general, in terms of adenosine A2a antagonism, the compounds of the invention demonstrate an IC₅₀ of 1 μM or less in the assay described in Biological Example 1, with preferred compounds of the invention demonstrating an IC₅₀ of 200 nM or less and the most preferred compounds of the invention demonstrating an IC₅₀ of 50 nM or less.

In general, in terms of adenosine A2b antagonism, the compounds of the invention demonstrate an IC₅₀ of 1 μM or less in the assay described in Biological Example 1, with preferred compounds of the invention demonstrating an IC₅₀ of 200 nM or less and the most preferred compounds of the invention demonstrating an IC₅₀ of 50 nM or less.

Suitably the IC₅₀ at the adenosine A1 or A3 receptors of the compounds of the invention in the assay described in Biological Example 1 is at least two-fold higher than the IC₅₀ at the adenosine A2a receptor, and more suitably it is at least 5-fold higher, and even more suitably it is at least 10-fold higher.

### Pharmaceutical Compositions

According to a further aspect of the invention there is provided a pharmaceutical composition which comprises a compound of the invention as defined hereinbefore, or a pharmaceutically acceptable salt, hydrate or solvate thereof, in association with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy is an amount sufficient to treat or prevent a proliferative condition referred to herein, slow its progression and/or reduce the symptoms associated with the condition.

The amount of active ingredient that is combined with one or more excipients to produce a single dosage form will necessarily vary depending upon the individual treated and the particular route of administration. For Example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the formula I will naturally vary according to the nature and severity of the conditions, the age and sex of the animal or patient and the route of administration, according to well-known principles of medicine.

In using a compound of the invention for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of a compound of this invention.

### Therapeutic Uses and Applications

The present invention provides compounds that function as antagonists of adenosine A2 receptors, especially adenosine A2a receptors.

According to a further aspect of the present invention, there is provided a method of antagonising adenosine A2a receptors in vitro or in vivo, said method comprising contacting a cell with an effective amount of a compound or a pharmaceutically acceptable salt, hydrate or solvate thereof as defined herein.

According to a further aspect of the present invention, there is provided a method of selectively antagonising adenosine A2a receptors in vitro or in vivo, said method comprising contacting a cell with an effective amount of a compound, or a pharmaceutically acceptable salt, hydrate or solvate thereof, as defined herein.

According to a further aspect of the present invention, there is provided a method of inhibiting cell proliferation, in vitro or in vivo, said method comprising contacting a cell with an effective amount of a compound or a pharmaceutically acceptable salt, hydrate or solvate thereof as defined herein, or a pharmaceutical composition as defined herein. Suitably, the compound or pharmaceutical composition is administered in combination with one or more additional antiproliferative agents (e.g. checkpoint inhibitors and/or cytotoxic agents).

According to a further aspect of the present invention, there is provided a method of treating a disease or disorder in which adenosine A2a receptor activity is implicated in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a method of treating a proliferative disorder in a patient in need of such treatment, said method comprising administering to said patient a therapeutically effective amount of a compound or a pharmaceutically acceptable salt, hydrate or solvate thereof as defined herein, or a pharmaceutical composition as defined herein. Suitably, the compound or pharmaceutical composition is administered in combination with one or more additional antiproliferative agents (e.g. checkpoint inhibitors and/or cytotoxic agents).

According to a further aspect of the present invention, there is provided a method of treating cancer in a patient in need of such treatment, said method comprising administering to said patient a therapeutically effective amount of a compound or a pharmaceutically acceptable salt, hydrate or solvate thereof as defined herein, or a pharmaceutical composition as defined herein. Suitably, the compound or pharmaceutical composition is administered in combination with one or more additional anticancer agents (e.g. checkpoint inhibitors and/or cytotoxic agents).

According to a further aspect of the present invention, there is provided a compound, or a pharmaceutically acceptable salt, hydrate or solvate thereof, or a pharmaceutical composition as defined herein for use in therapy.

According to a further aspect of the present invention, there is provided a compound or a pharmaceutically acceptable salt, hydrate or solvate thereof as defined herein, or a pharmaceutical composition as defined herein, for use in the treatment of a proliferative condition. Suitably, the compound or pharmaceutical composition is administered in combination with one or more additional antiproliferative agents (e.g. checkpoint inhibitors and/or cytotoxic agents).

According to a further aspect of the present invention, there is provided a compound, or a pharmaceutically acceptable salt, hydrate or solvate thereof, or a pharmaceutical composition as defined herein for use in the treatment of cancer. In a particular embodiment, the cancer is human cancer. Suitably, the compound or pharmaceutical composition is administered in combination with one or more additional anticancer agents (e.g. checkpoint inhibitors and/or cytotoxic agents).

According to a further aspect of the present invention, there is provided a compound, or a pharmaceutically acceptable salt, hydrate or solvate thereof, as defined herein for use as an adenosine A2a antagonist. In an embodiment, the compounds of the invention are selective adenosine A2a antagonists. In an alternative embodiment, certain compounds of the invention are selective adenosine A2a and adenosine A2b antagonists.

According to a further aspect of the present invention, there is provided a compound, or a pharmaceutically acceptable salt, hydrate or solvate thereof, as defined herein for use in the treatment of a disease or disorder in which adenosine A2a is implicated.

According to a further aspect of the present invention, there is provided the use of a compound, or a pharmaceutically acceptable salt, hydrate or solvate thereof, as defined herein in the manufacture of a medicament for the treatment of a proliferative condition.

Suitably, the compound or pharmaceutical composition is administered in combination with one or more additional antiproliferative agents (e.g. checkpoint inhibitors and/or cytotoxic agents).

According to a further aspect of the present invention, there is provide the use of a compound, or a pharmaceutically acceptable salt, hydrate or solvate thereof, as defined herein in the manufacture of a medicament for the treatment of cancer. Suitably, the cancer is a human cancer. Suitably, the compound or pharmaceutical composition is administered in combination with one or more additional anticancer agents (e.g. checkpoint inhibitors and/or cytotoxic agents).

According to a further aspect of the present invention, there is provided a use of a compound, or a pharmaceutically acceptable salt, hydrate or solvate thereof, as defined herein in the manufacture of a medicament for use as an adenosine A2a antagonist.

According to a further aspect of the present invention, there is provided a use of a compound, or a pharmaceutically acceptable salt, hydrate or solvate thereof, as defined herein in the manufacture of a medicament for use as an adenosine A2a antagonist.

According to a further aspect of the present invention, there is provided a use of a compound, or a pharmaceutically acceptable salt, hydrate or solvate thereof, as defined herein in the manufacture of a medicament for the treatment of a disease or disorder in which adenosine A2a receptor activity is implicated.

The term “proliferative disorder” are used interchangeably herein and pertain to an unwanted or uncontrolled cellular proliferation of excessive or abnormal cells which is undesired, such as, neoplastic or hyperplastic growth, whether in vitro or in vivo. Examples of proliferative conditions include, but are not limited to, pre-malignant and malignant cellular proliferation, including but not limited to, malignant neoplasms and tumours, cancers, leukemias, psoriasis, bone diseases, fibroproliferative disorders (e.g., of connective tissues), and atherosclerosis. Any type of cell may be treated, including but not limited to, lung, colon, breast, ovarian, prostate, liver, pancreas, brain, and skin.

The anti-proliferative effects of the compounds of the present invention have particular application in the treatment of human cancers (by virtue of their adenosine A2a antagonist activity).

More specifically, there is provided a compound of general formula (I) for use in the treatment of cancer, particularly solid tumours, for example non-small cell or small cell lung cancer, head and neck squamous cancer and urothelial cancer.

More specifically, there is provided a compound of general formula (I) for use in the treatment of cancer, for example, lung cancer, such as small cell lung cancer or non-small cell lung cancer.

There is also provided the use of a compound of general formula (I) in the manufacture of a medicament for the treatment of cancer, particularly solid tumours, for example non-small cell or small cell lung cancer, head and neck squamous cancer and urothelial cancer.

The invention further provides a method for the treatment of cancer, particularly solid tumours, for example non-small cell or small cell lung cancer, head and neck squamous cancer and urothelial cancer, the method comprising administering to a patient in need of such treatment an effective amount of a compound of general formula (I).

The patient to be treated is suitably a mammal and more suitably a human.

### Routes of Administration

The compounds of the invention or pharmaceutical compositions comprising these compounds may be administered to a subject by any convenient route of administration, whether systemically/peripherally or topically (i.e., at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, intrasternal, and intratumoural; by implant of a depot or reservoir, for example, subcutaneously or intramuscularly.

The compounds of the invention or pharmaceutical compositions comprising these compounds may be administered by intratumoural delivery.

### Combination Therapies

The compounds of formula I are useful for the treatment and/or prophylaxis of proliferative disorders, such as, for example, cancer. A compound of formula I defined herein may be used in combination with one or more additional antiproliferative/anticancer therapies, such as, for example, chemotherapy with one or more additional antiproliferative/anticancer agents, radiotherapy and/or conventional surgery.

An additional antiproliferative/anticancer agent may be included in the pharmaceutical composition with a compound of formula (I) as defined herein or, alternatively, it may be administered separately, either at the same time as the compound of formula (I) or at an earlier or later time.

Therefore, in a further aspect of the present invention there is provided a product comprising a compound of general formula (I) and an additional agent useful in the treatment or prevention of cancer as a combined preparation for simultaneous, sequential or separate use in the treatment of cancer.

The present invention also provides a compound of general formula (I) in combination with one or more additional antiproliferative/anticancer agents for use in the treatment of cancer as a combined preparation for simultaneous, sequential or separate use in the treatment of treatment of cancer.

In particular, the combination therapy defined herein is suitable for the treatment of solid tumours for example non-small cell or small cell lung cancer, head and neck squamous cancer and urothelial cancer.

Suitable additional antiproliferative/anti-cancer agents that may be used in combination with a compound of formula I defined herein [either separately or as part of a combined pharmaceutical composition or a combined preparation with the compounds of general formula (I)] include:
1) other forms of cancer immunotherapy and anti-cancer chemotherapeutic agents;
2) adenosine pathway modulators, including, but not limited to A2b antagonists, CD73 inhibitors and CD39 inhibitors;
3) anti-PD-1 and PDL-1 antibodies including, but not limited to, cetrelimab pembrolizumab, nivolumab, durvalumab, avelumab and atezolizumab; and 4) anti-CTLA4 antibodies including, but not limited to, ipilimumab.

The compounds of formula I defined herein are particularly suited to use in combination with anti-PD-1 and PDL-1 antibodies including, but not limited to, Cetrelimab, pembrolizumab, nivolumab, durvalumab, avelumab and atezolizumab. Suitably, the anti-PD1 antibody is one of the antibodies disclosed in U.S. Publication No. 2019/0225689 or U.S. Publication No. 2017/0121409 (incorporated herein by reference in their entireties), such as cetrelimab. Cetrelimab (JNJ-63723283, CET) is a fully human immunoglobulin (Ig) G4 kappa monoclonal antibody that binds to programmed death receptor-1 (PD-1) with high affinity and specificity. Cetrelimab has shown activity in solid tumors. Rutkowski P, et al. Journal of Clinical Oncology. 2019; 37(8):31.

The compounds of formula I defined herein are particularly suited to use in combination with adenosine pathway modulators, including, but not limited to A2b antagonists, CD73 inhibitors and CD39 inhibitors.

The A2a antagonists of general formula (I) can also be used in combination with cell-based immunotherapy and cancer vaccines that include, but are not limited to CAR-T cell therapy.

Examples of the additional antiproliferative/anticancer chemotherapeutic agents include, but are not limited to, any one or more of the following: MEK (e.g. MEK1, MEK2, or MEK1 and MEK2) inhibitors (e.g. XL518, CI-1040, PD035901, selumetinib/AZD6244, GSK1 120212/trametinib, GDC-0973, ARRY-162, ARRY-300, AZD8330, PD0325901, U0126, PD98059, TAK-733, PD3 18088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds or platinum containing agents (e.g. cisplatin, oxaloplatin, arboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin Ill derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cisporphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fiudarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatinA; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzyl guanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-basedimmune modulator; protein kinase C inhibitor; protein kinase C inhibitors, microalgal; proteintyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Rₑ 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stemcell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin 2 (including recombinant interleukin 2, or rIL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP—XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-3 10705), 21-hydroxyepothilone D (i.e. Desoxyepothilone F and dEpoF), 26-fluoroepothilone, Auristatin PE (i.e. NSC-654663), Soblidotin (i.e. TZT-1027), Vincristine sulfate, Cryptophycin 52 (i.e. LY-355703), Vitilevuamide, Tubulysin A, Canadensol, Centaureidin (i.e. NSC-106969), Oncocidin A1 (i.e. BTO-956 and DF E), Fijianolide B, Laulimalide, Narcosine (also known as NSC-5366), Nascapine, Hemiasterlin, Vanadocene acetylacetonate, Monsatrol, Inanocine (i.e. NSC-698666), Eleutherobins (such as Desmethyleleutherobin, Desaetyleleutherobin, Isoeleutherobin A, and ZEleutherobin), Caribaeoside, Caribaeolin, Halichondrin B, Diazonamide A, Taccalonolide A, Diozostatin, (−)-Phenylahistin (i.e. NSCL-96F037), Myoseverin B, Resverastatin phosphate sodium, steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guerin (BCG), levamisole, interleukin-2, alpha-interferon, etc.), monoclonal antibodies (e.g., anti-CD20, anti-F£R2, anti-CD52, anti-ULA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to In, 0Y, or I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™) panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, hormonal therapies, or the like.

As indicated above, the combination therapy of the present invention may be achieved by way of the simultaneous, sequential or separate dosing of the individual components of the treatment. Such combination products employ the compounds of this invention within the dosage range described hereinbefore and the other pharmaceutically-active agent within its approved dosage range.

According to this aspect of the invention there is provided a combination for use in the treatment of a cancer (for example a cancer involving a solid tumour) comprising a compound of the invention as defined hereinbefore, or a pharmaceutically acceptable salt, hydrate or solvate thereof, and one or more additional antiproliferative/anticancer agents.

According to this aspect of the invention there is also provided a combination for use in the treatment of a proliferative condition, such as cancer (for example a cancer involving a solid tumour), comprising a compound of the invention as defined hereinbefore, or a pharmaceutically acceptable salt, hydrate or solvate thereof, and one or more additional antiproliferative/anticancer agents selected from those listed above.

In a further aspect of the invention there is provided a compound of the invention or a pharmaceutically acceptable salt, hydrate or solvate thereof, for use in the treatment of cancer in combination with another anti-tumour agent, optionally selected from one listed herein above.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

According to a further aspect of the invention there is provided a pharmaceutical composition which comprises a compound of the invention, or a pharmaceutically acceptable salt, hydrate or solvate thereof, in combination with an anti-tumour agent (optionally selected from one listed herein above), in association with a pharmaceutically acceptable diluent or carrier.

### EXAMPLES

### General Conditions:

Mass spectra were run on LC-MS systems using electrospray ionization. These were run using either a Waters Acquity H-Class UPLC with PDA and QDa mass detection, an Acquity UPLC (binary pump/PDA detector)+ZQ Mass Spectrometer or Acquity i-Class (quaternary pump/PDA detector)+Quattro Micro Mass Spectrometer, a Waters Acquity UPLC system with Waters PDA and ELS detectors or a Shimadzu LC-MS-2010EV system. [M+H]+ refers to mono-isotopic molecular weights.

NMR spectra were run on either a Bruker Ultrashield 500 MHz NMR spectrometer or a Bruker Avance Ill HD 400 MHz NMR spectrometer. Spectra were recorded at 298K and were referenced using the solvent peak.

The following examples are intended to illustrate the invention and are not to be construed as being limitations thereon. Temperatures are given in degrees centigrade. If not mentioned otherwise, all evaporations are performed in vacuo, preferably between about 15 mm Hg and 100 mm Hg (═20-133 mbar). The structure of final products, intermediates and starting materials is confirmed by standard analytical methods, e.g., microanalysis and spectroscopic characteristics, e.g., MS and NMR. Abbreviations used are those conventional in the art. If not defined, the terms have their generally accepted meanings.

### Abbreviation

br broad
CDI 1,1′-carbonyldiimidazole
d doublet
dd doublet of doublets
DCM dichloromethane
DIPEA diisopropylethylamine
DMA dimethylacetamide
DMAP 4-dimethylaminopyridine
DMF N,N-dimethylformamide
DMSO dimethyl sulfoxide
EtOAc ethyl acetate
HPLC high pressure liquid chromatography
LC-MS liquid chromatography and mass spectrometry
MeOH MeOH
MeCN acetonitrile
MS mass spectrometry
m multiplet
min(s) minute(s)
mL milliliter(s)
m/z mass to charge ratio
NBS N-bromosuccinimide
NMP N-methyl-2-pyrrolidone
NMR nuclear magnetic resonance
Pd(dppf)Cl₂ [1,1′-bis(diphenylphosphino)ferrocene]palladium(II) dichloride
Rt retention time
s singlet
t triplet
TFAA trifluoroacetic anhydride
TFA trifluoroacetic acid
THF tetrahydrofuran

Referring to the examples that follow, compounds of the preferred embodiments were synthesized using the methods described herein, or other methods, which are known in the art.

The various starting materials, intermediates, and compounds of the preferred embodiments may be isolated and purified, where appropriate, using conventional techniques such as precipitation, filtration, crystallization, evaporation, distillation, and chromatography. Unless otherwise stated, all starting materials are obtained from commercial suppliers and used without further purification. Salts may be prepared from compounds by known salt-forming procedures.

If not indicated otherwise, the analytical HPLC conditions are as follows:

### Instrument: LC-MS-1:

### Method 2A

| Column: | Acquity UPLC BEH C18 2.1 × 50 mm 1.7 μm | |
|---|---|---|
| Column Temp: | 50° | C. |
| Flow rate: | 0.8 | mL/min. |

| | |
|---|---|
| Eluents: | A: H₂O, 0.1% formic acid, B: MeCN |
| Gradient: | 0.0-1.8 min 2-98% B, 1.8-2.1 min 98% |
| | B, 2.1-2.5 98% A. |

### Method 2B

| Column: | Acquity UPLC BEH C18 2.1 × 50 mm 1.7 μm | |
|---|---|---|
| Column Temp: | 50° | C. |
| Flow rate: | 0.8 | mL/min. |

| | |
|---|---|
| Eluents: | A: H₂O, 0.1% ammonia B: MeCN |
| Gradient: | 0.0-1.8 min 2-98% B, 1.8-2.1 min |
| | 98% B, 2.1-2.5 98% A. |

### Method 3A

| Column: | Acquity UPLC BEH C18 2.1 × 50 mm 1.7 μm | |
|---|---|---|
| Column Temp: | 50° | C. |
| Eluents: A: | H₂O, 0.1% formic acid, B: MeCN | |
| Flow Rate: | 0.6 | mL/min |

| | |
|---|---|
| Gradient: | 0.0-2.5 min 2-98% B, 2.5-3.3 min 98% B, |
| | 3.3-3.5 98% A. |

### Method 3B

| Column: | Acquity UPLC BEH C18 2.1 × 50 mm 1.7 μm | |
|---|---|---|
| Column Temp: | 50° | C. |
| Eluents: A: | H₂O, 0.1% ammonia B: MeCN | |
| Flow Rate: | 0.6 | mL/min |

| | |
|---|---|
| Gradient: | 0.2-2.5 min 2-98% B, 2.5-3.3 min 98% B, |
| | 3.3-3.5 98% A. |

### Method 5A

| | |
|---|---|
| Column: | YMC-Triart C18 50 × 2 mm, 5 μm. |
| Column Temp: | Room temperature |
| Flow rate: | 0.8 mL/min. |
| Eluents: | A: H₂O, B: MeCN, C: 50% H₂O/50% MeCN + 1.0% |
| | formic acid |
| Gradient: | 0.0-4.0 mins 0-95% B, 5% C; 4.0-4.4 mins 95% B, |
| | 5% C 4.4-5.0 mins 95% A, 5% B. |

### Method 8A

| | |
|---|---|
| Column: | Acquity UPLC BEH C18 2.1 × 50 mm 1.7 μm |
| Column Temp: | 50° C. |
| Eluents: | A: H₂O, 0.1% formic acid, B: MeCN |
| Flow Rate: | 0.6 mL/min |
| Gradient: | 0.5-6.5 min 2-98% B, 6.5-7.6 min 98% B, 7.6-8.0 |
| | 98% A. |

### Method 8B

| | |
|---|---|
| Column: | Acquity UPLC BEH C18 2.1 × 50 mm 1.7 μm |
| Column Temp: | 50° C. |
| Eluents: A: | H₂O, 0.1% ammonia, B: MeCN |
| Flow Rate: | 0.6 mL/min |
| Gradient: | 0.5-6.5 min 2-98% B, 6.5-7.6 min 98% B, |
| | 7.6-8.0 98% A. |

### Instrument: LC-MS-2:

### Method 2A

| | |
|---|---|
| Column: | Acquity UPLC BEH C18 2.1 × 50 mm 1.7 μm |
| Column Temp: | 50° C. |
| Flow rate: | 0.8 mL/min. |
| Eluents: | A: H₂O, B: MeCN, C: 50% H₂O/50% MeCN + 2.0% |
| | formic acid |
| Gradient: | 0.0-1.7 mins 0-95% B, 5% C; 1.7-2.1 mins 95% B, |
| | 5% C 2.1-2.5 mins 95% A, 5% C. |

### Method 2B

| | |
|---|---|
| Column: | Acquity UPLC BEH C18 2.1 × 50 mm 1.7 μm |
| Column Temp: | 50° C. |
| Flow rate: | 0.8 mL/min. |
| Eluents: A: | H₂O, B: MeCN, C: 50% H₂O/50% MeCN + 2.0% |
| | ammonia (aq.) |
| Gradient: | 0.0-1.7 mins 0-95% B, 5% D; 1.7-2.1 mins 95% B, |
| | 5% D 2.1-2.5 mins 95% A, 5% D. |

### Method 8A

| | |
|---|---|
| Column: | Acquity UPLC BEH C18 2.1 × 50 mm 1.7 μm |
| Column Temp: | 50° C. |
| Flow rate: | 0.8 mL/min. |
| Eluents: | A: H₂O, B: MeCN, C: 50% H₂O/50% MeCN + 2.0% |
| | formic acid |
| Gradient: | 0.0-0.5 mins 95% A, 5% C; 0.5-6.5 mins 0-95% B, |
| | 5% C; 6.6-7.5 mins 95% B, 5% C; 7.5-7.6 mins 0-95% |
| | A, 5% C; 7.6-8.0 mins 95% A, 5% C. |

### Method 8B

| | |
|---|---|
| Column: | Acquity UPLC BEH C18 2.1 × 50 mm 1.7 μm |
| Column Temp: | 50° C. |
| Flow rate: | 0.8 mL/min. |
| Eluents: | A: H₂O, B: MeCN, C: 50% H₂O/50% MeCN + 2.0% |
| | ammonia (aq.) |
| Gradient: | 0.0-0.5 mins 95% A, 5% D; 0.5-6.5 mins 0-95% B, |
| | 5% D; 6.6-7.5 mins 95% B, 5% D; 7.5-7.6 mins 0-95% |
| | A, 5% D; 7.6-8.0 mins 95% A, 5% D. |

### Instrument: LC-MS

### Method 7A

| | |
|---|---|
| Column: | Phenomenex Kinetix-XB C18 2.1 × 100 mm, 1.7 μm |
| Column Temp | 40° C. |
| Eluents: | A: H₂O 0.1% formic acid, B: acetonitrile, 0.1% |
| | formic acid |
| Flow Rate: | 0.6 mL/mins |
| Gradient: | 0-5.3 mins 5-100% B, 5.3-5.8 mins 100% B, 5.8-5.82 |
| | mins 100-5% B, 5.82-7.00 mins 5% B |

### Method 7B

| | |
|---|---|
| Column: | Waters UPLC ® BEH ™ C18, 2.1 mm × 100 mm, |
| | 1.7 μm column |
| Column Temp: | 55° C. |
| Eluents: | A: 2 mM ammonium bicarbonate buffered to pH 10, |
| | B: acetonitrile |
| Flow Rate: | 0.6 mL/mins |
| Gradient: | 0-5.3 mins 5-100% B, 5.3-5.8 mins 100% B, 5.8-5.82 |
| | mins 100-5% B, 5.82-7.00 mins 5% B |

### Instrument: LCMS-W

### Method 2A

| | |
|---|---|
| Column: | Waters UPLCTM BEHTM C18 2.1 × 50 mm, 1.7 μm |
| Column Temp | 40° C. |
| Eluents: | A: H2O 0.1% formic acid, B: acetonitrile, 0.1% |
| | formic acid |
| Flow Rate: | 0.9 mL/mins |
| Gradient: | 0-1.10 mins 5-100% B, 1.10-1.35 mins 100% B, |
| | 1.35-1.40 mins 100-5% B, 1.40-1.50 mins 5% B |

### Instrument: LCMS-S

### Method 2A

| | |
|---|---|
| Column: | Kinetex Core-Shell C18, 2.1 mm × 50 mm, 5 μm |
| Column Temp | 40° C. |
| Eluents: | A: H2O 0.1% formic acid, B: acetonitrile, |
| | 0.1% formic acid |
| Flow Rate: | 1.2 mL/mins |
| Gradient: | 0-1.20 mins 5-100% B, 1.20-1.30 mins 100% B, |
| | 1.30-1.31 mins 100-5% B, 1.31-1.70 mins 5% B |

### Example 1

### (3S)-3-Cyano-N-[4-(3-cyanophenyl)-5-(4-methylquinazolin-6-yl)thiazol-2-yl]-3-methyl-pyrrolidine-1-carboxamide

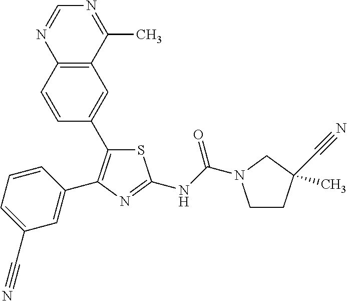 [SMILES: Cc1ncnc2ccc(-c3sc(NC(=O)N4CC[C@](C)(C#N)C4)nc3-c3cccc(C#N)c3)cc12]

A mixture of 3-[2-amino-5-(4-methylquinazolin-6-yl)thiazol-4-yl]benzonitrile (Intermediate A)(100 mg, 0.29 mmol), CDI (94 mg, 0.58 mmol) and DIPEA (152 μL, 0.87 mmol) in DMA (1 mL) was stirred at 50° C. for 4 h. (3S)-3-Methylpyrrolidine-3-carbonitrile hydroiodide (Intermediate B)(139 mg, 0.58 mmol) and additional DIPEA (152 μL, 0.87 mmol) were added and the mixture heated for 15 mins. The resulting mixture was allowed to cool to room temperature and partitioned between EtOAc (20 mL) and H₂O (20 mL). The organic portion was separated and the aqueous further extracted with EtOAc (2×20 mL). The combined organics were washed with brine (5×20 mL), dried over Na₂SO₄ and the solvent removed in vacuo. Purification by chromatography on silica eluting with a gradient of 85% EtOAc in DCM followed by 3% MeOH in DCM afforded a yellow glassy solid. The solid was dried in a vacuum oven at 50° C. for 64 h to afford the title compound as a yellow solid.

LC-MS-1 (Method 8A): Rt 3.27 mins; MS m/z 480.4=[M+H]+

1H NMR (500 MHz, DMSO-d6) δ 11.25 (s, 1H), 9.12 (s, 1H), 8.22 (d, J=2.0 Hz, 1H), 7.96-7.89 (m, 2H), 7.80 (d, J=7.8 Hz, 1H), 7.77 (dd, J=8.7, 2.0 Hz, 1H), 7.71 (d, J=7.8 Hz, 1H), 7.52 (t, J=7.8 Hz, 1H), 4.00-3.88 (m, 1H), 3.75-3.66 (m, 1H), 3.66-3.58 (m, 1H), 3.47-3.40 (m, 1H), 2.84 (s, 3H), 2.45-2.39 (m, 1H), 2.12-2.04 (m, 1H), 1.48 (s, 3H).

### Example 2

### N-[4-(3-Cyanophenyl)-5-(4-methylquinazolin-6-yl)thiazol-2-yl]-4-oxa-1,9-diazaspiro[5.5]undecane-9-carboxamide

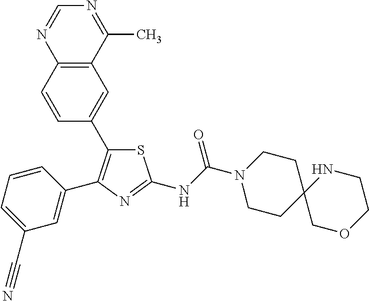 [SMILES: Cc1ncnc2ccc(-c3sc(NC(=O)N4CCC5(CC4)COCCN5)nc3-c3cccc(C#N)c3)cc12]

### Step 1: tert-Butyl 9-[[4-(3-cyanophenyl)-5-(4-methylquinazolin-6-yl)thiazol-2-yl]carbamoyl]-4-oxa-1,9-diazaspiro[5.5]undecane-1-carboxylate

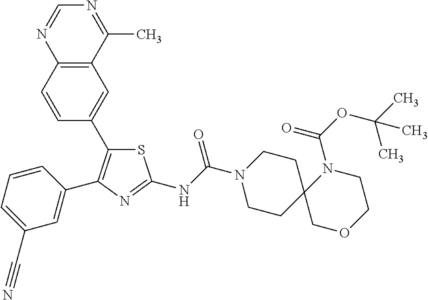 [SMILES: Cc1ncnc2ccc(-c3sc(NC(=O)N4CCC5(CC4)COCCN5C(=O)OC(C)(C)C)nc3-c3cccc(C#N)c3)cc12]

A mixture of 3-[2-amino-5-(4-methylquinazolin-6-yl)thiazol-4-yl]benzonitrile (Intermediate A) (250 mg, 0.73 mmol), CDI (236 mg, 1.46 mmol) and DIPEA (380 μL, 2.18 mmol) in DMA (2.5 mL) was stirred at 50 00 for 5.5 h. tert-Butyl 4-oxa-1,9-diazaspiro[5.5]undecane-1-carboxylate (373 mg, 1.46 mmol) was added and the mixture heated at 50° C. for 30 mins. The resulting mixture was allowed to cool to room temperature and was partitioned between EtOAc (20 mL) and H₂O (20 mL). The organic portion was separated and the aqueous further extracted with EtOAc (2×20 mL). The combined organics were washed with brine (5×20 mL), dried over Na₂SO₄ and the solvent removed in vacuo. Purification by chromatography on silica eluting with 3% MeOH in DCM followed by a second purification by chromatography on silica eluting with 80% EtOAc in DCM afforded the title compound as a yellow solid.

LC-MS-1 (Method 2B): Rt 1.49 mins; MS m/z 626.5=[M+H]+

1H NMR (500 MHz, DMSO-d6) δ 11.19 (s, 1H), 9.11 (s, 1H), 8.21 (d, J=2.0 Hz, 1H), 7.95-7.90 (m, 2H), 7.81-7.75 (m, 2H), 7.71 (dt, J=7.9, 1.4 Hz, 1H), 7.52 (t, J=7.9 Hz, 1H), 3.97 (apr dt, J=11.4, 6.8 Hz, 2H), 3.74-3.68 (m, 4H), 3.48 (t, J=5.4 Hz, 2H), 3.18 (t, J=12.8 Hz, 2H), 2.84 (s, 3H), 1.64 (dt, J=13.6, 3.6 Hz, 2H), 1.38 (s, 9H). 1×CH₂ signal not observed.

### Step 2: N-[4-(3-Cyanophenyl)-5-(4-methylquinazolin-6-yl)thiazol-2-yl]-4-oxa-1,9-diazaspiro[5.5]undecane-9-carboxamide

To a solution of tert-butyl 9-[[4-(3-cyanophenyl)-5-(4-methylquinazolin-6-yl)thiazol-2-yl]carbamoyl]-4-oxa-1,9-diazaspiro[5.5]undecane-1-carboxylate (step 1) (216 mg, 0.35 mmol) in DCM (5 mL) was added TFA (0.53 mL, 6.9 mmol) dropwise and the mixture stirred at room temperature for 3 h. The volatile solvent was removed under a flow of nitrogen and the resulting mixture diluted with MeOH and loaded onto a 2.5 g Isolute® SCX cartridge (pre-wetted with MeOH). The column was flushed with 5 column volumes of MeOH, and the product was eluted with 4:1 7M NH₃ in MeOH in MeOH (approx. 20 mL). The solvent was removed in vacuo and purification by C18 reverse phase chromatography eluting with 0.1% NH₄OH in MeCN/water followed by chromatography on silica eluting with 5% 7M NH₃ in MeOH/DCM afforded a yellow oil. Et₂O (2 mL) was added and a yellow solid formed which was collected by filtration, washed with Et₂O (1 mL) and dried in a vacuum oven at 60° C. to afford the title compound as a yellow solid.

LC-MS-1 (Method 8B): Rt 2.56 mins; MS m/z 526.5=[M+H]+

1H NMR (500 MHz, DMSO-d6) δ 11.15 (s, 1H), 9.12 (s, 1H), 8.21 (d, J=2.0 Hz, 1H), 7.96-7.90 (m, 2H), 7.80 (d, J=7.8 Hz, 1H), 7.77 (dd, J=8.8, 2.0 Hz, 1H), 7.71 (d, J=7.8 Hz, 1H), 7.52 (t, J=7.8 Hz, 1H), 3.77-3.68 (m, 2H), 3.52-3.45 (m, 2H), 3.45-3.38 (m, 2H), 2.84 (s, 3H), 2.75-2.70 (m, 2H), 1.66-1.58 (m, 2H), 1.45-1.37 (m, 2H). 1×CH₂ and morpholine NH not observed.

### Example 2.1

### N-[4-(3-Cyanophenyl)-5-(4-methylquinazolin-6-yl)thiazol-2-yl]-2-(cyclopropylmethyl)piperazine-1-carboxamide

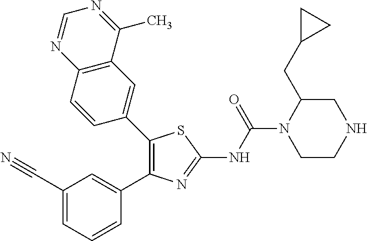 [SMILES: Cc1ncnc2ccc(-c3sc(NC(=O)N4CCNCC4CC4CC4)nc3-c3cccc(C#N)c3)cc12]

### Step 1: tert-Butyl 4-[[4-(3-cyanophenyl)-5-(4-methylquinazolin-6-yl)thiazol-2-yl]carbamoyl]-3-(cyclopropylmethyl)piperazine-1-carboxylate

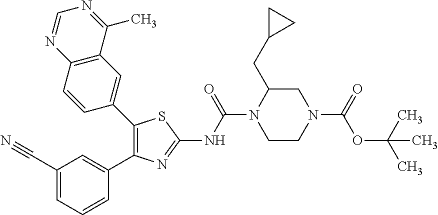 [SMILES: Cc1ncnc2ccc(-c3sc(NC(=O)N4CCN(C(=O)OC(C)(C)C)CC4CC4CC4)nc3-c3cccc(C#N)c3)cc12]

The title compound was prepared from 3-[2-amino-5-(4-methylquinazolin-6-yl)thiazol-4-yl]benzonitrile (Intermediate A) and tert-butyl 3-(cyclopropylmethyl)piperazine-1-carboxylate analogously to Example 2 step 1.

LC-MS-W (Method 2A): Rt 1.09 mins; MS m/z 610.3=[M+H]+

1H NMR (400 MHz, CDCl3) δ 9.18 (s, 1H), 8.09 (d, J=2.0 Hz, 1H), 7.93 (d, J=8.8 Hz, 1H), 7.88 (t, J=1.7 Hz, 1H), 7.68 (dd, J=8.7, 2.0 Hz, 1H), 7.63-7.56 (m, 2H), 7.35 (dd, J=7.8, 7.8 Hz, 1H), 4.14 (d, J=13.3 Hz, 3H), 4.08-3.96 (m, 2H), 3.90-3.77 (m, 1H), 3.18 (t, J=12.5 Hz, 1H), 3.09 (d, J=13.1 Hz, 1H), 2.97-2.89 (m, 1H), 2.87 (s, 3H), 1.45 (d, J=1.2 Hz, 9H), 0.79-0.69 (m, 1H), 0.62-0.50 (m, 2H), 0.24-0.12 (m, 2H). NH not observed.

### Step 2: N-[4-(3-Cyanophenyl)-5-(4-methylquinazolin-6-yl)thiazol-2-yl]-2-(cyclopropylmethyl)piperazine-1-carboxamide

The title compound was prepared from tert-butyl 4-[[4-(3-cyanophenyl)-5-(4-methylquinazolin-6-yl)thiazol-2-yl]carbamoyl]-3-(cyclopropylmethyl)piperazine-1-carboxylate (step 1) and TFA analogously to Example 2 step 2.

LC-MS (Method 7A): Rt 2.35 mins; MS m/z 510.1=[M+H]+ 1H NMR (400 MHz, MeOD) δ 9.07 (s, 1H), 8.25 (d, J=2.0 Hz, 1H), 7.97-7.93 (m, 2H), 7.86 (dd, J=8.8, 2.0 Hz, 1H), 7.71 (dt, J=8.0, 1.4 Hz, 1H), 7.66 (dt, J=7.7, 1.3 Hz, 1H), 7.44 (dd, J=7.8, 7.8 Hz, 1H), 4.38-4.31 (m, 1H), 3.98-3.91 (m, 1H), 3.08 (d, J=13.1 Hz, 1H), 3.04-2.98 (m, 1H), 2.93 (dd, J=13.1, 4.2 Hz, 1H), 2.88 (s, 3H), 2.73 (td, J=12.6, 3.7 Hz, 2H), 1.76-1.64 (m, 2H), 0.75-0.68 (m, 1H), 0.49-0.44 (m, 2H), 0.16 (dd, J=5.0, 1.8 Hz, 2H). 2×NH not observed.

### Example 2.2

### N-[4-(3-Cyanophenyl)-5-(4-methyl-1H-indazol-6-yl)thiazol-2-yl]-2-oxa-6-azaspiro[3.3]heptane-6-carboxamide

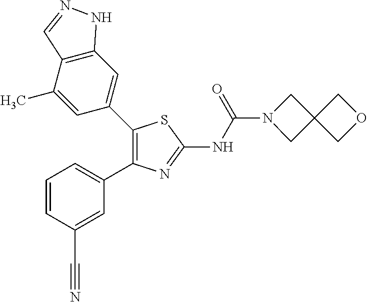 [SMILES: Cc1cc(-c2sc(NC(=O)N3CC4(COC4)C3)nc2-c2cccc(C#N)c2)cc2[nH]ncc12]

### Step 1: N-[4-(3-Cyanophenyl)-5-(4-methyl-1-trityl-indazol-6-yl)thiazol-2-yl]-2-oxa-6-azaspiro[3.3]heptane-6-carboxamide

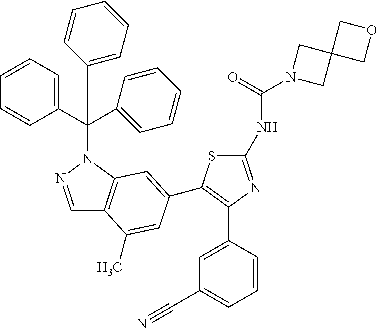 [SMILES: Cc1cc(-c2sc(NC(=O)N3CC4(COC4)C3)nc2-c2cccc(C#N)c2)cc2c1cnn2C(c1ccccc1)(c1ccccc1)c1ccccc1]

The title compound was prepared from 3-[2-amino-5-(4-methyl-1-trityl-indazol-6-yl)thiazol-4-yl]benzonitrile (Intermediate AO) and 2-oxa-6-azaspiro[3.3]heptane analogously to Example 2 step 1.

LC-MS-2 (Method 2B): Rt 1.99 mins; MS m/z 697.3=[M−H]-1H NMR (500 MHz, DMSO-d6) δ 11.20 (s, 1H), 8.05 (s, 1H), 7.86 (s, 1H), 7.75 (dd, J=12.3, 7.9 Hz, 2H), 7.50 (t, J=7.9 Hz, 1H), 7.42-7.36 (m, 10H), 7.11 (dd, J=7.2, 2.6 Hz, 6H), 6.69 (s, 1H), 4.67 (s, 4H), 4.20 (s, 4H), 2.34 (s, 3H).

### Step 2: N-[4-(3-Cyanophenyl)-5-(4-methyl-1H-indazol-6-yl)thiazol-2-yl]-2-oxa-6-azaspiro[3.3]heptane-6-carboxamide

The title compound was prepared from N-[4-(3-cyanophenyl)-5-(4-methyl-1-trityl-indazol-6-yl)thiazol-2-yl]-2-oxa-6-azaspiro[3.3]heptane-6-carboxamide (step 1) and TFA analogously to Example 2 step 2.

LC-MS-2 (Method 8B): Rt 3.24 mins; MS m/z 457.1=[M+H]+

1H NMR (500 MHz, DMSO-d6) δ 13.03 (d, J=1.6 Hz, 1H), 11.22 (s, 1H), 8.13 (d, J=1.3 Hz, 1H), 7.88 (t, J=1.8 Hz, 1H), 7.73 (dt, J=7.6, 1.4 Hz, 1H), 7.67 (dt, J=8.0, 1.4 Hz, 1H), 7.48 (t, J=7.8 Hz, 1H), 7.24 (s, 1H), 6.82 (s, 1H), 4.68 (s, 4H), 4.20 (s, 4H). CH₃ not observed.

### Example 2.3

### N-[4-(3-Cyanophenyl)-5-(7-methyl-3H-benzotriazol-5-yl)thiazol-2-yl]-2-oxa-6-azaspiro[3.3]heptane-6-carboxamide

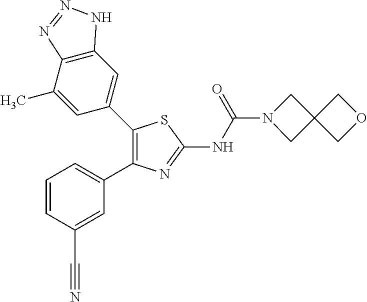 [SMILES: Cc1cc(-c2sc(NC(=O)N3CC4(COC4)C3)nc2-c2cccc(C#N)c2)cc2[nH]nnc12]

### Step 1: N-[4-(3-Cyanophenyl)-5-(7-methyl-3-trityl-benzotriazol-5-yl)thiazol-2-yl]-2-oxa-6-azaspiro[3.3]heptane-6-carboxamide

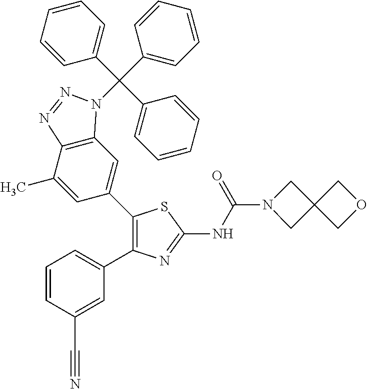 [SMILES: Cc1cc(-c2sc(NC(=O)N3CC4(COC4)C3)nc2-c2cccc(C#N)c2)cc2c1nnn2C(c1ccccc1)(c1ccccc1)c1ccccc1]

The title compound was prepared from 3-[2-amino-5-(7-methyl-3-trityl-benzotriazol-5-yl)thiazol-4-yl]benzonitrile (Intermediate AP) and 2-oxa-6-azaspiro[3.3]heptane analogously to Example 2 step 1.

LC-MS-2 (Method 2B): Rt 1.85 mins; MS m/z 698.3=[M−H]—. 1H NMR (500 MHz, DMSO) δ 11.21 (s, 1H), 7.69 (dt, J=7.0, 1.8 Hz, 1H), 7.61 (d, J=1.8 Hz, 1H), 7.42-7.34 (m, 2H), 7.36-7.27 (m, 9H), 7.11 (d, J=1.3 Hz, 1H), 7.06-6.98 (m, 6H), 5.92 (s, 1H), 4.67 (s, 4H), 4.18 (s, 4H), 2.67 (s, 3H).

### Step 2: N-[4-(3-Cyanophenyl)-5-(7-methyl-3H-benzotriazol-5-yl)thiazol-2-yl]-2-oxa-6-azaspiro[3.3]heptane-6-carboxamide

The title compound was prepared from N-[4-(3-cyanophenyl)-5-(7-methyl-3-trityl-benzotriazol-5-yl)thiazol-2-yl]-2-oxa-6-azaspiro[3.3]heptane-6-carboxamide (step 1) and TFA analogously to Example 2 step 2.

LC-MS-2 (Method 8B): Rt 2.33 mins; MS m/z 458.0=[M+H]+

1H NMR (500 MHz, MeOD) δ 7.92 (t, J=1.7 Hz, 1H), 7.67 (dt, J=8.0, 1.5 Hz, 1H), 7.63 (s, 1H), 7.61 (dt, J=7.8, 1.4 Hz, 1H), 7.39 (t, J=7.9 Hz, 1H), 7.18 (s, 1H), 4.84 (s, 4H), 4.33 (s, 4H), 2.66 (s, 3H). 2×NH not observed.

### Example 2.4

### N-[4-(3-Cyanophenyl)-5-(1H-pyrrolo[2,3-b]pyridin-4-yl)thiazol-2-yl]-2-oxa-6-azaspiro[3.3]heptane-6-carboxamide

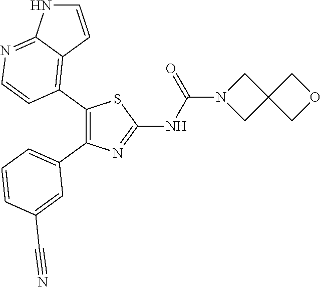 [SMILES: N#Cc1cccc(-c2nc(NC(=O)N3CC4(COC4)C3)sc2-c2ccnc3[nH]ccc23)c1]

### Step 1: tert-Butyl 4-[4-(3-cyanophenyl)-2-(2-oxa-6-azaspiro[3.3]heptane-6-carbonylamino)thiazol-5-yl]pyrrolo[2,3-b]pyridine-1-carboxylate

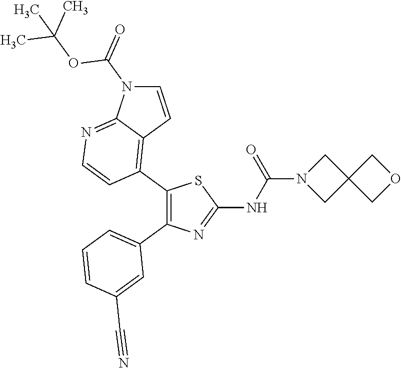 [SMILES: CC(C)(C)OC(=O)n1ccc2c(-c3sc(NC(=O)N4CC5(COC5)C4)nc3-c3cccc(C#N)c3)ccnc21]

The title compound was prepared from tert-butyl 4-[2-amino-4-(3-cyanophenyl)thiazol-5-yl]pyrrolo[2,3-b]pyridine-1-carboxylate (Intermediate AQ) and 2-oxa-6-azaspiro[3.3]heptane analogously to Example 2 step 1.

LC-MS-1 (Method 3B): Rt 1.78 mins; MS m/z 543.2=[M+H]+

1H NMR (500 MHz, DMSO) δ 11.42 (s, 1H), 8.39 (d, J=4.9 Hz, 1H), 7.86 (t, J=1.8 Hz, 1H), 7.77-7.69 (m, 1H), 7.55 (dt, J=8.0, 1.5 Hz, 1H), 7.44 (t, J=7.9 Hz, 2H), 7.21 (d, J=5.0 Hz, 1H), 6.24 (d, J=4.1 Hz, 1H), 4.69 (s, 4H), 4.22 (s, 4H), 1.61 (s, 9H).

### Step 2: N-[4-(3-Cyanophenyl)-5-(1H-pyrrolo[2,3-b]pyridin-4-yl)thiazol-2-yl]-2-oxa-6-azaspiro[3.3]heptane-6-carboxamide

The title compound was prepared from tert-butyl 4-[4-(3-cyanophenyl)-2-(2-oxa-6-azaspiro[3.3]heptane-6-carbonylamino)thiazol-5-yl]pyrrolo[2,3-b]pyridine-1-carboxylate (step 1) and TFA analogously to Example 2 step 2.

LC-MS-1 (Method 8B): Rt 3.49 mins; MS m/z 443.2=[M+H]+ 1H NMR (500 MHz, DMSO) δ 11.83 (s, 1H), 11.36 (s, 1H), 8.20 (d, J=5.0 Hz, 1H), 7.81 (t, J=1.8 Hz, 1H), 7.72 (dt, J=7.7, 1.5 Hz, 1H), 7.62 (dt, J=8.0, 1.5 Hz, 1H), 7.47-7.41 (m, 2H), 6.97 (d, J=5.0 Hz, 1H), 6.05 (dd, J=3.5, 1.7 Hz, 1H), 4.69 (s, 4H), 4.22 (s, 4H).

### Example 2.5

### N-[4-(3-Cyanophenyl)-5-(6-methyl-1H-pyrazolo[3,4-b]pyridin-4-yl)thiazol-2-yl]-2-oxa-6-azaspiro[3.3]heptane-6-carboxamide

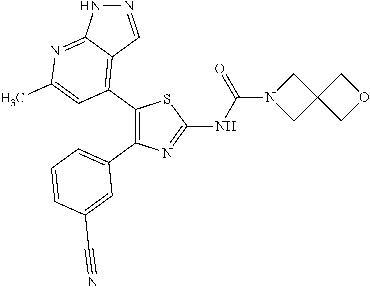 [SMILES: Cc1cc(-c2sc(NC(=O)N3CC4(COC4)C3)nc2-c2cccc(C#N)c2)c2cn[nH]c2n1]

### Step 1: N-[4-(3-Cyanophenyl)-5-(6-methyl-1-trityl-pyrazolo[3,4-b]pyridin-4-yl)thiazol-2-yl]-2-oxa-6-azaspiro[3.3]heptane-6-carboxamide

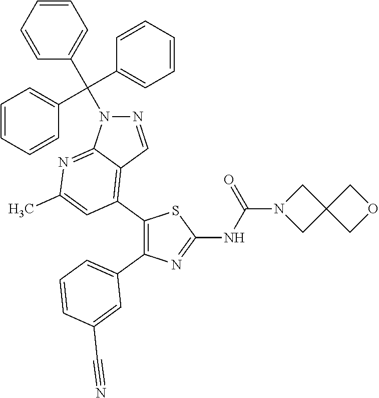 [SMILES: Cc1cc(-c2sc(NC(=O)N3CC4(COC4)C3)nc2-c2cccc(C#N)c2)c2cnn(C(c3ccccc3)(c3ccccc3)c3ccccc3)c2n1]

The title compound was prepared from 3-[2-amino-5-(6-methyl-1-trityl-pyrazolo[3,4-b]pyridin-4-yl)thiazol-4-yl]benzonitrile (Intermediate S) and 2-oxa-6-azaspiro[3.3]heptane analogously to Example 2 step 1.

LC-MS-2 (Method 2B): Rt 1.93 mins; MS m/z 700.3=[M+H]+

1H NMR (500 MHz, DMSO) δ 11.48 (s, 1H), 7.77 (dt, J=7.7, 1.4 Hz, 1H), 7.66 (dd, J=7.3, 1.4 Hz, 2H), 7.55-7.40 (m, 1H), 7.33 (s, 1H), 7.30-7.17 (m, 9H), 7.14-7.06 (m, 6H), 7.03 (s, 1H), 4.68 (s, 4H), 4.23 (s, 4H), 2.20 (s, 3H).

### Step 2: N-[4-(3-Cyanophenyl)-5-(6-methyl-1H-pyrazolo[3,4-b]pyridin-4-yl)thiazol-2-yl]-2-oxa-6-azaspiro[3.3]heptane-6-carboxamide

The title compound was prepared from N-[4-(3-cyanophenyl)-5-(6-methyl-1-trityl-pyrazolo[3,4-b]pyridin-4-yl)thiazol-2-yl]-2-oxa-6-azaspiro[3.3]heptane-6-carboxamide (step 1) and TFA analogously to Example 2 step 2.

LC-MS-2 (Method 8B): Rt 2.62 mins; MS m/z 458.0=[M+H]+

1H NMR (500 MHz, DMSO) δ 13.52 (s, 1H), 11.46 (s, 1H), 7.85 (t, J=1.7 Hz, 1H), 7.76 (dt, J=7.7, 1.4 Hz, 1H), 7.62 (dt, J=8.0, 1.5 Hz, 1H), 7.46 (t, J=7.9 Hz, 1H), 7.36 (s, 1H), 7.06 (s, 1H), 4.69 (s, 4H), 4.23 (s, 4H), 2.56 (s, 3H).

### Example 3

### N-[4-(3-Cyanophenyl)-5-(4-methylquinazolin-6-yl)thiazol-2-yl]-4-piperazin-1-yl-piperidine-1-carboxamide

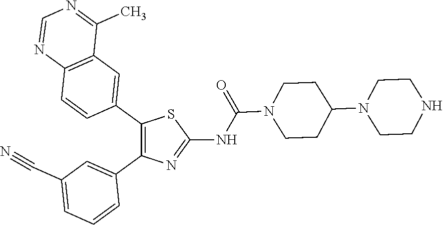 [SMILES: Cc1ncnc2ccc(-c3sc(NC(=O)N4CCC(N5CCNCC5)CC4)nc3-c3cccc(C#N)c3)cc12]

### Step 1: tert-Butyl 4-[1-[[4-(3-cyanophenyl)-5-(4-methylquinazolin-6-yl)thiazol-2-yl]carbamoyl]-4-piperidyl]piperazine-1-carboxylate

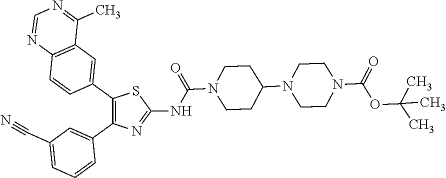 [SMILES: Cc1ncnc2ccc(-c3sc(NC(=O)N4CCC(N5CCN(C(=O)OC(C)(C)C)CC5)CC4)nc3-c3cccc(C#N)c3)cc12]

A solution of 3-[2-amino-5-(4-methylquinazolin-6-yl)thiazol-4-yl]benzonitrile (Intermediate A)(50 mg, 0.15 mmol) and CDI (59 mg, 0.36 mmol) in DMA (0.5 mL) was stirred at 50° C. in a sealed vial for 22 h. To this mixture was added tert-butyl 4-(4-piperidyl)piperazine-1-carboxylate (78 mg, 0.29 mmol) and heating continued at 50° C. for 2 h. The resulting mixture was allowed to cool to room temperature and diluted with water (10 mL), then sonicated. A precipitate formed which was collected under vacuum filtration and dried in a vacuum oven to afford the title compound as a beige solid.

LC-MS-W (Method 2A): Rt 0.72 mins; MS m/z 639.2=[M+H]+

1H NMR (500 MHz, CDCl3) 5 9.18 (s, 1H), 8.49 (s, 1H), 8.09 (d, J=2.0 Hz, 1H), 7.93 (d, J=8.8 Hz, 1H), 7.91-7.85 (m, 1H), 7.69 (dd, J=8.7, 2.0 Hz, 1H), 7.62-7.56 (m, 2H), 7.35 (dd, J=7.8, 7.8 Hz, 1H), 4.28-4.15 (m, 2H), 3.65-3.44 (m, 4H), 3.05-2.97 (m, 2H), 2.87 (s, 3H), 2.73-2.53 (m, 4H), 2.07-1.97 (m, 2H), 1.67-1.60 (m, 2H), 1.46 (s, 1 OH).

### Step 2: N-[4-(3-Cyanophenyl)-5-(4-methylquinazolin-6-yl)thiazol-2-yl]-4-piperazin-1-yl-piperidine-1-carboxamide

A solution of tert-butyl 4-[1-[[4-(3-cyanophenyl)-5-(4-methylquinazolin-6-yl)thiazol-2-yl]carbamoyl]-4-piperidyl]piperazine-1-carboxylate (step 1) (88 mg, 0.13 mmol) in DCM (5 mL) and MeOH (0.5 mL) was treated with 4M HCl in 1,4-dioxane (319 μL, 1.28 mmol) and stirred for 24 h. After standing at room temperature for a further 24 h, the mixture was concentrated in vacuo, then free-based using a 2 g Isolute® SCX cartridge, eluting in 7M NH₃ in MeOH. The resulting yellow gum was dissolved in 3:1:1 DMSO:MeCN:H₂O (1.4 mL) and purified using preparative HPLC under acidic conditions. Further purification using C18 reverse phase chromatography eluting with 0.1% NH₄OH in MeCN/water afforded the title compound as a yellow powder.

LC-MS (Method 7A): Rt 1.87 mins; MS m/z 539.2=[M+H]+

1H NMR (400 MHz, DMSO) δ 9.11 (s, 1H), 8.19 (d, J=2.0 Hz, 1H), 7.95-7.89 (m, 2H), 7.81-7.73 (m, 2H), 7.70 (dt, J=8.1, 1.4 Hz, 1H), 7.51 (dd, J=7.8, 7.8 Hz, 1H), 4.29-4.21 (m, 2H), 2.90-2.79 (m, 5H), 2.75-2.69 (m, 4H), 2.47-2.40 (m, 5H), 1.81-1.73 (m, 2H), 1.40-1.27 (m, 2H). 2×NH not observed.

### Example 3.1

### N-[4-(3-Cyanophenyl)-5-(4-methylquinazolin-6-yl)thiazol-2-yl]-1-oxa-4,9-diazaspiro[5.5]undecane-9-carboxamide

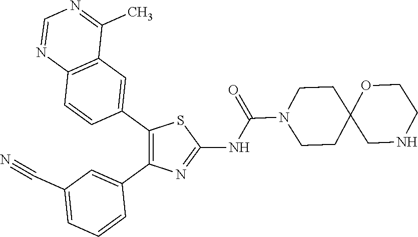 [SMILES: Cc1ncnc2ccc(-c3sc(NC(=O)N4CCC5(CC4)CNCCO5)nc3-c3cccc(C#N)c3)cc12]

### Step 1: tert-butyl 9-[[4-(3-cyanophenyl)-5-(4-methylquinazolin-6-yl)thiazol-2-yl]carbamoyl]-1-oxa-4,9-diazaspiro[5.5]undecane-4-carboxylate

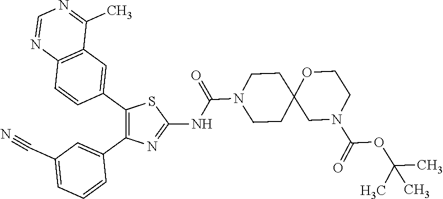 [SMILES: Cc1ncnc2ccc(-c3sc(NC(=O)N4CCC5(CC4)CN(C(=O)OC(C)(C)C)CCO5)nc3-c3cccc(C#N)c3)cc12]

The title compound was prepared from 3-[2-amino-5-(4-methylquinazolin-6-yl)thiazol-4-yl]benzonitrile (Intermediate A) and tert-butyl 1-oxa-4,9-diazaspiro[5.5]undecane-4-carboxylate analogously to Example 3 step 1.

LC-MS-W (Method 2A): Rt 0.99 mins; MS m/z 626.2=[M+H]+

1H NMR (500 MHz, DMSO) δ 11.23 (s, 1H), 9.11 (s, 1H), 8.20 (s, 1H), 7.91 (d, J=1.9 Hz, 1H), 7.79 (d, J=7.7 Hz, 1H), 7.78-7.75 (m, 1H), 7.71 (dt, J=8.3, 1.2 Hz, 1H), 7.52 (dd, J=7.8, 7.8 Hz, 1H), 3.87-3.80 (m, 2H), 3.62 (t, J=5.0 Hz, 2H), 3.30-3.20 (m, 4H), 2.83 (s, 2H), 1.76-1.71 (m, 2H), 1.49 (t, J=11.4 Hz, 2H), 1.41 (s, 9H), 1.26-1.21 (m, 2H). 1×CH₂ not observed.

### Step 2: N-[4-(3-Cyanophenyl)-5-(4-methylquinazolin-6-yl)thiazol-2-yl]-1-oxa-4,9-diazaspiro[5.5]undecane-9-carboxamide

The title compound was prepared from tert-butyl 9-[[4-(3-cyanophenyl)-5-(4-methylquinazolin-6-yl)thiazol-2-yl]carbamoyl]-1-oxa-4,9-diazaspiro[5.5]undecane-4-carboxylate (step 1) and 4M HCl in 1,4-dioxane analogously to Example 3 step 2.

LC-MS (Method 7A): Rt 2.18 mins; MS m/z 526.1=[M+H]+ 1H NMR (500 MHz, MeOD) δ 9.07 (s, 1H), 8.24 (d, J=2.0 Hz, 1H), 7.97-7.93 (m, 2H), 7.85 (dd, J=8.7, 1.9 Hz, 1H), 7.71 (dt, J=7.9, 1.4 Hz, 1H), 7.66 (dt, J=7.7, 1.4 Hz, 1H), 7.43 (dd, J=7.9, 7.9 Hz, 1H), 3.98-3.92 (m, 2H), 3.73-3.68 (m, 2H), 3.36-3.34 (m, 2H), 2.88 (s, 3H), 2.84-2.77 (m, 2H), 2.69 (s, 2H), 2.10-2.04 (m, 2H), 1.56-1.49 (m, 2H). 2×NH not observed.

### Example 4

### N-[4-(3-Cyanophenyl)-5-(4-methylquinazolin-6-yl)thiazol-2-yl]-6-oxa-2,9-diazaspiro[4.5]decane-2-carboxamide

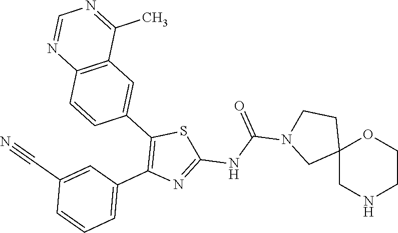 [SMILES: Cc1ncnc2ccc(-c3sc(NC(=O)N4CCC5(CNCCO5)C4)nc3-c3cccc(C#N)c3)cc12]

### Step 1: tert-Butyl 2-[[4-(3-cyanophenyl)-5-(4-methylquinazolin-6-yl)thiazol-2-yl]carbamoyl]-6-oxa-2,9-diazaspiro[4.5]decane-9-carboxylate

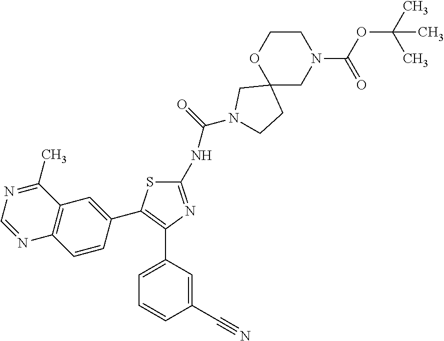 [SMILES: Cc1ncnc2ccc(-c3sc(NC(=O)N4CCC5(C4)CN(C(=O)OC(C)(C)C)CCO5)nc3-c3cccc(C#N)c3)cc12]

To a solution of 3-[2-amino-5-(4-methylquinazolin-6-yl)thiazol-4-yl]benzonitrile (Intermediate A)(75 mg, 0.22 mmol) and CDI (107 mg, 0.66 mmol) in DMA (1 mL) was added DIPEA (0.12 mL, 0.69 mmol) and the reaction mixture was stirred at 50° C. for 3.5 h. tert-Butyl 6-oxa-2,9-diazaspiro[4.5]decane-9-carboxylate (106 mg, 0.44 mmol) was added and mixture was stirred at 50° C. for 1 h. The resulting mixture was allowed to cool to room temperature and partitioned between EtOAc (40 mL) and H₂O (40 mL). The phases were separated and the aqueous portion was further extracted with EtOAc (2×40 mL). The organics were combined and washed with 90% brine (80 mL), 50% brine (80 mL) and brine (50 mL) dried over MgSO₄ and concentrated in vacuo to give a yellow oil. Purification by chromatography on silica eluting with a gradient of 50 to 100% EtOAc in petroleum ether followed by 0 to 10% MeOH in EtOAc afforded the title compound as a pale yellow solid.

LC-MS-1 (Method 2B): Rt 1.45 mins; MS m/z 612.5=[M+H]+

1H NMR (500 MHz, DMSO-d6) δ 11.10 (s, 1H), 9.12 (s, 1H), 8.21 (d, J=2.0 Hz, 1H), 8.14 (s, 1H), 7.93-7.91 (m, 1H), 7.79 (dd, J=7.7, 1.4 Hz, 1H), 7.77 (dd, J=8.8, 2.1 Hz, 1H), 7.71 (dt, J=8.1, 1.5 Hz, 1H), 7.52 (t, J=7.8 Hz, 1H), 3.68-3.61 (m, 6H), 3.57 (d, J=10.1 Hz, 4H), 2.84 (s, 3H), 2.09 (dt, J=12.4, 5.9 Hz, 2H), 1.42 (s, 9H).

### Step 2: N-[4-(3-Cyanophenyl)-5-(4-methylquinazolin-6-yl)thiazol-2-yl]-6-oxa-2,9-diazaspiro[4.5]decane-2-carboxamide

To a solution of tert-butyl 2-[[4-(3-cyanophenyl)-5-(4-methylquinazolin-6-yl)thiazol-2-yl]carbamoyl]-6-oxa-2,9-diazaspiro[4.5]decane-9-carboxylate (step 1) (90 mg, 0.093 mmol) in chloroform (3 mL) was added trimethylsilyl Iodide (84 μL, 0.59 mmol) and the mixture was stirred at room temperature for 1 h. The resulting mixture was concentrated under a flow of nitrogen, chloroform (2 mL) was added and the mixture concentrated once again.

The crude mixture was taken up in DMSO (1 mL) and purified by C18 reverse phase chromatography eluting with 0.1% NH₄OH in MeCN/water. The product was freeze dried overnight then triturated with Et₂O to afford the title compound as a pale yellow solid.

LC-MS-1 (Method 8B): Rt 2.76 mins; MS m/z 512.5=[M+H]+ 1H NMR (500 MHz, DMSO-d6) δ 11.06 (s, 1H), 9.12 (s, 1H), 8.21 (d, J=2.0 Hz, 1H), 7.93 (d, J=9.6 Hz, 2H), 7.81-7.75 (m, 2H), 7.71 (d, J=8.0 Hz, 1H), 7.52 (t, J=7.9 Hz, 1H), 3.70-3.56 (m, 4H), 3.50 (s, 2H), 2.84 (s, 3H), 2.78 (d, J=16.0 Hz, 2H), 2.72 (q, J=5.2, 4.4 Hz, 2H), 2.09 (s, 1H), 1.91 (s, 1H). Morpholine NH not observed.

The compounds of the following tabulated Examples (Table Ex4) were prepared analogously to Example 4 steps 1 and 2 from 3-[2-amino-5-(4-methylquinazolin-6-yl)thiazol-4-yl]benzonitrile (Intermediate A) and the appropriate amine.

TABLE Ex4

| Ex. | Intermediate | Product |
|---|---|---|
| 4.1 | 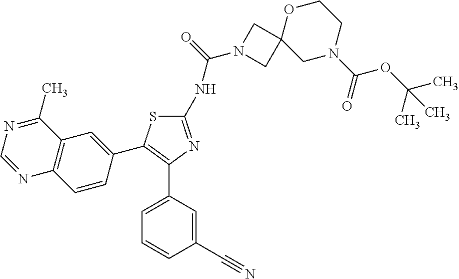 [SMILES: Cc1ncnc2ccc(-c3sc(NC(=O)N4CC5(C4)CN(C(=O)OC(C)(C)C)CCO5)nc3-c3cccc(C#N)c3)cc12] | 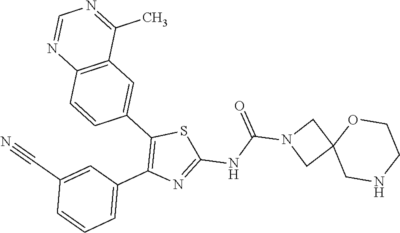 [SMILES: Cc1ncnc2ccc(-c3sc(NC(=O)N4CC5(CNCCO5)C4)nc3-c3cccc(C#N)c3)cc12] |
| | tert-Butyl 2-[[4-(3-cyanophenyl)-5-(4- | N-[4-(3-Cyanophenyl)-5-(4- |
| | methylquinazolin-6-yl)thiazol-2- | methylquinazolin-6-yl)thiazol-2-yl]-5-oxa- |
| | yl]carbamoyl]-5-oxa-2,8- | 2,8-diazaspiro[3.5]nonane-2-carboxamide |
| | diazaspiro[3.5]nonane-8-carboxylate | LC-MS-1 (Method 8B): Rt 2.52 mins; MS |
| | LC-MS-2 (Method 2B): Rt 1.55 mins; | m/z 498.5 = [M + H]+ |
| | MS m/z 598.3 = [M + H]+ | 1H NMR (500 MHz, DMSO-d6) δ 11.45 (s, |
| | 1H NMR (500 MHz, DMSO-d6) δ | 1H), 9.12 (s, 1H), 8.21 (d, J = 2.0 Hz, 1H), |
| | 11.44 (s, 1H), 9.12 (s, 1H), 8.22 (d, | 7.94 (d, J = 8.8 Hz, 1H), 7.90 (s, 1H), 7.80 |
| | J = 2.1 Hz, 1H), 7.94 (d, J = 8.8 Hz, | (d, J = 7.7 Hz, 1H), 7.77 (dd, J = 8.6, 2.0 |
| | 1H), 7.90 (d, J = 1.7 Hz, 1H), 7.81- | Hz, 1H), 7.70 (d, J = 8.0 Hz, 1H), 7.52 (t, J = |
| | 7.79 (m, 1H), 7.78 (dd, J = 8.8, 2.1 | 7.9 Hz, 1H), 4.05 (br s, 2H), 3.90 (br s, 2H), |
| | Hz, 1H), 7.71 (dt, J = 8.0, 1.4 Hz, 1H), | 3.69 (br s, 2H), 3.11 (br s, 2H), 2.88 (br s, |
| | 7.52 (t, J = 7.9 Hz, 1H), 3.95 (s, 2H), | 2H), 2.83 (s, 3H). Morpholine NH not |
| | 3.90 (d, J = 9.2 Hz, 2H), 3.60 (t, J = | observed. |
| | 4.9 Hz, 2H), 3.52 (s, 2H), 2.84 (s, 3H), | |
| | 1.44 (s, 9H). 1 x CH₂ not observed. | |
| 4.2 | 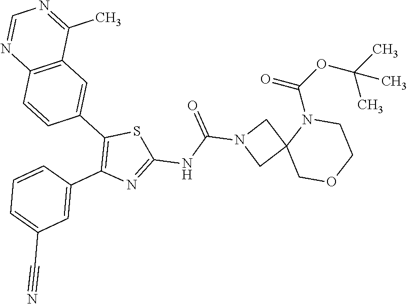 [SMILES: Cc1ncnc2ccc(-c3sc(NC(=O)N4CC5(COCCN5C(=O)OC(C)(C)C)C4)nc3-c3cccc(C#N)c3)cc12] | 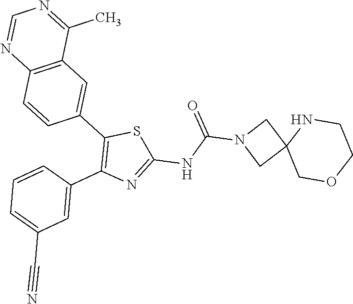 [SMILES: Cc1ncnc2ccc(-c3sc(NC(=O)N4CC5(COCCN5)C4)nc3-c3cccc(C#N)c3)cc12] |
| | tert-butyl 2-[[4-(3-cyanophenyl)-5-(4- | N-[4-(3-Cyanophenyl)-5-(4- |
| | methylquinazolin-6-yl)thiazol-2- | methylquinazolin-6-yl)thiazol-2-yl]-8-oxa- |
| | yl]carbamoyl]-8-oxa-2,5- | 2,5-diazaspiro[3.5]nonane-2-carboxamide |
| | diazaspiro[3.5]nonane-5-carboxylate | LC-MS-1 (Method 8A): Rt 2.40 mins; MS |
| | LC-MS-1 (Method 2B): Rt 1.37 mins; | m/z 498.5 = [M + H]+ |
| | MS m/z 598.5 = [M + H]+ | 1H NMR (500 MHz, DMSO) δ 11.33 (br s, |
| | 1H NMR (500 MHz, DMSO-d6) δ | 1H), 9.11 (s, 1H), 8.20 (d, J = 2.0 Hz, 1H), |
| | 11.46 (s, 1H), 9.13 (s, 1H), 8.22 (d, | 7.93 (d, J = 8.7 Hz, 1H), 7.90 (s, 1H), 7.81- |
| | J = 2.0 Hz, 1H), 7.94 (d, J = 8.7 Hz, | 7.75 (m, 2H), 7.70 (d, J = 8.0 Hz, 1H), 7.52 |
| | 1H), 7.90 (t, J = 1.6 Hz, 1H), 7.80 (dt, | (apr t, J = 7.8 Hz, 1H), 3.86 (br s, 2H), 3.76 |
| | J = 7.8, 1.6 Hz, 1H), 7.78 (dd, J = 8.7, | (br s, 2H), 3.57 (s, 2H), 3.51 (t, J = 4.6 Hz, |
| | 2.0 Hz, 1H), 7.71 (dt, J = 7.8, 1.6 Hz, | 2H), 3.06 (br s, 1H), 2.83 (s, 3H), 2.68 (t, |
| | 1H), 7.53 (t, J = 7.8 Hz, 1H), 4.23 (br | J = 4.6 Hz, 2H) |
| | d, J = 8.5 Hz, 2H), 3.94 (s, 2H), 3.72 | |
| | (s, 2H), 3.53 (t, J = 4.8 Hz, 2H), 2.84 | |
| | (s, 3H), 1.45 (s, 9H). 1 x CH₂ signal | |
| | not observed. | |
| 4.3 | 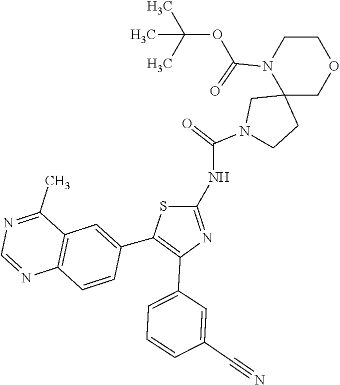 [SMILES: Cc1ncnc2ccc(-c3sc(NC(=O)N4CCC5(COCCN5C(=O)OC(C)(C)C)C4)nc3-c3cccc(C#N)c3)cc12] | 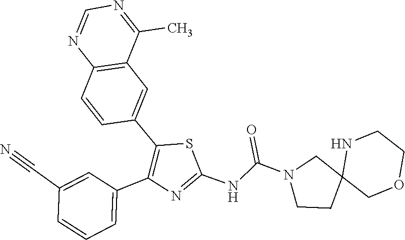 [SMILES: Cc1ncnc2ccc(-c3sc(NC(=O)N4CCC5(COCCN5)C4)nc3-c3cccc(C#N)c3)cc12] |
| | tert-Butyl 2-[[4-(3-cyanophenyl)-5-(4- | N-[4-(3-Cyanophenyl)-5-(4- |
| | methylquinazolin-6-yl)thiazol-2- | methylquinazolin-6-yl)thiazol-2-yl]-9-oxa- |
| | yl]carbamoyl]-9-oxa-2,6- | 2,6-diazaspiro[4.5]decane-2-carboxamide |
| | diazaspiro[4.5]decane-6-carboxylate | LC-MS-1 (Method 8B): Rt 2.73 mins; MS |
| | LC-MS-1 (Method 2B): Rt 1.48 mins; | m/z 512.5 = [M + H]+ |
| | MS m/z 612.5 = [M + H]+ | 1H NMR (500 MHz, DMSO-d6) δ 11.03 (br |
| | 1H NMR (500 MHz, DMSO-d6) δ | s, 2H), 9.12 (s, 1H), 8.21 (d, J = 2.0 Hz, |
| | 11.07 (s, 1H), 9.12 (s, 1H), 8.21 (d, | 1H), 7.94 (d, J = 8.6 Hz, 2H), 7.82-7.76 |
| | J = 2.0 Hz, 1H), 7.93 (d, J = 8.6 Hz, | (m, 2H), 7.72 (d, J = 8.0 Hz, 1H), 7.52 (t, J = |
| | 2H), 7.81-7.76 (m, 2H), 7.71 (dt, J = | 7.9 Hz, 1H), 3.57 (s, 2H), 3.48 (d, J = 10.7 |
| | 8.0, 1.5 Hz, 1H), 7.52 (t, J = 7.9 Hz, | Hz, 4H), 3.41-3.38 (m, 2H), 2.84 (s, 3H), |
| | 1H), 3.82 (s, 1H), 3.69 (dt, J = 10.0, | 2.77-2.72 (m, 2H), 1.89 (br s, 2H). |
| | 4.7 Hz, 2H), 3.65 (s, 2H), 3.55-3.46 | |
| | (m, 4H), 3.44 (d, J = 6.1 Hz, 1H), 2.84 | |
| | (s, 3H), 2.41 (dd, J = 12.5, 9.1 Hz, | |
| | 1H), 2.11 (s, 1H), 1.41 (s, 9H). | |

### Example 5

### (3S)—N-[4-(3-Cyanophenyl)-5-(4-methylquinazolin-6-yl)thiazol-2-yl]-3-(1-hydroxy-1-methyl-ethyl)pyrrolidine-1-carboxamide

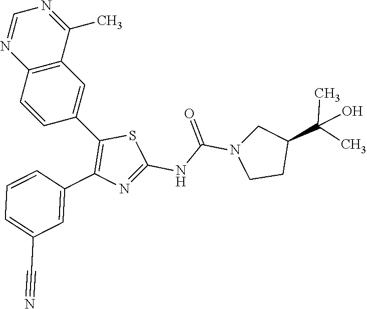 [SMILES: Cc1ncnc2ccc(-c3sc(NC(=O)N4CC[C@H](C(C)(C)O)C4)nc3-c3cccc(C#N)c3)cc12]

Anhydrous DMA (15 mL) and DIPEA (2.28 mL, 13.1 mmol) were added to a mixture of 3-[2-amino-5-(4-methylquinazolin-6-yl)thiazol-4-yl]benzonitrile (Intermediate A)(1.5 g, 4.37 mmol) and CDI (1.42 g, 8.74 mmol) in a sealed flask and the mixture was stirred at 50° C. for 18 h. A solution of 2-[(3S)-pyrrolidin-3-yl]propan-2-ol (1.13 g, 8.74 mmol) in anhydrous DMA (3 mL) was added and the mixture stirred at 50° C. for a further 30 mins. The resulting mixture was allowed to cool to room temperature, diluted with EtOAc (200 mL) and washed with 80% brine (200 mL), 50% brine (3×200 mL). The organic portion was dried over Na₂SO₄ and concentrated in vacuo. Purification by chromatography on silica eluting with a gradient of 0 to 5% MeOH in DCM afforded a pale yellow foam/glass which was mechanically broken up into a powder and dried in a vacuum oven at 50° C. overnight to afford the title compound as a pale yellow solid.

LC-MS-2 (Method 8A): Rt 3.33 mins; MS m/z 499.2=[M+H]+

1H NMR (500 MHz, DMSO) δ 10.95 (s, 1H), 9.11 (s, 1H), 8.20 (d, J=2.0 Hz, 1H), 7.95-7.90 (m, 2H), 7.81-7.75 (m, 2H), 7.71 (br dt, J=8.0, 1.5 Hz, 1H), 7.51 (apr t, J=7.8 Hz, 1H), 4.38 (s, 1H), 3.65 (br s, 1H), 3.57 (br s, 1H), 3.27 (br s, 2H), 2.83 (s, 3H), 2.22 (br s, 1H), 1.84 (br s, 2H), 1.12 (s, 6H).

### Example 6

### 4-Cyano-N-[4-(3-cyanophenyl)-5-(4-methylquinazolin-6-yl)thiazol-2-yl]-4-methyl-piperidine-1-carboxamide

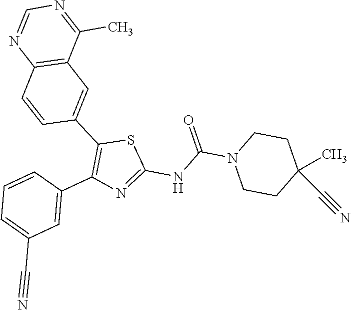 [SMILES: Cc1ncnc2ccc(-c3sc(NC(=O)N4CCC(C)(C#N)CC4)nc3-c3cccc(C#N)c3)cc12]

Anhydrous DMA (2 mL) and DIPEA (228 μL, 1.31 mmol) were added to a mixture of 3-[2-amino-5-(4-methylquinazolin-6-yl)thiazol-4-yl]benzonitrile (Intermediate A)(150 mg, 0.44 mmol) and CDI (14 mg, 0.87 mmol) in a sealed tube under nitrogen and the resulting orange-brown solution stirred at 50° C. for 18 h. 4-Methylpiperidine-4-carbonitrile hydrochloride (140 mg, 0.87 mmol) and additional DIPEA (0.23 mL, 1.31 mmol) were added and the mixture stirred at 50° C. for a further 3 h. The resulting mixture was allowed to cool to room temperature, diluted with EtOAc (20 mL) and washed with 80% brine (20 mL) then 50% brine (3×50 mL). The organic portion was dried over Na₂SO₄ and concentrated in vacuo. Purification by chromatography on silica eluting with a gradient of 0 to 3% MeOH in DCM afforded a pale yellow solid. The material was azeotroped twice with MeCN and the glassy residue re-dissolved in DMSO (2 mL) and 2 drops of water added. This mixture was re-purified by C18 reverse phase chromatography eluting with 0.1% NH₄OH in MeCN/water. The product fractions were combined and concentrated in vacuo to afford a wet residue which was azeotroped in a 1:1 mixture of MeCN and MeOH to afford a clear yellow glass. This material was mechanically broken up into a powder then vacuum dried to afford the title compound as a yellow solid.

LC-MS-1 (Method 8A): Rt 3.49 mins; MS m/z 494.4=[M+H]+

1H NMR (500 MHz, DMSO) δ 11.33 (br s, 1H), 9.11 (s, 1H), 8.20 (d, J=2.0 Hz, 1H), 7.94-7.90 (m, 2H), 7.79 (br dt, J=7.6, 1.5 Hz, 1H), 7.76 (dd, J=8.7, 2.0 Hz, 1H), 7.71 (br dt, J=8.2, 1.5 Hz, 1H), 7.52 (apr t, J=7.8 Hz, 1H), 4.25 (d, J=14.1 Hz, 2H), 3.05 (t, J=13.1 Hz, 2H), 2.83 (s, 3H), 1.93 (d, J=13.6 Hz, 2H), 1.54 (td, J=13.1, 4.0 Hz, 2H), 1.38 (s, 3H).

### Example 7

### (3R)—N-[4-(3-Cyanophenyl)-5-(4-methylquinazolin-6-yl)thiazol-2-yl]-3-(1-hydroxy-1-methyl-ethyl)pyrrolidine-1-carboxamide

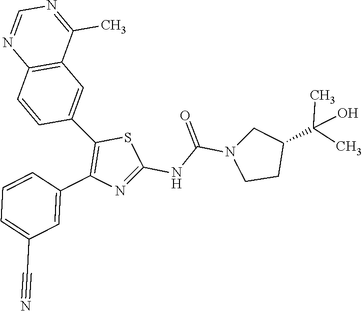 [SMILES: Cc1ncnc2ccc(-c3sc(NC(=O)N4CC[C@@H](C(C)(C)O)C4)nc3-c3cccc(C#N)c3)cc12]

Anhydrous DMA (5.8 mL) and DIPEA (609 μL, 3.49 mmol) were added to a mixture of 3-[2-amino-5-(4-methylquinazolin-6-yl)thiazol-4-yl]benzonitrile (Intermediate A)(400 mg, 1.16 mmol) and CDI (378 mg, 2.33 mmol) in a sealed tube under nitrogen and the and the mixture was stirred at 50° C. overnight. 2-[(3R)-Pyrrolidin-3-yl]propan-2-ol (301 mg, 2.33 mmol) was added and the mixture stirred at 50° C. for a further 90 mins. The resulting mixture was allowed to cool to room temperature and then partitioned between EtOAc (50 mL) and H₂O (50 mL). The organic portion was separated and the aqueous further extracted with EtOAc (2×30 mL). The combined organics were washed with brine (5×40 mL), dried over Na₂SO₄ and the solvent removed in vacuo. Purification by chromatography on silica eluting in 0 to 5% MeOH in DCM afforded a pale yellow glassy solid. The solid was azeotroped and triturated with Et₂O to afford the title compound as a pale yellow solid.

LC-MS-1 (Method 8A): Rt 3.19 mins; MS m/z 499.4=[M+H]+ 1H NMR (500 MHz, Methanol-d4) δ 9.07 (s, 1H), 8.24 (d, J=2.0 Hz, 1H), 7.98-7.91 (m, 2H), 7.85 (dd, J=8.8, 2.0 Hz, 1H), 7.73-7.68 (m, 1H), 7.68-7.63 (m, 1H), 7.43 (t, J=7.9 Hz, 1H), 3.73 (t, J=9.4 Hz, 1H), 3.66 (dd, J=10.2, 8.1 Hz, 1H), 3.45 (s, 1H), 3.39 (t, J=10.1 Hz, 1H), 2.88 (s, 3H), 2.40 (s, 1H), 2.04 (s, 1H), 1.94 (s, 1H), 1.27 (d, J=2.1 Hz, 6H). NH and OH protons not observed.

The compounds of the following tabulated Examples (Table Ex7) were prepared analogously to Examples 5-7 from 3-[2-amino-5-(4-methylquinazolin-6-yl)thiazol-4-yl]benzonitrile (Intermediate A) and the appropriate amine.

### Table Ex7

| | | Retention Time, [M + H]+, ¹H |
|---|---|---|
| Ex. | Structure and Name | NMR |
| 7.1 | 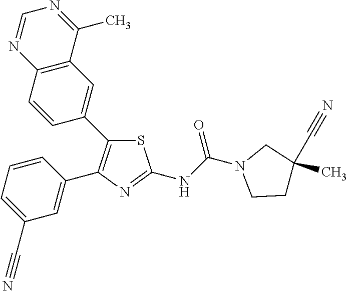 [SMILES: Cc1ncnc2ccc(-c3sc(NC(=O)N4CC[C@@](C)(C#N)C4)nc3-c3cccc(C#N)c3)cc12] | LC-MS-1 (Method 8A): Rt 3.27 mins; MS m/z 480.4 = [M + H]+ 1H NMR (500 MHz, DMSO-d6) δ 11.25 (s, 1H), 9.12 (s, 1H), 8.22 (d, J = 2.0 Hz, 1H), 7.95-7.90 (m, 2H), 7.82-7.75 (m, 2H), 7.71 (d, J = 7.9 Hz, 1H), 7.52 (t, J = 7.9 Hz, 1H), 3.98-3.89 (m, 1H), 3.74- 3.66 (m, 1H), 3.66-3.57 (m, 1H), 3.47-3.40 (m, 1H), 2.84 (s, 3H), 2.44-2.38 (m, 1H), 2.13- 2.05 (m, 1H), 1.48 (s, 3H). |
| | (3R)-3-Cyano-N-[4-(3-cyanophenyl)- | |
| | 5-(4-methylquinazolin-6-yl)thiazol-2- | |
| | yl]-3-methyl-pyrrolidine-1- | |
| | carboxamide | |
| | (Amine used—Intermediate BA) | |
| 7.2 | 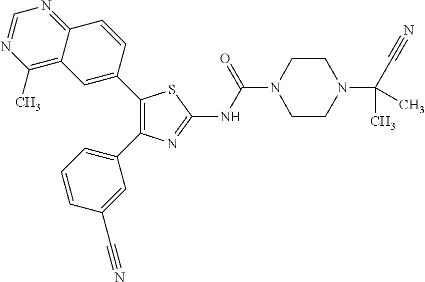 [SMILES: Cc1ncnc2ccc(-c3sc(NC(=O)N4CCN(C(C)(C)C#N)CC4)nc3-c3cccc(C#N)c3)cc12] | LC-MS-2 (Method 8A): Rt 3.46 mins; MS m/z 523.5 = [M + H]+ 1H NMR (500 MHz, Methanol-d4) δ 9.07 (s, 1H), 8.25 (d, J = 2.0 Hz, 1H), 7.97-7.93 (m, 2H), 7.86 (dd, J = 8.7, 2.0 Hz, 1H), 7.71 (dt, J = 7.9, 1.5 Hz, 1H), 7.66 (dt, J = 7.9, 1.5 Hz, 1H), 7.43 (t, J = 7.9 Hz, 1H), 3.70-3.65 (m, 4H), 2.88 (s, 3H), 2.75-2.70 (m, 4H), 1.56 (s, 6H). NH not observed |
| | 4-(1-Cyano-1-methyl-ethyl)-N-[4-(3- | |
| | cyanophenyl)-5-(4-methylquinazolin- | |
| | 6-yl)thiazol-2-yl]piperazine-1- | |
| | carboxamide | |
| | (Amine used—Intermediate C) | |
| 7.3 | 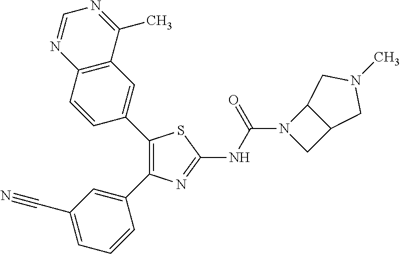 [SMILES: Cc1ncnc2ccc(-c3sc(NC(=O)N4CC5CN(C)CC54)nc3-c3cccc(C#N)c3)cc12] | LC-MS (Method 7A): Rt 1.79 mins; MS m/z 482.1 = [M + H]+ 1H NMR (400 MHz, MeOD) δ 9.07 (s, 1H), 8.24 (d, J = 1.9 Hz, 1H), 7.95 (d, J = 8.8 Hz, 1H), 7.93- 7.92 (m, 1H), 7.85 (dd, J = 8.7, 2.0 Hz, 1H), 7.70 (dt, J = 8.0, 1.4 Hz, 1H), 7.65 (dt, J = 7.9, 1.3 Hz, 1H), 7.42 (dd, J = 7.8, 7.8 Hz, 1H), 4.68-4.51 (m, 1H), 4.20 (t, J = 8.3 Hz, 1H), 3.90-3.82 (m, 1H), 3.41 (d, J = 11.3 Hz, 1H), 3.18-3.12 (m, 1H), 3.09 (d, J = 10.4 Hz, 1H), 2.87 (s, 3H), 2.46 (s, 3H), 2.19 (dd, J = 10.3, 6.0 Hz, 1H), 2.12 (dd, J = 11.2, 4.1 Hz, 1H). NH not observed |
| | N-[4-(3-Cyanophenyl)-5-(4- | |
| | methylquinazolin-6-yl) thiazol-2-yl]-3- | |
| | methyl-3,6- | |
| | diazabicyclo[3.2.0]heptane-6- | |
| | carboxamide | |
| 7.4 | 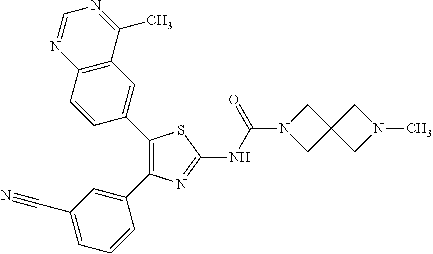 [SMILES: Cc1ncnc2ccc(-c3sc(NC(=O)N4CC5(CN(C)C5)C4)nc3-c3cccc(C#N)c3)cc12] | LC-MS (Method 7A): Rt 1.81 mins; MS m/z 482.1 = [M + H]+ 1H NMR (400 MHz, MeOD) δ 9.07 (s, 1H), 8.23 (dd, J = 2.0, 0.6 Hz, 1H), 7.94 (d, J = 8.8 Hz, 1H), 7.93-7.91 (m, 1H), 7.84 (dd, J = 8.7, 2.0 Hz, 1H), 7.71-7.68 (m, 1H), 7.65 (dt, J = 7.8, 1.3 Hz, 1H), 7.43 (dd, J = 7.9, 7.7 Hz, 1H), 4.21 (s, 4H), 3.46 (s, 4H), 2.87 (s, 3H), 2.34 (s, 3H). NH not observed |
| | N-[4-(3-Cyanophenyl)-5-(4- | |
| | methylquinazolin-6-yl)thiazol-2-yl]-6- | |
| | methyl-2,6-diazaspiro[3.3]heptane-2- | |
| | carboxamide | |
| 7.5 | 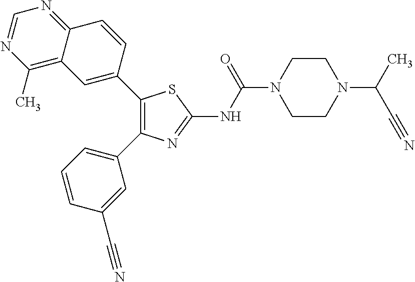 [SMILES: Cc1ncnc2ccc(-c3sc(NC(=O)N4CCN(C(C)C#N)CC4)nc3-c3cccc(C#N)c3)cc12] | LC-MS-1 (Method 8A): Rt 3.30 mins; MS m/z 509.5 = [M + H]+ 1H NMR (500 MHz, DMSO) δ 11.32 (s, 1H), 9.13 (s, 1H), 8.23 (d, J = 2.0 Hz, 1H), 7.97-7.89 (m, 2H), 7.84-7.75 (m, 2H), 7.72 (d, J = 8.0 Hz, 1H), 7.53 (t, J = 7.8 Hz, 1H), 4.06 (q, J = 7.1 Hz, 1H), 3.74-3.51 (m, 4H), 2.85 (s, 3H), 2.66-2.59 (m, 2H), 2.46-2.41 (m, 2H), 1.40 (d, J = 7.2 Hz, 3H). |
| | 4-(1-Cyanoethyl)-N-[4-(3- | |
| | cyanophenyl)-5-(4-methylquinazolin- | |
| | 6-yl)thiazol-2-yl]piperazine-1- | |
| | carboxamide | |
| 7.6 | 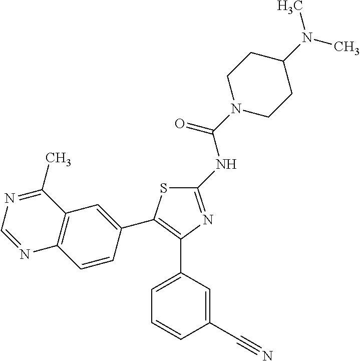 [SMILES: Cc1ncnc2ccc(-c3sc(NC(=O)N4CCC(N(C)C)CC4)nc3-c3cccc(C#N)c3)cc12] | LC-MS-1 (Method 8B): Rt 3.12 mins; MS m/z 496.5 = [M − H]− 1H NMR (500 MHz, DMSO-d6) δ 11.17 (br s, 1H), 9.11 (s, 1H), 8.21 (s, 1H), 7.93 (d, J = 10.0 Hz, 2H), 7.78 (dd, J = 15.1, 8.3 Hz, 2H), 7.71 (d, J = 7.9 Hz, 1H), 7.52 (t, J = 7.9 Hz, 1H), 4.22 (d, J = 13.3 Hz, 2H), 2.89 (t, J = 12.8 Hz, 2H), 2.84 (s, 3H), 2.33 (s, 1H), 2.18 (s, 6H), 1.82-1.74 (m, 2H), 1.36-1.25 (m, 2H). |
| | N-[4-(3-Cyanophenyl)-5-(4- | |
| | methylquinazolin-6-yl)thiazol-2-yl]-4- | |
| | (dimethylamino)piperidine-1- | |
| | carboxamide | |
| 7.7 | 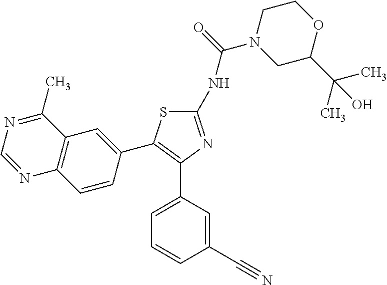 [SMILES: Cc1ncnc2ccc(-c3sc(NC(=O)N4CCOC(C(C)(C)O)C4)nc3-c3cccc(C#N)c3)cc12] | LC-MS-1 (Method 8A): Rt 3.26 mins; MS m/z 515.5 = [M + H]+ 1H NMR (500 MHz, DMSO-d6) δ 11.34 (s, 1H), 9.12 (s, 1H), 8.21 (s, 1H), 7.95-7.90 (m, 2H), 7.78 (dd, J = 13.7, 8.2 Hz, 2H), 7.71 (d, J = 7.9 Hz, 1H), 7.52 (t, J = 7.8 Hz, 1H), 4.50 (s, 1H), 4.27 (d, J = 13.3 Hz, 1H), 4.10-4.05 (m, 1H), 3.94 (d, J = 11.3 Hz, 1H), 3.45 (s, 2H), 3.13-3.09 (m, 1H), 2.99- 2.92 (m, 2H), 2.82-2.77 (m, 2H), 1.14 (s, 3H), 1.10 (s, 3H). |
| | N-[4-(3-Cyanophenyl)-5-(4- | |
| | methylquinazolin-6-yl)thiazol-2-yl]-2- | |
| | (1-hydroxy-1-methyl- | |
| | ethyl)morpholine-4-carboxamide | |
| 7.8 | 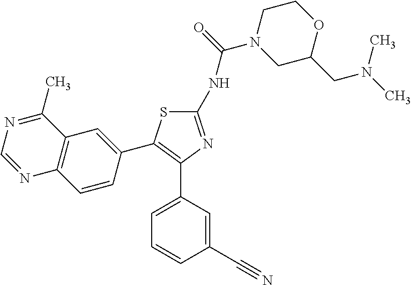 [SMILES: Cc1ncnc2ccc(-c3sc(NC(=O)N4CCOC(CN(C)C)C4)nc3-c3cccc(C#N)c3)cc12] | LC-MS-2 (Method 8A): Rt 2.52 mins; MS m/z 514.2 = [M + H]+ 1H NMR (500 MHz, DMSO-d6) δ 9.11 (s, 1H), 8.20 (d, J = 2.0 Hz, 1H), 7.94-7.90 (m, 2H), 7.79 (d, J = 7.7 Hz, 1H), 7.77 (dd, J = 8.7, 2.1 Hz, 1H), 7.71 (d, J = 8.0 Hz, 1H), 7.52 (t, J = 7.9 Hz, 1H), 4.18 (d, J = 13.0 Hz, 1H), 4.05 (d, J = 13.0 Hz, 1H), 3.86 (dd, J = 11.1, 3.2 Hz, 1H), 3.54-3.50 (m, 1H), 3.46 (dd, J = 11.7, 9.0 Hz, 1H), 2.98 (t, J = 12.6 Hz, 1H), 2.83 (s, 3H), 2.70 (t, J = 11.9 Hz, 1H), 2.32 (d, J = 5.9 Hz, 2H), 2.19 (s, 6H). NH not observed. |
| | N-[4-(3-Cyanophenyl)-5-(4- | |
| | methylquinazolin-6-yl)thiazol-2-yl]-2- | |
| | [(dimethylamino)methyl]morpholine- | |
| | 4-carboxamide | |
| 7.9 | 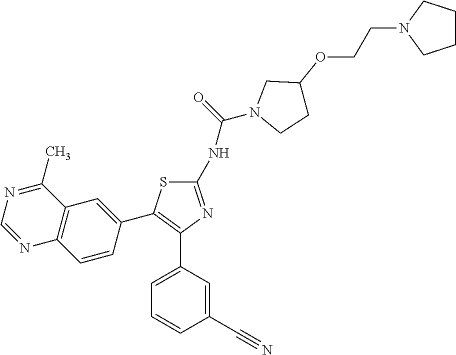 [SMILES: Cc1ncnc2ccc(-c3sc(NC(=O)N4CCC(OCCN5CCCC5)C4)nc3-c3cccc(C#N)c3)cc12] | LC-MS-2 (Method 8A): Rt 2.61 mins; MS m/z 554.2 = [M + H]+ 1H NMR (500 MHz, DMSO-d6) δ 11.05 (s, 1H), 9.12 (s, 1H), 8.21 (d, J = 2.0 Hz, 1H), 7.94 (d, J = 8.8 Hz, 2H), 7.81-7.79 (m, 1H), 7.78 (dd, J = 8.7, 2.1 Hz, 1H), 7.72 (dt, J = 8.0, 1.4 Hz, 1H), 7.52 (t, J = 7.9 Hz, 1H), 4.14 (s, 1H), 3.60-3.47 (m, 6H), 2.84 (s, 3H), 2.57 (q, J = 5.9 Hz, 2H), 2.45 (dt, J = 6.4, 3.7 Hz, 4H), 2.00 (s, 2H), 1.66 (p, J = 3.1 Hz, 4H). |
| | N-[4-(3-Cyanophenyl)-5-(4- | |
| | methylquinazolin-6-yl)thiazol-2-yl]-3- | |
| | (2-pyrrolidin-1-ylethoxy)pyrrolidine-1- | |
| | carboxamide | |
| 7.10 | 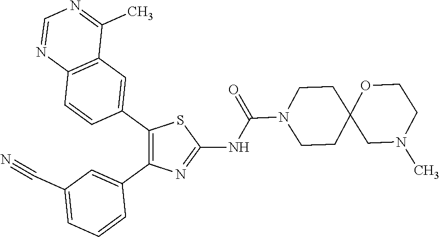 [SMILES: Cc1ncnc2ccc(-c3sc(NC(=O)N4CCC5(CC4)CN(C)CCO5)nc3-c3cccc(C#N)c3)cc12] | LC-MS (Method 7B): Rt 1.93 mins; MS m/z 540.1 = [M + H]+ 1H NMR (500 MHz, MeOD) δ 9.07 (s, 1H), 8.24 (d, J = 1.9 Hz, 1H), 7.97-7.93 (m, 2H), 7.85 (dd, J = 8.7, 2.0 Hz, 1H), 7.71 (dt, J = 8.0, 1.4 Hz, 1H), 7.66 (dt, J = 7.8, 1.4 Hz, 1H), 7.43 (dd, J = 7.8, 7.8 Hz, 1H), 3.99-3.90 (m, 2H), 3.79-3.74 (m, 2H), 3.36-3.33 (m, 2H), 2.88 (s, 3H), 2.42-2.37 (m, 2H), 2.29 (s, 2H), 2.24 (s, 3H), 2.04 (d, J = 13.9 Hz, 2H), 1.61-1.54 (m, 2H). NH not observed. |
| | N-[4-(3-Cyanophenyl)-5-(4- | |
| | methylquinazolin-6-yl)thiazol-2-yl]-4- | |
| | methyl-1-oxa-4,9- | |
| | diazaspiro[5.5]undecane-9- | |
| | carboxamide | |
| 7.11 | 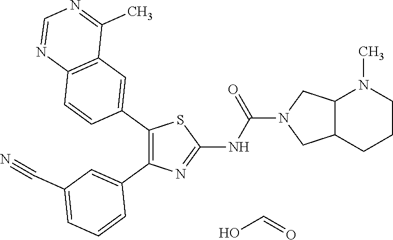 [SMILES: Cc1ncnc2ccc(-c3sc(NC(=O)N4CC5CCCN(C)C5C4)nc3-c3cccc(C#N)c3)cc12 ; O=CO] | LC-MS (Method 7B): Rt 2.94 mins; MS m/z 510.4 = [M + H]+ 1H NMR (400 MHz, MeOD) δ 9.07 (s, 1H), 8.35 (s, 1H), 8.24 (d, J = 1.9 Hz, 1H), 7.97-7.94 (m, 2H), 7.85 (dd, J = 8.8, 2.0 Hz, 1H), 7.70 (dt, J = 8.0, 1.5 Hz, 1H), 7.66 (dt, J = 7.8, 1.3 Hz, 1H), 7.43 (dd, J = 7.9, 7.9 Hz, 1H), 3.98- 3.90 (m, 1H), 3.62 (q, J = 11.2 Hz, 2H), 3.53 (t, J = 9.8 Hz, 1H), 3.15-3.07 (m, 1H), 3.03-2.94 (m, 1H), 2.88 (s, 3H), 2.51 (s, 3H), 2.50-2.45 (m, 1H), 1.81 (d, J = 30.0 Hz, 5H). NH not observed. |
| | N-[4-(3-Cyanophenyl)-5-(4- | |
| | methylquinazolin-6-yl)thiazol-2-yl]-1- | |
| | methyl-3,4,4a,5,7,7a-hexahydro-2H- | |
| | pyrrolo[3,4-b]pyridine-6-carboxamide | |
| | formate salt | |
| 7.12 | 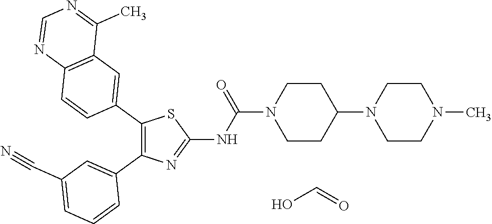 [SMILES: Cc1ncnc2ccc(-c3sc(NC(=O)N4CCC(N5CCN(C)CC5)CC4)nc3-c3cccc(C#N)c3)cc12 ; O=CO] | LC-MS (Method 7B): Rt 2.50 mins; MS m/z 553.4 = [M + H]+ 1H NMR (400 MHz, MeOD) δ 9.07 (s, 1H), 8.47 (s, 1H), 8.23 (d, J = 1.9 Hz, 1H), 7.97-7.92 (m, 2H), 7.84 (dd, J = 8.7, 2.0 Hz, 1H), 7.71-7.68 (m, 1H), 7.66 (dt, J = 7.8, 1.3 Hz, 1H), 7.43 (dd, J = 7.8, 7.8 Hz, 1H), 4.34-4.25 (m, 2H), 3.06-2.88 (m, 6H), 2.87 (s, 3H), 2.86-2.73 (m, 4H), 2.72- 2.65 (m, 1H), 2.61 (s, 3H), 2.02- 1.94 (m, 2H), 1.53 (qd, J = 12.3, 4.1 Hz, 2H). NH not observed. |
| | N-[4-(3-Cyanophenyl)-5-(4- | |
| | methylquinazolin-6-yl)thiazol-2-yl]-4- | |
| | (4-methylpiperazin-1-yl)piperidine-1- | |
| | carboxamide formate salt | |
| 7.13 | 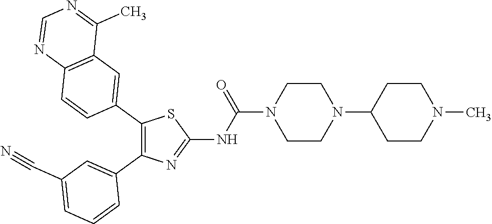 [SMILES: Cc1ncnc2ccc(-c3sc(NC(=O)N4CCN(C5CCN(C)CC5)CC4)nc3-c3cccc(C#N)c3)cc12] | LC-MS (Method 7A): Rt 1.44 mins; MS m/z 553.2 = [M + H]+ 1H NMR (400 MHz, MeOD) δ 9.07 (s, 1H), 8.24 (d, J = 1.9 Hz, 1H), 7.98-7.92 (m, 2H), 7.85 (dd, J = 8.8, 2.0 Hz, 1H), 7.70 (dt, J = 8.0, 1.4 Hz, 1H), 7.66 (dt, J = 7.8, 1.3 Hz, 1H), 7.43 (dd, J = 7.8, 7.8 Hz, 1H), 3.63 (t, J = 5.0 Hz, 4H), 3.05-2.97 (m, 2H), 2.88 (s, 3H), 2.69-2.64 (m, 4H), 2.43- 2.34 (m, 1H), 2.33 (s, 3H), 2.20- 2.11 (m, 2H), 1.96-1.88 (m, 2H), 1.67-1.56 (m, 2H). NH not observed. |
| | N-[4-(3-Cyanophenyl)-5-(4- | |
| | methylquinazolin-6-yl)thiazol-2-yl]-4- | |
| | (1-methyl-4-piperidyl)piperazine-1- | |
| | carboxamide | |
| 7.14 | 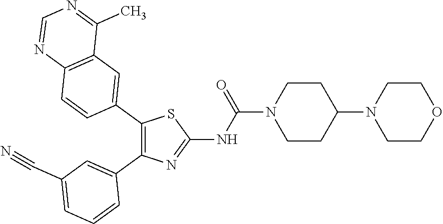 [SMILES: Cc1ncnc2ccc(-c3sc(NC(=O)N4CCC(N5CCOCC5)CC4)nc3-c3cccc(C#N)c3)cc12] | LC-MS (Method 7A): Rt 1.83 mins; MS m/z 540.1 = [M + H]+ 1H NMR (500 MHz, DMSO) δ 11.23 (s, 1H), 9.11 (s, 1H), 8.20 (d, J = 2.0 Hz, 1H), 7.96-7.89 (m, 2H), 7.79 (dt, J = 7.7, 1.4 Hz, 1H), 7.76 (dd, J = 8.7, 2.0 Hz, 1H), 7.70 (dt, J = 8.0, 1.4 Hz, 1H), 7.52 (dd, J = 7.8, 7.8 Hz, 1H), 4.28-4.19 (m, 2H), 3.64-3.52 (m, 4H), 2.89 (t, J = 12.6 Hz, 2H), 2.84 (s, 3H), 2.49-2.44 (m, 4H), 2.42-2.37 (m, 1H), 1.86-1.77 (m, 2H), 1.33 (qd, J = 12.5, 3.7 Hz, 2H). |
| | N-[4-(3-Cyanophenyl)-5-(4- | |
| | methylquinazolin-6-yl)thiazol-2-yl]-4- | |
| | morpholino-piperidine-1- | |
| | carboxamide | |
| 7.15 | 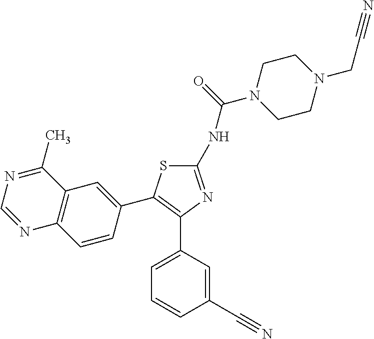 [SMILES: Cc1ncnc2ccc(-c3sc(NC(=O)N4CCN(CC#N)CC4)nc3-c3cccc(C#N)c3)cc12] | LC-MS-2 (Method 8B): Rt 3.11 mins; MS m/z 495.2 = [M + H]+ 1H NMR (500 MHz, Methanol-d4) δ 9.07 (s, 1H), 8.24 (d, J = 2.0 Hz, 1H), 7.95 (d, J = 8.5 Hz, 2H), 7.85 (dd, J = 8.8, 2.0 Hz, 1H), 7.71 (d, J = 8.0 Hz, 1H), 7.66 (d, J = 7.7 Hz, 1H), 7.43 (t, J = 7.8 Hz, 1H), 3.74 (s, 2H), 3.69 (t, J = 5.1 Hz, 4H), 2.88 (s, 3H), 2.65 (t, J = 5.0 Hz, 4H). NH not observed. |
| | 4-(Cyanomethyl)-N-[4-(3- | |
| | cyanophenyl)-5-(4-methylquinazolin- | |
| | 6-yl)thiazol-2-yl]piperazine-1- | |
| | carboxamide | |
| 7.16 | 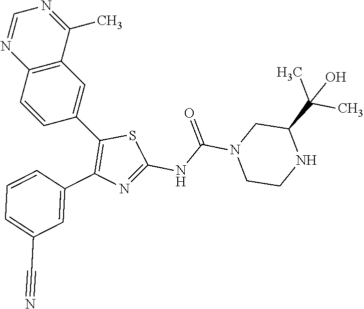 [SMILES: Cc1ncnc2ccc(-c3sc(NC(=O)N4CCN[C@H](C(C)(C)O)C4)nc3-c3cccc(C#N)c3)cc12] | LC-MS-2 (Method 8B): Rt 2.58 mins; MS m/z 514.2 = [M + H]+ 1H NMR (500 MHz, DMSO-d6) δ 11.25 (s, 1H), 9.12 (s, 1H), 8.21 (d, J = 2.1 Hz, 1H), 7.95-7.91 (m, 2H), 7.80 (dt, J = 7.8, 1.5 Hz, 1H), 7.77 (dd, J = 8.7, 2.1 Hz, 1H), 7.72 (dt, J = 7.8, 1.5 Hz, 1H), 7.53 (t, J = 7.8 Hz, 1H), 4.44 (s, 1H), 4.21 (apr d, J = 12.4 Hz, 1H), 4.11 (apr d, J = 12.4 Hz, 1H), 3.04-2.96 (m, 1H), 2.84 (s, 3H), 2.83-2.74 (m, 1H), 2.62-2.54 (m, 2H), 2.37-2.33 (m, 1H), 1.16 (s, 3H), 1.12 (s, 3H). piperazine NH not observed. |
| | (3S)-N-[4-(3-Cyanophenyl)-5-(4- | |
| | methylquinazolin-6-yl)thiazol-2-yl]-3- | |
| | (1-hydroxy-1-methyl- | |
| | ethyl)piperazine-1-carboxamide | |
| | (Amine used—Intermediate D) | |
| 7.17 | 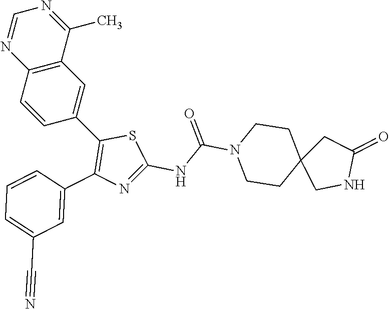 [SMILES: Cc1ncnc2ccc(-c3sc(NC(=O)N4CCC5(CC4)CNC(=O)C5)nc3-c3cccc(C#N)c3)cc12] | LC-MS-2 (Method 8A): Rt 3.10 mins; MS m/z 524.2 = [M + H]+ 1H NMR (500 MHz, DMSO-d6) δ 11.24 (s, 1H), 9.11 (s, 1H), 8.20 (d, J = 2.0 Hz, 1H), 7.95-7.90 (m, 2H), 7.79 (dt, J = 7.7, 1.6 Hz, 1H), 7.76 (dd, J = 8.8, 2.0 Hz, 1H), 7.71 (dt, J = 8.0, 1.5 Hz, 1H), 7.55 (s, 1H), 7.52 (t, J = 7.9 Hz, 1H), 3.68-3.61 (m, 2H), 3.49- 3.42 (m, 2H), 3.09 (s, 2H), 2.83 (s, 3H), 2.12 (s, 2H), 1.56 (t, J = 5.7 Hz, 4H). |
| | N-[4-(3-Cyanophenyl)-5-(4- | |
| | methylquinazolin-6-yl)thiazol-2-yl]-3- | |
| | oxo-2,8-diazaspiro[4.5]decane-8- | |
| | carboxamide | |
| 7.18 | 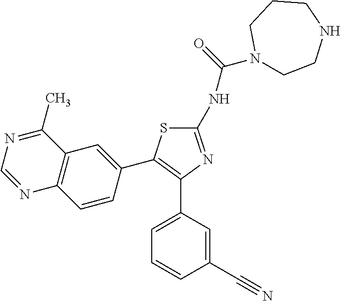 [SMILES: Cc1ncnc2ccc(-c3sc(NC(=O)N4CCCNCC4)nc3-c3cccc(C#N)c3)cc12] | LC-MS-2 (Method 8A): Rt 2.47 mins; MS m/z 470.1 = [M + H]+ 1H NMR (500 MHz, DMSO-d6) δ 9.11 (s, 1H), 8.20 (d, J = 2.0 Hz, 1H), 7.92 (dd, J = 5.2, 3.3 Hz, 2H), 7.79 (dt, J = 7.7, 1.4 Hz, 1H), 7.76 (dd, J = 8.7, 2.0 Hz, 1H), 7.71 (dt, J = 8.0, 1.4 Hz, 1H), 7.51 (t, J = 7.8 Hz, 1H), 3.64-3.51 (m, 4H), 2.84 (s, 3H), 2.83-2.80 (m, 2H), 2.71 (t, J = 5.8 Hz, 2H), 1.74-1.69 (m, 2H). 2 x NH not observed). |
| | N-[4-(3-Cyanophenyl)-5-(4- | |
| | methylquinazolin-6-yl)thiazol-2-yl]- | |
| | 1,4-diazepane-1-carboxamide | |
| 7.19 | 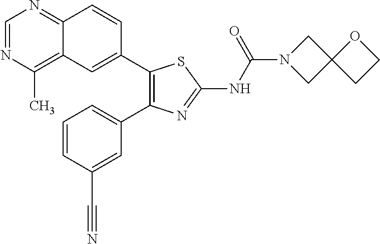 [SMILES: Cc1ncnc2ccc(-c3sc(NC(=O)N4CC5(CCO5)C4)nc3-c3cccc(C#N)c3)cc12] | LC-MS-2 (Method 8A): Rt 3.03 mins; MS m/z 469.4 = [M + H]+ 1H NMR (500 MHz, DMSO) δ 11.43 (s, 1H), 9.18 (s, 1H), 8.27 (d, J = 2.0 Hz, 1H), 7.99 (d, J = 8.7 Hz, 1H), 7.96 (t, J = 1.8 Hz, 1H), 7.86 (d, J = 7.9 Hz, 1H), 7.83 (dd, J = 8.8, 2.1 Hz, 1H), 7.76 (d, J = 8.0 Hz, 1H), 7.58 (t, J = 7.8 Hz, 1H), 4.49 (t, J = 7.5 Hz, 2H), 4.37 (d, J = 10.4 Hz, 2H), 4.21 (d, J = 10.4 Hz, 2H), 2.95-2.85 (m, 5H). |
| | N-[4-(3-Cyanophenyl)-5-(4- | |
| | methylquinazolin-6-yl)thiazol-2-yl]-1- | |
| | oxa-6-azaspiro[3.3]heptane-6- | |
| | carboxamide | |
| 7.20 | 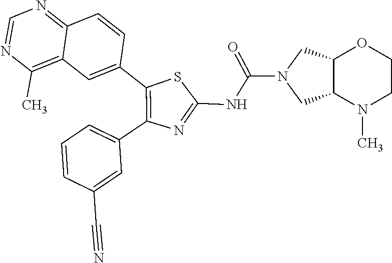 [SMILES: Cc1ncnc2ccc(-c3sc(NC(=O)N4C[C@@H]5OCCN(C)[C@@H]5C4)nc3-c3cccc(C#N)c3)cc12] | LC-MS-1 (Method 8A): Rt 2.42 mins; MS m/z 510.4 = [M − H]− 1H NMR (500 MHz, MeOD) δ 9.07 (s, 1H), 8.24 (d, J = 1.9 Hz, 1H), 7.96-7.90 (m, 2H), 7.85 (dd, J = 8.7, 1.9 Hz, 1H), 7.70 (dt, J = 8.0, 1.5 Hz, 1H), 7.65 (dt, J = 7.8, 1.4 Hz, 1H), 7.43 (t, J = 7.8 Hz, 1H), 4.21-4.05 (m, 1H), 3.96- 3.84 (m, 1H), 3.82-3.37 (m, 6H), 2.88 (s, 3H), 2.82-2.74 (m, 1H), 2.52 (d, J = 12.2 Hz, 1H), 2.45 (s, 3H). NH not observed. |
| | (4aR,7aS)-N-[4-(3-Cyanophenyl)-5- | |
| | (4-methylquinazolin-6-yl)thiazol-2-yl]- | |
| | 4-methyl-2,3,4a,5,7,7a- | |
| | hexahydropyrrolo[3,4-b][1,4]oxazine- | |
| | 6-carboxamide | |
| 7.21 | 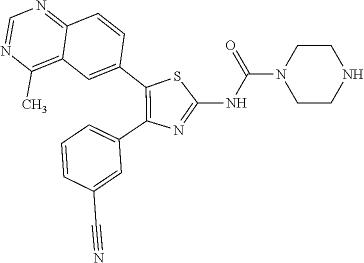 [SMILES: Cc1ncnc2ccc(-c3sc(NC(=O)N4CCNCC4)nc3-c3cccc(C#N)c3)cc12] | LC-MS-1 (Method 8A): Rt 2.31 mins; MS m/z 454.4 = [M − H]− 1H NMR (500 MHz, DMSO) δ 9.11 (s, 1H), 8.20 (d, J = 1.9 Hz, 1H), 7.97-7.88 (m, 2H), 7.79 (dt, J = 7.7, 1.5 Hz, 1H), 7.76 (dd, J = 8.7, 2.0 Hz, 1H), 7.71 (dt, J = 8.0, 1.5 Hz, 1H), 7.52 (t, J = 7.9 Hz, 1H), 3.47 (t, J = 5.0 Hz, 4H), 2.83 (s, 3H), 2.71 (t, J = 4.9 Hz, 4H). 2 x NH not observed. |
| | N-[4-(3-Cyanophenyl)-5-(4- | |
| | methylquinazolin-6-yl)thiazol-2- | |
| | yl]piperazine-1-carboxamide | |
| 7.22 | 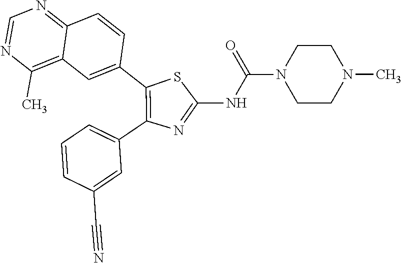 [SMILES: Cc1ncnc2ccc(-c3sc(NC(=O)N4CCN(C)CC4)nc3-c3cccc(C#N)c3)cc12] | LC-MS-1 (Method 8A): Rt 2.34 mins; MS m/z 468.4 = [M − H]− 1H NMR (500 MHz, DMSO) δ 11.27 (s, 1H), 9.12 (s, 1H), 8.22 (d, J = 2.0 Hz, 1H), 7.99-7.90 (m, 2H), 7.80 (dt, J = 7.7, 1.4 Hz, 1H), 7.77 (dd, J = 8.7, 2.0 Hz, 1H), 7.71 (dt, J = 8.0, 1.5 Hz, 1H), 7.53 (t, J = 7.8 Hz, 1H), 3.55 (t, J = 5.0 Hz, 4H), 2.84 (s, 3H), 2.34 (t, J = 5.0 Hz, 4H), 2.21 (s, 3H). |
| | N-[4-(3-Cyanophenyl)-5-(4- | |
| | methylquinazolin-6-yl)thiazol-2-yl]-4- | |
| | methyl-piperazine-1-carboxamide | |
| 7.23 | 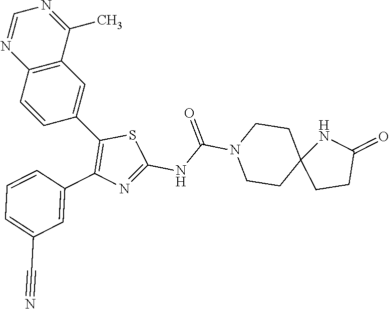 [SMILES: Cc1ncnc2ccc(-c3sc(NC(=O)N4CCC5(CCC(=O)N5)CC4)nc3-c3cccc(C#N)c3)cc12] | LC-MS-2 (Method 8A): Rt 3.07 mins; MS m/z 524.2 = [M + H]+ 1H NMR (500 MHz, DMSO) δ 11.26 (br s, 1H), 9.11 (s, 1H), 8.20 (d, J = 2.0 Hz, 1H), 8.04 (s, 1H), 7.95-7.89 (m, 2H), 7.81- 7.74 (m, 2H), 7.70 (br dt, J = 8.1, 1.5 Hz, 1H), 7.52 (apr t, J = 7.8 Hz, 1H), 3.73-3.64 (m, 2H), 3.55- 3.46 (m, 2H), 2.84 (s, 3H), 2.22 (br t, J = 8.0 Hz, 2H), 1.89 (br t, J = 8.0 Hz, 2H), 1.64-1.54 (m, 4H) |
| | N-[4-(3-Cyanophenyl)-5-(4- | |
| | methylquinazolin-6-yl)thiazol-2-yl]-2- | |
| | oxo-1,8-diazaspiro[4.5]decane-8- | |
| | carboxamide | |
| 7.24 | 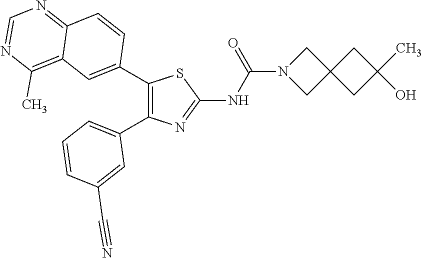 [SMILES: Cc1ncnc2ccc(-c3sc(NC(=O)N4CC5(C4)CC(C)(O)C5)nc3-c3cccc(C#N)c3)cc12] | LC-MS-2 (Method 8B): Rt 2.93 mins; MS m/z 497.1 = [M + H]+ 1H NMR (500 MHz, DMSO) δ 11.26 (s, 1H), 9.12 (s, 1H), 8.21 (d, J = 2.0 Hz, 1H), 7.93 (d, J = 8.7 Hz, 1H), 7.90 (t, J = 1.9 Hz, 1H), 7.79 (dt, J = 7.6, 1.4 Hz, 1H), 7.77 (dd, J = 8.7, 2.0 Hz, 1H), 7.70 (dt, J = 7.9, 1.5 Hz, 1H), 7.52 (t, J = 7.8 Hz, 1H), 4.92 (s, 1H), 4.03 (d, J = 19.0 Hz, 4H), 2.84 (s, 3H), 2.20 (s, 4H), 1.18 (s, 3H). |
| | N-[4-(3-Cyanophenyl)-5-(4- | |
| | methylquinazolin-6-yl)thiazol-2-yl]-6- | |
| | hydroxy-6-methyl-2- | |
| | azaspiro[3.3]heptane-2-carboxamide | |
| 7.25 | 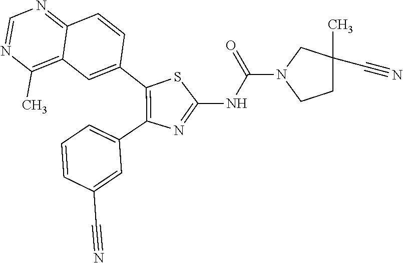 [SMILES: Cc1ncnc2ccc(-c3sc(NC(=O)N4CCC(C)(C#N)C4)nc3-c3cccc(C#N)c3)cc12] | LC-MS-2 (Method 8B): Rt 2.96 mins; MS m/z 480.1 = [M + H]+ 1H NMR (500 MHz, DMSO) δ 11.31 (s, 1H), 9.18 (s, 1H), 8.28 (d, J = 2.0 Hz, 1H), 8.08-7.96 (m, 2H), 7.86 (d, J = 8.0 Hz, 1H), 7.84 (dd, J = 8.7, 2.0 Hz, 1H), 7.78 (dt, J = 8.1, 1.5 Hz, 1H), 7.59 (t, J = 7.8 Hz, 1H), 4.21-3.88 (m, 1H), 3.84-3.63 (m, 2H), 3.61- 3.45 (m, 1H), 2.90 (s, 3H), 2.50- 2.45 (m, 1H), 2.25-2.03 (m, 1H), 1.54 (s, 3H). |
| | 3-Cyano-N-[4-(3-cyanophenyl)-5-(4- | |
| | methylquinazolin-6-yl)thiazol-2-yl]-3- | |
| | methyl-pyrrolidine-1-carboxamide | |
| 7.26 | 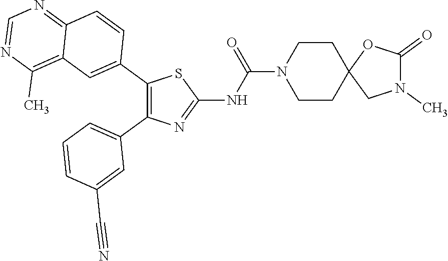 [SMILES: Cc1ncnc2ccc(-c3sc(NC(=O)N4CCC5(CC4)CN(C)C(=O)O5)nc3-c3cccc(C#N)c3)cc12] | LC-MS-2 (Method 8B): Rt 2.75 mins; MS m/z 540.2 = [M + H]+ 1H NMR (500 MHz, MeOD) δ 9.09 (s, 1H), 8.25 (d, J = 1.9 Hz, 1H), 8.00-7.94 (m, 2H), 7.86 (dd, J = 8.7, 2.0 Hz, 1H), 7.72 (dt, J = 8.0, 1.5 Hz, 1H), 7.68 (dt, J = 7.8, 1.5 Hz, 1H), 7.45 (t, J = 7.8 Hz, 1H), 4.02 (dt, J = 14.1, 4.4 Hz, 2H), 3.51 (ddd, J = 14.0, 11.1, 3.2 Hz, 2H), 3.46 (s, 2H), 2.90 (s, 3H), 2.89 (s, 3H), 2.07-1.95 (m, 2H), 1.90 (ddd, J = 14.2, 10.7, 4.4 Hz, 2H). NH not observed. |
| | N-[4-(3-Cyanophenyl)-5-(4- | |
| | methylquinazolin-6-yl)thiazol-2-yl]-3- | |
| | methyl-2-oxo-1-oxa-3,8- | |
| | diazaspiro[4.5]decane-8- | |
| | carboxamide | |
| 7.27 | 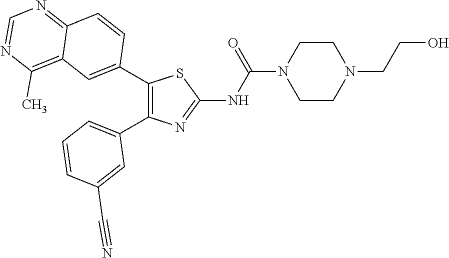 [SMILES: Cc1ncnc2ccc(-c3sc(NC(=O)N4CCN(CCO)CC4)nc3-c3cccc(C#N)c3)cc12] | LC-MS-2 (Method 8B): Rt 2.73 mins; MS m/z 500.2 = [M + H]+ 1H NMR (500 MHz, DMSO) δ 11.26 (s, 1H), 9.12 (s, 1H), 8.21 (d, J = 2.0 Hz, 1H), 7.96-7.90 (m, 2H), 7.80 (dt, J = 7.7, 1.5 Hz, 1H), 7.77 (dd, J = 8.7, 2.1 Hz, 1H), 7.71 (dt, J = 8.1, 1.5 Hz, 1H), 7.52 (t, J = 7.8 Hz, 1H), 4.44 (t, J = 5.4 Hz, 1H), 3.58-3.50 (m, 6H), 2.84 (s, 3H), 2.46-2.40 (m, 6H). |
| | N-[4-(3-Cyanophenyl)-5-(4- | |
| | methylquinazolin-6-yl)thiazol-2-yl]-4- | |
| | (2-hydroxyethyl)piperazine-1- | |
| | carboxamide | |
| 7.28 | 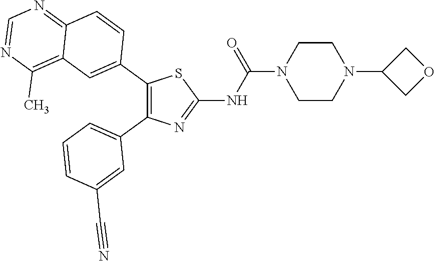 [SMILES: Cc1ncnc2ccc(-c3sc(NC(=O)N4CCN(C5COC5)CC4)nc3-c3cccc(C#N)c3)cc12] | LC-MS-2 (Method 8B): Rt 2.57 mins; MS m/z 512.2 = [M + H]+ 1H NMR (500 MHz, DMSO) δ 11.27 (s, 1H), 9.12 (s, 1H), 8.21 (d, J = 2.0 Hz, 1H), 7.96-7.90 (m, 2H), 7.80 (dt, J = 7.6, 1.5 Hz, 1H), 7.77 (dd, J = 8.7, 2.0 Hz, 1H), 7.71 (dt, J = 7.9, 1.5 Hz, 1H), 7.52 (t, J = 7.8 Hz, 1H), 4.56 (t, J = 6.5 Hz, 2H), 4.47 (t, J = 6.0 Hz, 2H), 3.59 (t, J = 4.9 Hz, 4H), 3.44 (p, J = 6.3 Hz, 1H), 2.84 (s, 3H), 2.30 (t, J = 5.0 Hz, 4H). |
| | N-[4-(3-Cyanophenyl)-5-(4- | |
| | methylquinazolin-6-yl)thiazol-2-yl]-4- | |
| | (oxetan-3-yl)piperazine-1- | |
| | carboxamide | |
| 7.29 | 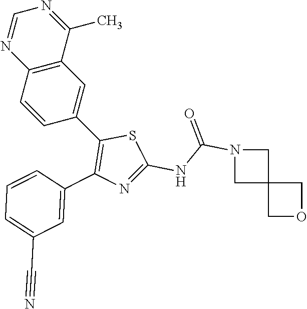 [SMILES: Cc1ncnc2ccc(-c3sc(NC(=O)N4CC5(COC5)C4)nc3-c3cccc(C#N)c3)cc12] | LC-MS2 (Method 8B): Rt 3.00 mins; MS m/z 469.1 = [M + H]+ 1H NMR (500 MHz, DMSO-d6) δ 11.35 (s, 1H), 9.11 (s, 1H), 8.20 (d, J = 2.0 Hz, 1H), 7.92 (d, J = 8.7 Hz, 1H), 7.89 (t, J = 1.8 Hz, 1H), 7.79 (d, J = 7.8 Hz, 1H), 7.76 (dd, J = 8.7, 2.0 Hz, 1H), 7.69 (d, J = 7.8 Hz, 1H), 7.51 (t, J = 7.8 Hz, 1H), 4.68 (s, 4H), 4.22 (s, 4H), 2.83 (s, 3H). |
| | N-[4-(3-Cyanophenyl)-5-(4- | |
| | methylquinazolin-6-yl)thiazol-2-yl]-2- | |
| | oxa-6-azaspiro[3.3]heptane-6- | |
| | carboxamide | |

### Example 7.7a and 7.7b

Chiral separation of racemic N-[4-(3-cyanophenyl)-5-(4-methylquinazolin-6-yl)thiazol-2-yl]-2-(1-hydroxy-1-methyl-ethyl)morpholine-4-carboxamide (Example 7.7) using Supercritical Fluid Chromatography was carried out using the following conditions to afford the individual enantiomers:
Purification Conditions—Column Details: Chiralpak IH (20 mm×250 mm, 5 μm); Column Temperature: 40° C.; Flow Rate: 50 mL/min; BPR: 100 BarG; Detector Wavelength: 210 nm; Injection Volume: 500 μL (15.5 mg); Isocratic Conditions: 30:70 EtOH:CO₂.
Chiral Purity Analysis Conditions—Column Details Chiralpak IH (4.6 mm×250 mm, 5 μm); Column Temperature: 40° C.; Flow Rate: 4 mL/min; Detector Wavelength: 210-400 nm; Injection Volume: 1.0 μL; BPR: 125 BarG; Isocratic Conditions: 30:70 EtOH:CO₂ (0.1%% v/v NH₃).

### Example 7.7a: (2S)—N-[4-(3-Cyanophenyl)-5-(4-methylquinazolin-6-yl)thiazol-2-yl]-2-(1-hydroxy-1-methyl-ethyl)morpholine-4-carboxamide or (2R)—N-[4-(3-cyanophenyl)-5-(4-methylquinazolin-6-yl)thiazol-2-yl]-2-(1-hydroxy-1-methyl-ethyl)morpholine-4-carboxamide

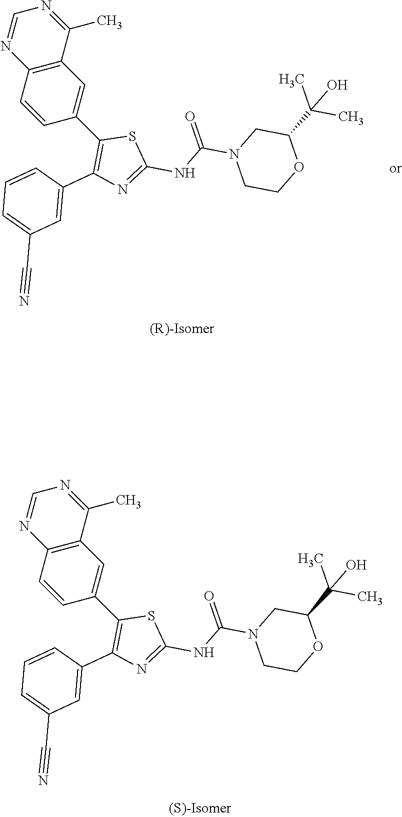 [SMILES: Cc1ncnc2ccc(-c3sc(NC(=O)N4CCO[C@@H](C(C)(C)O)C4)nc3-c3cccc(C#N)c3)cc12 ; Cc1ncnc2ccc(-c3sc(NC(=O)N4CCO[C@H](C(C)(C)O)C4)nc3-c3cccc(C#N)c3)cc12]

First Eluted Peak: SFC Retention time=2.53 mins LC-MS-1 (Method 8A): Rt 3.20 mins; MS m/z 515.5=[M+H]+

1H NMR (500 MHz, DMSO) δ 11.34 (br s, 1H), 9.12 (s, 1H), 8.21 (br s, 1H), 7.95-7.90 (m, 2H), 7.80 (br d, J=7.7 Hz, 1H), 7.77 (br dd, J=8.6, 2.0 Hz, 1H), 7.71 (br d, J=8.6 Hz, 1H), 7.52 (apr t, J=7.8 Hz, 1H), 4.50 (s, 1H), 4.26 (br d, J=13.1 Hz, 1H), 4.07 (br d, J=13.1 Hz, 1H), 3.94 (dd, J=11.3, 3.3 Hz, 1H), 3.45 (td, J=11.7, 2.7 Hz, 1H), 3.11 (dd, J=10.9, 2.4 Hz, 1H), 2.99-2.92 (m, 1H), 2.83 (s, 3H), 2.80 (br t, J=12.0 Hz, 1H), 1.14 (s, 3H), 1.10 (s, 3H).

### Example 7.7b: (2S)—N-[4-(3-Cyanophenyl)-5-(4-methylquinazolin-6-yl)thiazol-2-yl]-2-(1-hydroxy-1-methyl-ethyl)morpholine-4-carboxamide or (2R)—N-[4-(3-cyanophenyl)-5-(4-methylquinazolin-6-yl)thiazol-2-yl]-2-(1-hydroxy-1-methyl-ethyl)morpholine-4-carboxamide

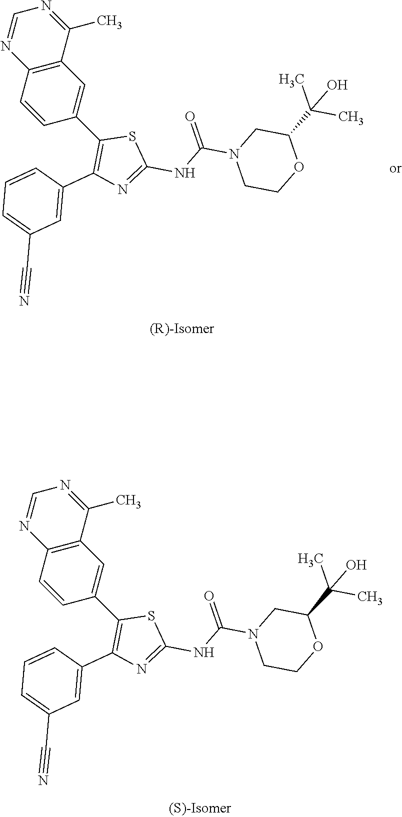 [SMILES: Cc1ncnc2ccc(-c3sc(NC(=O)N4CCO[C@@H](C(C)(C)O)C4)nc3-c3cccc(C#N)c3)cc12 ; Cc1ncnc2ccc(-c3sc(NC(=O)N4CCO[C@H](C(C)(C)O)C4)nc3-c3cccc(C#N)c3)cc12]

Second Eluted Peak: SFC Retention time=3.05 mins LC-MS-1 (Method 8A): Rt 3.20 mins; MS m/z 515.5=[M+H]+

1H NMR (500 MHz, DMSO) δ 11.34 (br s, 1H), 9.12 (s, 1H), 8.21 (br s, 1H), 7.95-7.90 (m, 2H), 7.80 (br d, J=7.8 Hz, 1H), 7.77 (br dd, J=8.8, 2.0 Hz, 1H), 7.71 (br d, J=8.1 Hz, 1H), 7.52 (apr t, J=7.8 Hz, 1H), 4.50 (s, 1H), 4.26 (br d, J=13.1 Hz, 1H), 4.07 (br d, J=13.1 Hz, 1H), 3.94 (dd, J=11.3, 3.3 Hz, 1H), 3.45 (td, J=11.7, 2.8 Hz, 1H), 3.11 (dd, J=10.9, 2.4 Hz, 1H), 2.99-2.92 (m, 1H), 2.83 (s, 3H), 2.80 (br t, J=11.9 Hz, 1H), 1.14 (s, 3H), 1.10 (s, 3H)

### Example 8

### N-[4-(3-Cyanophenyl)-5-(2-methylquinazolin-7-yl)thiazol-2-yl]-2-oxa-6-azaspiro[3.3]heptane-6-carboxamide

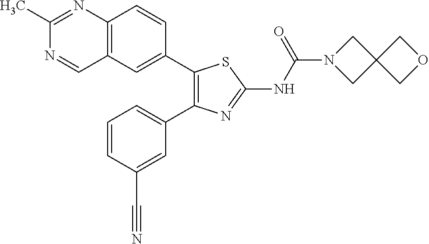 [SMILES: Cc1ncc2cc(-c3sc(NC(=O)N4CC5(COC5)C4)nc3-c3cccc(C#N)c3)ccc2n1]

The title compound was prepared from 3-[2-amino-5-(2-methylquinazolin-7-yl)thiazol-4-yl]benzonitrile (Intermediate AA) and 2-oxa-6-azaspiro[3.3]heptane analogously to Example 7.

LC-MS-1 (Method 8A): Rt 2.62 mins; MS m/z 469.4=[M+H]+ 1H NMR (500 MHz, DMSO-d6) δ 11.32 (s, 1H), 9.50 (s, 1H), 8.15 (d, J=2.2 Hz, 1H), 7.86 (d, J=2.0 Hz, 1H), 7.85 (d, J=8.6 Hz, 1H), 7.77 (dd, J=7.8, 1.5 Hz, 1H), 7.72 (dd, J=8.7, 2.1 Hz, 1H), 7.67 (dt, J=8.0, 1.5 Hz, 1H), 7.50 (t, J=7.8 Hz, 1H), 4.68 (s, 4H), 4.21 (s, 4H), 2.78 (s, 3H).

### Example 9

### N-[4-(3-Cyanophenyl)-5-(2,4-dimethylquinazolin-6-yl)thiazol-2-yl]-2-oxa-6-azaspiro[3.3]heptane-6-carboxamide

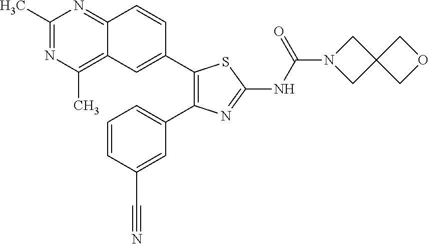 [SMILES: Cc1nc(C)c2cc(-c3sc(NC(=O)N4CC5(COC5)C4)nc3-c3cccc(C#N)c3)ccc2n1]

The title compound was prepared from 3-[2-amino-5-(2,4-dimethylquinazolin-6-yl)thiazol-4-yl]benzonitrile (Intermediate AB) and 2-oxa-6-azaspiro[3.3]heptane analogously to Example 7.

LC-MS-1 (Method 8B): Rt 2.63 mins; MS m/z 483.8=[M+H]+

1H NMR (500 MHz, DMSO-d6) δ 11.19 (s, 1H), 8.14 (d, J=2.0 Hz, 1H), 7.87 (t, J=1.8 Hz, 1H), 7.82 (d, J=8.7 Hz, 1H), 7.78 (dt, J=7.6, 1.5 Hz, 1H), 7.72-7.66 (m, 2H), 7.50 (t, J=7.9 Hz, 1H), 4.68 (s, 4H), 4.21 (s, 4H), 2.78 (s, 3H), 2.70 (s, 3H).

### Example 10

### N-[4-(3-Cyanophenyl)-5-quinazolin-6-yl-thiazol-2-yl]-2-oxa-6-azaspiro[3.3]heptane-6-carboxamide

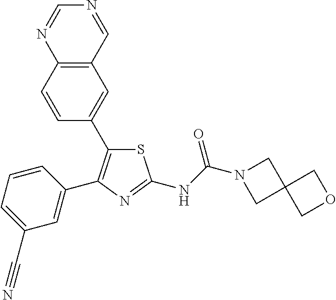 [SMILES: N#Cc1cccc(-c2nc(NC(=O)N3CC4(COC4)C3)sc2-c2ccc3ncncc3c2)c1]

The title compound was prepared from 3-(2-amino-5-quinazolin-6-yl-thiazol-4-yl)benzonitrile (Intermediate AC) and 2-oxa-6-azaspiro[3.3]heptane analogously to Example 7.

LC-MS-1 (Method 8B): Rt 2.59 mins; MS m/z 455.4=[M+H]+

1H NMR (500 MHz, DMSO-d6) δ 11.36 (s, 1H), 9.61 (s, 1H), 9.30 (s, 1H), 8.22 (s, 1H), 7.96 (d, J=8.7 Hz, 1H), 7.89 (s, 1H), 7.79 (d, J=8.1 Hz, 2H), 7.68 (d, J=8.1 Hz, 1H), 7.51 (t, J=8.1 Hz, 1H), 4.68 (s, 4H), 4.22 (s, 4H).

### Example 11

### N-[4-(3-Cyanophenyl)-5-(4-methyl-6-quinolyl)thiazol-2-yl]-2-oxa-6-azaspiro[3.3]heptane-6-carboxamide

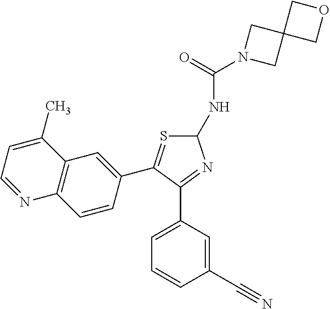

The title compound was prepared from 3-[2-amino-5-(4-methyl-6-quinolyl)thiazol-4-yl]benzonitrile (Intermediate AD) and 2-oxa-6-azaspiro[3.3]heptane analogously to Example 7.

LC-MS-1 (Method 8A): Rt 3.19 mins; MS m/z 468.4=[M+H]+

1H NMR (500 MHz, DMSO-d6) δ 11.22 (s, 1H), 8.76 (d, J=4.3 Hz, 1H), 8.02 (d, J=2.0 Hz, 1H), 7.96 (d, J=8.7 Hz, 1H), 7.89 (t, J=1.8 Hz, 1H), 7.77 (dt, J=7.6, 1.5 Hz, 1H), 7.71 (dt, J=8.1, 1.4 Hz, 1H), 7.56 (dd, J=8.7, 2.0 Hz, 1H), 7.51 (t, J=7.9 Hz, 1H), 7.42-7.38 (m, 1H), 4.68 (s, 4H), 4.21 (s, 4H), 2.59-2.56 (m, 3H).

### Example 12

### N-[4-(3-Cyanophenyl)-5-(4-methoxyquinazolin-6-yl)thiazol-2-yl]-2-oxa-6-azaspiro[3.3]heptane-6-carboxamide

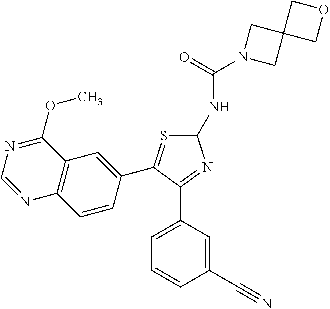

The title compound was prepared from 3-[2-amino-5-(4-methoxyquinazolin-6-yl)thiazol-4-yl]benzonitrile (Intermediate AE) and 2-oxa-6-azaspiro[3.3]heptane analogously to Example 7.

LC-MS-2 (Method 8A): Rt 3.28 mins; MS m/z 485.1=[M+H]+

1H NMR (500 MHz, DMSO-d6) δ 11.34 (s, 1H), 8.81 (s, 1H), 8.07 (d, J=2.1 Hz, 1H), 7.90-7.84 (m, 2H), 7.78 (d, J=7.7 Hz, 1H), 7.73-7.67 (m, 2H), 7.51 (t, J=7.8 Hz, 1H), 4.68 (s, 4H), 4.20 (s, 4H), 4.12 (s, 3H).

### Example 13

### N-[4-(3-Cyanophenyl)-5-(4-ethylquinazolin-6-yl)thiazol-2-yl]-2-oxa-6-azaspiro[3.3]heptane-6-carboxamide

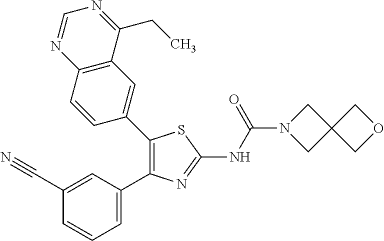 [SMILES: CCc1ncnc2ccc(-c3sc(NC(=O)N4CC5(COC5)C4)nc3-c3cccc(C#N)c3)cc12]

The title compound was prepared from 3-[2-amino-5-(4-ethylquinazolin-6-yl)thiazol-4-yl]benzonitrile (Intermediate AF) and 2-oxa-6-azaspiro[3.3]heptane analogously to Example 7.

LC-MS-2 (Method 8B): Rt 3.19 mins; MS m/z 483.1=[M+H]+

1H NMR (500 MHz, DMSO) δ 11.30 (br s, 1H), 9.17 (s, 1H), 8.19 (d, J=2.1 Hz, 1H), 7.95 (d, J=8.7 Hz, 1H), 7.89 (br t, J=1.5 Hz, 1H), 7.82-7.76 (m, 2H), 7.70 (br dt, J=8.0, 1.5 Hz, 1H), 7.51 (apr t, J=7.8 Hz, 1H), 4.68 (s, 4H), 4.22 (s, 4H), 3.20 (q, J=7.5 Hz, 2H), 1.24 (t, J=7.5 Hz, 3H).

### Example 14

### 4-Cyano-N-[4-(3-cyanophenyl)-5-(2,4-dimethyloxazol-5-yl)thiazol-2-yl]-4-methyl-piperidine-1-carboxamide

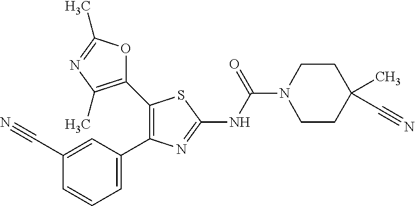 [SMILES: Cc1nc(C)c(-c2sc(NC(=O)N3CCC(C)(C#N)CC3)nc2-c2cccc(C#N)c2)o1]

The title compound was prepared from 3-[2-amino-5-(2,4-dimethyloxazol-5-yl)thiazol-4-yl]benzonitrile (Intermediate AG) and 4-methylpiperidine-4-carbonitrile hydrochloride analogously to Example 7.

LC-MS-2 (Method 8B): Rt 3.00 mins; MS m/z 447.1=[M+H]+

1H NMR (500 MHz, DMSO) δ 11.36 (s, 1H), 7.92 (t, J=1.5 Hz, 1H), 7.81 (dt, J=7.6, 1.5 Hz, 1H), 7.76 (dt, J=8.1, 1.5 Hz, 1H), 7.60 (apr t, J=7.8 Hz, 1H), 4.22 (d, J=14.1 Hz, 2H), 3.03 (t, J=13.1 Hz, 2H), 2.38 (s, 3H), 1.92 (d, J=13.5 Hz, 2H), 1.79 (s, 3H), 1.53 (td, J=13.5, 4.1 Hz, 2H), 1.37 (s, 3H).

### Example 15

### N-[4-(3-Cyanophenyl)-5-(2,4-dimethyloxazol-5-yl)thiazol-2-yl]-2-oxa-6-azaspiro[3.3]heptane-6-carboxamide

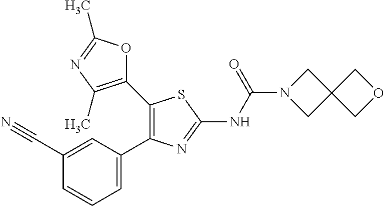 [SMILES: Cc1nc(C)c(-c2sc(NC(=O)N3CC4(COC4)C3)nc2-c2cccc(C#N)c2)o1]

The title compound was prepared from 3-[2-amino-5-(2,4-dimethyloxazol-5-yl)thiazol-4-yl]benzonitrile (Intermediate AG) and 2-oxa-6-azaspiro[3.3]heptane analogously to Example 7.

LC-MS-1 (Method 8B): Rt 2.69 mins; MS m/z 422.1=[M+H]+ 1H NMR (500 MHz, DMSO) δ 11.41 (s, 1H), 7.90 (t, J=1.5 Hz, 1H), 7.81 (dt, J=7.7, 1.5 Hz, 1H), 7.75 (dt, J=8.1, 1.5 Hz, 1H), 7.59 (apr t, J=7.8 Hz, 1H), 4.67 (s, 4H), 4.20 (s, 4H), 2.38 (s, 3H), 1.76 (s, 3H).

### Example 16

### N-[4-(3-Cyanophenyl)-5-(1-methylpyrrolo[2,3-b]pyridin-3-yl)thiazol-2-yl]-2-oxa-6-azaspiro[3.3]heptane-6-carboxamide

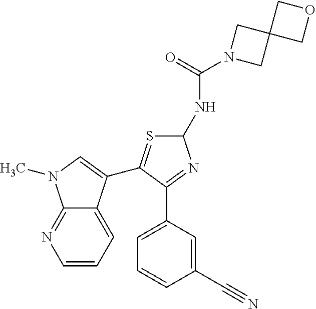

The title compound was prepared from 3-[2-amino-5-(1-methylpyrrolo[2,3-b]pyridin-3-yl)thiazol-4-yl]benzonitrile (Intermediate AK) and 2-oxa-6-azaspiro[3.3]heptane analogously to Example 7.

LC-MS-1 (Method 8B): Rt 3.01 mins; MS m/z 457.3=[M+H]+

1H NMR (500 MHz, DMSO-d6) δ 11.17 (s, 1H), 8.29 (dd, J=4.6, 1.6 Hz, 1H), 7.92 (d, J=1.8 Hz, 1H), 7.78 (s, 1H), 7.73 (dt, J=8.0, 1.5 Hz, 1H), 7.67 (dt, J=7.8, 1.5 Hz, 1H), 7.41 (t, J=7.9 Hz, 1H), 7.30 (dd, J=7.9, 1.6 Hz, 1H), 6.99 (dd, J=7.9, 4.6 Hz, 1H), 4.68 (s, 4H), 4.20 (s, 4H), 3.87 (s, 3H).

### Example 17

### N-[4-(3-Cyanophenyl)-5-(1H-pyrrolo[2,3-b]pyridin-3-yl)thiazol-2-yl]-2-oxa-6-azaspiro[3.3]heptane-6-carboxamide

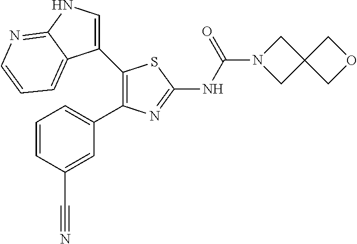 [SMILES: N#Cc1cccc(-c2nc(NC(=O)N3CC4(COC4)C3)sc2-c2c[nH]c3ncccc23)c1]

### Step 1: tert-Butyl 3-[2-amino-4-(3-cyanophenyl)thiazol-5-yl]pyrrolo[2,3-b]pyridine-1-carboxylate

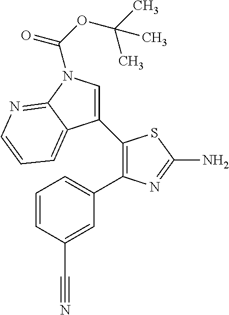 [SMILES: CC(C)(C)OC(=O)n1cc(-c2sc(N)nc2-c2cccc(C#N)c2)c2cccnc21]

Bis(tri-tert-butylphosphine)palladium(0) (55 mg, 0.11 mmol) was added to a degassed solution of tert-butyl 3-(4,4,5,5-tetramethyl-1,3,2-dioxaborolan-2-yl)pyrrolo[2,3-b]pyridine-1-carboxylate (442 mg, 1.29 mmol), 3-(2-amino-5-bromo-thiazol-4-yl)benzonitrile (Intermediate Q) (300 mg, 1.07 mmol) and K₂CO₃ (592 mg, 4.28 mmol) in 1,4-dioxane (5 mL) and water (1 mL). The flask was evacuated, backfilled with nitrogen (3×cycles) and heated at 50° C. for 1 h. The resulting mixture was allowed to cool to room temperature and was partitioned between EtOAc (40 mL) and water (40 mL). The layers were separated and the aqueous layer further extracted with EtOAc (2×40 mL). The combined organic extracts were dried over MgSO₄ and concentrated in vacuo. Purification by chromatography on silica eluting with a gradient of 20 to 70% EtOAc in petroleum ether afforded the title compound as an orange solid.

LC-MS-2 (Method 2B): Rt 1.53 mins; MS m/z 418.0=[M+H]+

1H NMR (500 MHz, DMSO) δ 8.40 (dd, J=4.7, 1.6 Hz, 1H), 7.89 (t, J=1.8 Hz, 1H), 7.84 (s, 1H), 7.67 (ddt, J=7.5, 5.7, 1.4 Hz, 2H), 7.42-7.35 (m, 2H), 7.32 (d, J=4.3 Hz, 2H), 7.16 (dd, J=7.9, 4.7 Hz, 1H), 1.62 (s, 9H).

### Step 2: tert-Butyl 3-[4-(3-cyanophenyl)-2-(2-oxa-6-azaspiro[3.3]heptane-6-carbonylamino)thiazol-5-yl]pyrrolo[2,3-b]pyridine-1-carboxylate

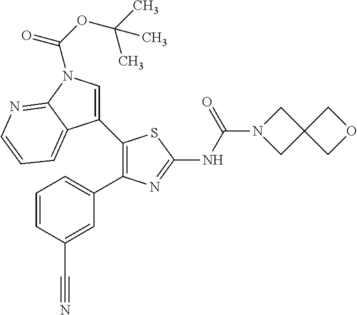 [SMILES: CC(C)(C)OC(=O)n1cc(-c2sc(NC(=O)N3CC4(COC4)C3)nc2-c2cccc(C#N)c2)c2cccnc21]

The title compound was prepared from tert-butyl 3-[2-amino-4-(3-cyanophenyl)thiazol-5-yl]pyrrolo[2,3-b]pyridine-1-carboxylate (step 1) and 2-oxa-6-azaspiro[3.3]heptane analogously to Example 7.

LC-MS-1 (Method 2B): Rt 1.32 mins; MS m/z 543.3=[M+H]+

### Step 3: N-[4-(3-Cyanophenyl)-5-(1H-pyrrolo[2,3-b]pyridin-3-yl)thiazol-2-yl]-2-oxa-6-azaspiro[3.3]heptane-6-carboxamide

To a stirred solution of tert-butyl 3-[4-(3-cyanophenyl)-2-(2-oxa-6-azaspiro[3.3]heptane-6-carbonylamino)thiazol-5-yl]pyrrolo[2,3-b]pyridine-1-carboxylate (crude product from step 2) (134 mg, 0.25 mmol) in DCM (5 mL) was added TFA (0.63 mL, 8.17 mmol) and the solution was stirred at room temperature for 1 h. The resulting mixture was diluted in DCM (20 mL) and washed with sat. aq. NaHCO₃ solution (20 mL) and brine (20 mL). The organic layer was dried over MgSO₄ and concentrated in vacuo to give a yellow solid. Purification by chromatography on silica eluting with a gradient of 2 to 5% MeOH in DCM afforded the title compound as a beige solid.

LC-MS-2 (Method 8B): Rt 2.84 mins; MS m/z 443.0=[M+H]+

1H NMR (500 MHz, DMSO) δ 12.06 (s, 1H), 11.16 (s, 1H), 8.24 (dd, J=4.7, 1.6 Hz, 1H), 7.89 (t, J=1.7 Hz, 1H), 7.74 (dt, J=8.0, 1.5 Hz, 1H), 7.68 (d, J=2.6 Hz, 1H), 7.66 (dt, J=7.7, 1.4 Hz, 1H), 7.42 (t, J=7.9 Hz, 1H), 7.35 (dd, J=8.0, 1.6 Hz, 1H), 6.97 (dd, J=7.9, 4.6 Hz, 1H), 4.68 (s, 4H), 4.20 (s, 4H).

### Example 18

### N-[4-(3-Cyanophenyl)-5-(3,8-dimethylimidazo[1,2-a]pyridin-6-yl)thiazol-2-yl]-2-oxa-6-azaspiro[3.3]heptane-6-carboxamide

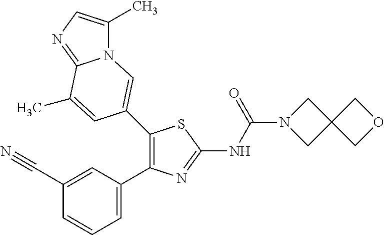 [SMILES: Cc1cc(-c2sc(NC(=O)N3CC4(COC4)C3)nc2-c2cccc(C#N)c2)cn2c(C)cnc12]

The title compound was prepared from 3-[2-amino-5-(3,8-dimethylimidazo[1,2-a]pyridin-6-yl)thiazol-4-yl]benzonitrile (Intermediate AH) and 2-oxa-6-azaspiro[3.3]heptane analogously to Example 7.

LC-MS-2 (Method 8B): Rt 2.83 mins; MS m/z 471.1=[M+H]+ 1H NMR (500 MHz, DMSO) δ 11.24 (s, 1H), 8.15 (s, 1H), 7.95 (t, J=1.6 Hz, 1H), 7.77-7.72 (m, 2H), 7.50 (apr t, J=7.9 Hz, 1H), 7.36 (d, J=1.1 Hz, 1H), 6.83 (t, J=1.6 Hz, 1H), 4.68 (s, 4H), 4.21 (s, 4H), 2.44-2.37 (m, 6H).

### Example 19

### 4-Cyano-N-[4-(3-cyanophenyl)-5-(5-methyl-1H-pyrazolo[4,3-b]pyridin-7-yl)thiazol-2-yl]-4-methyl-piperidine-1-carboxamide

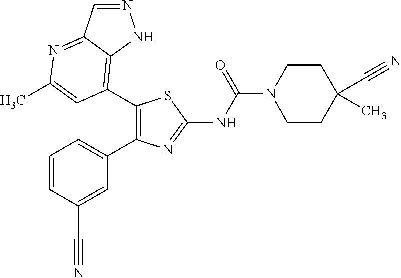 [SMILES: Cc1cc(-c2sc(NC(=O)N3CCC(C)(C#N)CC3)nc2-c2cccc(C#N)c2)c2[nH]ncc2n1]

The title compound was prepared from 3-[2-amino-5-(5-methyl-1H-pyrazolo[4,3-b]pyridin-7-yl)thiazol-4-yl]benzonitrile (Intermediate AI) and 4-methylpiperidine-4-carbonitrile hydrochloride analogously to Example 7.

LC-MS-1 (Method 8B): Rt 2.59 mins; MS m/z 483.3=[M+H]+

1H NMR (500 MHz, DMSO) δ 12.87 (s, 1H), 11.39 (s, 1H), 8.17 (s, 1H), 7.85 (s, 1H), 7.69 (d, J=7.7 Hz, 1H), 7.49 (d, J=8.0 Hz, 1H), 7.36 (t, J=7.9 Hz, 1H), 7.30 (s, 1H), 4.24 (d, J=14.0 Hz, 2H), 3.06 (t, J=13.0 Hz, 2H), 2.60 (s, 3H), 1.94 (d, J=13.6 Hz, 2H), 1.54 (td, J=13.6, 3.8 Hz, 2H), 1.39 (s, 3H).

### Example 20

### N-[4-(3-Cyanophenyl)-5-(4,8-dimethylquinazolin-6-yl)thiazol-2-yl]-2-oxa-6-azaspiro[3.3]heptane-6-carboxamide

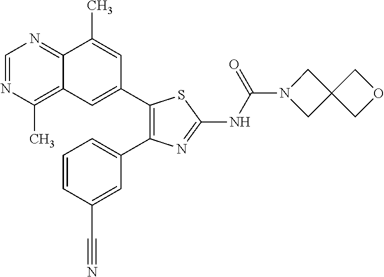 [SMILES: Cc1ncnc2c(C)cc(-c3sc(NC(=O)N4CC5(COC5)C4)nc3-c3cccc(C#N)c3)cc12]

The title compound was prepared from 3-[2-amino-5-(4,8-dimethylquinazolin-6-yl)thiazol-4-yl]benzonitrile (Intermediate AJ) and 2-oxa-6-azaspiro[3.3]heptane analogously to Example 7.

LC-MS-2 (Method 8B): Rt 3.12 mins; MS m/z 483.1=[M+H]+

1H NMR (500 MHz, DMSO) δ 11.34 (s, 1H), 9.14 (s, 1H), 8.00 (d, J=2.0 Hz, 1H), 7.93 (t, J=1.8 Hz, 1H), 7.80 (dt, J=7.7, 1.5 Hz, 1H), 7.75-7.65 (m, 2H), 7.51 (t, J=7.9 Hz, 1H), 4.69 (s, 4H), 4.23 (s, 4H), 2.78 (s, 3H), 2.61 (s, 3H).

### Example 21

### N-[4-(3-Cyanophenyl)-5-(1-methylpyrrolo[2,3-b]pyridin-4-yl)thiazol-2-yl]-2-oxa-6-azaspiro[3.3]heptane-6-carboxamide

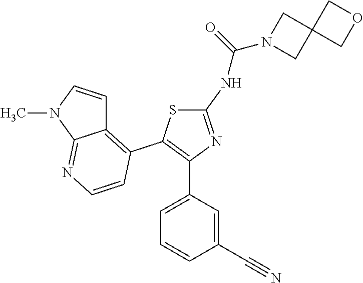 [SMILES: Cn1ccc2c(-c3sc(NC(=O)N4CC5(COC5)C4)nc3-c3cccc(C#N)c3)ccnc21]

The title compound was prepared from 3-[2-amino-5-(1-methylpyrrolo[2,3-b]pyridin-4-yl)thiazol-4-yl]benzonitrile (Intermediate AL) and 2-oxa-6-azaspiro[3.3]heptane analogously to Example 7.

LC-MS-2 (Method 8B): Rt 2.91 mins; MS m/z 457.1=[M+H]+ 1H NMR (500 MHz, DMSO-d6) δ 11.36 (s, 1H), 8.24 (d, J=4.9 Hz, 1H), 7.83 (t, J=1.8 Hz, 1H), 7.71 (dt, J=7.6, 1.4 Hz, 1H), 7.58 (dt, J=8.0, 1.4 Hz, 1H), 7.50 (d, J=3.5 Hz, 1H), 7.43 (t, J=7.8 Hz, 1H), 6.99 (d, J=5.0 Hz, 1H), 6.08 (d, J=3.5 Hz, 1H), 4.68 (s, 4H), 4.21 (s, 4H), 3.83 (s, 3H).

### Example 22

### N-[4-(3-Cyanophenyl)-5-(3-methylimidazo[1,2-a]pyridin-6-yl)thiazol-2-yl]-2-oxa-6-azaspiro[3.3]heptane-6-carboxamide

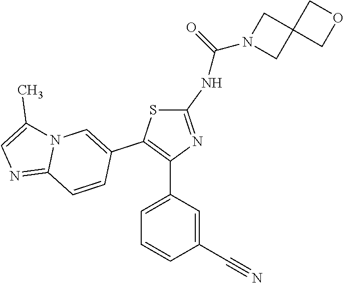 [SMILES: Cc1cnc2ccc(-c3sc(NC(=O)N4CC5(COC5)C4)nc3-c3cccc(C#N)c3)cn12]

The title compound was prepared from m-{2-amino-5-(9-methyl-1.7-diazabicyclo[4.3.0]nona-2,4,6,8-tetraen-3-yl)-1,3-thiazol-4-yl}benzonitrile (Intermediate AM) and 2-oxa-6-azaspiro[3.3]heptane analogously to Example 7.

LC-MS-2 (Method 8B): Rt 2.71 mins; MS m/z 457.1=[M+H]+ 1H NMR (500 MHz, DMSO-d6) δ 11.27 (s, 1H), 8.32 (s, 1H), 7.93-7.91 (m, 1H), 7.78-7.73 (m, 2H), 7.54-7.49 (m, 2H), 7.41 (s, 1H), 6.94 (dd, J=9.2, 1.8 Hz, 1H), 4.68 (s, 4H), 4.20 (s, 4H), 2.44 (s, 3H).

### Example 23

### N-[4-(3-Cyanophenyl)-5-(5-methyl-1H-pyrazolo[4,3-b]pyridin-7-yl)thiazol-2-yl]-2-oxa-6-azaspiro[3.3]heptane-6-carboxamide

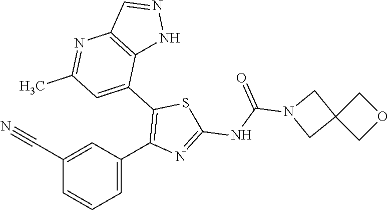 [SMILES: Cc1cc(-c2sc(NC(=O)N3CC4(COC4)C3)nc2-c2cccc(C#N)c2)c2[nH]ncc2n1]

The title compound was prepared from 3-[2-amino-5-(5-methyl-1H-pyrazolo[4,3-b]pyridin-7-yl)thiazol-4-yl]benzonitrile (Intermediate AI) and 2-oxa-6-azaspiro[3.3]heptane analogously to Example 7.

LC-MS-2 (Method 8B): Rt 2.45 mins; MS m/z 458.1=[M+H]+

1H NMR (500 MHz, DMSO) δ 12.85 (s, 1H), 11.42 (s, 1H), 8.16 (s, 1H), 7.83 (s, 1H), 7.68 (d, J=7.7 Hz, 1H), 7.48 (d, J=8.0 Hz, 1H), 7.36 (apr t, J=7.8 Hz, 1H), 7.30 (s, 1H), 4.69 (s, 4H), 4.23 (s, 4H), 2.60 (s, 3H).

### Example 24

### N-[4-(3-Cyanophenyl)-5-(1,6-dimethylpyrrolo[2,3-b]pyridin-4-yl)thiazol-2-yl]-2-oxa-6-azaspiro[3.3]heptane-6-carboxamide

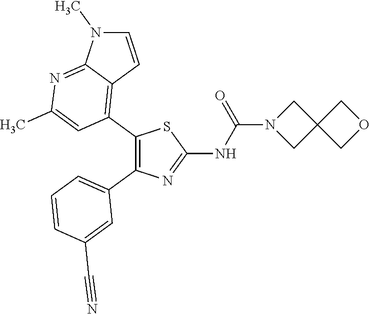 [SMILES: Cc1cc(-c2sc(NC(=O)N3CC4(COC4)C3)nc2-c2cccc(C#N)c2)c2ccn(C)c2n1]

The title compound was prepared from 3-[2-amino-5-(1,6-dimethylpyrrolo[2,3-b]pyridin-4-yl)thiazol-4-yl]benzonitrile (Intermediate AR) and 2-oxa-6-azaspiro[3.3]heptane analogously to Example 7.

LC-MS-2 (Method 8B): Rt 3.35 mins; MS m/z 471.1=[M+H]+

1H NMR (500 MHz, MeOD) δ 7.87 (d, J=1.8 Hz, 1H), 7.63 (dt, J=8.1, 1.5 Hz, 1H), 7.57 (dt, J=7.8, 1.4 Hz, 1H), 7.34 (t, J=7.9 Hz, 1H), 7.20 (d, J=3.5 Hz, 1H), 6.96 (s, 1H), 6.04 (d, J=3.5 Hz, 1H), 4.60 (s, 4H), 4.33 (s, 4H), 3.87 (s, 3H), 2.61 (s, 3H).

NH not observed.

### Example 25

### N-[4-(3-Cyanophenyl)-5-(3-methylbenzimidazol-5-yl)thiazol-2-yl]-2-oxa-6-azaspiro[3.3]heptane-6-carboxamide

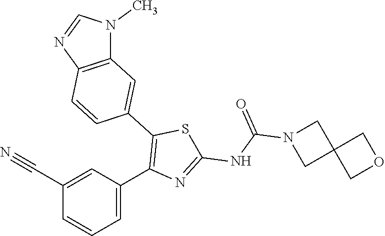 [SMILES: Cn1cnc2ccc(-c3sc(NC(=O)N4CC5(COC5)C4)nc3-c3cccc(C#N)c3)cc21]

The title compound was prepared from 3-[2-amino-5-(3-methylbenzimidazol-5-yl)thiazol-4-yl]benzonitrile (Intermediate AS) and 2-oxa-6-azaspiro[3.3]heptane analogously to Example 7.

LC-MS-2 (Method 8B): Rt 2.64 mins; MS m/z 457.1=[M+H]+

1H NMR (500 MHz, DMSO-d6) δ 11.19 (s, 1H), 8.23 (s, 1H), 7.84 (s, 1H), 7.71 (d, J=7.8 Hz, 1H), 7.67 (d, J=7.8 Hz, 1H), 7.64-7.58 (m, 2H), 7.46 (t, J=7.8 Hz, 1H), 7.06 (d, J=8.5 Hz, 1H), 4.68 (s, 4H), 4.20 (s, 4H), 3.81 (s, 3H).

### Example 26

### N-[4-(3-Cyanophenyl)-5-imidazo[1,2-a]pyridin-6-yl-thiazol-2-yl]-2-oxa-6-azaspiro[3.3]heptane-6-carboxamide

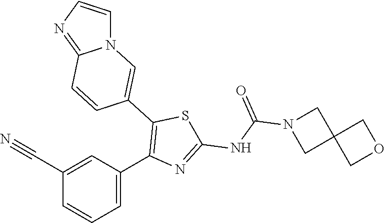 [SMILES: N#Cc1cccc(-c2nc(NC(=O)N3CC4(COC4)C3)sc2-c2ccc3nccn3c2)c1]

The title compound was prepared from 3-(2-amino-5-imidazo[1,2-a]pyridin-6-yl-thiazol-4-yl)benzonitrile (Intermediate AT) and 2-oxa-6-azaspiro[3.3]heptane analogously to Example 7.

LC-MS-1 (Method 8B): Rt 2.49 mins; MS m/z 443.2=[M+H]+

1H NMR (500 MHz, DMSO-d6) δ 11.30 (s, 1H), 8.70 (s, 1H), 7.95 (s, 1H), 7.92 (t, J=1.7 Hz, 1H), 7.78-7.71 (m, 2H), 7.60 (d, J=1.2 Hz, 1H), 7.57-7.49 (m, 2H), 6.97 (dd, J=9.4, 1.8 Hz, 1H), 4.68 (s, 4H), 4.19 (s, 4H).

### Example 27

### N-[4-(3-Cyanophenyl)-5-(7-methyl-1H-indazol-5-yl)thiazol-2-yl]-2-oxa-6-azaspiro[3.3]heptane-6-carboxamide

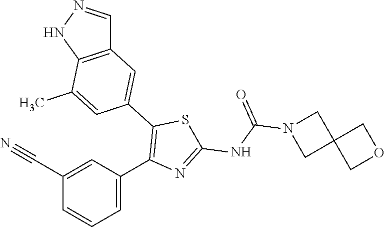 [SMILES: Cc1cc(-c2sc(NC(=O)N3CC4(COC4)C3)nc2-c2cccc(C#N)c2)cc2cn[nH]c12]

The title compound was prepared from tert-butyl 5-[2-amino-4-(3-cyanophenyl)thiazol-5-yl]-7-methyl-indazole-1-carboxylate (Intermediate AU) and 2-oxa-6-azaspiro[3.3]heptane analogously to Example 7 with the mixture heated for 16h after addition of the amine.

LC-MS-1 (Method 8B): Rt 3.03 mins; MS m/z 457.3=[M+H]+

1H NMR (500 MHz, DMSO-d6) δ 13.30 (s, 1H), 11.16 (s, 1H), 8.06 (d, J=1.5 Hz, 1H), 7.86 (t, J=1.7 Hz, 1H), 7.70 (dt, J=7.7, 1.4 Hz, 1H), 7.65 (dt, J=8.1, 1.5 Hz, 1H), 7.55 (d, J=1.5 Hz, 1H), 7.44 (t, J=7.9 Hz, 1H), 7.04 (s, 1H), 4.68 (s, 4H), 4.20 (s, 4H), 2.47 (s, 3H).

### Example 28

### N-[4-(3-Cyanophenyl)-5-(2,7-dimethylindazol-5-yl)thiazol-2-yl]-2-oxa-6-azaspiro[3.3]heptane-6-carboxamide

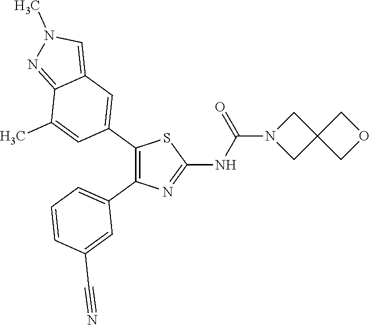 [SMILES: Cc1cc(-c2sc(NC(=O)N3CC4(COC4)C3)nc2-c2cccc(C#N)c2)cc2cn(C)nc12]

The title compound was prepared from 3-[2-amino-5-(2,7-dimethylindazol-5-yl)thiazol-4-yl]benzonitrile (Intermediate AV) and 2-oxa-6-azaspiro[3.3]heptane analogously to Example 7.

1H NMR (500 MHz, DMSO) δ 11.16 (s, 1H), 8.32 (s, 1H), 7.87 (t, J=1.8 Hz, 1H), 7.70 (tt, J=7.7, 1.4 Hz, 2H), 7.52 (d, J=1.6 Hz, 1H), 7.47 (t, J=7.9 Hz, 1H), 6.88 (d, J=1.5 Hz, 1H), 4.68 (s, 4H), 4.20 (s, 4H), 4.18 (s, 3H), 2.44 (s, 3H).

LC-MS-1 (Method 8B): Rt 3.82 mins; MS m/z 471.2=[M+H]+

### Example 29

### N-[4-(3-Cyanophenyl)-5-(8-methylimidazo[1,2-a]pyridin-6-yl)thiazol-2-yl]-2-oxa-6-azaspiro[3.3]heptane-6-carboxamide

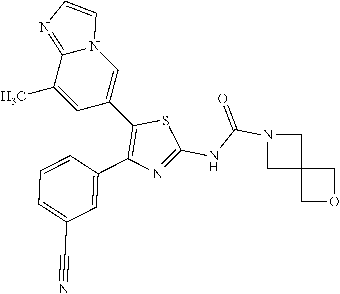 [SMILES: Cc1cc(-c2sc(NC(=O)N3CC4(COC4)C3)nc2-c2cccc(C#N)c2)cn2ccnc12]

The title compound was prepared from 3-[2-amino-5-(8-methylimidazo[1,2-a]pyridin-6-yl)thiazol-4-yl]benzonitrile (Intermediate AW) and 2-oxa-6-azaspiro[3.3]heptane analogously to Example 7.

LC-MS-1 (Method 8B): Rt 3.72 mins; MS m/z 457.2=[M+H]+

1H NMR (500 MHz, DMSO-d6) δ 11.26 (s, 1H), 8.54-8.52 (m, 1H), 7.95 (t, J=1.6 Hz, 1H), 7.92 (d, J=1.2 Hz, 1H), 7.76 (dt, J=7.8, 1.6 Hz, 1H), 7.73 (dt, J=7.8, 1.6 Hz, 1H), 7.56 (d, J=1.2 Hz, 1H), 7.51 (t, J=7.8 Hz, 1H), 6.86 (t, J=1.5 Hz, 1H), 4.68 (s, 4H), 4.21 (s, 4H), 2.42 (s, 3H).

### Example 30

### N-[4-(3-Cyanophenyl)-5-(1,7-dimethylindazol-5-yl)thiazol-2-yl]-2-oxa-6-azaspiro[3.3]heptane-6-carboxamide

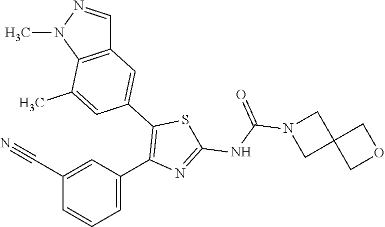 [SMILES: Cc1cc(-c2sc(NC(=O)N3CC4(COC4)C3)nc2-c2cccc(C#N)c2)cc2cnn(C)c12]

The title compound was prepared from 3-[2-amino-5-(1,7-dimethylindazol-5-yl)thiazol-4-yl]benzonitrile (Intermediate AX) and 2-oxa-6-azaspiro[3.3]heptane analogously to Example 7.

LC-MS-1 (Method 8B): Rt 4.22 mins; MS m/z 471.2=[M+H]+

1H NMR (500 MHz, DMSO) δ 11.16 (br s, 1H), 7.96 (s, 1H), 7.87 (t, J=1.5 Hz, 1H), 7.71 (dt, J=7.7, 1.5 Hz, 1H), 7.65 (dt, J=8.1, 1.5 Hz, 1H), 7.52 (br s, 1H), 7.45 (apr t, J=7.8 Hz, 1H), 7.03 (br s, 1H), 4.68 (s, 4H), 4.27 (s, 3H), 4.19 (s, 4H), 2.69 (s, 3H).

### Example 31

### N-[4-(3-Cyanophenyl)-5-(6-methylpyridazin-4-yl)thiazol-2-yl]-2-oxa-6-azaspiro[3.3]heptane-6-carboxamide

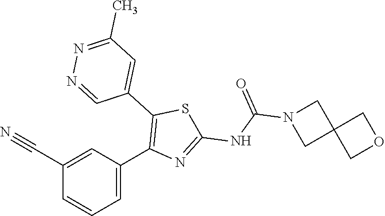 [SMILES: Cc1cc(-c2sc(NC(=O)N3CC4(COC4)C3)nc2-c2cccc(C#N)c2)cnn1]

The title compound was prepared from 3-[2-amino-5-(6-methylpyridazin-4-yl)thiazol-4-yl]benzonitrile (Intermediate AY) and 2-oxa-6-azaspiro[3.3]heptane analogously to Example 7.

LC-MS-1 (Method 8B): Rt 1.90 mins; MS m/z 419.1=[M+H]+ 1H NMR (500 MHz, DMSO-d6) δ 11.50 (s, 1H), 8.74 (d, J=2.3 Hz, 1H), 7.89 (t, J=1.8 Hz, 1H), 7.87 (dt, J=7.8, 1.5 Hz, 1H), 7.71 (dt, J=7.9, 1.4 Hz, 1H), 7.59 (t, J=7.8 Hz, 1H), 7.51 (s, 1H), 4.67 (s, 4H), 4.20 (s, 4H), 2.59 (s, 3H).

### Example 32

### N-[4-(3-Cyanophenyl)-5-[5-methyl-6-(triazol-2-yl)-3-pyridyl]thiazol-2-yl]-2-oxa-6-azaspiro[3.3]heptane-6-carboxamide

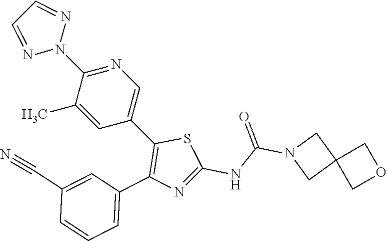 [SMILES: Cc1cc(-c2sc(NC(=O)N3CC4(COC4)C3)nc2-c2cccc(C#N)c2)cnc1-n1nccn1]

The title compound was prepared from 3-[2-amino-5-[5-methyl-6-(triazol-2-yl)-3-pyridyl]thiazol-4-yl]benzonitrile (Intermediate AN) and 2-oxa-6-azaspiro[3.3]heptane analogously to Example 7.

LC-MS-1 (Method 8A): Rt 3.27 mins; MS m/z 485.1=[M+H]+ 1H NMR (500 MHz, DMSO-d6) δ 10.97 (br s, 1H), 8.24 (d, J=2.3 Hz, 1H), 8.15 (s, 2H), 7.94-7.91 (m, 2H), 7.80 (dd, J=7.8, 1.5 Hz, 1H), 7.73 (dt, J=7.8, 1.5 Hz, 1H), 7.56 (t, J=7.8 Hz, 1H), 4.68 (s, 4H), 4.18 (s, 4H), 2.25 (s, 3H).

### Example 33

### N-[4-(3-Cyanophenyl)-5-pyrazolo[1,5-a]pyridin-5-yl-thiazol-2-yl]morpholine-4-carboxamide

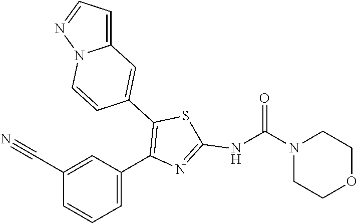 [SMILES: N#Cc1cccc(-c2nc(NC(=O)N3CCOCC3)sc2-c2ccn3nccc3c2)c1]

### Step 1: N-[4-(3-Cyanophenyl)thiazol-2-yl]morpholine-4-carboxamide

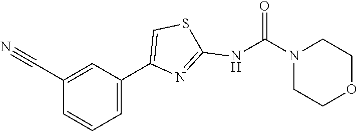 [SMILES: N#Cc1cccc(-c2csc(NC(=O)N3CCOCC3)n2)c1]

To a stirred solution of 3-(2-aminothiazol-4-yl)benzonitrile (Intermediate P)(653 mg, 3.08 mmol) in THF (10 mL) at 0° C. was added sodium hydride (60% in mineral oil) (150 mg, 3.75 mmol). After stirring at room temperature for 20 mins, morpholine-4-carbonyl chloride (520 μL, 4.55 mmol) was added and stirring continued for 18 h. The resulting mixture was diluted with THF (10 mL) then retreated with sodium hydride (60% in mineral oil) (150 mg, 3.75 mmol). After stirring for 10 mins, morpholine-4-carbonyl chloride (520 μL, 4.55 mmol) was added and stirring continued for 4 h. The reaction was quenched with water (20 mL) and extracted with EtOAc (2×40 mL). The aqueous layer was acidified to pH 5 using 1M HCl and extracted with EtOAc (2×30 mL). The combined organic extracts were washed with brine (30 mL), dried over MgSO4 and concentrated in vacuo. The resulting brown oil was purified by chromatography on silica eluting with 0-100% EtOAc in heptanes to afford the title compound as a beige solid.

LC-MS-S (Method 2A): Rt 1.06 mins; MS m/z 315.1=[M+H]+

1H NMR (400 MHz, DMSO-d6) δ 11.05 (s, 1H), 8.31 (t, J=1.5 Hz, 1H), 8.21 (dt, J=7.9, 1.3 Hz, 1H), 7.76 (dt, J=7.7, 1.3 Hz, 1H), 7.73 (s, 1H), 7.63 (t, J=7.8 Hz, 1H), 3.67-3.58 (m, 4H), 3.56-3.48 (m, 4H).

### Step 2: N-[5-Bromo-4-(3-cyanophenyl)thiazol-2-yl]morpholine-4-carboxamide

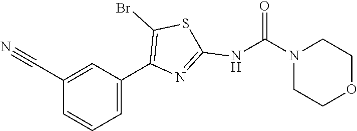 [SMILES: N#Cc1cccc(-c2nc(NC(=O)N3CCOCC3)sc2Br)c1]

To a solution of N-[4-(3-cyanophenyl)thiazol-2-yl]morpholine-4-carboxamide (step 1) (790 mg, 2.34 mmol) in THF (20 mL) was added NBS (499 mg, 2.80 mmol) and the reaction mixture was stirred at room temperature for 30 mins. The resulting mixture was diluted with EtOAc (50 mL) and washed with water (30 mL), sat. aq. NaHCO₃ solution (2×30 mL) and brine (30 mL). The organic layer was dried over MgSO₄ and concentrated in vacuo to afford the title compound as a yellow solid.

LC-MS-S (Method 2A): Rt 1.18 mins; MS m/z 393.0/395.0=[M+H]+

1H NMR (500 MHz, DMSO-d6) δ 11.35 (s, 1H), 8.25 (t, J=1.5 Hz, 1H), 8.21 (dt, J=8.0, 1.4 Hz, 1H), 7.88 (dt, J=7.7, 1.3 Hz, 1H), 7.71 (t, J=7.9 Hz, 1H), 3.63-3.56 (m, 4H), 3.55-3.48 (m, 4H).

### Step 3: N-[4-(3-Cyanophenyl)-5-pyrazolo[1,5-a]pyridin-5-yl-thiazol-2-yl]morpholine-4-carboxamide

A stirred solution of N-[5-bromo-4-(3-cyanophenyl)thiazol-2-yl]morpholine-4-carboxamide (step 2) (75 mg, 0.19 mmol), 5-(4,4,5,5-tetramethyl-1,3,2-dioxaborolan-2-yl)pyrazolo[1,5-a]pyridine (186 mg, 0.76 mmol) and cesium carbonate (186 mg, 0.57 mmol) in monoglyme (4 mL) and water (0.5 mL) was degassed with nitrogen for 5 mins.

Bis(triphenylphosphine)palladium(II) dichloride (27 mg, 0.04 mmol) was added and the sealed vessel was heated to 85° C. under nitrogen for 17 h. The resulting mixture was allowed to cool to room temperature, diluted with water (2 mL) and extracted with EtOAc (4×3 mL). The combined organic extracts were filtered through a phase separating cartridge and concentrated in vacuo. Purification of the crude product by C18 reverse phase chromatography eluting with 0.1% formic acid in MeCN/water afforded a solid which was triturated in MeCN, filtered and the filtrate concentrated in vacuo to give a yellow solid.

Purification by preparative HPLC (conditions below) afforded the title compound as a yellow solid.

Purification conditions—Column Details: Waters XBridge C18 column (19 mm×100 mm, 5 μm); Column Temperature: Room Temperature; Flow Rate: 20 ml/min; Detector Wavelength: 215 nm; Solvent Gradient: 5% B (A=0.2% ammonium hydroxide in water; B=0.2% ammonium hydroxide in acetonitrile) for 2.0 min, then a gradient of 5-55% B over 20 min, then a gradient of 55-100% B over 0.5 min and held for 2.0 min.

LC-MS (Method 7B): Rt 2.65 mins; MS m/z 431.2=[M+H]+

1H NMR (500 MHz, DMSO-d6) δ 8.55 (d, J=7.2 Hz, 1H), 7.97 (d, J=2.0 Hz, 1H), 7.91 (t, J=1.4 Hz, 1H), 7.77-7.71 (m, 2H), 7.59 (s, 1H), 7.51 (t, J=7.8 Hz, 1H), 6.57 (s, 1H), 6.54 (dd, J=7.1, 1.7 Hz, 1H), 3.61-3.54 (m, 4H), 3.53-3.47 (m, 4H). NH not observed.

### Example 34

### N-[4-(3-Cyanophenyl)-5-(6-methyl-1H-pyrrolo[2,3-b]pyridin-4-yl)thiazol-2-yl]-2-oxa-6-azaspiro[3.3]heptane-6-carboxamide

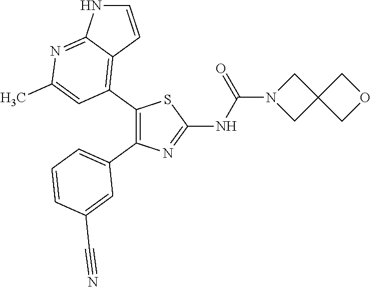 [SMILES: Cc1cc(-c2sc(NC(=O)N3CC4(COC4)C3)nc2-c2cccc(C#N)c2)c2cc[nH]c2n1]

### Step 1: tert-Butyl 4-[2-amino-4-(3-cyanophenyl)thiazol-5-yl]-6-methyl-pyrrolo[2,3-b]pyridine-1-carboxylate

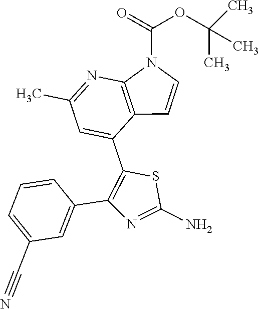 [SMILES: Cc1cc(-c2sc(N)nc2-c2cccc(C#N)c2)c2ccn(C(=O)OC(C)(C)C)c2n1]

The title compound was prepared from tert-butyl 6-methyl-4-(4,4,5,5-tetramethyl-1,3,2-dioxaborolan-2-yl)pyrrolo[2,3-b]pyridine-1-carboxylate (Intermediate R) and 3-(2-amino-5-bromo-thiazol-4-yl)benzonitrile (Intermediate Q) analogously to Example 17 step 1.

LC-MS-2 (Method 2B): Rt 1.64 mins; MS m/z 432.1=[M+H]+

1H NMR (500 MHz, DMSO-d6) δ 7.79 (t, J=1.5 Hz, 1H), 7.69 (dt, J=7.7, 1.4 Hz, 1H), 7.56 (d, J=4.1 Hz, 1H), 7.50 (dt, J=8.0, 1.5 Hz, 1H), 7.49-7.43 (m, 2H), 7.39 (t, J=7.8 Hz, 1H), 7.05 (s, 1H), 6.07 (d, J=4.1 Hz, 1H), 1.59 (s, 9H). 1×CH₃ not observed.

### Step 2: tert-Butyl 4-[4-(3-cyanophenyl)-2-(2-oxa-6-azaspiro[3.3]heptane-6-carbonylamino)thiazol-5-yl]-6-methyl-pyrrolo[2,3-b]pyridine-1-carboxylate

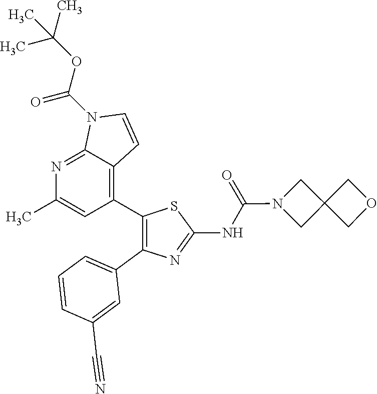 [SMILES: Cc1cc(-c2sc(NC(=O)N3CC4(COC4)C3)nc2-c2cccc(C#N)c2)c2ccn(C(=O)OC(C)(C)C)c2n1]

The title compound was prepared from tert-butyl 4-[2-amino-4-(3-cyanophenyl)thiazol-5-yl]-6-methyl-pyrrolo[2,3-b]pyridine-1-carboxylate (step 1) and 2-oxa-6-azaspiro[3.3]heptane analogously to Example 7.

LC-MS-1 (Method 3B): Rt 1.83 mins; MS m/z 557.3=[M+H]+

1H NMR (500 MHz, DMSO-d6) δ 11.40 (s, 1H), 7.87 (s, 1H), 7.73 (d, J=8.0 Hz, 1H), 7.61-7.57 (m, 1H), 7.54 (d, J=8.0 Hz, 1H), 7.42 (t, J=8.0 Hz, 1H), 7.15 (s, 1H), 6.09-6.04 (m, 1H), 4.68 (s, 4H), 4.22 (s, 4H), 1.60 (s, 9H). 1×CH₃ not observed.

### Step 3: N-[4-(3-Cyanophenyl)-5-(6-methyl-1H-pyrrolo[2,3-b]pyridin-4-yl)thiazol-2-yl]-2-oxa-6-azaspiro[3.3]heptane-6-carboxamide

The title compound was prepared from tert-Butyl 4-[4-(3-cyanophenyl)-2-(2-oxa-6-azaspiro[3.3]heptane-6-carbonylamino)thiazol-5-yl]-6-methyl-pyrrolo[2,3-b]pyridine-1-carboxylate (step 2) and TFA analogously to Example 17 step 3.

LC-MS-2 (Method 8B): Rt 2.89 mins; MS m/z 457.1=[M+H]+

1H NMR (500 MHz, Methanol-d4) δ 7.86 (t, J=1.5 Hz, 1H), 7.63 (dt, J=7.8, 1.5 Hz, 1H), 7.55 (dt, J=7.8, 1.5 Hz, 1H), 7.33 (t, J=7.8 Hz, 1H), 7.21 (d, J=3.5 Hz, 1H), 6.95 (s, 1H), 6.03 (d, J=3.5 Hz, 1H), 4.83 (s, 4H), 4.31 (s, 4H), 2.55 (s, 3H). 2×NH not observed.

### Example 35

### N-[5-(1,3-benzoxazol-6-yl)-4-(3-cyanophenyl)thiazol-2-yl]-2-oxa-6-azaspiro[3.3]heptane-6-carboxamide

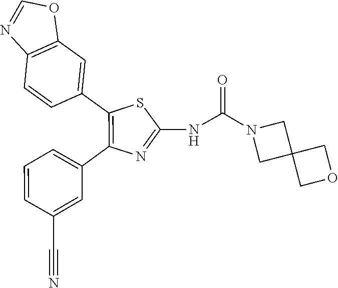 [SMILES: N#Cc1cccc(-c2nc(NC(=O)N3CC4(COC4)C3)sc2-c2ccc3ncoc3c2)c1]

The title compound was prepared from 3-[2-amino-5-(1,3-benzoxazol-6-yl)thiazol-4-yl]benzonitrile (Intermediate AZ) and 2-oxa-6-azaspiro[3.3]heptane analogously to Example 7.

LC-MS-2 (Method 2B): Rt 2.84 mins; MS m/z 444.2=[M+H]+

1H NMR (500 MHz, DMSO-d6) δ 11.27 (s, 1H), 8.79 (s, 1H), 7.83 (t, J=1.7 Hz, 1H), 7.80 (d, J=8.2 Hz, 1H), 7.76 (d, J=1.6 Hz, 1H), 7.75-7.73 (m, 1H), 7.64 (dt, J=8.0, 1.4 Hz, 1H), 7.48 (t, J=7.8 Hz, 1H), 7.30 (dd, J=8.3, 1.7 Hz, 1H), 4.68 (s, 4H), 4.20 (s, 4H).

### Preparation of Intermediates

### Intermediate A

### 3-[2-Amino-5-(4-methylquinazolin-6-yl)thiazol-4-yl]benzonitrile CH₃

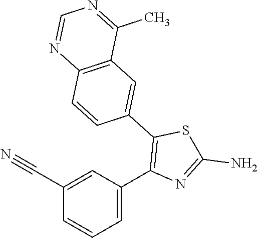 [SMILES: Cc1ncnc2ccc(-c3sc(N)nc3-c3cccc(C#N)c3)cc12]

### Step 1: 4-Methyl-6-(4,4,5,5-tetramethyl-1,3,2-dioxaborlon-2-yl)quinazoline

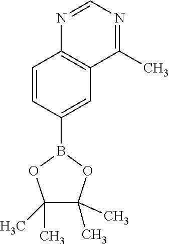

A solution of 6-bromo-4-methyl-quinazoline (5.0 g, 22.41 mmol) and bis(pinacolato)diboron (6.83 g, 26.9 mmol) in 1,4-dioxane (100 mL) was de-oxygenated via nitrogen sparging for 10 mins. Pd(dppf)Cl₂ (1.64 g, 2.24 mmol) and KOAc (5.5 g, 56.04 mmol) were added, a reflux condenser fitted, and the system evacuated and backfilled with nitrogen (3×cycles). The reaction mixture was stirred at 80° C. for 20 h and then allowed to cool to room temperature. The resulting mixture was diluted with EtOAc (100 mL) and filtered through a plug of Celite® (filter material), eluting with EtOAc. The filtrate was concentrated in vacuo to afford the title compound as a dark brown residue.

LC-MS-1 (Method 2B): Rt 0.70 mins; MS m/z 271.3=[M+H]+

1H NMR (500 MHz, DMSO-d6) δ 9.14 (s, 1H), 8.52 (t, J=1.0 Hz, 1H), 8.16 (dd, J=8.3, 1.0 Hz, 1H), 7.96 (d, J=8.3 Hz, 1H), 2.96 (s, 3H), 1.17 (s, 12H).

### Step 2: 3-[2-Amino-5-(4-methylquinazolin-6-yl)thiazol-4-yl]benzonitrile

A solution of K₂CO₃ (6.45 g, 46.67 mmol) in water (35 mL) was added to a solution of 3-(2-amino-5-bromo-thiazol-4-yl)benzonitrile (Intermediate Q)(5.23 g, 18.67 mmol) and crude 4-methyl-6-(4,4,5,5-tetramethyl-1,3,2-dioxaborolan-2-yl)quinazoline (step 1) (6.05 g, 22.4 mmol) in 1,4-dioxane (140 mL) and the mixture was de-oxygenated via nitrogen sparging for 10 mins. bis(tri-tert-butylphosphine)palladium(0) (954 mg, 1.87 mmol) was added, the flask sealed and the contents evacuated and backfilled with nitrogen (3×cycles). The reaction mixture was stirred at 80° C. for 75 mins and then allowed to cool to room temperature. The resulting mixture was diluted with EtOAc (400 mL) and washed with water (400 mL) and brine (2×200 mL). The organic portion was dried over Na₂SO₄ and concentrated in vacuo to afford a dark brown residue. This material was triturated in 2% MeOH in DCM (50 mL) via sonication and the suspension collected by filtration. The solids were washed with DCM (2×5 mL) and vacuum dried to afford a yellow-tan solid. The material was triturated with 10:1 MeOH/MeCN (30 mL) under sonication and the suspension was filtered. The solids were washed with 10:1 MeOH/MeCN (10 mL) and vacuum dried to afford the title compound as a yellow-tan solid.

LC-MS-2 (Method 2B): Rt 1.22 mins; MS m/z 344.0=[M+H]+

1H NMR (500 MHz, DMSO) δ 9.08 (s, 1H), 8.06 (d, J=2.0 Hz, 1H), 7.88 (d, J=8.7 Hz, 1H), 7.83 (br t, J=1.5 Hz, 1H), 7.76 (br dt, J=7.7, 1.5 Hz, 1H), 7.71 (dd, J=8.7, 2.0 Hz, 1H), 7.66 (br dt, J=8.0, 1.5 Hz, 1H), 7.48 (apr t, J=7.8 Hz, 1H), 7.43 (s, 2H), 2.78 (s, 3H).

The intermediates of the following Table were prepared analogously to Intermediate A steps 1 and 2 from the appropriate starting compounds.

TABLE Int AX

| Int | Product of step 1 | Product of step 2 |
|---|---|---|
| AA | 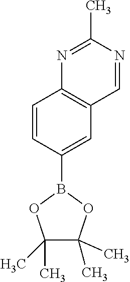 [SMILES: Cc1ncc2cc(B3OC(C)(C)C(C)(C)O3)ccc2n1] | 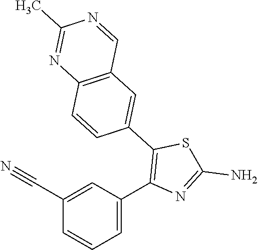 [SMILES: Cc1ncc2cc(-c3sc(N)nc3-c3cccc(C#N)c3)ccc2n1] |
| | 2-Methyl-6-(4,4,5,5-tetramethyl- | 3-[2-Amino-5-(2-methylquinazolin-6- |
| | 1,3,2-dioxaborolan-2-yl)quinazoline | yl)thiazol-4-yl]benzonitrile |
| | LC-MS-1 (Method 2B): Rt 0.69 mins; | LC-MS-1 (Method 2B): Rt 1.13 mins; MS m/z |
| | MS m/z 271.3 = [M + H]+ | 344.3 = [M + H]+ |
| | 1H NMR (500 MHz, DMSO) δ 9.60 (s, | 1H NMR (500 MHz, DMSO) δ 9.46 (s, 1H), |
| | 1H), 8.50 (s, 1H), 8.13 (dd, J = 8.4, | 8.03 (d, J = 2.1 Hz, 1H), 7.83-7.79 (m, 2H), |
| | 1.4 Hz, 1H), 7.88 (d, J = 8.4 Hz, 1H), | 7.75 (dt, J = 7.7, 1.5 Hz, 1H), 7.68 (dd, J = |
| | 2.79 (s, 3H), 1.35 (s, 12H). | 8.7, 2.1 Hz, 1H), 7.64 (dt, J = 8.0, 1.5 Hz, |
| | Prepared from 6-bromo-2-methyl- | 1H), 7.47 (t, J = 7.9 Hz, 1H), 7.43-7.40 (m, |
| | quinazoline and | 2H), 2.77 (s, 3H). |
| | bis(pinacolato)diboron | |
| AB | 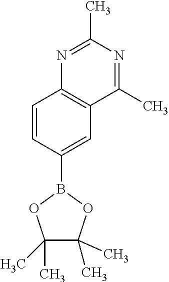 | 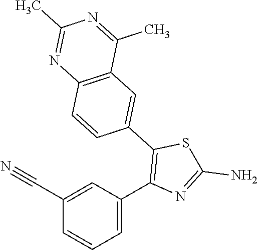 [SMILES: Cc1nc(C)c2cc(-c3sc(N)nc3-c3cccc(C#N)c3)ccc2n1] |
| | 2,4-Dimethyl-6-(4,4,5,5-tetramethyl- | 3-[2-Amino-5-(2,4-dimethylquinazolin-6- |
| | 1,3,2-dioxaborolan-2-yl)quinazoline | yl)thiazol-4-yl]benzonitrile |
| | LC-MS-2 (Method 2B): Rt 1.37 mins; | LC-MS-1 (Method 2B): Rt 1.15 mins; MS m/z |
| | MS m/z 285.1 = [M + H]+ | 358.3 = [M + H]+ |
| | 1H NMR (500 MHz, DMSO) δ 8.47 | 1H NMR (500 MHz, DMSO) δ 8.03 (d, J = 2.0 |
| | (d, J = 1.4 Hz, 1H), 8.10 (dd, J = 8.4, | Hz, 1H), 7.82 (t, J = 1.8 Hz, 1H), 7.78 (d, J = |
| | 1.3 Hz, 1H), 7.85 (d, J = 8.4 Hz, 1H), | 8.7 Hz, 1H), 7.75 (dt, J = 7.7, 1.5 Hz, 1H), |
| | 2.91 (s, 3H), 2.72 (s, 3H), 1.35 (s, | 7.66 (dd, J = 8.5, 1.9 Hz, 2H), 7.48 (t, J = 7.8 |
| | 12H). | Hz, 1H), 7.42-7.34 (m, 2H), 2.74 (s, 3H), |
| | Prepared from 6-bromo-2,4- | 2.70 (s, 3H). |
| | dimethyl-quinazoline (Intermediate | |
| | G) and bis(pinacolato)diboron | |
| AC | 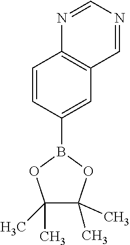 [SMILES: CC1(C)OB(c2ccc3ncncc3c2)OC1(C)C] | 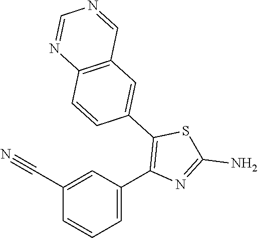 [SMILES: N#Cc1cccc(-c2nc(N)sc2-c2ccc3ncncc3c2)c1] |
| | 6-(4,4,5,5-Tetramethyl-1,3,2- | 3-(2-Amino-5-quinazolin-6-yl-thiazol-4- |
| | dioxaborolan-2-yl)quinazoline | yl)benzonitrile |
| | Commercially available | LC-MS-1 (Method 2B): Rt 1.07 mins; MS m/z |
| | | 330.2 = [M + H]+ |
| | | 1H NMR (500 MHz, DMSO-d6) δ 9.56 (s, |
| | | 1H), 9.28 (s, 1H), 8.08 (d, J = 2.1 Hz, 1H), |
| | | 7.91 (d, J = 8.8 Hz, 1H), 7.83 (s, 1H), 7.76 (d, |
| | | J = 7.8 Hz, 1H), 7.73 (dd, J = 8.8, 2.1 Hz, 1H), |
| | | 7.65 (d, J = 7.8 Hz, 1H), 7.48 (t, J = 7.8 Hz, |
| | | 1H), 7.45 (s, 2H). |
| AD | 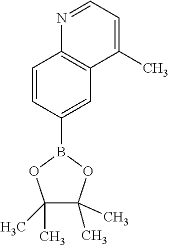 | 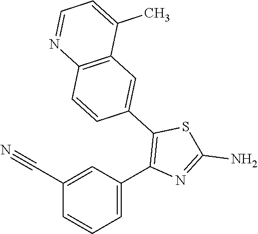 [SMILES: Cc1ccnc2ccc(-c3sc(N)nc3-c3cccc(C#N)c3)cc12] |
| | 4-Methyl-6-(4,4,5,5-tetramethyl- | 3-[2-Amino-5-(4-methyl-6-quinolyl)thiazol-4- |
| | 1,3,2-dioxaborolan-2-yl)quinoline | ylbenzonitrile |
| | Commercially available | LC-MS-2 (Method 2B): Rt 1.38 mins; MS m/z |
| | | 343.0 = [M + H]+ |
| | | 1H NMR (500 MHz, DMSO-d6) δ 8.72 (d, J = |
| | | 4.3 Hz, 1H), 7.93-7.89 (m, 2H), 7.83 (t, J = |
| | | 1.8 Hz, 1H), 7.74 (dt, J = 7.7, 1.5 Hz, 1H), |
| | | 7.67 (dt, J = 7.9, 1.5 Hz, 1H), 7.51 (dd, J = |
| | | 8.6, 2.1 Hz, 1H), 7.47 (t, J = 7.9 Hz, 1H), 7.38- |
| | | 7.36 (m, 1H), 7.36 (s, 2H), 2.53 (s, 3H). |
| AE | 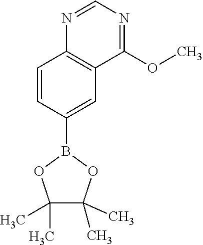 | 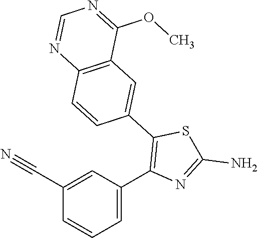 [SMILES: COc1ncnc2ccc(-c3sc(N)nc3-c3cccc(C#N)c3)cc12] |
| | 4-Methoxy-6-(4,4,5,5-tetramethyl- | 3-[2-Amino-5-(4-methoxyquinazolin-6- |
| | 1,3,2-dioxaborolan-2-yl)quinazoline | yl)thiazol-4-yl]benzonitrile |
| | LC-MS-1 (Method 2B): Rt 1.28 mins; | LC-MS-2 (Method 2B): Rt 1.34 mins; MS m/z |
| | MS m/z 287.3 = [M + H]+ | 360.1 = [M + H]+ |
| | 1H NMR (500 MHz, DMSO-d6) δ | 1H NMR (500 MHz, DMSO-d6) δ 8.79 (s, |
| | 8.85 (s, 1H), 8.50 (d, J = 1.5 Hz, 1H), | 1H), 7.96 (d, J = 2.1 Hz, 1H), 7.83-7.80 (m, |
| | 8.12 (dd, J = 8.3, 1.5 Hz, 1H), 7.89 (d, | 2H), 7.75 (dt, J = 7.7, 1.5 Hz, 1H), 7.65 (dq, |
| | J = 8.3 Hz, 1H), 4.16 (s, 3H), 1.34 (s, | J = 8.1, 1.9, 1.5 Hz, 2H), 7.48 (t, J = 7.8 Hz, |
| | 12H). | 1H), 7.41 (s, 2H), 4.11 (s, 3H). |
| | Prepared from 6-bromo-4-methoxy- | |
| | quinazoline and | |
| | bis(pinacolato)diboron | |
| AF | 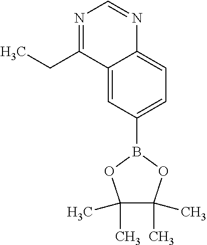 [SMILES: CCc1ncnc2ccc(B3OC(C)(C)C(C)(C)O3)cc12] | 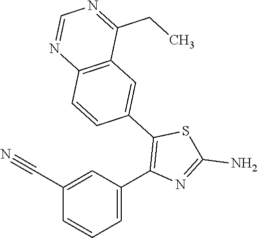 [SMILES: CCc1ncnc2ccc(-c3sc(N)nc3-c3cccc(C#N)c3)cc12] |
| | 4-Ethyl-6-(4,4,5,5-tetramethyl-1,3,2- | 3-[2-Amino-5-(4-ethylquinazolin-6-yl)thiazol- |
| | dioxaborolan-2-yl)quinazoline | 4-yl]benzonitrile |
| | LC-MS-2 (Method 2B): Rt 1.54 mins; | LC-MS-1 (Method 2B): Rt 1.20 mins; MS m/z |
| | MS m/z 285.1 = [M + H]+ | 358.3 = [M + H]+ |
| | 1H NMR (500 MHz, DMSO) δ 9.20 (s, | 1H NMR (500 MHz, DMSO) δ 9.13 (s, 1H), |
| | 1H), 8.55 (br s, 1H), 8.16 (dd, J = 8.4, | 8.06 (d, J = 2.0 Hz, 1H), 7.91 (d, J = 8.7 Hz, |
| | 1.3 Hz, 1H), 7.97 (d, J = 8.4 Hz, 1H), | 1H), 7.84 (br t, J = 1.5 Hz, 1H), 7.77-7.73 |
| | 3.37 (q, J = 7.5 Hz, 3H), 1.40-1.34 | (m, 2H), 7.67 (br dt, J = 8.0, 1.5 Hz, 1H), 7.48 |
| | (m, 2H), 1.35 (s, 12H) | (apr t, J = 7.8 Hz, 1H), 7.46-7.39 (m, 2H), |
| | Prepared from 6-bromo-4-ethyl- | 3.14 (q, J = 7.5 Hz, 2H), 1.22 (t, J = 7.5 Hz, |
| | quinazoline (Intermediate E) and | 3H) |
| | bis(pinacolato)diboron | |
| AG | 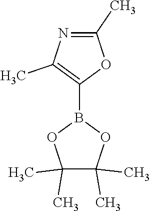 | 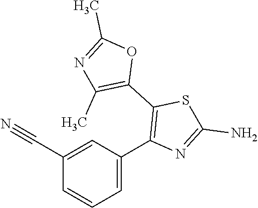 [SMILES: Cc1nc(C)c(-c2sc(N)nc2-c2cccc(C#N)c2)o1] |
| | 2,4-Dimethyl-5-(4,4,5,5-tetramethyl- | 3-[2-Amino-5-(2,4-dimethyloxazol-5- |
| | 1,3,2-dioxaborolan-2-yl)oxazole | yl)thiazol-4-yl]benzonitrile |
| | Intermediate O | LC-MS-1 (Method 2B): Rt 1.10 mins; MS m/z |
| | | 297.1 = [M + H]+ |
| | | 1H NMR (500 MHz, DMSO) δ 7.81 (t, J = 1.5 |
| | | Hz, 1H), 7.76 (dt, J = 7.7, 1.5 Hz, 1H), 7.68 |
| | | (dt, J = 8.0, 1.5 Hz, 1H), 7.54 (apr t, J = 7.8 |
| | | Hz, 1H), 7.49 (s, 2H), 2.37 (s, 3H), 1.70 (s, |
| | | 3H) |
| AH | 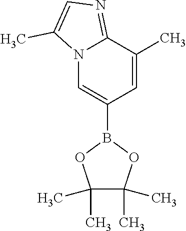 | 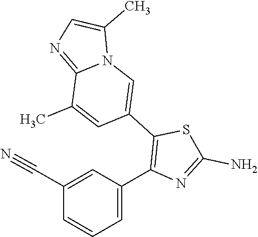 [SMILES: Cc1cc(-c2sc(N)nc2-c2cccc(C#N)c2)cn2c(C)cnc12] |
| | 3,8-Dimethyl-6-(4,4,5,5-tetramethyl- | 3-[2-Amino-5-(3,8-dimethylimidazo[1,2-a] |
| | 1,3,2-dioxaborolan-2-yl)imidazo[1,2- | pyridine-6-yl)thiazol-4-yl]benzonitrile |
| | a]pyridine | LC-MS-1 (Method 2B): Rt 1.12 mins; MS m/z |
| | LC-MS-2 (Method 2B): Rt 1.21 mins; | 346.2 = [M + H]+ |
| | MS m/z 273.0 = [M + H]+ | 1H NMR (500 MHz, DMSO) δ 8.06 (d, J = 1.6 |
| | 1H NMR (500 MHz, DMSO) δ 8.20 (s, | Hz, 1H), 7.88 (t, J = 1.6 Hz, 1H), 7.72-7.67 |
| | 1H), 7.34 (s, 1H), 7.14 (s, 1H), 2.48 | (m, 2H), 7.46 (apr t, J = 7.8 Hz, 1H), 7.34 (d, |
| | (s, 3H), 2.46 (s, 3H), 1.32 (s, 12H) | J = 1.0 Hz, 1H), 7.28 (s, 2H), 6.82 (t, J = 1.6 |
| | Prepared from 6-bromo-3,8- | Hz, 1H), 2.40 (s, 3H), 2.37 (d, J = 1.0 Hz, 3H) |
| | dimethyl-imidazo[1,2-a]pyridine | |
| | (Intermediate H) and | |
| | bis(pinacolato)diboron | |
| AI | 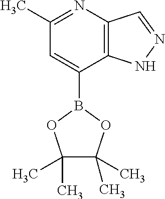 [SMILES: Cc1cc(B2OC(C)(C)C(C)(C)O2)c2[nH]ncc2n1] | 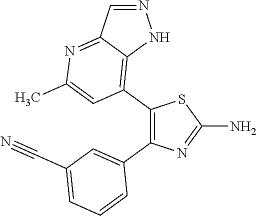 [SMILES: Cc1cc(-c2sc(N)nc2-c2cccc(C#N)c2)c2[nH]ncc2n1] |
| | 5-Methyl-7-(4,4,5,5-tetramethyl- | 3-[2-Amino-5-(5-methyl-1H-pyrazolo[4,3- |
| | 1,3,2-dioxaborolan-2-yl)-1H- | b]131yridine-7-yl)thiazol-4-yl]benzonitrile |
| | pyrazolo[4,3-b]pyridine | LC-MS-2 (Method 2B): Rt 1.03 mins; MS m/z |
| | 1H NMR (500 MHz, DMSO) δ 12.83 | 333.0 = [M + H]+ |
| | (s, 1H), 8.21 (s, 1H), 7.46 (s, 1H), | 1H NMR (500 MHz, DMSO) δ 12.85 (s, 1H), |
| | 2.63 (s, 3H), 1.40 (s, 12H). | 8.15 (d, J = 1.5 Hz, 1H), 7.77 (d, J = 1.8 Hz, |
| | Prepared from 7-chloro-5-methyl-1H- | 1H), 7.66 (dt, J = 7.8, 1.5 Hz, 1H), 7.50 (d, |
| | pyrazolo[4,3-b]pyridine and | J = 3.8 Hz, 2H), 7.45 (dt, J = 8.1, 1.5 Hz, 1H), |
| | bis(pinacolato)diboron | 7.33 (t, J = 7.8 Hz, 1H), 7.20 (s, 1H), 2.56 (s, |
| | | 3H). |
| AJ | 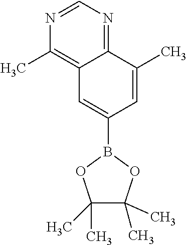 | 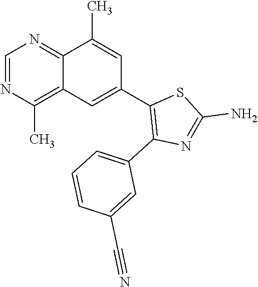 [SMILES: Cc1ncnc2c(C)cc(-c3sc(N)nc3-c3cccc(C#N)c3)cc12] |
| | 4,8-Dimethyl-6-(4,4,5,5-tetramethyl- | 3-[2-Amino-5-(4,8-dimethylquinazolin-6- |
| | 1,3,2-dioxaborolan-2-yl)quinazoline | yl)thiazol-4-yl]benzonitrile |
| | LC-MS-2 (Method 2B): Rt 1.10 mins; | LC-MS-2 (Method 2B): Rt 1.34 mins; MS m/z |
| | MS m/z 285.0 = [M + H]+ | 358.0 = [M + H]+ |
| | 1H NMR (500 MHz, DMSO) δ 9.17 (s, | 1H NMR (500 MHz, DMSO) δ 9.11 (s, 1H), |
| | 1H), 8.36 (s, 1H), 8.03 (s, 1H), 2.95 | 7.89-7.84 (m, 2H), 7.76 (dt, J = 7.7, 1.4 Hz, |
| | (s, 3H), 2.69 (s, 3H), 1.36 (s, 12H). | 1H), 7.67 (dt, J = 8.0, 1.5 Hz, 1H), 7.65-7.62 |
| | Prepared from 6-bromo-4,8- | (m, 1H), 7.48 (t, J = 7.8 Hz, 1H), 7.44-7.40 |
| | dimethyl-quinazoline (Intermediate I) | (m, 2H), 2.73 (s, 3H), 2.59 (s, 3H). |
| | and bis(pinacolato)diboron | |
| AK | 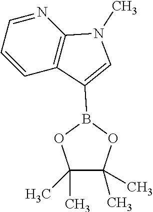 [SMILES: Cn1cc(B2OC(C)(C)C(C)(C)O2)c2cccnc21] | 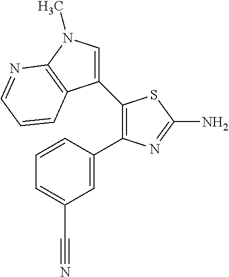 [SMILES: Cn1cc(-c2sc(N)nc2-c2cccc(C#N)c2)c2cccnc21] |
| | 1-Methyl-3-(4,4,5,5-tetramethyl- | 3-[2-Amino-5-(1-methylpyrrolo[2,3-b]pyridin- |
| | 1,3,2-dioxaborolan-2-yl)pyrrolo[2,3- | 3-yl)thiazol-4-yl]benzonitrile |
| | b]pyridine | LC-MS-2 (Method 2B): Rt 1.32 mins; MS m/z |
| | Commercially available | 332.0 = [M + H]+ |
| | | 1H NMR (500 MHz, DMSO-d6) δ 8.27 (dd, |
| | | J = 4.6, 1.6 Hz, 1H), 7.84 (t, J = 1.8 Hz, 1H), |
| | | 7.71-7.65 (m, 2H), 7.62 (dt, J = 7.7, 1.5 Hz, |
| | | 1H), 7.39-7.31 (m, 2H), 7.18-7.14 (m, 2H), |
| | | 6.99 (dd, J = 7.9, 4.6 Hz, 1H), 3.84 (s, 3H). |
| AL | 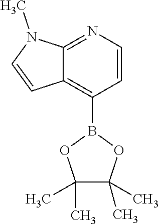 [SMILES: Cn1ccc2c(B3OC(C)(C)C(C)(C)O3)ccnc21] | 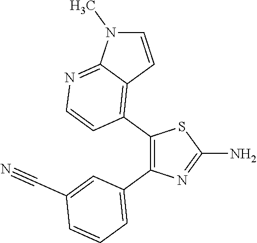 [SMILES: Cn1ccc2c(-c3sc(N)nc3-c3cccc(C#N)c3)ccnc21] |
| | 1-Methyl-4-(4,4,5,5-tetramethyl- | 4-(m-Fluorophenyl)-5-(9-methyl-1.7- |
| | 1,3,2-dioxaborolan-2-yl)pyrrolo[2,3- | diazabicyclo[4.3.0]nona-2,4,6,8-tetraen-3- |
| | b]pyridine | yl)-1,3-thiazol-2-ylamine |
| | LC-MS-2 (Method 2B): Rt 0.99 mins; | LC-MS-2 (Method 2B): Rt 1.31 mins; MS m/z |
| | MS m/z 259.0 = [M + H]+ | 332.0 = [M + H]+ |
| | 1H NMR (500 MHz, DMSO-d6) δ | 1H NMR (500 MHz, DMSO-d6) δ 8.18 (d, J = |
| | 8.27 (d, J = 4.6 Hz, 1H), 7.56 (d, J = | 4.9 Hz, 1H), 7.76 (d, J = 1.8 Hz, 1H), 7.68 (d, |
| | 3.4 Hz, 1H), 7.31 (d, J = 4.6 Hz, 1H), | J = 7.7 Hz, 1H), 7.56 (d, J = 8.0 Hz, 1H), 7.47 |
| | 6.66 (d, J = 3.4 Hz, 1H), 3.82 (s, 3H), | (d, J = 3.5 Hz, 1H), 7.43-7.37 (m, 3H), 6.90 |
| | 1.34 (s, 12H). | (d, J = 5.0 Hz, 1H), 6.10 (d, J = 3.6 Hz, 1H), |
| | Prepared from 4-bromo-1-methyl- | 3.81 (s, 3H). |
| | pyrrolo[2,3-b]pyridine (Intermediate | |
| | J) and bis(pinacolato)diboron | |
| AM | 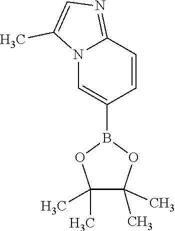 | 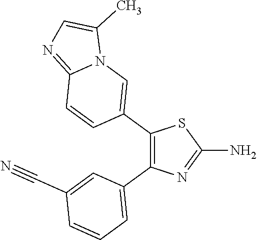 [SMILES: Cc1cnc2ccc(-c3sc(N)nc3-c3cccc(C#N)c3)cn12] |
| | 3-Methyl-6-(4,4,5,5-tetramethyl- | m-{2-Amino-5-(9-methyl-1.7- |
| | 1,3,2-dioxaborolan-2-yl)imidazo[1,2- | diazabicyclo[4.3.0]nona-2,4,6,8-tetraen-3- |
| | a]pyridine | yl)-1,3-thiazol-4-yl}benzonitrile |
| | LC-MS-2 (Method 2B): Rt 0.41 mins; | LC-MS-2 (Method 2B): Rt 1.21 mins; MS m/z |
| | MS m/z 176.9 = [M − C₆H₁₀]+ | 332.0 = [M + H]+ |
| | 1H NMR (500 MHz, DMSO-d6) δ | 1H NMR (500 MHz, DMSO-d6) δ 8.23 (s, |
| | 8.34 (s, 1H), 7.52-7.49 (m, 1H), 7.38 | 1H), 7.85 (s, 1H), 7.72-7.68 (m, 2H), 7.49 |
| | (d, J = 1.1 Hz, 1H), 7.31 (dd, J = 9.0, | (d, J = 6.4 Hz, 1H), 7.48-7.45 (m, 1H), 7.39 |
| | 1.3 Hz, 1H), 2.08 (s, 3H), 1.33 (s, | (s, 1H), 7.30 (s, 2H), 6.93 (dd, J = 9.3, 1.8 |
| | 12H). | Hz, 1H), 2.41 (s, 3H). |
| | Prepared from 6-bromo-3-methyl- | |
| | imidazo[1,2-a]pyridine and | |
| | bis(pinacolato)diboron | |
| AN | 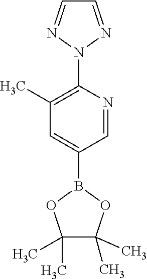 | 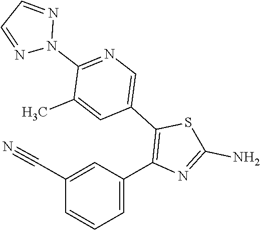 [SMILES: Cc1cc(-c2sc(N)nc2-c2cccc(C#N)c2)cnc1-n1nccn1] |
| | 3-Methyl-5-(4,4,5,5-tetramethyl- | 3-[2-Amino-5-[5-methyl-6-(triazol-2-yl)-3- |
| | 1,3,2-dioxaborolan-2-yl)-2-(triazol-2- | pyridyl]thiazol-4-yl]benzonitrile |
| | yl)pyridine | LC-MS-1 (Method 5B): Rt 2.61 mins; MS m/z |
| | LC-MS-1 (Method 5A): Rt 1.61 mins; | 360.1 = [M + H]+ |
| | MS m/z 204.9 = [M − C₆H₁₀]+ | 1H NMR (500 MHz, DMSO-d6) δ 8.18 (d, J = |
| | 1H NMR (500 MHz, Chloroform-d) δ | 2.3 Hz, 1H), 8.13 (s, 2H), 7.87 (dd, J = 1.8, |
| | 8.80 (br s, 1H), 8.13 (s, 1H), 8.08- | 1.4 Hz, 1H), 7.85-7.82 (m, 1H), 7.78 (ddd, |
| | 7.81 (m, 2H), 2.52 (s, 3H), 1.39 (s, | J = 7.8, 1.8, 1.4 Hz, 1H), 7.69 (ddd, J = 7.8, |
| | 12H). | 1.8, 1.4 Hz 1H), 7.54 (t, J = 7.8 Hz, 1H), 7.47 |
| | Prepared from 5-bromo-3-methyl-2- | (s, 2H), 2.22 (s, 3H). |
| | (triazol-2-yl)pyridine (Intermediate F) | |
| | and bis(pinacolato)diboron | |
| AO | 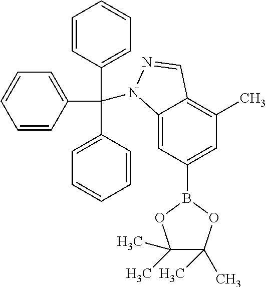 [SMILES: Cc1cc(B2OC(C)(C)C(C)(C)O2)cc2c1cnn2C(c1ccccc1)(c1ccccc1)c1ccccc1] | 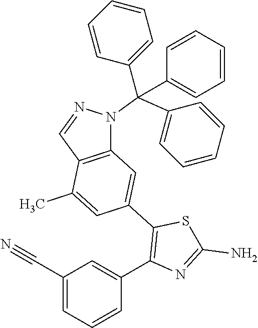 [SMILES: Cc1cc(-c2sc(N)nc2-c2cccc(C#N)c2)cc2c1cnn2C(c1ccccc1)(c1ccccc1)c1ccccc1] |
| | 4-Methyl-6-(4,4,5,5-tetramethyl- | m-[2-Amino-5-(4-methyl-1-trityl-1H-indazol- |
| | 1,3,2-dioxaborolan-2-yl)-1-trityl- | 6-yl)-1,3-thiazol-4-yl]benzonitrile |
| | indazole | LC-MS-2 (Method 2B): Rt 2.00 mins; MS m/z |
| | 1H NMR (500 MHz, DMSO-d6) δ | 572.2 = [M − H]− |
| | 7.98 (d, J = 1.0 Hz, 1H), 7.78 (s, 1H), | 1H NMR (500 MHz, DMSO-d6) δ 8.01 (s, |
| | 7.41-7.36 (m, 9H), 7.14-7.07 (m, | 1H), 7.79 (d, J = 1.7 Hz, 1H), 7.70 (t, J = 8.6 |
| | 6H), 7.05 (t, J = 1.1 Hz, 1H), 2.39 (s, | Hz, 2H), 7.46 (t, J = 7.8 Hz, 1H), 7.40-7.35 |
| | 3H), 1.30 (s, 12H). | (m, 10H), 7.22 (s, 1H), 7.10 (dd, J = 7.4, 2.3 |
| | Prepared from 6-bromo-4-methyl-1- | Hz, 7H), 6.62 (s, 1H), 2.30 (s, 3H). |
| | trityl-indazole (Intermediate K, isomer | |
| | 1) and bis(pinacolato)diboron | |
| AP | 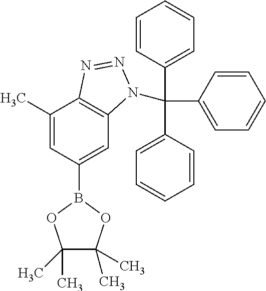 [SMILES: Cc1cc(B2OC(C)(C)C(C)(C)O2)cc2c1nnn2C(c1ccccc1)(c1ccccc1)c1ccccc1] | 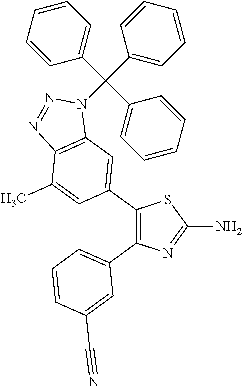 [SMILES: Cc1cc(-c2sc(N)nc2-c2cccc(C#N)c2)cc2c1nnn2C(c1ccccc1)(c1ccccc1)c1ccccc1] |
| | 4-Methyl-6-(4,4,5,5-tetramethyl- | 3-[2-Amino-5-(7-methyl-3-trityl-benzotriazol- |
| | 1,3,2-dioxaborolan-2-yl)-1-trityl- | 5-yl)thiazol-4-yl]benzonitrile |
| | benzotriazole | LC-MS-1 (Method 2B): Rt 1.58 mins; MS m/z |
| | 1H NMR (500 MHz, DMSO) δ 7.43- | 573.4 = [M − H]− |
| | 7.34 (m, 9H), 7.32-7.28 (m, 1H), | 1H NMR (500 MHz, DMSO) δ 7.65 (dt, J = |
| | 7.23-7.19 (m, 1H), 7.13-7.01 (m, | 7.5, 1.6 Hz, 1H), 7.56 (t, J = 1.8 Hz, 1H), 7.39- |
| | 6H), 2.70 (s, 3H), 1.19 (s, 12H). | 7.28 (m, 11H), 7.27-7.23 (m, 2H), 7.05- |
| | Prepared from 6-bromo-4-methyl-1- | 6.98 (m, 7H), 5.89 (s, 1H), 2.64 (s, 3H). |
| | trityl-benzotriazole (Intermediate L) | |
| | and 4,4,5,5-tetramethyl-2-(3,3,4,4- | |
| | tetramethylborolan-1-yl)-1,3,2- | |
| | dioxaborolane | |
| AQ | 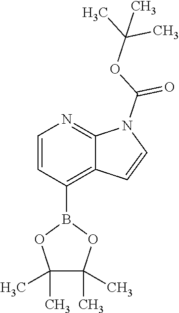 [SMILES: CC(C)(C)OC(=O)n1ccc2c(B3OC(C)(C)C(C)(C)O3)ccnc21] | 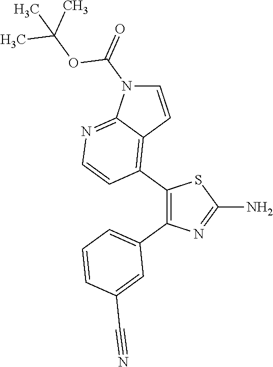 [SMILES: CC(C)(C)OC(=O)n1ccc2c(-c3sc(N)nc3-c3cccc(C#N)c3)ccnc21] |
| | tert-Butyl 4-(4,4,5,5-tetramethyl- | tert-Butyl 4-[2-amino-4-(3- |
| | 1,3,2-dioxaborolan-2-yl)pyrrolo[2,3- | cyanophenyl)thiazol-5-yl]pyrrolo[2,3- |
| | b]pyridine-1-carboxylate | b]pyridine-1-carboxylate |
| | Commercially available | LC-MS-1 (Method 3B): Rt 1.89 mins; MS m/z |
| | | 416.3 = [M − H]− |
| | | 1H NMR (500 MHz, DMSO) δ 8.33 (d, J = 5.0 |
| | | Hz, 1H), 7.79 (t, J = 1.7 Hz, 1H), 7.74-7.68 |
| | | (m, 2H), 7.55-7.49 (m, 3H), 7.41 (t, J = 7.8 |
| | | Hz, 1H), 7.12 (d, J = 5.0 Hz, 1H), 6.24 (d, J = |
| | | 4.1 Hz, 1H), 1.60 (s, 9H). |
| AR | 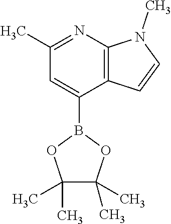 [SMILES: Cc1cc(B2OC(C)(C)C(C)(C)O2)c2ccn(C)c2n1] | 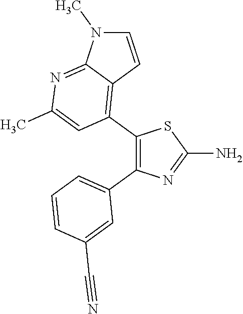 [SMILES: Cc1cc(-c2sc(N)nc2-c2cccc(C#N)c2)c2ccn(C)c2n1] |
| | 1,6-Dimethyl-4-(4,4,5,5-tetramethyl- | 3-[2-Amino-5-(1,6-dimethylpyrrolo[2,3- |
| | 1,3,2-dioxaborolan-2-yl)pyrrolo[2,3- | b]pyridin-4-yl)thiazol-4-yl]benzonitrile |
| | b]pyridine | LC-MS-1 (Method 2B): Rt 1.13 mins; MS |
| | LC-MS-2 (Method 2B): Rt 1.70 mins; | m/z 346.2 = [M + H]+ |
| | MS m/z 273.0 = [M + H]+ | 1H NMR (500 MHz, DMSO) δ 7.77 (t, J = |
| | 1H NMR (500 MHz, DMSO) δ 7.43 | 1.8 Hz, 1H), 7.68 (dt, J = 7.7, 1.4 Hz, 1H), |
| | (d, J = 3.4 Hz, 1H), 7.20 (s, 1H), | 7.56 (dt, J = 8.0, 1.5 Hz, 1H), 7.43-7.36 |
| | 6.58 (d, J = 3.4 Hz, 1H), 3.78 (s, | (m, 3H), 7.33 (d, J = 3.5 Hz, 1H), 6.83 (s, |
| | 3H), 2.55 (s, 3H), 1.33 (s, 12H). | 1H), 5.94 (d, J = 3.5 Hz, 1H), 3.77 (s, 3H), |
| | Prepared from 4-bromo-1,6- | 2.49 (s, 3H). |
| | dimethyl-pyrrolo[2,3-b]pyridine | |
| | (Intermediate M) | |
| | and bis(pinacolato)diboron | |
| AS | 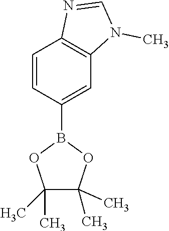 | 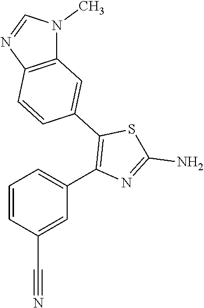 [SMILES: Cn1cnc2ccc(-c3sc(N)nc3-c3cccc(C#N)c3)cc21] |
| | 1-Methyl-6-(4,4,5,5-tetramethyl- | 3-[2-Amino-5-(3-methylbenzimidazol-5- |
| | 1,3,2-dioxaborolan-2- | yl)thiazol-4-yl]benzonitrile |
| | yl)benzimidazole | LC-MS-1 (Method 3B): Rt 1.32 mins; MS m/z |
| | Commercially available | 332.1 = [M + H]+ |
| | | 1H NMR (500 MHz, DMSO-d6) δ 8.21 (s, |
| | | 1H), 7.77 (t, J = 1.5 Hz, 1H), 7.66 (dt, J = 7.7, |
| | | 1.5 Hz, 1H), 7.63-7.61 (m, 1H), 7.58 (d, J = |
| | | 8.3 Hz, 1H), 7.52 (dd, J = 4.8, 1.3 Hz, 1H), |
| | | 7.41 (t, J = 7.8 Hz, 1H), 7.20 (s, 2H), 7.04 |
| | | (dd, J = 8.3, 1.6 Hz, 1H), 3.87 (s, 3H). |
| AT | 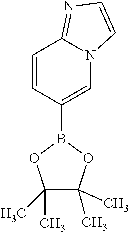 [SMILES: CC1(C)OB(c2ccc3nccn3c2)OC1(C)C] | 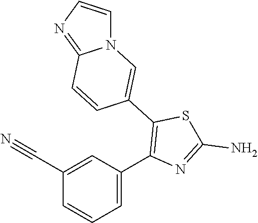 [SMILES: N#Cc1cccc(-c2nc(N)sc2-c2ccc3nccn3c2)c1] |
| | 6-(4,4,5,5-Tetramethyl-1,3,2- | 3-(2-Amino-5-imidazo[1,2-a]pyridin-6-yl- |
| | dioxaborolan-2-yl)imidazo[1,2- | thiazol-4-yl)benzonitrile |
| | a]pyridine | LC-MS-1 (Method 3B): Rt 1.67 mins; MS m/z |
| | Commercially available | 318.1 = [M + H]+ |
| | | 1H NMR (500 MHz, DMSO-d6) δ 8.61 (t, J = |
| | | 1.4 Hz, 1H), 7.92 (s, 1H), 7.85 (t, J = 1.8 Hz, |
| | | 1H), 7.72 (dt, J = 7.7, 1.4 Hz, 1H), 7.68 (dt, |
| | | J = 8.1, 1.5 Hz, 1H), 7.59 (d, J = 1.2 Hz, 1H), |
| | | 7.51 (d, J = 9.3 Hz, 1H), 7.47 (t, J = 7.8 Hz, |
| | | 1H), 7.32 (s, 2H), 6.96 (dd, J = 9.3, 1.9 Hz, |
| | | 1H). |
| AU | 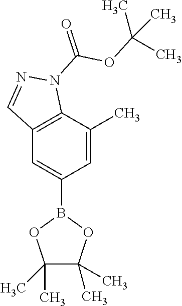 [SMILES: Cc1cc(B2OC(C)(C)C(C)(C)O2)cc2cnn(C(=O)OC(C)(C)C)c12] | 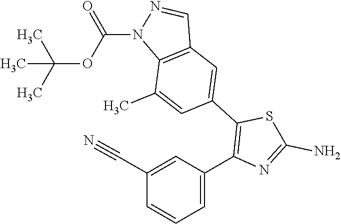 [SMILES: Cc1cc(-c2sc(N)nc2-c2cccc(C#N)c2)cc2cnn(C(=O)OC(C)(C)C)c12] |
| | tert-Butyl 7-methyl-5-(4,4,5,5- | tert-Butyl 5-[2-amino-4-(3- |
| | tetramethyl-1,3,2-dioxaborolan-2- | cyanophenyl)thiazol-5-yl]-7-methyl-indazole- |
| | yl)indazole-1-carboxylate | 1-carboxylate |
| | LC-MS-1 (Method 3B): Rt 2.35 mins; | LC-MS-1 (Method 5B): Rt 3.29 mins; MS m/z |
| | MS m/z 381.2 = [M + Na]+ | 432.0 = [M + H]+ |
| | 1H NMR (500 MHz, DMSO) δ 8.93 (s, | 1H NMR (500 MHz, DMSO-d6) δ 8.82 (s, |
| | 1H), 8.01 (s, 1H), 7.29 (m, 1H), 3.30 | 1H), 7.79 (t, J = 1.8 Hz, 1H), 7.70 (dt, J = 7.7, |
| | (s, 3H), 1.65 (s, 9H), 1.30 (s, 12H). | 1.4 Hz, 1H), 7.66 (dt, J = 8.1, 1.5 Hz, 1H), |
| | Prepared from tert-butyl 5-bromo-7- | 7.47-7.42 (m, 2H), 7.25 (s, 2H), 6.93 (t, J = |
| | methyl-indazole-1-carboxylate | 1.4 Hz, 1H), 2.42 (s, 3H), 1.64 (s, 9H). |
| | (Intermediate N) and | |
| | bis(pinacolato)diboron | |
| AV | 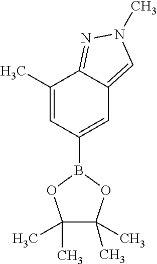 | 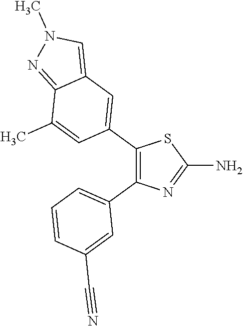 [SMILES: Cc1cc(-c2sc(N)nc2-c2cccc(C#N)c2)cc2cn(C)nc12] |
| | 2,7-Dimethyl-5-(4,4,5,5-tetramethyl- | 3-[2-Amino-5-(2,7-dimethylindazol-5- |
| | 1,3,2-dioxaborolan-2-yl)indazole | yl)thiazol-4-yl]benzonitrile |
| | LC-MS-1 (Method 3B): Rt 2.85 mins; | LC-MS-1 (Method 5B): Rt 2.58 mins; MS m/z |
| | MS m/z 273.1 = [M + H]+ | 346.0 = [M + H]+ |
| | 1H NMR (500 MHz, DMSO) δ 8.36 (s, | 1H NMR (500 MHz, DMSO) δ 8.28 (s, 1H), |
| | 1H), 7.96 (s, 1H), 7.22 (t, J = 1.2 Hz, | 7.79 (t, J = 1.7 Hz, 1H), 7.65 (ddt, J = 7.9, |
| | 1H), 4.18 (s, 3H), 2.48 (d, J = 1.0 Hz, | 6.2, 1.4 Hz, 2H), 7.50-7.38 (m, 2H), 7.16 (s, |
| | 3H), 1.30 (s, 12H). | 2H), 6.84 (t, J = 1.4 Hz, 1H), 4.17 (s, 3H), |
| | Prepared from 5-bromo-2,7-dimethyl- | 2.42 (s, 3H). |
| | indazole and bis(pinacolato)diboron | |
| AW | 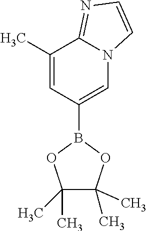 [SMILES: Cc1cc(B2OC(C)(C)C(C)(C)O2)cn2ccnc12] | 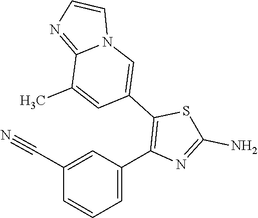 [SMILES: Cc1cc(-c2sc(N)nc2-c2cccc(C#N)c2)cn2ccnc12] |
| | 8-Methyl-6-(4,4,5,5-tetramethyl- | 3-[2-Amino-5-(8-methylimidazo[1,2- |
| | 1,3,2-dioxaborolan-2-yl)imidazo[1,2- | a]pyridin-6-yl)thiazol-4-yl]benzonitrile |
| | a]pyridine | LC-MS-1 (Method 3B): Rt 1.43 mins; MS m/z |
| | LC-MS-1 (Method 5B): Rt 1.24 mins; | 332.4 = [M + H]+ |
| | MS m/z 177.0 = [M − C₆H₁₀]+ | 1H NMR (500 MHz, DMSO-d6) δ 8.43 (d, J = |
| | 1H NMR (500 MHz, DMSO-d6) δ | 1.8 Hz, 1H), 7.89 (d, J = 1.2 Hz, 1H), 7.87 (t, |
| | 8.71 (s, 1H), 7.97 (d, J = 1.2 Hz, 1H), | J = 1.6 Hz, 1H), 7.71 (dt, J = 7.8, 1.6 Hz, 1H), |
| | 7.54 (d, J = 1.2 Hz, 1H), 7.13 (t, J = | 7.67 (dt, J = 7.8, 1.6 Hz, 1H), 7.54 (d, J = 1.2 |
| | 1.3 Hz, 1H), 3.57 (s, 3H), 1.31 (s, | Hz, 1H), 7.46 (t, J = 7.8 Hz, 1H), 7.29 (s, 2H), |
| | 12H). | 6.84 (t, J = 1.5 Hz, 1H), 2.41 (s, 3H). |
| | Prepared from 6-bromo-8-methyl- | |
| | imidazo[1,2-a]pyridine and | |
| | bis(pinacolato)diboron | |
| AX | 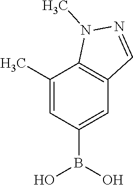 | 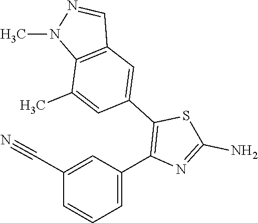 [SMILES: Cc1cc(-c2sc(N)nc2-c2cccc(C#N)c2)cc2cnn(C)c12] |
| | (1,7-Dimethylindazol-5-yl)boronic | 3-[2-Amino-5-(1,7-dimethylindazol-5- |
| | acid | yl)thiazol-4-yl]benzonitrile |
| | Commercially available | LC-MS-1 (Method 3B): Rt 1.72 mins; MS |
| | | m/z 346.1 = [M + H]+ |
| | | 1H NMR (500 MHz, DMSO) δ 7.93 (s, 1H), |
| | | 7.80 (t, J = 1.6 Hz, 1H), 7.66 (dt, J = 7.7, 1.6 |
| | | Hz, 1H), 7.60 (dt, J = 8.0, 1.6 Hz, 1H), 7.46 |
| | | (br s, 1H), 7.40 (apr t, J = 7.8 Hz, 1H), 7.17 |
| | | (br s, 2H), 6.98 (br s, 1H), 4.26 (s, 3H), 2.67 |
| | | (s, 3H) |
| AY | 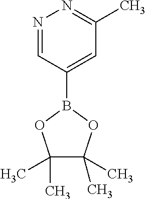 | 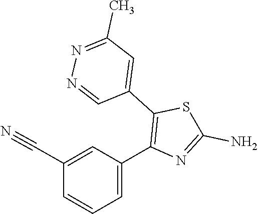 [SMILES: Cc1cc(-c2sc(N)nc2-c2cccc(C#N)c2)cnn1] |
| | 3-Methyl-5-(4,4,5,5-tetramethyl- | 3-[2-Amino-5-(6-methylpyridazin-4- |
| | 1,3,2-dioxaborolan-2-yl)pyridazine | yl)thiazol-4-yl]benzonitrile |
| | Commercially available | LC-MS-1 (Method 5B): Rt 1.92 mins; MS m/z |
| | | 294.0 = [M + H]+ |
| | | 1H NMR (500 MHz, DMSO-d6) δ 8.63 (d, J = |
| | | 2.3 Hz, 1H), 7.87-7.83 (m, 2H), 7.71-7.67 |
| | | (m, 3H), 7.58 (t, J = 8.0 Hz, 1H), 7.31 (d, J = |
| | | 2.3 Hz, 1H), 2.54 (s, 3H) |
| AZ | 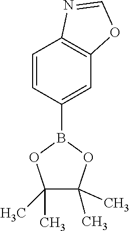 [SMILES: CC1(C)OB(c2ccc3ncoc3c2)OC1(C)C] | 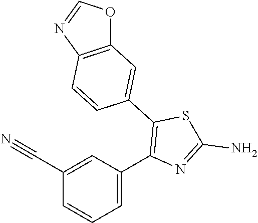 [SMILES: N#Cc1cccc(-c2nc(N)sc2-c2ccc3ncoc3c2)c1] |
| | 6-(4,4,5,5-Tetramethyl-1,3,2- | 3-[2-Amino-5-(1,3-benzoxazol-6-yl)thiazol-4- |
| | dioxaborolan-2-yl)-1,3-benzoxazole | yl]benzonitrile |
| | LC-MS-2 (Method 2B): Rt 1.56 mins; | LC-MS-2 (Method 2B): Rt 1.16 mins; MS |
| | MS m/z 246.0 = [M + H]+ | m/z 319.0 = [M + H]+ |
| | 1H NMR (500 MHz, DMSO-d6) δ | 1H NMR (500 MHz, DMSO-d6) δ 8.76 (s, |
| | 8.82 (s, 1H), 7.96 (s, 1H), 7.81 (d, | 1H), 7.77 (t, J = 1.7 Hz, 1H), 7.75 (d, J = 8.2 |
| | J = 7.9 Hz, 1H), 7.70 (dd, J = 7.9, 1.0 | Hz, 1H), 7.70 (dt, J = 7.7, 1.4 Hz, 1H), 7.67 |
| | Hz, 1H), 1.32 (s, 12H). | (d, J = 1.6 Hz, 1H), 7.60 (dt, J = 8.0, 1.4 Hz, |
| | Prepared from 6-bromo-1,3- | 1H), 7.44 (t, J = 7.8 Hz, 1H), 7.29 (s, 1H), |
| | benzoxazole and | 7.29 (s, 1H), 7.25 (dd, J = 8.3, 1.7 Hz, 1H). |
| | bis(pinacolato)diboron | |

### Intermediate B

### (3S)-3-Methylpyrrolidine-3-carbonitrile hydroiodide

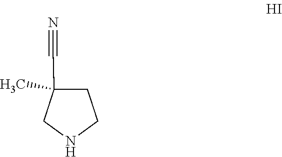

### Step 1: Benzyl (3S)-3-carbamoyl-3-methyl-pyrrolidine-1-carboxylate

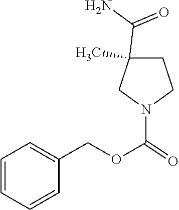 [SMILES: C[C@]1(C(N)=O)CCN(C(=O)OCc2ccccc2)C1]

A mixture of (3S)-1-Benzyloxycarbonyl-3-methyl-pyrrolidine-3-carboxylic acid (5.0 g, 18.99 mmol) and CDI (3.39 g, 20.89 mmol) in anhydrous THF (100 mL) under nitrogen was stirred at room temperature for 2 h. Ammonium hydroxide (35% in water) (43.21 mL, 379.81 mmol) was added and stirring continued for 30 mins. The THF was removed in vacuo and the aqueous residues were extracted with EtOAc (100 mL). The organic fraction was washed with aqueous HCl (100 mL), sat. aq. NaHCO₃ solution (100 mL) and brine (100 mL) before being dried over MgSO₄ and concentrated in vacuo to afford the title compound as a white solid.

LC-MS-2 (Method 2B): Rt 1.17 mins; MS m/z 263.0=[M+H]+

1H NMR (500 MHz, DMSO) δ 7.44-7.26 (m, 6H), 6.97 (s, 1H), 5.06 (s, 2H), 3.69 (dd, J=25.8, 10.6 Hz, 1H), 3.46-3.34 (m, 1H), 3.31-3.28 (m, 1H), 3.11 (dd, J=13.3, 10.6 Hz, 1H), 2.31-2.12 (m, 1H), 1.78-1.61 (m, 1H), 1.23 (s, 3H).

### Step 2: Benzyl (3S)-3-cyano-3-methyl-pyrrolidine-1-carboxylate

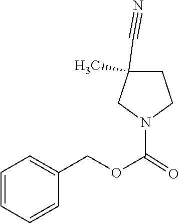 [SMILES: C[C@]1(C#N)CCN(C(=O)OCc2ccccc2)C1]

To a solution of benzyl (3S)-3-carbamoyl-3-methyl-pyrrolidine-1-carboxylate (step 1) (1.0 g, 3.81 mmol) in DCE (15 mL) was added thionyl chloride (1.38 mL, 19.06 mmol) and the mixture heated at 60° C. for 4 h. The resulting mixture was allowed to cool to room temperature and the solvent removed in vacuo. The crude product was dissolved in EtOAc (20 mL) and washed with sat. aq. NaHCO₃ solution (20 mL). The organic layer was separated and the aqueous further extracted with EtOAc (2×20 mL). The combined organics were dried over Na₂SO₄ and concentrated in vacuo to afford the title compound as a yellow oil.

LC-MS-1 (Method 2B): Rt 1.30 mins; MS m/z 267.2=[M+Na]+1H NMR (500 MHz, DMSO-d6) δ 7.40-7.35 (m, 4H), 7.35-7.29 (m, 1H), 5.09 (s, 2H), 3.78 (dd, J=22.8, 10.9 Hz, 1H), 3.58-3.39 (m, 2H), 3.31-3.24 (m, 1H), 2.35-2.26 (m, 1H), 2.06-1.94 (m, 1H), 1.43 (d, J=3.1 Hz, 3H).

### Step 3: (3S)-3-Methylpyrrolidine-3-carbonitrile hydroiodide

Trimethylsilyl iodide (0.42 mL, 2.95 mmol) was added dropwise via syringe to a stirred solution of benzyl (3S)-3-cyano-3-methyl-pyrrolidine-1-carboxylate (step 2) (300 mg, 1.23 mmol) in DCM (12 mL) under nitrogen at 0° C. The resulting solution was warmed to room temperature and stirred for 1 h. MeOH (0.5 mL, 12.28 mmol) was added dropwise, causing a pale solid to precipitate and the resulting mixture was stirred at room temperature for 30 mins followed by stirring at 0° C. for 15 mins. The suspension was filtered, the solids washed with cold DCM (2×5 mL) and vacuum dried to afford the title compound as a white solid. 1H NMR (500 MHz, DMSO-d6) δ 9.10 (s, 2H), 3.66 (d, J=12.2 Hz, 1H), 3.40 (ddd, J=12.0, 8.0, 5.4 Hz, 1H), 3.35-3.28 (m, 1H), 3.18 (d, J=12.3 Hz, 1H), 2.44 (ddd, J=13.1, 7.6, 5.4 Hz, 1H), 2.06 (dt, J=13.3, 8.1 Hz, 1H), 1.50 (s, 3H).

### Intermediate BA

### (3R)-3-Methylpyrrolidine-3-carbonitrile hydroiodide

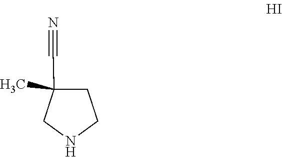

### Step 1: Benzyl (3R)-3-carbamoyl-3-methyl-pyrrolidine-1-carboxylate

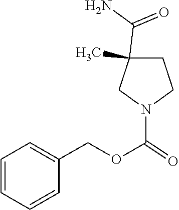 [SMILES: C[C@@]1(C(N)=O)CCN(C(=O)OCc2ccccc2)C1]

The title compound was prepared from (3R)-1-benzyloxycarbonyl-3-methyl-pyrrolidine-3-carboxylic acid analogously to Intermediate B, step 1.

LC-MS-2 (Method 2B): Rt 1.17 mins; MS m/z 263.0=[M+H]+

1H NMR (500 MHz, DMSO) δ 7.40-7.28 (m, 6H), 6.97 (s, 1H), 5.05 (s, 2H), 3.68 (dd, J=25.8, 10.6 Hz, 1H), 3.42-3.33 (m, 1H), 3.31-3.25 (m, 1H), 3.11 (dd, J=13.2, 10.6 Hz, 1H), 2.24-2.15 (m, 1H), 1.75-1.66 (m, 1H), 1.22 (s, 3H).

### Step 2: Benzyl (3R)-3-cyano-3-methyl-pyrrolidine-1-carboxylate

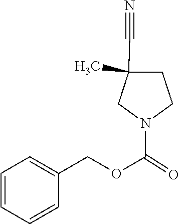 [SMILES: C[C@@]1(C#N)CCN(C(=O)OCc2ccccc2)C1]

Benzyl (3R)-3-carbamoyl-3-methyl-pyrrolidine-1-carboxylate (step 1) (996 mg, 3.8 mmol) and Burgess reagent (1.36 g, 5.7 mmol) were dissolved in DCM (9.5 mL) in a sealed tube under nitrogen and stirred at 35° C. for 17 h. The resulting mixture was allowed to cool to room temperature, diluted with DCM (40 mL) and washed with 1M HCl (50 mL) then brine (25 mL). The organic portion was dried over Na₂SO₄ and concentrated in vacuo. Purification by chromatography on silica eluting with a gradient of 10 to 50% EtOAc in petroleum ether afforded the title compound as a clear, colourless syrup.

LC-MS-2 (Method 2B): Rt 1.39 mins; MS m/z 245.0=[M+H]+

1H NMR (500 MHz, DMSO) δ 7.40-7.28 (m, 5H), 5.09 (s, 2H), 3.78 (dd, J=22.9, 10.9 Hz, 1H), 3.57-3.39 (m, 2H), 3.30-3.25 (m, 1H), 2.35-2.27 (m, 1H), 2.05-1.94 (m, 1H), 1.43 (d, J=3.1 Hz, 3H).

### Step 3: (3R)-3-Methylpyrrolidine-3-carbonitrile hydroiodide

The title compound was prepared from benzyl (3R)-3-cyano-3-methyl-pyrrolidine-1-carboxylate (step 2) and trimethylsilyl iodide analogously to Intermediate B step 3.

1H NMR (500 MHz, DMSO) δ 9.10 (br s, 2H), 3.66 (d, J=12.2 Hz, 1H), 3.40 (ddd, J=11.9, 8.0, 5.4 Hz, 1H), 3.35-3.29 (m, 1H), 3.19 (d, J=12.2 Hz, 1H), 2.43 (ddd, J=13.2, 7.6, 5.4 Hz, 1H), 2.06 (dt, J=13.2, 8.1 Hz, 1H), 1.50 (s, 3H).

### Intermediate C

### 2-Methyl-2-piperazin-1-yl-propanenitrile hydrochloride

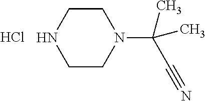

To a solution of tert-butyl 4-(1-cyano-1-methyl-ethyl)piperazine-1-carboxylate (250 mg, 0.99 mmol) in DCM (7.5 mL) was added 4M HCl in 1,4-dioxane (4.93 mL, 19.74 mmol) and the mixture stirred for 16 h. The solvent was removed in vacuo to afford a white solid which was suspended in Et₂O and collected by filtration. The solid was dried under high vacuum to afford the title compound as a white solid. 1H NMR (500 MHz, DMSO-d6) δ 9.16 (s, 2H), 3.10 (apr p, J=4.8 Hz, 4H), 2.78 (apr t, J=5.0 Hz, 4H), 1.46 (s, 6H).

Intermediate D 2-[(2S)-Piperazin-2-yl]propan-2-ol dihydrochloride

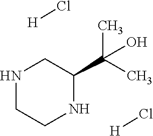

### Step 1: 1,4-Di-tert-butyl 2-methyl (2S)-piperazine-1,2,4-tricarboxylate

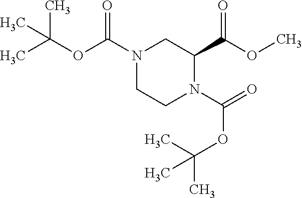 [SMILES: COC(=O)[C@@H]1CN(C(=O)OC(C)(C)C)CCN1C(=O)OC(C)(C)C]

A mixture of di-tert-butyl dicarbonate (479 mg, 2.19 mmol), 1-tert-butyl 2-methyl (2S)-piperazine-1,2-dicarboxylate (487 mg, 1.99 mmol) and triethylamine (0.39 mL, 2.79 mmol) in DCM (15 mL) was stirred at room temperature for 18 h. The reaction mixture was diluted with DCM (40 mL) and washed with sat. aq. NaHCO₃ solution (20 mL). The organic phase was dried over MgSO₄ and concentrated in vacuo to afford the title compound as a clear oil which solidified on standing. 1H NMR (500 MHz, DMSO-d6) δ 4.56 (d, J=37.3 Hz, 1H), 4.28 (dd, J=31.7, 13.8 Hz, 1H), 3.83 (s, 1H), 3.68 (s, 3H), 3.23-2.72 (m, 4H), 1.44-1.31 (m, 18H).

### Step 2: Di-tert-butyl (2S)-2-(1-hydroxy-1-methyl-ethyl)piperazine-1,4-dicarboxylate

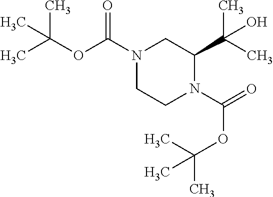 [SMILES: CC(C)(C)OC(=O)N1CCN(C(=O)OC(C)(C)C)[C@H](C(C)(C)O)C1]

To a solution of 1,4-di-tert-butyl 2-methyl (2S)-piperazine-1,2,4-tricarboxylate (step 1)(712 mg, 2.07 mmol) in THF (20 mL) at 0° C. was added MeMgCl (3M in THF) (1.72 mL, 5.17 mmol) dropwise. The reaction was allowed to warm to room temperature and was stirred at this temperature for 3 h. The reaction was diluted with DCM (70 mL) and washed with sat. NH₄Cl solution (20 mL). The aq. layer was extracted with DCM (20 mL) and the combined organic extracts were dried over MgSO₄ and concentrated in vacuo. Purification by chromatography on silica eluting with a gradient of 1 to 2% MeOH in DCM afforded the title compound as a clear oil.

1H NMR (500 MHz, DMSO-d6) δ 4.39 (s, 1H), 4.03-3.43 (m, 4H), 3.17-3.04 (m, 2H), 3.01-2.82 (m, 1H), 1.39 (s, 18H), 1.17-1.01 (m, 6H)

### Step 3: 2-[(2S)-Piperazin-2-yl]propan-2-ol dihydrochloride

To a solution of di-tert-butyl (2S)-2-(1-hydroxy-1-methyl-ethyl)piperazine-1,4-dicarboxylate (step 2)(331 mg, 0.96 mmol) in MeOH (8 mL) was added 4N HCl in 1,4-dioxane (2.4 mL, 9.60 mmol) and the reaction mixture was stirred at room temperature for 6 h before being concentrated in vacuo to afford the title compound as a white solid. 1H NMR (500 MHz, DMSO-d6) δ 9.67 (s, 2H), 9.41 (s, 1H), 9.07 (s, 1H), 5.57 (s, 1H), 3.62-3.53 (m, 1H), 3.49-3.38 (m, 3H), 3.29-3.21 (m, 2H), 2.98 (t, J=12.8 Hz, 1H), 1.26 (s, 3H), 1.19 (s, 3H).

Intermediate E 6-Bromo-4-ethyl-quinazoline

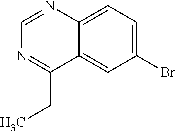

### Step 1: 1-(2-Amino-5-bromo-phenyl)propan-1-one

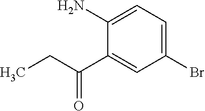

The following was prepared according to the procedure of WO2018/130184, pages 74-75,

To a stirred flask of 2-amino-5-bromo-benzonitrile (5.0 g, 25.38 mmol) in THF (100 mL) at 0° C. was added ethylmagnesium bromide (3M in Et₂O) (25.38 mL, 76.13 mmol) and the solution was stirred at 0° C. for 15 mins. The mixture was allowed to warm to room temperature and was stirred overnight. The resulting solution was re-cooled to 0° C. and treated with sat. aq. NH₄Cl solution (100 mL). EtOAc (300 mL) was added and the layers separated. The aqueous layer was extracted with EtOAc (300 mL) and the combined organic extracts were washed with brine (300 mL), dried over MgSO₄ and concentrated in vacuo. Purification by chromatography on silica eluting with a gradient of 0 to 40% EtOAc in petroleum ether afforded the title compound as a yellow solid.

LC-MS-2 (Method 2B): Rt 1.54 mins; MS m/z 227.9/229.9=[M+H]+

1H NMR (500 MHz, DMSO) δ 7.85 (d, J=2.4 Hz, 1H), 7.35 (dd, J=8.9, 2.4 Hz, 1H), 7.30 (s, 2H), 6.74 (d, J=8.9 Hz, 1H), 2.96 (q, J=7.2 Hz, 2H), 1.04 (t, J=7.2 Hz, 3H).

### Step 2: 6-Bromo-4-ethyl-quinazoline

The following was prepared according to the procedure of WO2018/130184, pages 74-75,

A solution of 1-(2-amino-5-bromo-phenyl)propan-1-one (step 1) (1.3 g, 5.7 mmol) and ammonium acetate (1.32 g, 17.1 mmol) in triethyl orthoformate (2.84 mL, 17.1 mmol) was stirred at 110° C. for 1 h. The resulting mixture was allowed to cool to room temperature and partitioned between EtOAc (50 mL) and saturated aq. NaHCO₃ solution (50 mL). The layers were separated and the aqueous layer back extracted with EtOAc (2×50 mL). The combined organic extracts were dried over MgSO₄ and concentrated in vacuo.

Purification by chromatography on silica eluting with a gradient of 20 to 40% EtOAc in petroleum ether afforded the title compound as a yellow solid.

LC-MS-1 (Method 2B): Rt 1.47 mins; MS m/z 237.1/239.1=[M+H]+

1H NMR (500 MHz, DMSO) δ 9.21 (s, 1H), 8.54 (d, J=2.2 Hz, 1H), 8.11 (dd, J=8.9, 2.2 Hz, 1H), 7.94 (d, J=8.9 Hz, 1H), 3.42-3.24 (m, 2H), 1.34 (t, J=7.4 Hz, 3H).

### Intermediate F

### 5-Bromo-3-methyl-2-(triazol-2-yl)pyridine

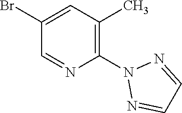

To a solution of 5-bromo-2-chloro-3-methyl-pyridine (2.0 g, 9.69 mmol) in DMF (15 mL) was added K₂CO₃ (2.68 g, 19.37 mmol) and 2H-triazole (0.84 mL, 14.53 mmol) and the mixture was heated to 90° C. for 17 h and then at 100° C. for an additional 2 h. Additional 2H-triazole (0.84 mL, 14.53 mmol) and K₂CO₃ (2.68 g, 19.37 mmol) were added and stirring continued at 100° C. for 18 h. The resulting mixture was diluted with 90% brine (100 mL) and EtOAc (100 mL) and the layers separated. The organics were washed with 50% brine (3×100 mL), dried over Na₂SO₄ and concentrated in vacuo. Purification by chromatography on silica eluting with a gradient of 0 to 20% EtOAc in petroleum ether afforded the title compound as a clear oil.

LC-MS-1 (Method 5A): Rt 2.56 mins; MS m/z 240.9=[M+H]+

1H NMR (500 MHz, Chloroform-d) δ 8.54-8.49 (m, 1H), 7.95-7.87 (m, 3H), 2.51-2.45 (m, 3H).

### Intermediate G

### 6-bromo-2,4-dimethyl-quinazoline

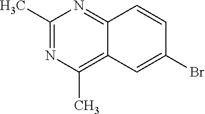

The following was prepared according to the procedure of Raju et al, ChemistrySelect, 2018, 3, 32, 9388-9392.

A mixture of 1-(2-amino-5-bromo-phenyl)ethanone (2.5 g, 11.68 mmol), acetylacetone (1.44 mL, 14.01 mmol) and ammonium acetate (2.7 g, 35.04 mmol) were heated at 100° C. for 3.5 h. The mixture was cooled to room temperature and was partitioned between EtOAc (100 mL) and H2O (100 mL). The organic fraction was separated and the aqueous further extracted with EtOAc (2×50 mL) before the combined organics were washed with brine (2×50 mL), dried over Na₂SO₄ and the solvent removed in vacuo. Purification by chromatography on silica eluting with 70% EtOAc in DCM afforded a solid which was suspended in Et₂O, collected by filtration and dried to afford the title compound as a cream solid.

LC-MS-1 (Method 2B): Rt 1.22 mins; MS m/z 237.1/239.1=[M+H]+

1H NMR (500 MHz, Chloroform-d) δ 8.20 (d, J=2.1 Hz, 1H), 7.90 (dd, J=9.0, 2.1 Hz, 1H), 7.80 (d, J=9.0 Hz, 1H), 2.89 (s, 3H), 2.83 (s, 3H).

### Intermediate H

### 6-Bromo-3,8-dimethyl-imidazo[1,2-a]pyridine

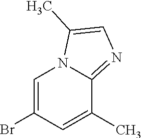

### Step 1: tert-Butyl N-(5-bromo-3-methyl-2-pyridyl)-N-tert-butoxycarbonyl-carbamate

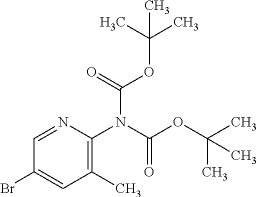 [SMILES: Cc1cc(Br)cnc1N(C(=O)OC(C)(C)C)C(=O)OC(C)(C)C]

To a solution of 5-bromo-6-methyl-pyridin-2-amine (2.0 g, 10.69 mmol) in DCM (50 mL) was added di-tert-butyl dicarbonate (2.8 g, 12.83 mmol), triethylamine (2.24 mL, 16.04 mmol) and DMAP (65 mg, 0.53 mmol) and the mixture stirred at room temperature for 4 h. Additional di-tert-butyl dicarbonate (2.8 g, 12.83 mmol), triethylamine (2.24 mL, 16.04 mmol) and DMAP (65.32 mg, 0.53 mmol) were added and stirring continued at room temperature for a further 16 h. The resulting mixture was washed with water (50 mL) and the aqueous fraction further extracted with DCM (2×30 mL). The combined organics were dried over MgSO₄ and the solvent removed in vacuo to give a viscous brown oil which solidified to afford the title compound a brown solid.

LC-MS-1 (Method 2B): Rt 1.56 mins; MS m/z 387.2/389.2=[M+H]+

1H NMR (500 MHz, Chloroform-d) δ 8.35-8.31 (m, 1H), 7.67-7.64 (m, 1H), 2.15 (t, J=0.8 Hz, 3H), 1.34 (s, 18H).

### Step 2: tert-Butyl N-(5-bromo-3-methyl-2-pyridyl)carbamate

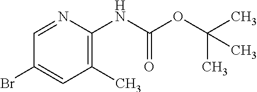 [SMILES: Cc1cc(Br)cnc1NC(=O)OC(C)(C)C]

To a solution of crude tert-butyl N-(5-bromo-3-methyl-2-pyridyl)-N-tert-butoxycarbonyl-carbamate (step 1) (1.0 g, 2.58 mmol) in DCM (10 mL) was added dropwise TFA (0.4 mL, 5.16 mmol) and the mixture was stirred at room temperature for 65 h. Additional TFA (0.2 mL, 2.58 mmol) was added and stirring continued for 20 h. 3 further portions of TFA (0.2 mL, 2.58 mmol) were added at 20h intervals and the mixture was stirred for a further 48 h after the final addition. The resulting mixture was washed with sat. aq. NaHCO₃ solution (3×20 mL), dried over MgSO₄ and the solvent removed in vacuo. Purification by chromatography on silica eluting with 0.5% MeOH in DCM afforded the title compound as a white solid.

LC-MS-2 (Method 2B): Rt 1.51 mins; MS m/z 286.9/288.9=[M+H]+

1H NMR (500 MHz, CDCl3) δ 8.30 (d, J=2.3 Hz, 1H), 7.66-7.62 (m, 1H), 6.67 (s, 1H), 2.27 (s, 3H), 1.51 (s, 9H).

### Step 3: tert-Butyl N-(5-bromo-3-methyl-2-pyridyl)-N-prop-2-ynyl-carbamate

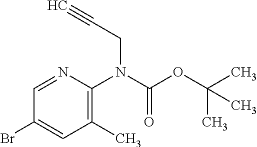 [SMILES: C#CCN(C(=O)OC(C)(C)C)c1ncc(Br)cc1C]

A cooled (0° C.) solution of tert-butyl N-(5-bromo-3-methyl-2-pyridyl)carbamate (step 2) (257 mg, 0.90 mmol) in anhydrous DMF (3.6 mL) was treated with NaH (60% dispersion in mineral oil) (43 mg, 1.07 mmol) in one portion and the mixture stirred at 0° C. for 15 mins. Propargyl bromide (80 wt % in toluene) (120 μL, 1.07 mmol) was added dropwise and stirring continued at 0° C. for 10 mins and then at room temperature for a further 2 h 20 mins.

The reaction was quenched by careful addition of water (1 mL). The resulting mixture was diluted with EtOAc (40 mL) and the organic fraction washed with 90% brine (20 mL) and 50% brine (3×20 mL) before being dried over Na₂SO₄ and concentrated in vacuo to afford the title compound as a yellow syrup.

LC-MS-2 (Method 2B): Rt 1.72 mins; MS m/z 324.9/326.9=[M+H]+

1H NMR (500 MHz, CDCl3) δ 8.37 (d, J=2.4 Hz, 1H), 7.71 (dd, J=2.4, 0.8 Hz, 1H), 4.82-4.25 (m, 2H), 2.29 (s, 3H), 2.12 (br t, J=2.5 Hz, 1H), 1.43 (s, 9H).

### Step 4: 5-Bromo-3-methyl-N-prop-2-ynyl-pyridin-2-amine

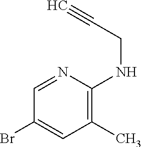

TFA (2.0 mL, 26.12 mmol) was added to a solution of tert-butyl N-(5-bromo-3-methyl-2-pyridyl)-N-prop-2-ynyl-carbamate (step 3) (260 mg, 0.80 mmol) in DCM (4 mL) and the mixture stirred at room temperature for 40 mins. The resulting mixture was diluted with DCM (25 mL) and saturated aq. NaHCO₃ solution (50 mL) added, with the mixture stirred until effervescence had ceased. The layers were separated and the aqueous portion extracted further with DCM (25 mL). The combined organic extracts were dried over Na₂SO₄ and concentrated in vacuo to afford the title compound as a cloudy pale brown oil.

LC-MS-2 (Method 2B): Rt 1.46 mins; MS m/z 224.9/226.9=[M+H]+

1H NMR (500 MHz, CDCl3) δ 8.08 (d, J=2.4 Hz, 1H), 7.36 (dd, J=2.4, 1.0 Hz, 1H), 4.29 (br s, 1H), 4.24 (dd, J=5.0, 2.5 Hz, 2H), 2.23 (t, J=2.5 Hz, 1H), 2.10 (s, 3H).

### Step 5: 6-Bromo-3,8-dimethyl-imidazo[1,2-a]pyridine

The following was prepared according to the procedure of Adimurthy et al, J.

Org. Chem. 2013, 78, 1266-1272.

Water (4 mL) was added to 5-bromo-3-methyl-N-prop-2-ynyl-pyridin-2-amine (step 4)(177 mg, 0.79 mmol) in a sealed tube under nitrogen and the mixture stirred at 120° C. for 1 h. The resulting mixture was allowed to cool to room temperature before being partitioned between DCM (20 mL) and sat. aq. NaHCO₃ solution (20 mL). The organic fraction was separated and the aqueous further extracted with DCM (2×20 mL). The combined organic portions were dried over Na₂SO₄ and concentrated in vacuo. Purification by chromatography on silica eluting with EtOAc afforded a straw coloured syrup which slowly crystallised to afford the title compound an off-white waxy solid.

LC-MS-2 (Method 2B): Rt 1.32 mins; MS m/z 224.9/226.9=[M+H]+

1H NMR (500 MHz, MeOD) δ 8.28 (s, 1H), 7.34 (s, 1H), 7.20 (s, 1H), 2.53 (s, 3H), 2.48 (s, 3H).

### Intermediate I

### 6-Bromo-4,8-dimethyl-quinazoline

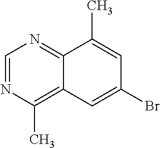

### Step 1: 1-(2-Amino-5-bromo-3-methyl-phenyl)ethanone

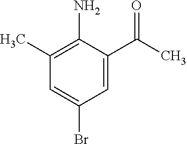

The following was prepared according to the procedure of WO2020/011220, page 41,

Methyl magnesium bromide (3.4M in 2-Me-THF) (13.5 mL, 0.05 mmol) was added dropwise to a solution of 2-amino-5-bromo-3-methyl-benzonitrile (1.94 g, 0.01 mmol) in THF (92 mL) at −10° C. and the mixture was stirred overnight while warming to room temperature. 10% HCl (92 mL, 0.01 mmol) was added dropwise and the resulting mixture was stirred for 1 h before being extracted with EtOAc (3×100 mL). The combined organic extracts were washed with brine (90 mL), dried over Na₂SO₄ and concentrated in vacuo. Purification by chromatography on silica eluting with a gradient of 0 to 50% EtOAc in petroleum ether followed by trituration of the product with MeOH afforded the title compound as a yellow solid.

LC-MS-2 (Method 2B): Rt 1.53 mins; MS m/z 226.1/228.1=[M−H]-1H NMR (500 MHz, DMSO-d6) δ 7.76 (s, 1H), 7.35 (s, 1H), 7.18 (s, 2H), 2.53 (s, 3H), 2.10 (s, 3H).

### Step 2: 6-Bromo-4,8-dimethyl-quinazoline

The following was prepared according to the procedure of WO2020/011220, page 42,

A mixture of 1-(2-amino-5-bromo-3-methyl-phenyl)ethanone (step 1)(1.25 g, 5.47 mmol) and ammonium acetate (2.53 g, 32.8 mmol) in triethyl orthoformate (0.91 mL, 5.47 mmol) was heated to 130° C. overnight. The resulting mixture was cooled to room temperature before being concentrated in vacuo. The residue was dissolved in EtOAc (150 mL) and washed with brine (2×80 mL), dried over MgSO₄ and concentrated in vacuo. The crude mixture was purified by chromatography on silica eluting with a gradient of 0 to 50% EtOAc in petroleum ether followed by a second chromatographic purification on silica eluting with 0 to 40% EtOAc in DCM to afford the title compound as a brown solid.

LC-MS-2 (Method 2B): Rt 1.46 mins; MS m/z 236.9/238.9=[M+H]+

1H NMR (500 MHz, DMSO) δ 9.17 (s, 1H), 8.33 (d, J=2.2 Hz, 1H), 8.02 (s, 1H), 2.90 (s, 3H), 2.68 (s, 3H).

### Intermediate J

### 4-Bromo-1-methyl-pyrrolo[2,3-b]pyridine

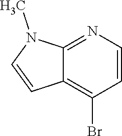

NaH (60% dispersion in mineral oil)(122 mg, 3.05 mmol) was added to a cooled suspension of 4-bromo-1H-pyrrolo[2,3-b]pyridine (550 mg, 2.79 mmol) in DMF (3 mL) in an ice bath. After 30 mins, iodomethane (0.17 mL, 2.79 mmol) was added and stirring continued for a further 30 mins. The reaction was quenched with water (30 mL) and taken up in EtOAc (30 mL). The layers were separated and the aqueous was extracted with EtOAc (30 mL). The combined organic extracts was washed with 90% brine (30 mL), 50% brine (2×30 mL), dried over MgSO₄ and concentrated in vacuo. Purification by chromatography on silica eluting with a gradient of 30 to 50% EtOAc in hexanes afforded the title compound as an off white solid.

LC-MS-2 (Method 2B): Rt 1.44 mins; MS m/z 210.9/212.9=[M+H]+

1H NMR (500 MHz, DMSO-d6) δ 8.13 (d, J=5.2 Hz, 1H), 7.66 (d, J=3.5 Hz, 1H), 7.36 (d, J=5.1 Hz, 1H), 6.43 (d, J=3.5 Hz, 1H), 3.83 (s, 3H).

### Intermediate K

### 6-Bromo-4-methyl-1-trityl-indazole

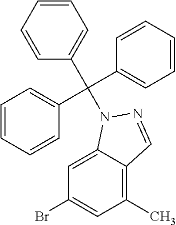 [SMILES: Cc1cc(Br)cc2c1cnn2C(c1ccccc1)(c1ccccc1)c1ccccc1]

6-Bromo-4-methyl-1H-indazole (200 mg, 0.95 mmol) in DMF (4.74 mL) was added dropwise to a stirred suspension of NaH (60% dispersion in mineral oil) (46 mg, 1.14 mmol) in DMF (4.74 mL) at 0° C. and stirred for 1 h. Triphenylmethyl chloride (291 mg, 1.04 mmol) was added and the solution was allowed to warm to room temperature and stirred for 1 h. The reaction was quenched by careful addition of water (10 mL). The resulting mixture was diluted with EtOAc (50 mL) and the layers separated. The aqueous layer was extracted with EtOAc (2×50 mL) and the combined organic extracts were washed with 90% brine (50 mL), 50% brine (2×50 mL), dried over MgSO₄ and concentrated in vacuo. Purification by chromatography on silica eluting with 20% EtOAc in hexane afforded the title compound (and the corresponding regioisomer).

Isomer 1: LC-MS-1 (Method 2B): Rt 1.93 mins; MS m/z 451.1/453.1=[M−H]-1H NMR (500 MHz, DMSO-d6) δ 8.08 (d, J=1.0 Hz, 1H), 7.71 (s, 1H), 7.40-7.36 (m, 9H), 7.11-7.08 (m, 6H), 6.96 (t, J=1.4 Hz, 1H), 2.40 (s, 3H).

Isomer 2: LC-MS-1 (Method 2B): Rt 1.90 mins; MS m/z 209.1/211.1=[M−H-trityl]-1H NMR (500 MHz, DMSO-d6) δ 8.26 (s, 1H), 7.37-7.28 (m, 9H), 7.22-7.19 (m, 1H), 7.14-7.10 (m, 6H), 7.04-7.02 (m, 1H), 2.54 (s, 3H).

### Intermediate L

### 6-Bromo-4-methyl-1-trityl-benzotriazole

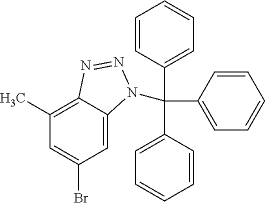 [SMILES: Cc1cc(Br)cc2c1nnn2C(c1ccccc1)(c1ccccc1)c1ccccc1]

The title compound was prepared from 5-bromo-7-methyl-1H-benzotriazole and triphenylmethyl chloride analogously to Intermediate K.

LC-MS-2 (Method 2B): Rt 2.09 mins; MS m/z 212.0=[M−H-trityl]+

1H NMR (500 MHz, DMSO) δ 7.43-7.36 (m, 9H), 7.33 (t, J=1.4 Hz, 1H), 7.23-7.17 (m, 1H), 7.14-7.08 (m, 6H), 2.70 (s, 3H).

### Intermediate M

### 4-Bromo-1,6-dimethyl-pyrrolo[2,3-b]pyridine

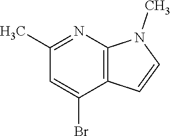

The title compound was prepared from 4-bromo-6-methyl-1H-pyyrolo[2,3-b]pyridine and iodomethane analogously to Intermediate J.

LC-MS-2 (Method 2B): Rt 1.57 mins; MS m/z 224.9/226.9=[M+H]+

1H NMR (500 MHz, DMSO) δ 7.53 (d, J=3.5 Hz, 1H), 7.26 (s, 1H), 6.36 (d, J=3.5 Hz, 1H), 3.79 (s, 3H), 2.55 (s, 3H).

### Intermediate N

### tert-Butyl 5-bromo-7-methyl-indazole-1-carboxylate

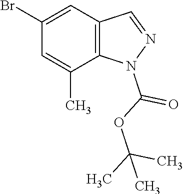 [SMILES: Cc1cc(Br)cc2cnn(C(=O)OC(C)(C)C)c12]

To a solution of di-tert-butyl dicarbonate (2.59 g, 11.84 mmol) and 5-bromo-7-methyl-1H-indazole (2.0 g, 9.48 mmol) in MeCN (20 mL) was added triethylamine (1.52 mL, 10.9 mmol) and DMAP (0.12 g, 0.95 mmol) and the mixture was stirred at room temperature overnight. The resulting mixture was concentrated in vacuo and the residue taken up in EtOAc: petroleum ether (7:3, 150 mL). The solution was washed with water (75 ml), the organic fraction separated and the aqueous extracted with EtOAc: Pet (1:1, 2×50 mL). The combined organic fractions were washed with water (100 mL), sat. aq. NH₄Cl solution (50 mL), water (2×50 mL), sat. aq. NaHCO₃ solution (2×75 mL), brine (50 mL) and were dried over MgSO₄ before being concentrated in vacuo. Purification by chromatography on silica eluting with a gradient of 1 to 30% EtOAc in petroleum ether afforded the title compound as an orange solid.

LC-MS-1 (Method 3B): Rt 2.27 mins; MS m/z 211.0/212.9=[M+H-Boc]+1H NMR (500 MHz, Chloroform-d) δ 8.49 (s, 1H), 7.63 (dd, J=1.7, 0.8 Hz, 1H), 7.15 (p, J=1.3 Hz, 1H), 2.62 (t, J=1.0 Hz, 3H), 1.71 (s, 9H).

### Intermediate O

### 2,4-Dimethyl-5-(4,4,5,5-tetramethyl-1,3,2-dioxaborolan-2-yl)oxazole

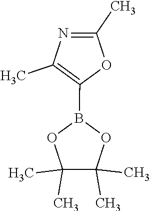

Bis(pinacolato)diboron (654 mg, 2.57 mmol), methoxy(cyclooctadiene)iridium(I) dimer (51 mg, 0.08 mmol) and 4,4′-di-tert-butyl-2,2′-dipyridyl (41 mg, 0.15 mmol) were placed in a vial sealed with a teflon cap and the contents evacuated and backfilled with nitrogen (3×cycles). Freshly de-oxygenated anhydrous cyclohexane (10 mL) was added and the resulting red-brown solution stirred vigorously at room temperature for 20 mins. 2,4-dimethyloxazole (0.26 mL, 2.57 mmol) was then added and the resulting mixture stirred at room temperature overnight. The mixture was concentrated in vacuo to afford the title compound as a dark red-brown oil.

LC-MS-2 (Method 2B): Rt 0.74 mins; MS m/z 224.1=[M+H]+

1H NMR (500 MHz, DMSO) δ 2.38 (s, 3H), 2.18 (s, 3H), 1.27 (s, 12H).

### Intermediate P

### 3-(2-Aminothiazol-4-yl)benzonitrile hydrobromide

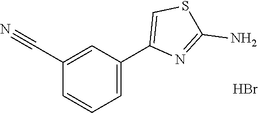 [SMILES: N#Cc1cccc(-c2csc(N)n2)c1 ; Br]

The following was prepared according to the procedure of WO2012/100734, page 24, Example 15 step 2.

To a flask containing 3-(2-bromoacetyl)benzonitrile (5 g, 22.32 mmol) in EtOH (50 mL) was added thiourea (1.87 g, 24.55 mmol) and the mixture was stirred at room temperature for 30 mins. The volume of solvent was reduced by ˜50% in vacuo resulting in the formation of a suspension. The suspension was sonicated, filtered and dried to afford the title compound as a white solid.

LC-MS-1 (Method 3B): Rt 1.62 mins; MS m/z 200.0=[M−H]1H NMR (500 MHz, DMSO-d6) δ 8.22 (t, J=1.8 Hz, 1H), 8.08 (d, J=8.1 Hz, 1H), 7.82 (d, J=7.7 Hz, 1H), 7.65 (t, J=7.9 Hz, 1H), 7.37 (s, 1H). NH2 not observed. HBr salt assigned on basis of mass of compound obtained.

3-(2-Aminothiazol-4-yl)benzonitrile may also be prepared as follows:

To a flask containing thiourea (2.04 g, 26.78 mmol) in pyridine (50 mL) under nitrogen was added 3-(2-bromoacetyl)benzonitrile (3.0 g, 13.39 mmol) and the mixture was stirred at room temperature for 1 h. The resulting mixture was diluted with water (100 mL) and extracted with EtOAc (2×100 mL). The combined organic extracts were washed with brine (50 mL), dried over MgSO4 and concentrated in vacuo. The crude product was purified by chromatography on silica eluting with 0-100% EtOAc in DCM. The resulting solid was recrystallised from EtOAc to afford 3-(2-aminothiazol-4-yl)benzonitrile as a pale yellow solid LC-MS (Method 7A): Rt 1.75 mins; MS m/z 202.1=[M+H]+, 243.3=[M+H+MeCN]+1H NMR (400 MHz, DMSO-d6) δ 8.20 (t, J=1.5 Hz, 1H), 8.11 (dt, J=7.9, 1.4 Hz, 1H), 7.70 (dt, J=7.7, 1.3 Hz, 1H), 7.58 (t, J=7.6 Hz, 1H), 7.26 (s, 1H), 7.14 (s, 2H).

### Intermediate 0

### 3-(2-Amino-5-bromo-thiazol-4-yl)benzonitrile

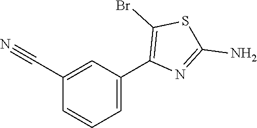 [SMILES: N#Cc1cccc(-c2nc(N)sc2Br)c1]

The following was prepared according to the procedure of WO2012/100734, page 24-25, Example 15 step 3.

To a solution of 3-(2-aminothiazol-4-yl)benzonitrile hydrobromide (Intermediate P)(2.6 g, 9.21 mmol) in THF (40 mL) was added NBS (1.97 g, 11.06 mmol) and the reaction mixture was stirred at room temperature for 1 h. The resulting mixture was diluted with EtOAc (60 mL) and washed with water (100 mL). The organic portion was separated and the aqueous further extracted with EtOAc (2×50 mL). The combined organics were dried over MgSO4 and concentrated in vacuo to yield an orange solid. The solid was suspended in DCM (20 mL), filtered, washed with DCM and dried to afford the title compound as an off-white solid.

LC-MS-1 (Method 3B): Rt 1.64 mins; MS m/z 279.9/281.9=[M+H]+

1H NMR (500 MHz, DMSO-d6) δ 8.16 (t, J=1.7 Hz, 1H), 8.15 (dt, J=7.9, 1.7 Hz, 1H), 7.83 (d, J=7.9 Hz, 1H), 7.66 (t, J=7.9 Hz, 1H), 7.42 (s, 2H).

### Intermediate R

### tert-Butyl 6-methyl-4-(4,4,5,5-tetramethyl-1,3,2-dioxaborolan-2-yl)pyrrolo[2,3-b]pyridine-1-carboxylate

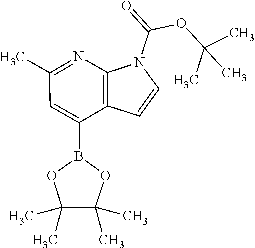 [SMILES: Cc1cc(B2OC(C)(C)C(C)(C)O2)c2ccn(C(=O)OC(C)(C)C)c2n1]

### Step 1: tert-Butyl 4-bromo-6-methyl-pyrrolo[2,3-b]pyridine-1-carboxylate

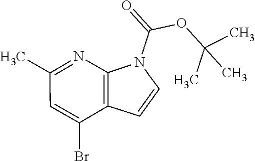 [SMILES: Cc1cc(Br)c2ccn(C(=O)OC(C)(C)C)c2n1]

The title compound was prepared from 4-bromo-6-methyl-1H-pyrrolo[2,3-b]pyridine and di-tert-butyl decarbonate analogously to Intermediate N.

LC-MS-2 (Method 2B): Rt 1.86 mins; MS m/z 310.9/312.9=[M+H]+

1H NMR (500 MHz, DMSO-d6) δ 7.79 (d, J=4.0 Hz, 1H), 7.49 (s, 1H), 6.59 (d, J=4.0 Hz, 1H), 2.55 (s, 3H), 1.61 (s, 9H).

### Step 2: tert-Butyl 6-methyl-4-(4,4,5,5-tetramethyl-1,3,2-dioxaborolan-2-yl)pyrrolo[2,3-b]pyridine-1-carboxylate

The title compound was prepared from tert-butyl 4-bromo-6-methyl-pyrrolo[2,3-b]pyridine-1-carboxylate (step 1) and bis(pinacolato)diboron analogously to Intermediate A step 1.

LC-MS-2 (Method 2B): Rt 1.72 mins; MS m/z 359.1=[M+H]+

1H NMR (500 MHz, DMSO-d6) δ 7.72 (d, J=4.0 Hz, 1H), 7.36 (s, 1H), 6.78 (d, J=4.0 Hz, 1H), 2.56 (s, 3H), 1.61 (s, 9H), 1.34 (s, 12H).

### Intermediate S

### 3-[2-Amino-5-(6-methyl-1-trityl-pyrazolo[3,4-b]pyridin-4-yl)thiazol-4-yl]benzonitrile

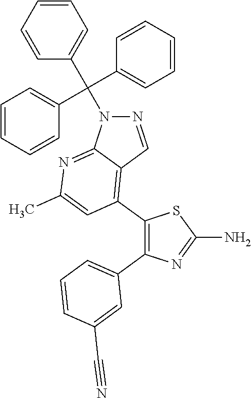 [SMILES: Cc1cc(-c2sc(N)nc2-c2cccc(C#N)c2)c2cnn(C(c3ccccc3)(c3ccccc3)c3ccccc3)c2n1]

### Step 1: 4-Bromo-6-methyl-1-trityl-pyrazolo[3,4-b]pyridine

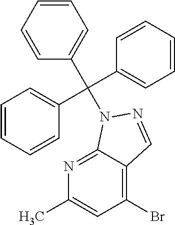 [SMILES: Cc1cc(Br)c2cnn(C(c3ccccc3)(c3ccccc3)c3ccccc3)c2n1]

4-Bromo-6-methyl-1H-pyrazolo[3,4-b]pyridine (500 mg, 2.36 mmol) in DMF (10 mL) was added dropwise to a stirred suspension of sodium hydride (60% in mineral oil) (113 mg, 2.83 mmol) in DMF (10 mL) at 0° C. The solution was stirred for 45 mins at 0° C. and triphenylmethyl chloride (723.08 mg, 2.59 mmol) was added. The solution was allowed to warm to room temperature and was stirred for 1 h. The solution was re-cooled to 0° C. and water (10 mL) was added slowly. The solution was diluted with EtOAc (50 mL) and the layers separated.

The aqueous layer was extracted with EtOAc (2×50 mL) and the combined organic extracts were washed with 90% brine (50 mL), 50% brine (2×50 mL), dried over Na₂SO₄ and concentrated in vacuo. Purification by chromatography on silica eluting with a gradient of 0-30% EtOAc in petrol afforded the title compound as an orange solid.

LC-MS-2 (Method 2B): Rt 2.14 mins; MS m/z 454.0/456.0=[M+H]+

1H NMR (500 MHz, DMSO) δ 8.16 (s, 1H), 7.33 (s, 1H), 7.34-7.18 (m, 9H), 7.19-7.12 (m, 6H), 2.19 (s, 3H).

### Step 2: 6-Methyl-4-(4,4,5,5-tetramethyl-1,3,2-dioxaborolan-2-yl)-1-trityl-pyrazolo[3,4-b]pyridine

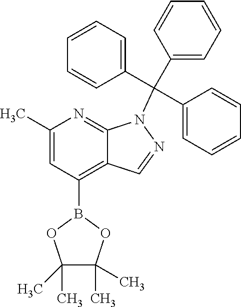 [SMILES: Cc1cc(B2OC(C)(C)C(C)(C)O2)c2cnn(C(c3ccccc3)(c3ccccc3)c3ccccc3)c2n1]

The title compound was prepared from 4-bromo-6-methyl-1-trityl-pyrazolo[3,4-b]pyridine (step 1) and bis(pinacolato)diboron analogously to Intermediate A step 1.

LC-MS-2 (Method 2B): Rt 1.57 mins; MS m/z 420.1=[M-C₆H₁₀]+

1H NMR (500 MHz, DMSO) δ 8.17 (s, 1H), 7.93 (s, 1H), 7.25-7.16 (m, 15H), 2.22 (s, 3H), 1.34 (s, 12H).

### Step 3: 3-[2-Amino-5-(6-methyl-1-trityl-pyrazolo[3,4-b]pyridin-4-yl)thiazol-4-yl]benzonitrile

The title compound was prepared from 3-(2-amino-5-bromo-thiazol-4-yl)benzonitrile (Intermediate Q) and crude 6-methyl-4-(4,4,5,5-tetramethyl-1,3,2-dioxaborolan-2-yl)-1-trityl-pyrazolo[3,4-b]pyridine (step 2) analogously to Intermediate A step 2.

LC-MS-2 (Method 2B): Rt 1.96 mins; MS m/z 575.1=[M+H]+

1H NMR (500 MHz, DMSO) δ 7.73 (dt, J=7.7, 1.4 Hz, 1H), 7.64 (dt, J=8.0, 1.5 Hz, 1H), 7.61 (t, J=1.7 Hz, 1H), 7.57 (s, 2H), 7.46 (t, J=7.8 Hz, 1H), 7.32 (s, 1H), 7.31-7.19 (m, 9H), 7.15-7.02 (m, 6H), 6.90 (s, 1H), 2.16 (s, 3H).

### BIOLOGICAL EXAMPLES

### Biological Example 1—Adenosine Receptor Time-Resolved Fluorescence Resonance Energy Transfer (TRFRET) Binding Assay

All FRET binding experiments were conducted at room temperature in white 384-well plates, in assay binding buffer containing 1× LabMed (Cisbio, France), 100 μg/mL saponin, 1% DMSO and 0.02% pluronic acid. Binding of the fluorescently labelled Adenosine receptor antagonist XAC (CA200645, FRET acceptor) to terbium-labelled A1, A2a, A2b and A3 adenosine receptors (FRET donors) was detected by time-resolved FRET due to the close proximity of the donor and acceptor in a binding event. To investigate the ability of unlabelled test compounds to bind to Adenosine A1, A2a, A2b and A3 receptors, dose response curves were constructed that determined the ability of a range of concentrations to inhibit the binding of 30 nM CA200645 to the A2b receptor and 100 nM CA200645 to the A1, A2a, and A3 receptor.

Serial dilution (1:3 dilutions) of test compounds in neat DMSO and transfer of a 400 nL sample of test compound into the assay plate was carried out using the Mosquito (TTP Labtech, UK). The compound samples were incubated for 2 hours at room temperature with a fixed concentration of CA200645 defined for each receptor (see above) and CHO cell membranes containing the human Adenosine A1 (0.5 μg/well), A2a (0.3 μg/well), A2b (1 μg/well) or A3 (1 μg/well) receptor in 40 μL of assay buffer. Total and non-specific binding of CA200645 was determined in the absence and presence of 10 μM XAC, respectively. Following 2 hours incubation, the level of CA200645 binding was detected on a Pherastar FSX (BMG Labtech, Germany) using standard TR-FRET settings. The terbium donor was excited with three laser flashes at a wavelength of 337 nm, and donor and acceptor emission was detected at 620 nm and 665 nm wavelengths, respectively. FRET ratios were obtained by multiplying the acceptor/donor ratio value by 10,000. Specific binding was determined by subtracting the non-specific binding FRET ratio from the total binding FRET ratio. Compound IC50 curves were analysed using GraphPad Prism 7.0 (GraphPad, USA) and Ki affinity values were determined from the obtained IC50 values using the method of Cheng and Prusoff. The results are presented in Table 1.

TABLE 1

| Example | A1 Ki | A2a Ki | A2b Ki | A3 Ki |
|---|---|---|---|---|
| Number | (nM) | (nM) | (nM) | (nM) |
| 1 | 2 | 0.12 | 0.9 | |
| 2 | 70 | 0.32 | 2.8 | |
| 2.1 | 12 | 0.25 | 2.5 | |
| 2.2 | 2 | 2.69 | 3.3 | 820 |
| 2.3 | 1 | 1.37 | 1.7 | 466 |
| 2.4 | 1 | 1.00 | 0.5 | 290 |
| 2.5 | 14 | 3.3 | 0.9 | 2600 |
| 3 | 554 | 2.26 | 7.7 | |
| 3.1 | 48 | 0.15 | 1.3 | |
| 4 | 27 | 0.19 | 1.9 | |
| 4.1 | 42 | 0.14 | 1.1 | |
| 4.2 | 34 | 0.35 | 3.0 | |
| 4.3 | 30 | 0.3 | 2.4 | |
| 5 | 21 | 0.17 | 1.7 | |
| 6 | 3 | 0.07 | 0.8 | |
| 7 | 11 | 0.10 | 1.4 | |
| 7.1 | 121 | 36.85 | 0.9 | 828 |
| 7.2 | 9 | 0.20 | 1.5 | |
| 7.3 | 19 | 2.42 | 10.5 | |
| 7.4 | 345 | 1.83 | 6.4 | |
| 7.5 | 9 | 0.21 | 1.2 | |
| 7.6 | 62 | 0.55 | 2.5 | |
| 7.7 | 9 | 0.26 | 3.6 | |
| 7.7a | 4 | 0.10 | 1.1 | |
| 7.7b | 9 | 0.20 | 1.5 | |
| 7.8 | 13 | 0.14 | 2.4 | |
| 7.9 | 21 | 0.10 | 1.9 | |
| 7.10 | 12 | 0.42 | 2.8 | |
| 7.11 | 98 | 0.54 | 5.4 | |
| 7.12 | 221 | 2.05 | 6.5 | |
| 7.13 | 125 | 1.01 | 4.8 | |
| 7.14 | 150 | 2.61 | 5.4 | |
| 7.15 | 14 | 0.18 | 1.1 | |
| 7.16 | 17 | 0.18 | 2.6 | |
| 7.17 | 13 | 0.20 | 1.9 | |
| 7.18 | 11 | 0.38 | 2.2 | |
| 7.19 | 13 | 0.08 | 0.9 | |
| 7.20 | 57 | 0.20 | 1.4 | |
| 7.21 | 9 | 0.30 | 1.6 | |
| 7.22 | 20 | 0.42 | 3.0 | |
| 7.23 | 10 | 0.13 | 1.1 | |
| 7.24 | 18 | 0.14 | 1.3 | |
| 7.25 | 3 | 0.12 | 1.1 | |
| 7.26 | 19 | 0.13 | 1.8 | |
| 7.27 | 27 | 0.48 | 3.1 | |
| 7.28 | 65 | 0.64 | 3.3 | |
| 7.29 | 3 | 0.09 | 0.5 | |
| 8 | 21 | 8.40 | 6.7 | 628 |
| 9 | 70 | 5.45 | 6.0 | |
| 10 | 7 | 0.85 | 4.7 | |
| 11 | 0.4 | 0.04 | 0.5 | |
| 12 | 0.2 | 0.17 | 0.8 | |
| 13 | 2 | 0.16 | 1.7 | |
| 14 | 12 | 0.42 | 2.8 | |
| 15 | 14 | 1.74 | 6.2 | |
| 16 | 122 | 5.17 | 9.8 | 2134 |
| 17 | 1 | 3.11 | 0.8 | 305 |
| 18 | 1 | 0.11 | 0.9 | 2632 |
| 19 | 240 | 2.00 | 1.8 | |
| 20 | 8 | 0.18 | 1.0 | |
| 21 | 4 | 8.33 | 2.6 | 421 |
| 22 | 0.4 | 0.15 | 1.4 | 2547 |
| 23 | 467 | 6.56 | 1.6 | 126 |
| 24 | 245 | 10.47 | 7.2 | |
| 25 | 0.3 | 0.07 | 0.5 | 1732 |
| 26 | 0.3 | 0.33 | 0.5 | 904 |
| 27 | 0.5 | 0.08 | 2.9 | 907 |
| 28 | 21 | 8.40 | 6.7 | 628 |
| 29 | 4 | 1.88 | 1.3 | |
| 30 | 14 | 0.59 | 13.2 | |
| 31 | 57 | 4.31 | 38.7 | 1176 |
| 32 | 272 | 8.87 | 136.1 | 103 |
| 33 | 0.2 | 0.36 | 0.5 | 758 |
| 34 | 290 | 7.4 | 7.6 | 260 |
| 35 | 1.6 | 1.4 | 1.6 | 130 |

### Biological Example—CD3/CD28 Stimulated IL-2 Release NECA Reversal Assay in Human PBMCs

Blood is drawn from healthy volunteers using sodium citrate as the anticoagulant (0.3% final concentration). After centrifugation of the blood over Histopaque-1077, PBMCs are collected from the Histopaque/plasma interface and washed twice in PBS (300 g for 10 mins at room temp). Cells are plated at 50,000 cells/well in 150 μl RPMI/10% FCS in 96-well cell culture plates that have been precoated with 1 μg/ml CD3 antibody. 50 μl diluted compound mix is added to the cells, to obtain final concentrations of 1 μg/ml CD28 antibody, 1 uM NECA and 0.003-10 μM adenosine receptor antagonist. Assay plates are incubated for 24 hours at 37° C. in a humidified incubator. Culture supernatant is tested for IL-2 levels using the human IL-2 Tissue Culture Kit (Meso Scale Discovery). Data for dose-response curves is calculated as % inhibition with 100% inhibition defined from no agonist control wells (+CD3/28-NECA)

TABLE 2

| Example | IL-2 IC50 (□M) |
|---|---|
| 2.1 | 0.0018 |
| 4.1 | 0.0018 |
| 5 | 0.0004 |
| 6 | 0.0001 |
| 7 | 0.0003 |
| 7.4 | 0.003 |
| 7.17 | 0.0011 |
| 7.23 | 0.0008 |
| 7.24 | 0.0007 |
| 7.25 | 0.0003 |
| 7.26 | 0.0005 |
| 7.28 | 0.0009 |
| 20 | 0.0008 |
| 33 | 0.0064 |

### Biological Example—Measurement of pCREB in CD8+ T Cells in Human Whole Blood

Heparinised human whole blood was pre-incubated at 37° C. with serial dilutions of A2a antagonists for 20 min. and the phosphodiesterase inhibitor rolipram to amplify the pCREB response. The adenosine receptor agonist NECA is then added at a final concentration of 3 μM and following a 60 min incubation the blood is fixed and red blood cells lysed. White blood cells are isolated, permeabilized and stained with directly conjugated fluorescent antibodies to phospho-CREB (Alexa Fluor 488) and CD8 (Alexa Fluor 647) and the level of phospho-CREB in CD8+ T cells is measured by FACS using a BD Accuri C6 Flow Cytometer.

TABLE 3

| Example | pCREB IC50 (□M) |
|---|---|
| 2 | 0.02 |
| 2.1 | 0.05 |
| 3 | 0.07 |
| 3.1 | 0.01 |
| 4 | 0.01 |
| 4.1 | 0.008 |
| 5 | 0.015 |
| 6 | 0.011 |
| 7 | 0.01 |
| 7.4 | 0.06 |
| 7.17 | 0.004 |
| 7.23 | 0.03 |
| 7.24 | 0.012 |
| 7.25 | 0.007 |
| 7.26 | 0.015 |
| 7.28 | 0.022 |
| 7.29 | 0.005 |
| 19 | 0.216 |
| 20 | 0.105 |
| 33 | 0.219 |

MoDC pCREB Assay

Monocytes are isolated from human whole blood using CD14 microbeads (Miltenyi Biotec 130-050-201) and differentiated to immature monocyte-derived dendritic cells (MoDC) by culture for 5 days in Mo-DC Differentiation Medium (Miltenyi Biotec 130-094-812). MoDCs are seeded into fresh medium into U bottomed 96 well culture plates and pre-incubated at 37° C. with serial dilutions of A_{2A/B} antagonists for 15 min. At the same time the cells are co-incubated with the phosphodiesterase inhibitor rolipram (final concentration of 20 μM) to amplify the subsequent pCREB response. Adenosine receptor agonist NECA is then added at 1 μM final concentration, leading to an increase in CREB phosphorylation. Following a 45 min incubation at 37° C., the cells are fixed, permeabilized and stained with directly conjugated fluorescent antibodies to phospho-CREB (Alexa Fluor 488), CD209 (PerCP-Cy5.5) and CD86 (APC). Samples are analysed by FACS to measure fluorescence in corresponding channels, and also forward and Side scatter. The population of CD209+/CD86-low cells corresponding to immature dendritic cells is gated for analysis of pCREB levels. The adenosine receptor response in this cell population is largely specific to A_{2B}, based on specific response to the highly selective antagonist PSB-603.

Approximately 5% of the analysed cells are positive for detection of pCREB in the absence of agonist stimulation, representing the background signalling of all pathways upstream of CREB. Following NECA stimulation this is increased to ˜80% pCREB+ve, and this increase is essentially fully reversible by A_{2B} or dual A_{2A/B} antagonists.

IC50 is represented as the point of inflection of a sigmoidal curve

TABLE 4

| Example | pCREB IC50 (□M) |
|---|---|
| 2 | 0.003 |
| 2.1 | 0.003 |
| 3 | 0.006 |
| 4.1 | 0.002 |
| 5 | 0.003 |
| 6 | 0.002 |
| 7 | 0.003 |
| 7.4 | 0.004 |
| 7.17 | 0.002 |
| 7.23 | 0.003 |
| 7.24 | 0.002 |
| 7.25 | 0.002 |
| 7.26 | 0.002 |
| 7.28 | 0.005 |
| 20 | 0.003 |

### REFERENCES

1. Sukari A Nagasaka M Al-Hadidi A and Lum L G (2016). Anticancer Res. 36(11):5593-5606.
2. Vijayan D, Young A, Teng MWL, and Smyth M J (2017), Nat Rev Cancer. 17(12):709-724.
3. Houthuys, E, Marillier R, Deregnaucourt, T, Brouwer, M, Pirson, R, Marchante, J, et al (2016). SITC 2017 Conference, Maryland.
4. Gao Z W, Dong K, Zhang H Z (2014). “The roles of CD73 in cancer”. Biomed Res Int: 2014:460654.
5. Loi S, Pommey S, Haibe-Kains B, Beavis P A, Darcy P K, Smyth M J, et al. (2013), “CD73 promotes anthracycline resistance and poor prognosis in triple negative breast cancer” Proc Natl Acad Sci USA.; 110(27):11091-6.
6. Deaglio S, Dwyer K M, Gao W, Friedman D, Usheva A, Erat A et al (2007). J. Exp Med. 204, No. 6, Jun. 11, 2007 1257-1265.
7. Csóka B, Selmeczy Z, Koscsó B, Ne\acute{m}eth Z H, Pacher P, Murray P J, Kepka-Lenhart D, Morris S M Jr, Gause W C, Leibovich S J, Haskó G (2012). Adenosine promotes alternative macrophage activation via A2A and A2B receptors. FASEB J 26:376-86.
8. Ryzhov S, Novitskiy S V, Goldstein A E, Biktasova A, Blackburn M R, Biaggioni I, Dikov M M, Feoktistov I (2011). Adenosinergic regulation of the expansion and immunosuppressive activity of CD11b+Gr1+ cells. J Immunol. 187(11):6120-9.
9. Novitskiy S V, Ryzhov S, Zaynagetdinov R, Goldstein A E, Huang Y, Tikhomirov O Y, Blackburn M R, Biaggioni I, Carbone D P, Feoktistov I, Dikov M M (2008). Adenosine receptors in regulation of dendritic cell differentiation and function. Blood 2008 112:1822-31
10. Wilson J M, Ross W G, Agbai O N, Frazier R, Figler R A, Rieger J, Linden J, Ernst P B (2009). The A2B adenosine receptor impairs the maturation and immunogenicity of dendritic cells. J. Immunol. 18:4616-23.
11. Cekic C, Sag D, Li Y, Theodorescu D, Strieter R M, Linden J. J (2012). Adenosine A2B receptor blockade slows growth of bladder and breast tumors. J Immunol. 188:198-205.
12. Sorrentino C, Miele L, Porta A, Pinto A, Morello S (2015). Myeloid-derived suppressor cells contribute to A2B adenosine receptor-induced VEGF production and angiogenesis in a mouse melanoma model. Oncotarget. 6:27478-89.
13. Iannone R, Miele L, Maiolino P, Pinto A, Morello S (2013). Blockade of A2b adenosine receptor reduces tumor growth and immune suppression mediated by myeloid-derived suppressor cells in a mouse model of melanoma. Neoplasia. 2013 15(12):1400-9.

1. A compound, or pharmaceutically acceptable salt thereof, having the structural formula I shown below: 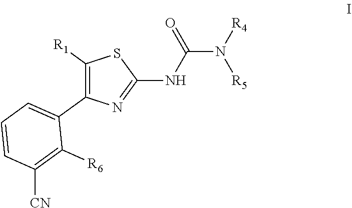
wherein:
R₁ is selected from: 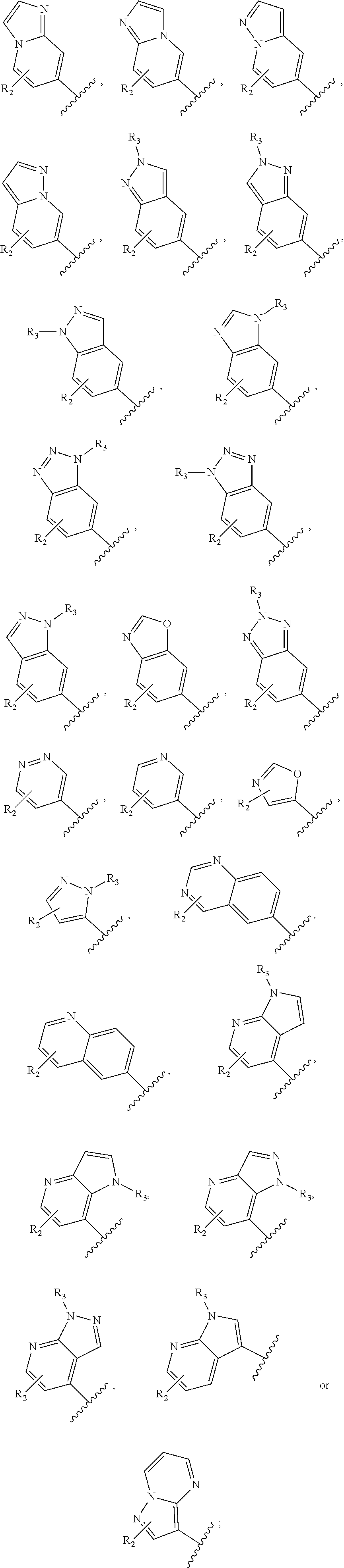
wherein:
any available C atoms in R₁ are optionally substituted by one or more R₂substituent groups, and
any available N atoms in R₁ are optionally in the form of a N-oxide or substituted by an R₃ group;
wherein
each R₂ is independently selected from (1-6C)alkyl, (3-6C)cycloalkyl, (3-6C)cycloalkyl(1-2C)alkyl, aryl, aryl(1-2C)alkyl, heterocyclyl, heterocyclyl(1-2C)alkyl, heteroaryl (e.g. a 5-membered heteroaryl), heteroaryl(1-2C)alkyl, halo, (1-3C)haloalkyl, (1-3C)haloalkoxy, (1-3C)hydroxyalkyl, cyano, —(CR_{1C}R_{1D})_{q1}—OR_{1A}, —(CR_{1C}R_{1D})_{q1}—C(O)R_{1A}, —(CR_{1C}R_{1D})_{q1}—C(O)OR_{1A}, —(CR_{1C}R_{1D})_{q1}—OC(O)R_{1A}, —(CR_{1C}R_{1D})_{q1}—C(O)N(R_{1B})R_{1A}, —(CR_{1C}R_{1D})_{q1}—N(R_{1B})C(O)R_{1A}, —(CR₁CR_{1D})_{q1}—S(O)ₚR_{1A} (where p is 0, 1 or 2), —(CR₁CR_{1D})_{q1}—SO₂N(R_{1B})R_{1A}, or —(CR_{1C}R_{1D})_{q1}—N(R_{1B})SO₂R_{1A},
wherein q1 is 0, 1, or 2; and
wherein R_{1A} and R_{1B} are each independently selected from hydrogen, (1-2C)alkyl, (3-4C)cycloalkyl or (3-4C)cycloalkyl(1-2C)alkyl;
wherein R_{1C} and R_{1D} are each independently selected from hydrogen or (1-2C)alkyl; and
R₃ is selected from hydrogen, (1-3C)alkyl, (3-6C)cycloalkyl, (3-6C)cycloalkyl(1-2C)alkyl, aryl, aryl(1-2C)alkyl, a C-linked heterocyclyl, heterocyclyl(1-2C)alkyl, heteroaryl, heteroaryl(1-2C)alkyl, (1-3C)haloalkyl, (1-3C)haloalkoxy, (1-3C)hydroxyalkyl, —(CR_{3C}R_{3D})₁₋₂-OR_{3A}, —(CR_{3C}R_{3D})_{q3}—C(O)R_{3A}, —(CR₃CR_{3D})_{q3}—C(O)OR_{3A}, —(CR₃CR_{3D})₁₋₂—OC(O)R_{3A}, —(CR₃CR_{3D})_{q3}—C(O)N(R_{3B})R_{q3}, —(CR₁CR_{1D})₁₋₂—N(R_{3B})C(O)R_{3A}, —(CR_{3C}R_{3D})—S(O)ₚ₃R_{3A} (where p3 is 0, 1 or 2), —(CR_{3C}R_{3D})₁₋₂-SO₂N(R_{3B})R_{3A}, or —(CR_{3D}R_{3D})₁₋₂—N(R_{3B})SO₂R_{3A};
wherein R_{3A} and R_{3B} are each independently selected from hydrogen, (1-2C)alkyl, (3-4C)cycloalkyl or (3-4C)cycloalkyl(1-2C)alkyl;
wherein R_{3C} and R_{3D} are each independently selected from hydrogen or (1-2C)alkyl; and
wherein q3 is 0, 1, or 2; and
R₄ and R₅ are linked such that, together with the nitrogen atom to which they are attached, they form a heterocyclic ring which is optionally substituted on any available carbon atom by one or more R_{10C} substituent groups,
wherein R_{10C} is selected from oxo, thioxo, halo, or cyano substituents, or a group of the formula:
—[CR₇ₐR_{7b}]ₙ-L-Z
wherein
n is an integer from 0 to 6;
R₇ₐ and R_{7b} are each independently selected from hydrogen, fluoro or (1-2C)alkyl;
L is absent or selected from —O—, —S—, —SO—, —SO₂—, —N(Rₐ)—, —C(O)—, —C(O)O—, —OC(O)—, —C(O)N(Rₐ)—, —N(Rₐ)C(O)—, —N(Rₐ)C(O)N(R_{b})—, —C(S)N(Rₐ)—, —N(Rₐ)C(S)—, —N(Rₐ)C(S)N(R_{b})—, —S(O)₂N(Rₐ)—, or —N(Rₐ)SO₂—, wherein Rₐ and R_{b} are each independently selected from hydrogen or (1-2C)alkyl; and
Z is selected from hydrogen, (1-6C)alkyl, (3-6C)cycloalkyl, (3-6C)cycloalkyl(1-2C)alkyl, aryl, aryl(1-2C)alkyl, heterocyclyl, heterocyclyl(1-2C)alkyl, heteroaryl or heteroaryl(1-2C)alkyl; and wherein Z is optionally substituted by one or more substituents selected from (1-6C)alkyl, halo, (1-6C)haloalkyl, (1-6C)haloalkoxy, cyano, nitro, —NR_{c}R_{d}, —OR_{c}, —C(O)R_{c}, —C(O)OR_{c}, —OC(O)R_{c}, —C(O)N(R_{c}) R_{d}, —N(R_{c})C(O)R_{d}, —S(O)_{y}Rₑ(where y is 0, 1 or 2), —SO₂N(R_{c})R_{d}, —N(R_{C})SO₂R_{d}, —(CH₂)_{z}NR_{c}R_{d} (where z is 1, 2 or 3) or oxo,
wherein any alkyl moiety in a substituent group on Z is optionally further substituted by cyano, halo, hydroxy, amino, oxo, (1-2C)alkyl or (1-2C)alkoxy and R_{c} and R_{d} are each independently selected from hydrogen, (1-6C)alkyl, (1-6C)haloalkyl or (3-6C)cycloalkyl;
and wherein any available N atoms in the ring formed by R₄ and R₅ are optionally in the form of a N-oxide or are optionally substituted by one or more R_{10N},
wherein R_{10N} is —S(O)₂NH₂ or selected from:
(i) —Z₁;
(ii) -L₁ₐZ₁; or
(iii) [CR₈ₐR_{8b}]₁₋₆-L_{1b}-Z₁;
wherein
R₈ₐ and R_{8b} are each independently selected from hydrogen, fluoro or (1-2C)alkyl;
L₁ₐ is selected from —C(O)—, —S(O)₂—, —C(O)O—, —C(O)N(Rₐ₁)—, —S(O)₂N(Rₐ₁)— or —N(Rₐ₁)—,
wherein Rₐ₁ is selected from hydrogen or (1-2C)alkyl;
L_{1b} is absent or selected from —O—, —S—, —SO—, —SO₂, —N(Rₐ₂)—, —C(O)—, —C(O)O—, —OC(O)—, —C(O)N(Rₐ₂)—, —N(Rₐ₂)C(O)—, —N(Rₐ₂)C(O)N(R_{b2})—, —C(S)N(Rₐ₂)—, —N(Rₐ₂)C(S)—, —N(Rₐ₂)C(S)N(R_{b2})—, —S(O)₂N(Rₐ₂)—, or —N(Rₐ₂)SO₂, wherein Rₐ₂ and R_{b2} are each independently selected from hydrogen or (1-2C)alkyl; and
Z₁ is selected from (1-6C)alkyl, (3-6C)cycloalkyl, aryl, heterocyclyl, heteroaryl, (3-6C)cycloalkyl(1-2C)alkyl, aryl(1-2C)alkyl, heterocyclyl(1-2C)alkyl or heteroaryl(1-2C)alkyl, with the proviso that when Z₁ is a heterocyclyl group directly linked to a N atom it is a carbon-linked heterocyclyl; and wherein Z₁ is optionally substituted by one or more substituents selected from (1-6C)alkyl, halo, (1-6C)haloalkoxy, cyano, nitro, —NRₑR_{f}, —ORₑ, —C(O)Rₑ, —C(O)ORₑ, —OC(O)Rₑ, —C(O)N(Rₑ)R_{f}, —N(Rₑ)C(O)R_{f}, —S(O)_{y}Rₑ (where y is 0, 1 or 2), —SO₂N(Rₑ)R_{f}, —N(Rₑ)SO₂R_{f}, —(CH₂)_{z}NRₑR_{f} (where z is 1, 2 or 3) or oxo, and wherein Rₑ and R_{f} are each independently selected from hydrogen, (1-6C)alkyl, (1-6C)haloalkyl or (3-6C)cycloalkyl;
and wherein any S atoms present in the heterocyclic ring may optionally be present as S(═O), S(═O)₂ or S(═O)(═NRₑ) wherein Rₑ is selected from hydrogen, (1-3C)alkyl or (2-3C)alkanoyl; and
R₆ is selected from hydrogen, halo, methyl, methoxy and trifluoromethyl.

2. A compound according to claim 1, or a pharmaceutically acceptable salt thereof, wherein R₁ is selected from: 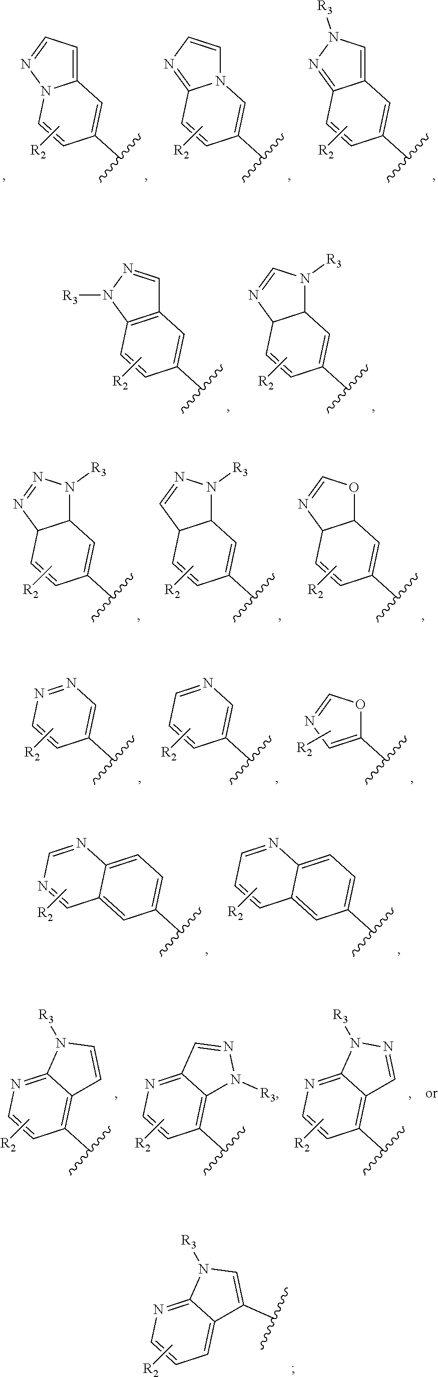
wherein R₁ is optionally substituted on any available carbon atom by one or more R₂ substituent groups; and R₂ and R₃ are as defined in claim 1.

3. A compound according to claim 1, or a pharmaceutically acceptable salt thereof, wherein R₁ is selected from: 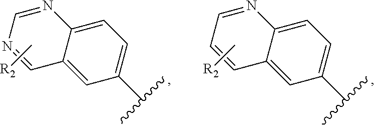
wherein R₁ is optionally substituted on any available carbon atom by one or more R₂, and R₂ is as defined in claim 1.

4. A compound according to claim 1, or a pharmaceutically acceptable salt thereof, wherein R₁ is: 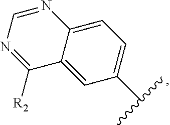
wherein R₂ is as defined in claim 1.

5. A compound according to claim 1, or a pharmaceutically acceptable salt thereof, wherein each R₂, if present, is independently selected from (1-2C)alkyl, a 5-membered heteroaryl, halo, (1-2C)haloalkyl, (1-2C)haloalkoxy, (1-2C)hydroxyalkyl, cyano, —(CR_{1C}R_{1D})_{q1}—OR_{1A};
wherein q1 is 0 or 1; and
wherein R_{1A} and R_{1B} are each independently selected from hydrogen or (1-2C)alkyl.

6. A compound according to claim 1, or a pharmaceutically acceptable salt thereof, wherein each R₂, if present, is independently selected from methyl, ethyl or methoxy.

7. A compound according to claim 1, or a pharmaceutically acceptable salt thereof, wherein R₃ is selected from hydrogen or (1-3C)alkyl.

8. A compound according to claim 1, or a pharmaceutically acceptable salt thereof, wherein R₄ and R₅ are linked such that, together with the nitrogen atom to which they are attached, they form a heterocyclic ring which is optionally substituted on any available carbon atom by one or more R_{10C} substituent groups,
wherein R_{10C} is selected from oxo, halo, or cyano substituents, or a group of the formula:
—[CH₂]ₙ-L-Z
wherein
n is 0 to 2;
L is absent or selected from —O—, —S—, —SO—, —SO₂—, —N(Rₐ₂)—, —C(O)—, —C(O)O—, —OC(O)—, —C(O)N(Rₐ₂)—, —N(Rₐ₂)C(O)—, —N(Rₐ₂)C(O)N(R_{b2})—, —S(O)₂N(Rₐ₂)—, or —N(Rₐ₂)SO₂—, wherein Rₐ₂ and R_{b2} are each independently selected from hydrogen or methyl; and
Z is selected from hydrogen, (1-6C)alkyl, (3-6C)cycloalkyl, (3-6C)cycloalkyl(1-2C)alkyl, aryl, heterocyclyl, heterocyclyl(1-2C)alkyl or heteroaryl; and wherein
Z is optionally substituted by one or more substituents selected from (1-2C) alkyl, halo, (1-2C)haloalkyl, (1-2C)haloalkoxy, cyano, —NR_{c}R_{d}, —OR_{c}, —C(O)R_{c}, —C(O)OR_{c}, —OC(O)R_{c}, —C(O)N(R_{c})R_{d}, —N(R_{c})C(O)R_{d}, —S(O)_{y}R₁ (where y is 0, 1 or 2), —SO₂N(R_{c})R_{d}, —N(R_{c})SO₂R_{d}, —(CH₂)_{z}NR_{c}R_{d} (where z is 1 or 2) or oxo;
wherein any alkyl moiety in a substituent group on Z₁ is optionally further substituted by cyano, halo, hydroxy, amino, oxo, (1-2C)alkyl or (1-2C)alkoxy and R_{c} and R_{d} are each independently selected from hydrogen, (1-2C)alkyl, (1-2C)haloalkyl or (3-6C)cycloalkyl;
and wherein any available N atoms are optionally in the form of a N-oxide or are optionally substituted by one or more R_{10N}, wherein R_{10N} is —S(O)₂NH₂ or selected from:
(i) —Z₁;
(ii) -L₁ₐ Z₁; or
(iii) —[CR₈ₐR_{8b}]₁₋₂-L_{1b}-Z₁;
wherein
R₈ₐ and R_{8b} are both hydrogen;
L₁ₐ is selected from —C(O)—, —S(O)₂— or —S(O)₂N(Rₐ₁)—, wherein Rₐ₁ is hydrogen or methyl;
L_{1b} is absent or selected from —O—, —S—, —SO—, —SO₂, —N(Rₐ₂)—, —C(O)—, —C(O)O—, —OC(O)—, —C(O)N(Rₐ₂)—, —N(Rₐ₂)C(O)—, —N(Rₐ₂)C(O)N(R_{b2})—, —S(O)₂N(Rₐ₂)—, or —N(Rₐ₂)SO₂, wherein Rₐ₂ and R_{b2} are each independently selected from hydrogen or methyl; and
Z₁ is selected from (1-6C)alkyl, (3-6C)cycloalkyl, aryl, heterocyclyl, heteroaryl, (3-6C)cycloalkyl(1-2C)alkyl or aryl(1-2C)alkyl, with the proviso that when Z₁ is a heterocyclyl group directly linked to a N atom it is a carbon-linked heterocyclyl; and wherein Z₁ is optionally substituted by one or more substituents selected from (1-2C)alkyl, halo, (1-2C)haloalkoxy, cyano, nitro, —NRₑR_{f}, —ORₑ, —C(O)Rₑ, —C(O)ORₑ, —OC(O)Rₑ, —C(O)N(Rₑ)R_{f}, —N(Rₑ)C(O)R_{f}, —S(O)_{y}Rₑ (where y is 0, 1 or 2), —SO₂N(Rₑ)R_{f}, —N(Rₑ)SO₂R_{f}, —(CH₂)_{z}NRₑR1 (where z is 1, 2 or 3) or oxo, and wherein Rₑ and R_{f} are each independently selected from hydrogen or (1-2C)alkyl;
and wherein any S atoms present in the heterocyclic ring may optionally be present as S(═O), S(═O)₂ or S(═O)(═NRₑ) wherein Rₑ is selected from hydrogen, (1-3C)alkyl or (2-3C)alkanoyl.

9. A compound according to claim 1, or a pharmaceutically acceptable salt thereof, wherein R₄ and R₅ are linked such that, together with the nitrogen atom to which they are attached, they form a heterocyclic ring which is optionally substituted on any available carbon atom by one or more R_{10C} substituent groups,
wherein R_{10C} is selected from oxo, halo, or cyano substituents, or a group of the formula:
—[CH₂]ₙ-L-Z
wherein
n is 0 to 2;
L is absent or selected from —O—, —S—, —SO—, —SO₂—, —N(Rₐ)—, —C(O)—, —C(O)O—, —OC(O)—, —C(O)N(Rₐ)—, —N(Rₐ)C(O)—, —S(O)₂N(Rₐ)—, or —N(Rₐ)SO₂—, wherein Rₐ and R_{b} are each independently selected from hydrogen or methyl; and
Z is selected from hydrogen, (1-4C)alkyl, (3-6C)cycloalkyl, (3-6C)cycloalkyl(1-2C)alkyl, phenyl, a 4-7 membered heterocyclyl, a 4-7 membered heterocyclyl(1-2C)alkyl or a 5 or 6-membered heteroaryl; and wherein Z is optionally substituted by one or more substituents selected from (1-2C)alkyl, halo, (1-2C)haloalkyl, (1-2C)haloalkoxy, cyano, —NR_{c}R_{d}, —OR_{c}, —C(O)R_{c}, —C(O)OR_{c}, —OC(O)R_{c}, —C(O)N(R_{c})R_{d}, —N(R_{c})C(O)R_{d}, —S(O)_{y}R, (where y is 0, 1 or 2), —SO₂N(R_{c})R_{d}, —N(R_{c})SO₂R_{d}, —(CH₂)_{z}NR_{c}R_{d}(where z is 1 or 2) or oxo;
wherein R_{c} and R_{d} are each independently selected from hydrogen or (1-2C)alkyl;
and wherein any available N atoms are optionally in the form of a N-oxide or are optionally substituted by one or more R_{10N}, wherein R_{10N} is —S(O)₂NH₂ or selected from:
(i) —Z₁;
(ii) -L₁a-Z₁; or
(iii) —[CR₈ₐR_{8b}]1 2-L_{1b}-Z₁;
wherein
R₈ₐ and R_{8b} are both hydrogen;
L₁ₐ is selected from —C(O)—, —S(O)₂— or —S(O)₂N(Rₐ₁)—, wherein Rₐ₁ is hydrogen;
L_{1b} is absent or selected from —O—, —S—, —SO—, —SO₂—, —N(Rₐ₂)—, —C(O)N(Rₐ₂)—, —N(Rₐ₂)C(O)—, —S(O)₂N(Rₐ₂)— or —N(Rₐ₂)SO₂, wherein Rₐ₂ is hydrogen; and
Z₁ is selected from (1-4C)alkyl, phenyl, a 4-7 membered heterocyclyl or a 5 or 6-membered heteroaryl, with the proviso that when Z₁ is a heterocyclyl group directly linked to a N atom it is a carbon-linked heterocyclyl; and wherein Z₁ is optionally substituted by one or more substituents selected from (1-2C)alkyl, halo, (1-2C)haloalkoxy, cyano, —NRₑR_{f}, —ORₑ, —C(O)Rₑ, —C(O)ORₑ, —OC(O)Rₑ, —C(O)N(Rₑ)R_{f}, —N(Rₑ)C(O)R_{f}, —S(O)_{y}Rₑ (where y is 0, 1 or 2), —SO₂N(Rₑ)R_{f}, —N(Rₑ)SO₂R_{f}, —(CH₂)_{z}NR_{c}R_{d} (where z is 1 or 2); and wherein Rₑ and R, are each independently selected from hydrogen or methyl;
and wherein any S atoms present in the heterocyclic ring may optionally be present as S(═O), S(═O)₂ or S(═O)(═NRₑ) wherein Rₑ is selected from hydrogen, (1-2C)alkyl or (2C)alkanoyl.

10. A compound according to claim 1, or a pharmaceutically acceptable salt thereof, wherein R₄ and R₅ are linked such that, together with the nitrogen atom to which they are attached, they form a heterocyclic ring which is optionally substituted on any available carbon atom by one or more R_{10C} substituent groups, R_{10C} is selected from oxo, halo, cyano, or a group of the formula:
-L-Z
wherein
L is absent, —O— or —N(Rₐ)—, wherein Rₐ is hydrogen or methyl;
and Z is selected from (1-4C)alkyl, 4-7 membered heterocyclyl or (3-6C)cycloalkyl(1-2C)alkyl,
wherein any (1-4C)alkyl, 4-7 membered heterocyclyl or (3-6C)cycloalkyl(1-2C)alkyl, is optionally substituted by one or more substituents selected from cyano, —NR_{c}R_{d} or —OR_{c}, wherein R_{c} and R_{d} are each independently selected from hydrogen or methyl;
and wherein any available N atoms are optionally in the form of a N-oxide or are optionally substituted by one or more R_{10N}, wherein R_{10N} is (1-4C)alkyl, and wherein any S atoms present in the heterocyclic ring may optionally be present as S(═O).

11. A compound according to claim 1, or a pharmaceutically acceptable salt thereof, wherein R₄ and R₅ are linked such that, together with the nitrogen atom to which they are attached, they form a heterocyclic ring selected from any one of the following options: 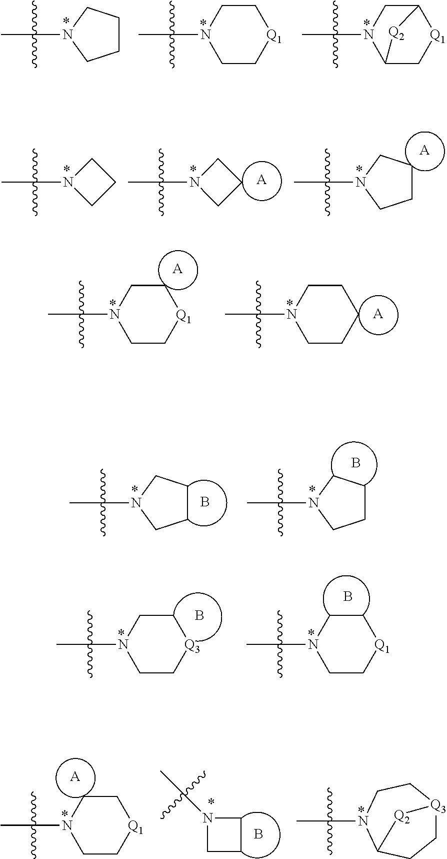
wherein * denotes the N atom to which R₄ and R₅ are attached;
Q₁ is O, NH, S, S(O), S(O)₂, S(O)(═NRₑ), N—R_{10N}, CH₂, CHR_{10C} or C(R_{10c})₂;
Q₂ is —CH₂—, —CHR_{10C}—, —C(R_{10c})₂—, —CHR_{10C}—CH₂—, —CH₂—CHR_{10C}—, —CHR_{10C}—CHR_{10C}—, —C(R_{10c})₂—CH₂—, or —CH₂—C(R_{10c})₂—;
Q₃ is CH, CR_{10C} or N;
Ring A is a spiro-fused 4, 5 or 6 membered carbocyclic or heterocyclic ring;
Ring B is a fused 4, 5 or 6 membered carbocyclic or heterocyclic ring;
wherein Rₑ is selected from hydrogen or methyl;
and wherein each of the heterocyclic ring systems is optionally substituted with one or more R_{10C} or R_{10N} substituent groups as defined in claim 1.

12. A compound according to claim 1, or a pharmaceutically acceptable salt thereof, wherein R₄ and R₅ are linked such that, together with the nitrogen atom to which they are attached, they form a heterocyclic ring selected from any one of the following options: 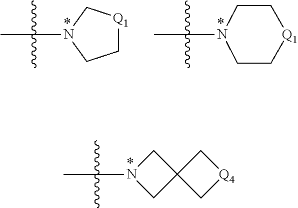
wherein * denotes the N atom to which R₄ and R₅ are attached;
Q₁ is O, NH, S, S(O), S(O)₂, S(O)(═NRₑ), N—R_{10N}, CH₂, CHR_{10C} or C(R_{10c})₂;
Q₄ is O, NH, S, S(O), S(O)₂, S(O)(═NRₑ), N—R_{10N}, CH₂, CHR_{10C} or C(R_{10c})₂;
wherein Rₑ is selected from hydrogen, (1-2C)alkyl or (2C)alkanoyl;
and wherein each of the heterocyclic ring systems is optionally substituted with one or more R_{10C} or R_{10N} substituent groups as defined in claim 1.

13. A compound according to claim 1, or a pharmaceutically acceptable salt thereof, wherein R₄ and R₅ are linked such that, together with the nitrogen atom to which they are attached, they form a heterocyclic ring of the structure: 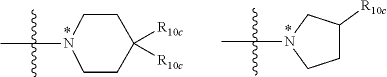
wherein * denotes the N atom to which R₄ and R₅ are attached;
and each R_{10c} is independently selected from the options defined in claim 1.

14. A compound according to claim 1, or a pharmaceutically acceptable salt thereof, wherein R₆ is selected from hydrogen, halo or methyl.

15. A compound according to claim 1, or a pharmaceutically acceptable salt thereof, wherein R₆ is hydrogen.

16. A compound of the formula IA, IB, IC, ID, IE or IF: 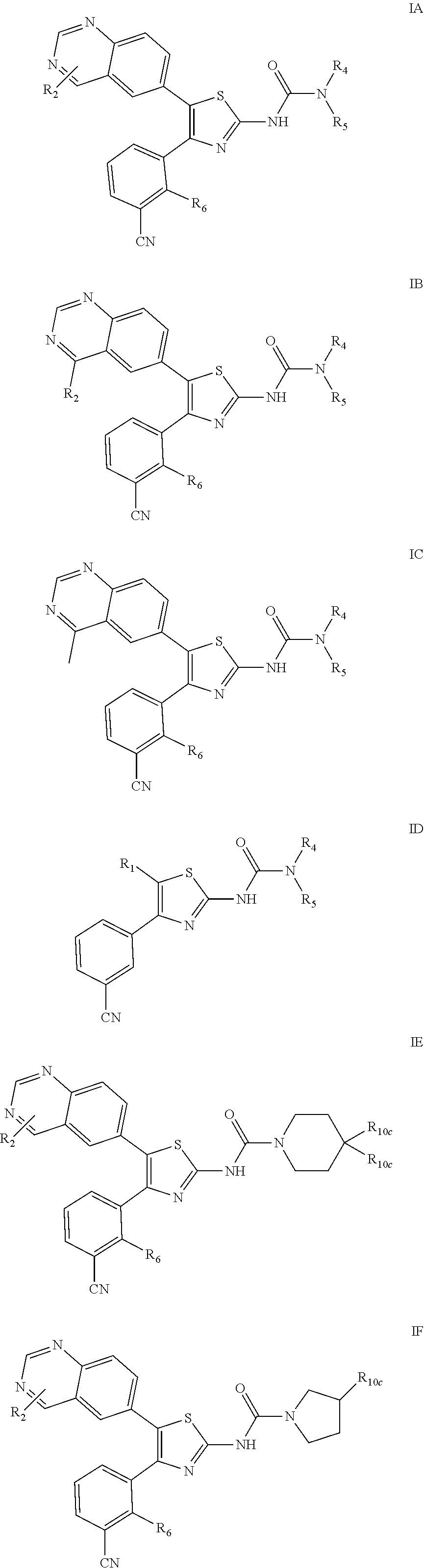
wherein R₂, R₄, R₅, R₆ and R_{10c} are each as defined in claim 1.

17. A compound, or a pharmaceutically acceptable salt thereof, selected from any one of the following:
(3S)-3-Cyano-N-[4-(3-cyanophenyl)-5-(4-methylquinazolin-6-yl)thiazol-2-yl]-3-methyl-pyrrolidine-1-carboxamide;
N-[4-(3-Cyanophenyl)-5-(4-methylquinazolin-6-yl)thiazol-2-yl]-4-oxa-1,9-diazaspiro[5.5]undecane-9-carboxamide;
N-[4-(3-Cyanophenyl)-5-(4-methylquinazolin-6-yl)thiazol-2-yl]-2-(cyclopropylmethyl)piperazine-1-carboxamide;
N-[4-(3-Cyanophenyl)-5-(4-methyl-1H-indazol-6-yl)thiazol-2-yl]-2-oxa-6-azaspiro[3.3]heptane-6-carboxamide;
N-[4-(3-Cyanophenyl)-5-(7-methyl-3H-benzotriazol-5-yl)thiazol-2-yl]-2-oxa-6-azaspiro[3.3]heptane-6-carboxamide;
N-[4-(3-Cyanophenyl)-5-(1H-pyrrolo[2,3-b]pyridin-4-yl)thiazol-2-yl]-2-oxa-6-azaspiro[3.3]heptane-6-carboxamide;
N-[4-(3-Cyanophenyl)-5-(6-methyl-1H-pyrazolo[3,4-b]pyridin-4-yl)thiazol-2-yl]-2-oxa-6-azaspiro[3.3]heptane-6-carboxamide;
N-[4-(3-Cyanophenyl)-5-(4-methylquinazolin-6-yl)thiazol-2-yl]-4-piperazin-1-yl-piperidine-1-carboxamide;
N-[4-(3-Cyanophenyl)-5-(4-methylquinazolin-6-yl)thiazol-2-yl]-1-oxa-4,9-diazaspiro[5.5]undecane-9-carboxamide;
N-[4-(3-Cyanophenyl)-5-(4-methylquinazolin-6-yl)thiazol-2-yl]-6-oxa-2,9-diazaspiro[4.5]decane-2-carboxamide;
N-[4-(3-Cyanophenyl)-5-(4-methylquinazolin-6-yl)thiazol-2-yl]-5-oxa-2,8-diazaspiro[3.5]nonane-2-carboxamide;
N-[4-(3-Cyanophenyl)-5-(4-methylquinazolin-6-yl)thiazol-2-yl]-8-oxa-2,5-diazaspiro[3.5]nonane-2-carboxamide;
N-[4-(3-Cyanophenyl)-5-(4-methylquinazolin-6-yl)thiazol-2-yl]-9-oxa-2,6-diazaspiro[4.5]decane-2-carboxamide;
(3S)—N-[4-(3-Cyanophenyl)-5-(4-methylquinazolin-6-yl)thiazol-2-yl]-3-(1-hydroxy-1-methyl-ethyl)pyrrolidine-1-carboxamide;
4-Cyano-N-[4-(3-cyanophenyl)-5-(4-methylquinazolin-6-yl)thiazol-2-yl]-4-methyl-piperidine-1-carboxamide;
(3R)—N-[4-(3-Cyanophenyl)-5-(4-methylquinazolin-6-yl)thiazol-2-yl]-3-(1-hydroxy-1-methyl-ethyl)pyrrolidine-1-carboxamide;
(3R)-3-Cyano-N-[4-(3-cyanophenyl)-5-(4-methylquinazolin-6-yl)thiazol-2-yl]-3-methyl-pyrrolidine-1-carboxamide;
4-(1-Cyano-1-methyl-ethyl)-N-[4-(3-cyanophenyl)-5-(4-methylquinazolin-6-yl)thiazol-2-yl]piperazine-1-carboxamide;
N-[4-(3-Cyanophenyl)-5-(4-methylquinazolin-6-yl)thiazol-2-yl]-3-methyl-3,6-diazabicyclo[3.2.0]heptane-6-carboxamide;
N-[4-(3-Cyanophenyl)-5-(4-methylquinazolin-6-yl)thiazol-2-yl]-6-methyl-2,6-diazaspiro[3.3]heptane-2-carboxamide;
4-(1-Cyanoethyl)-N-[4-(3-cyanophenyl)-5-(4-methylquinazolin-6-yl)thiazol-2-yl]piperazine-1-carboxamide;
N-[4-(3-Cyanophenyl)-5-(4-methylquinazolin-6-yl)thiazol-2-yl]-4-(dimethylamino)piperidine-1-carboxamide;
N-[4-(3-Cyanophenyl)-5-(4-methylquinazolin-6-yl)thiazol-2-yl]-2-(1-hydroxy-1-methyl-ethyl)morpholine-4-carboxamide;
N-[4-(3-Cyanophenyl)-5-(4-methylquinazolin-6-yl)thiazol-2-yl]-2-[(dimethylamino)methyl]morpholine-4-carboxamide;
N-[4-(3-Cyanophenyl)-5-(4-methylquinazolin-6-yl)thiazol-2-yl]-3-(2-pyrrolidin-1-ylethoxy)pyrrolidine-1-carboxamide;
N-[4-(3-Cyanophenyl)-5-(4-methylquinazolin-6-yl)thiazol-2-yl]-4-methyl-1-oxa-4,9-diazaspiro[5.5]undecane-9-carboxamide;
N-[4-(3-Cyanophenyl)-5-(4-methylquinazolin-6-yl)thiazol-2-yl]-1-methyl-3,4,4a,5,7,7a-hexahydro-2H-pyrrolo[3,4-b]pyridine-6-carboxamide;
N-[4-(3-Cyanophenyl)-5-(4-methylquinazolin-6-yl)thiazol-2-yl]-4-(4-methylpiperazin-1-yl)piperidine-1-carboxamide;
N-[4-(3-Cyanophenyl)-5-(4-methylquinazolin-6-yl)thiazol-2-yl]-4-(1-methyl-4-piperidyl)piperazine-1-carboxamide;
N-[4-(3-Cyanophenyl)-5-(4-methylquinazolin-6-yl)thiazol-2-yl]-4-morpholino-piperidine-1-carboxamide;
4-(Cyanomethyl)-N-[4-(3-cyanophenyl)-5-(4-methylquinazolin-6-yl)thiazol-2-yl]piperazine-1-carboxamide;
(3S)—N-[4-(3-Cyanophenyl)-5-(4-methylquinazolin-6-yl)thiazol-2-yl]-3-(1-hydroxy-1-methyl-ethyl)piperazine-1-carboxamide;
N-[4-(3-Cyanophenyl)-5-(4-methylquinazolin-6-yl)thiazol-2-yl]-3-oxo-2,8-diazaspiro[4.5]decane-8-carboxamide;
N-[4-(3-Cyanophenyl)-5-(4-methylquinazolin-6-yl)thiazol-2-yl]-1,4-diazepane-1-carboxamide;
N-[4-(3-Cyanophenyl)-5-(4-methylquinazolin-6-yl)thiazol-2-yl]-1-oxa-6-azaspiro[3.3]heptane-6-carboxamide;
(4aR,7aS)—N-[4-(3-Cyanophenyl)-5-(4-methylquinazolin-6-yl)thiazol-2-yl]-4-methyl-2,3,4a,5,7,7a-hexahydropyrrolo[3,4-b][1,4]oxazine-6-carboxamide;
N-[4-(3-Cyanophenyl)-5-(4-methylquinazolin-6-yl)thiazol-2-yl]piperazine-1-carboxamide;
N-[4-(3-Cyanophenyl)-5-(4-methylquinazolin-6-yl)thiazol-2-yl]-4-methyl-piperazine-1-carboxamide;
N-[4-(3-Cyanophenyl)-5-(4-methylquinazolin-6-yl)thiazol-2-yl]-2-oxo-1,8-diazaspiro[4.5]decane-8-carboxamide;
N-[4-(3-Cyanophenyl)-5-(4-methylquinazolin-6-yl)thiazol-2-yl]-6-hydroxy-6-methyl-2-azaspiro[3.3]heptane-2-carboxamide;
3-Cyano-N-[4-(3-cyanophenyl)-5-(4-methylquinazolin-6-yl)thiazol-2-yl]-3-methyl-pyrrolidine-1-carboxamide;
N-[4-(3-Cyanophenyl)-5-(4-methylquinazolin-6-yl)thiazol-2-yl]-3-methyl-2-oxo-1-oxa-3,8-diazaspiro[4.5]decane-8-carboxamide;
N-[4-(3-Cyanophenyl)-5-(4-methylquinazolin-6-yl)thiazol-2-yl]-4-(2-hydroxyethyl)piperazine-1-carboxamide;
N-[4-(3-Cyanophenyl)-5-(4-methylquinazolin-6-yl)thiazol-2-yl]-4-(oxetan-3-yl)piperazine-1-carboxamide;
N-[4-(3-Cyanophenyl)-5-(4-methylquinazolin-6-yl)thiazol-2-yl]-2-oxa-6-azaspiro[3.3]heptane-6-carboxamide;
(2S)—N-[4-(3-Cyanophenyl)-5-(4-methylquinazolin-6-yl)thiazol-2-yl]-2-(1-hydroxy-1-methyl-ethyl)morpholine-4-carboxamide;
(2R)—N-[4-(3-cyanophenyl)-5-(4-methylquinazolin-6-yl)thiazol-2-yl]-2-(1-hydroxy-1-methyl-ethyl)morpholine-4-carboxamide;
(2S)—N-[4-(3-Cyanophenyl)-5-(4-methylquinazolin-6-yl)thiazol-2-yl]-2-(1-hydroxy-1-methyl-ethyl)morpholine-4-carboxamide;
(2R)—N-[4-(3-cyanophenyl)-5-(4-methylquinazolin-6-yl)thiazol-2-yl]-2-(1-hydroxy-1-methyl-ethyl)morpholine-4-carboxamide;
N-[4-(3-Cyanophenyl)-5-(2-methylquinazolin-7-yl)thiazol-2-yl]-2-oxa-6-azaspiro[3.3]heptane-6-carboxamide;
N-[4-(3-Cyanophenyl)-5-(2,4-dimethylquinazolin-6-yl)thiazol-2-yl]-2-oxa-6-azaspiro[3.3]heptane-6-carboxamide;
N-[4-(3-Cyanophenyl)-5-quinazolin-6-yl-thiazol-2-yl]-2-oxa-6-azaspiro[3.3]heptane-6-carboxamide;
N-[4-(3-Cyanophenyl)-5-(4-methyl-6-quinolyl)thiazol-2-yl]-2-oxa-6-azaspiro[3.3]heptane-6-carboxamide;
N-[4-(3-Cyanophenyl)-5-(4-methoxyquinazolin-6-yl)thiazol-2-yl]-2-oxa-6-azaspiro[3.3]heptane-6-carboxamide;
N-[4-(3-Cyanophenyl)-5-(4-ethylquinazolin-6-yl)thiazol-2-yl]-2-oxa-6-azaspiro[3.3]heptane-6-carboxamide;
4-Cyano-N-[4-(3-cyanophenyl)-5-(2,4-dimethyloxazol-5-yl)thiazol-2-yl]-4-methyl-piperidine-1-carboxamide;
N-[4-(3-Cyanophenyl)-5-(2,4-dimethyloxazol-5-yl)thiazol-2-yl]-2-oxa-6-azaspiro[3.3]heptane-6-carboxamide;
N-[4-(3-Cyanophenyl)-5-(1-methylpyrrolo[2,3-b]pyridin-3-yl)thiazol-2-yl]-2-oxa-6-azaspiro[3.3]heptane-6-carboxamide;
N-[4-(3-Cyanophenyl)-5-(1H-pyrrolo[2,3-b]pyridin-3-yl)thiazol-2-yl]-2-oxa-6-azaspiro[3.3]heptane-6-carboxamide;
N-[4-(3-Cyanophenyl)-5-(3,8-dimethylimidazo[1,2-a]pyridin-6-yl)thiazol-2-yl]-2-oxa-6-azaspiro[3.3]heptane-6-carboxamide;
4-Cyano-N-[4-(3-cyanophenyl)-5-(5-methyl-1H-pyrazolo[4,3-b]pyridin-7-yl)thiazol-2-yl]-4-methyl-piperidine-1-carboxamide;
N-[4-(3-Cyanophenyl)-5-(4,8-dimethylquinazolin-6-yl)thiazol-2-yl]-2-oxa-6-azaspiro[3.3]heptane-6-carboxamide;
N-[4-(3-Cyanophenyl)-5-(1-methylpyrrolo[2,3-b]pyridin-4-yl)thiazol-2-yl]-2-oxa-6-azaspiro[3.3]heptane-6-carboxamide;
N-[4-(3-Cyanophenyl)-5-(3-methylimidazo[1,2-a]pyridin-6-yl)thiazol-2-yl]-2-oxa-6-azaspiro[3.3]heptane-6-carboxamide;
N-[4-(3-Cyanophenyl)-5-(5-methyl-1H-pyrazolo[4,3-b]pyridin-7-yl)thiazol-2-yl]-2-oxa-6-azaspiro[3.3]heptane-6-carboxamide;
N-[4-(3-Cyanophenyl)-5-(1,6-dimethylpyrrolo[2,3-b]pyridin-4-yl)thiazol-2-yl]-2-oxa-6-azaspiro[3.3]heptane-6-carboxamide;
N-[4-(3-Cyanophenyl)-5-(3-methylbenzimidazol-5-yl)thiazol-2-yl]-2-oxa-6-azaspiro[3.3]heptane-6-carboxamide;
N-[4-(3-Cyanophenyl)-5-imidazo[1,2-a]pyridin-6-yl-thiazol-2-yl]-2-oxa-6-azaspiro[3.3]heptane-6-carboxamide;
N-[4-(3-Cyanophenyl)-5-(7-methyl-1H-indazol-5-yl)thiazol-2-yl]-2-oxa-6-azaspiro[3.3]heptane-6-carboxamide;
N-[4-(3-Cyanophenyl)-5-(2,7-dimethylindazol-5-yl)thiazol-2-yl]-2-oxa-6-azaspiro[3.3]heptane-6-carboxamide;
N-[4-(3-Cyanophenyl)-5-(8-methylimidazo[1,2-a]pyridin-6-yl)thiazol-2-yl]-2-oxa-6-azaspiro[3.3]heptane-6-carboxamide;
N-[4-(3-Cyanophenyl)-5-(1,7-dimethylindazol-5-yl)thiazol-2-yl]-2-oxa-6-azaspiro[3.3]heptane-6-carboxamide;
N-[4-(3-Cyanophenyl)-5-(6-methylpyridazin-4-yl)thiazol-2-yl]-2-oxa-6-azaspiro[3.3]heptane-6-carboxamide;
N-[4-(3-Cyanophenyl)-5-[5-methyl-6-(triazol-2-yl)-3-pyridyl]thiazol-2-yl]-2-oxa-6-azaspiro[3.3]heptane-6-carboxamide;
N-[4-(3-Cyanophenyl)-5-pyrazolo[1,5-a]pyridin-5-yl-thiazol-2-yl]morpholine-4-carboxamide;
N-[4-(3-Cyanophenyl)-5-(6-methyl-1H-pyrrolo[2,3-b]pyridin-4-yl)thiazol-2-yl]-2-oxa-6-azaspiro[3.3]heptane-6-carboxamide; or
N-[5-(1,3-benzoxazol-6-yl)-4-(3-cyanophenyl)thiazol-2-yl]-2-oxa-6-azaspiro[3.3]heptane-6-carboxamide.

18. A pharmaceutical composition comprising a compound according to claim 1, or a pharmaceutically acceptable salt thereof, in admixture with a pharmaceutically acceptable diluent or carrier.

19. A compound according to claim 1, or a pharmaceutically acceptable salt thereof, for use in therapy.

20. A compound according to claim 1, or a pharmaceutically acceptable salt thereof, for use:
(i) in the treatment of a proliferative condition; or
(ii) in the treatment of cancer.

21. A compound according to claim 1, or a pharmaceutically acceptable salt thereof, for use
in the treatment of cancer, wherein the compound or pharmaceutical composition is administered in combination with one or more additional anticancer agents selected from the group consisting of:
1) other forms of cancer immunotherapy and anti-cancer chemotherapeutic agents;
2) adenosine pathway modulators:
3) anti-PD-1 and PDL-1 antibodies; and
4) anti-CTLA4 antibodies.

22. A method of treating a proliferative disorder in a patient in need of such treatment, the method comprising administering a therapeutically effective amount of a compound according to claim 1, or a pharmaceutically acceptable salt thereof, to said patient.

23. A method of treating cancer in a patient in need of such treatment, the method comprising administering a therapeutically effective amount of a compound according to claim 1, or a pharmaceutically acceptable salt thereof, to said patient.

24. A method according to claim 23, wherein the method further comprises administering said therapeutically effective amount of a pharmaceutical composition in combination with one or more additional anticancer agents selected from:
1) other forms of cancer immunotherapy and anti-cancer chemotherapeutic agents;
2) adenosine pathway modulators;
3) anti-PD-1 and PDL-1 antibodies; and
4) anti-CTLA4 antibodies.

25. A method of treating a proliferative disorder in a patient in need of such treatment, the method comprising administering a therapeutically effective amount of a compound according to claim 1, or a pharmaceutically acceptable salt thereof, to said patient in combination with one or more additional anticancer agents.

26. A method according to claim 25, wherein the one or more additional anticancer agents is selected from:
1) other forms of cancer immunotherapy and anti-cancer chemotherapeutic agents;
2) adenosine pathway modulators;
3) anti-PD-1 and PDL-1 antibodies; and
4) anti-CTLA4 antibodies.

27. A pharmaceutical composition according to claim 18 for use in therapy.

28. A pharmaceutical composition according to claim 18 for use
(i) in the treatment of a proliferative condition or (ii) in the treatment of cancer.

29. A pharmaceutical composition of claim 18 for use in the treatment of cancer, wherein the compound or pharmaceutical composition is administered in combination with one or more additional anticancer agents selected from:
1) other forms of cancer immunotherapy and anti-cancer chemotherapeutic agents;
2) adenosine pathway modulators;
3) anti-PD-1 and PDL-1 antibodies; and
4) anti-CTLA4 antibodies.

30. A method of treating a proliferative disorder in a patient in need of such treatment, the method comprising administering a therapeutically effective amount of a pharmaceutical composition according to claim 18, to said patient.

31. A method of treating cancer in a patient in need of such treatment, the method comprising administering a therapeutically effective amount of a pharmaceutical composition according to claim 18, to said patient.

32. A method of treating a proliferative disorder in a patient in need of such treatment, the method comprising administering a therapeutically effective amount of a pharmaceutical composition according to claim 18, to said patient, in combination with one or more additional anticancer agents.

32. A method according to claim 32, wherein the one or more additional anticancer agents is selected from:
1) other forms of cancer immunotherapy and anti-cancer chemotherapeutic agents;
2) adenosine pathway modulators;
3) anti-PD-1 and PDL-1 antibodies; and
4) anti-CTLA4 antibodies.
